(19) United States
(12) Patent Application Publication
(10) Pub. No.: US 2019/0229276 A1
(43) Pub. Date: Jul. 25, 2019

(54) CONDENSED CYCLIC COMPOUND, COMPOSITION INCLUDING THE CONDENSED CYCLIC COMPOUND, AND ORGANIC LIGHT-EMITTING DEVICE INCLUDING CONDENSED CYCLIC COMPOUND

(71) Applicants: Samsung Electronics Co., Ltd., Suwon-si (KR); Samsung SDI Co., Ltd., Yongin-si (KR)

(72) Inventors: Eunsuk KWON, Suwon-si (KR); Yongsik JUNG, Yongin-si (KR); Sangmo KIM, Hwaseong-si (KR); Joonghyuk KIM, Seoul (KR); Youngmok SON, Hwaseong-si (KR); Jhunmo SON, Yongin-si (KR); Soonok JEON, Suwon-si (KR); Yeonsook CHUNG, Seoul (KR)

(21) Appl. No.: 16/146,213

(22) Filed: Sep. 28, 2018

(30) Foreign Application Priority Data
Jan. 23, 2018 (KR) 10-2018-0008411

(51) Int. Cl. H01L 51/00; C07D 491/048; C09K 11/06

(52) U.S. Cl. CPC H01L 51/0071; C07D 491/048; H01L 51/5016; C09K 11/06; H01L 51/0074; H01L 51/0072

(57) ABSTRACT

A condensed cyclic compound represented by Formula 1:

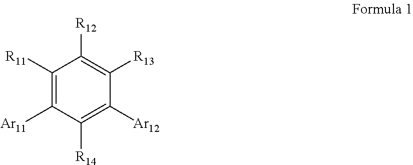

wherein in Formula 1, Ar₁₁, Ar₁₂, R₁₁, R₁₂, R₁₃, and R₁₄ are the same as described in the specification.

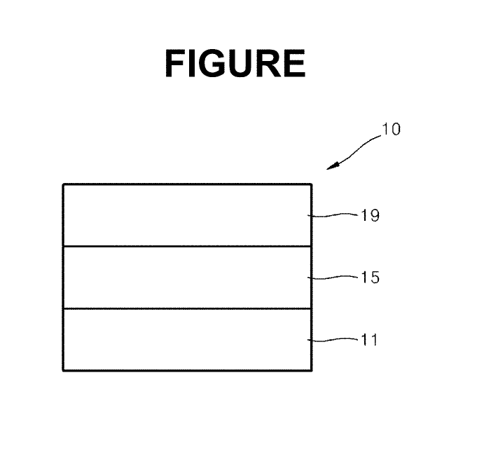

### CROSS-REFERENCE TO RELATED APPLICATION

This application claims priority to Korean Patent Application No. 10-2018-0008411, filed on Jan. 23, 2018, in the Korean Intellectual Property Office, and all the benefits accruing therefrom under 35 U.S.C. § 119, the content of which is incorporated herein in its entirety by reference.

### BACKGROUND

### 1. Field

The present disclosure relates to a condensed cyclic compound, a composition including the condensed cyclic compound, and an organic light-emitting device including the condensed cyclic compound.

### 2. Description of the Related Art

Organic light-emitting devices (OLEDs) are self-emission devices which have wide viewing angles, high contrast ratios, short response times, and excellent brightness, driving voltage, and response speed characteristics, and which produce full-color images.

Organic light-emitting devices include an anode, a cathode, and an organic layer located between the anode and the cathode and including an emission layer. A hole transport region may be located between the anode and the emission layer, and an electron transport region may be located between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. These excitons transit from an excited state to a ground state to thereby generate light.

Various types of organic light emitting devices are known. However, there still remains a need in OLEDs having low driving voltage, high efficiency, high brightness, and long lifespan.

### SUMMARY

Provided are a condensed cyclic compound, a composition including the condensed cyclic compound, and an organic light-emitting device including the condensed cyclic compound.

Additional aspects will be set forth in part in the description which follows and, in part, will be apparent from the description, or may be learned by practice of the presented embodiments.

According to an aspect of an embodiment, a condensed cyclic compound may be represented by Formula 1:

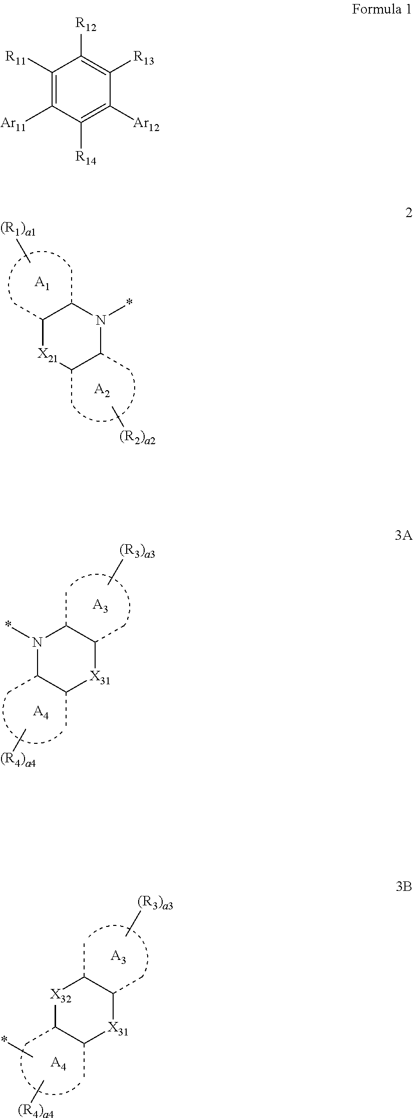

wherein in Formulae 1, 2, 3A, and 3B,

Ar₁₁ may be a group represented by Formula 2,

Ar₁₂ may be a group represented by Formula 3A or 3B,

A₁ may be a dibenzofuran group, a dibenzothiophene group, a carbazole group, a fluorene group, a dibenzosilole group, an azadibenzofuran group, an azadibenzothiophene group, an azacarbazole group, an azafluorene group, or an azadibenzosilole group,

A₂ to A₄ may each independently be a C₅-C₆₀ carbocyclic group or a C₁-C₆₀ heterocyclic group,

X₂₁ may be selected from a single bond, O, S, N(R₂₁), C(R₂₁)(R₂₂), and Si(R₂₁)(R₂₂),

X₃₁ may be selected from a single bond, O, S, N(R₃₁), C(R₃₁)(R₃₂), and Si(R₃₁)(R₃₂), and X₃₂ may be selected from a single bond, O, S, N(R₃₃), C(R₃₃)(R₃₄), and Si(R₃₃)(R₃₄), provided that at least one selected from X₃₁ and X₃₂ in Formula 3B is not a single bond,

R₁₁ to R₁₃ may each independently be selected from a phenyl group, a biphenyl group, a terphenyl group, and a tetraphenyl group, each unsubstituted or substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₁-C₆₀ alkoxy group, a phenyl group, a biphenyl group, a terphenyl group, and a tetraphenyl group, wherein at least one selected from R₁₁ to R₁₃ may independently be selected from a cyano group-containing phenyl group, a cyano group-containing biphenyl group, a cyano group-containing terphenyl group, and a cyano group-containing tetraphenyl group,

R₁ to R₄, R₁₄, R₂₁, R₂₂, and R₃₁ to R₃₄ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇),

a1 to a4 may each independently be an integer from 0 to 10,

* indicates a binding site to an adjacent atom, and

at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), and —B(Q₁₆)(Q₁₇);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), and —B(Q₂₆)(Q₂₇); and

—Si(Q₃₁)(Q₃₂)(Q₃₃), —N(Q₃₄)(Q₃₅), and —B(Q₃₆)(Q₃₇),

wherein Q₁ to Q₁₀, Q₁₁ to Q₁₇, Q₂₁ to Q₂₇, and Q₃₁ to Q₃₇ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₁₀ heteroaryl group, a substituted or unsubstituted monovalent non-aromatic condensed polycyclic group, and a substituted or unsubstituted monovalent non-aromatic condensed heteropolycyclic group.

According to an aspect of another embodiment, a composition may include a first compound and a second compound, the first compound may be the condensed cyclic compound, and the second compound may include at least one selected from a carbazole group, a dibenzofuran group, a dibenzothiophene group, an indenocarbazole group, an indolocarbazole group, a benzofurocarbazole group, a benzothienocarbazole group, an acridine group, a dihydroacridine group, and a triindolobenzene group and may not include an electron withdrawing group,

wherein the electron withdrawing group may be selected from

—F, —CFH₂, —CF₂H, —CF₃, —CN, and —NO₂;

a C₁-C₆₀ alkyl group substituted with at least one selected from —F, —CFH₂, —CF₂H, —CF₃, —CN, and —NO₂;

a C₁-C₆₀ heteroaryl group and a monovalent non-aromatic condensed polycyclic heterocyclic group, each including *═N—*′ as a ring-forming moiety; and

a C₁-C₆₀ heteroaryl group and a monovalent non-aromatic condensed polycyclic heterocyclic group, each including *═N—*′ as a ring-forming moiety and each substituted with at least one selected from deuterium, —F, —CFH₂, —CF₂H, —CF₃, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₁₀ heteroaryl group, a substituted or unsubstituted monovalent non-aromatic condensed polycyclic group, and a substituted or unsubstituted monovalent non-aromatic condensed heteropolycyclic group.

According to an aspect of another embodiment, an organic light-emitting device may include:

a first electrode;

a second electrode; and

an organic layer disposed between the first electrode and the second electrode,

wherein the organic layer may include an emission layer; and

wherein the organic layer may include at least one condensed cyclic compound represented by Formula 1 or the composition including the at least one condensed cyclic compound.

### BRIEF DESCRIPTION OF THE DRAWINGS

These and/or other aspects will become apparent and more readily appreciated from the following description of the embodiments, taken in conjunction with FIGURE which is a schematic cross-sectional view of an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

In an embodiment, a condensed cyclic compound is provided. The condensed cyclic compound may be represented by Formula 1:

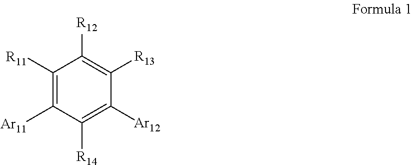

wherein, in Formula 1, Ar₁₁ may be a group represented by Formula 2, and Ar₁₂ may be a group represented by Formula 3A or 3B:

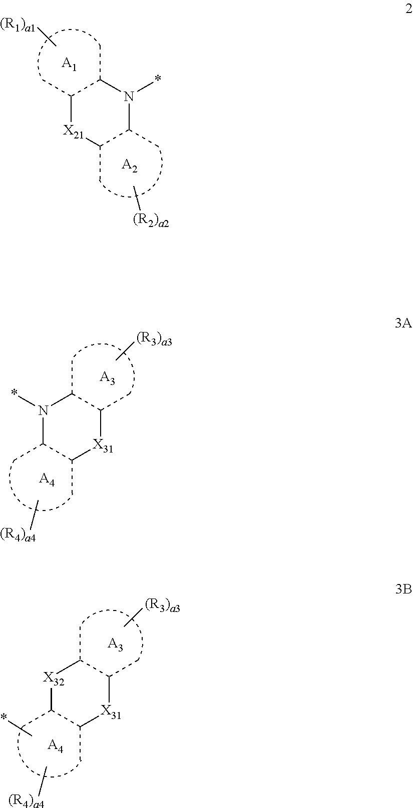

In Formula 2, A₁ may be a dibenzofuran group, a dibenzothiophene group, a carbazole group, a fluorene group, a dibenzosilole group, an azadibenzofuran group, an azadibenzothiophene group, an azacarbazole group, an azafluorene group, or an azadibenzosilole group, wherein A₂ to A₄ in Formulae 3A and 3B may each independently be a C₆-C₆₀ carbocyclic group or a C₁-C₆₀ heterocyclic group.

In Formula 2, X₂₁ may be selected from a single bond, O, S, N(R₂₁), C(R₂₁)(R₂₂), and Si(R₂₁)(R₂₂) and in Formulae 3A and 3B, X₃₁ may be selected from a single bond, O, S, N(R₃₁), C(R₃₁)(R₃₂), and Si(R₃₁)(R₃₂), and X₃₂ may be selected from a single bond, O, S, N(R₃₃), C(R₃₃)(R₃₄), and Si(R₃₃)(R₃₄). R₂₁, R₂₂, and R₃₁ to R₃₄ may respectively be understood by referring to the descriptions for those provided herein.

In Formula 3B, at least one selected from X₃₁ and X₃₂ may not be a single bond.

In some embodiments, in Formula 2, A₁ may be a dibenzofuran group, a dibenzothiophene group, a carbazole group, a fluorene group, or a dibenzosilole group, and A₂ to A₄ may each independently be a benzene group, a naphthalene group, a dibenzofuran group, a dibenzothiophene group, a carbazole group, a fluorene group, or a dibenzosilole group.

In an embodiment, in Formula 1, Ar₁₁ may be selected from groups represented by Formulae 2-1 to 2-6:

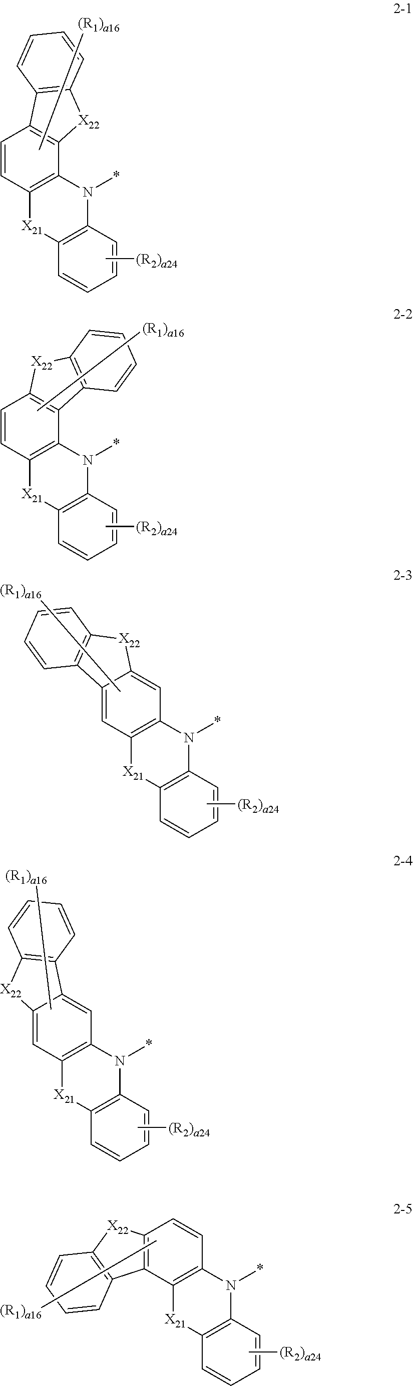

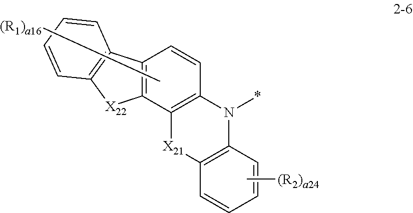

wherein, in Formulae 2-1 to 2-6,

X₂₂ may be selected from O, S, N(R₂₃), C(R₂₃)(R₂₄), and Si(R₂₃)(R₂₄),

X₂₁, R₁, and R₂ may respectively be understood by referring to the descriptions for those provided herein,

R₂₃ and R₂₄ may be understood by referring to the descriptions for R₂₁ provided herein,

a16 may be an integer from 0 to 6,

a24 may be an integer from 0 to 4, and

* indicates a binding site to an adjacent atom.

In some embodiments, in Formulae 2-1 to 2-6, X₂₁ may be a single bond, and X₂ may be O or S, but embodiments are not limited thereto.

In one or more embodiments, in Formula 1, Ar₁₂ may be selected from groups represented by Formulae 3A-1 to 3A-7 and 3B-1 to 3B-7:

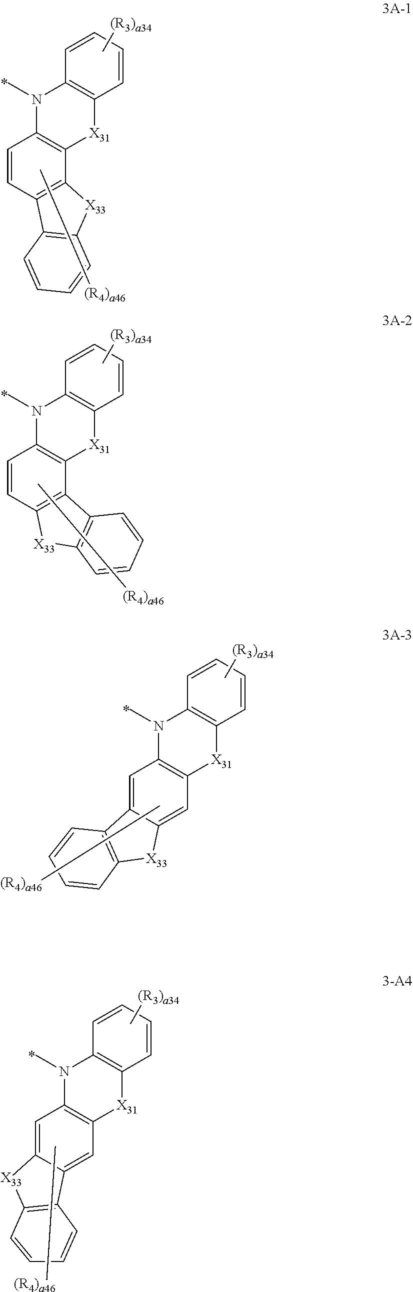

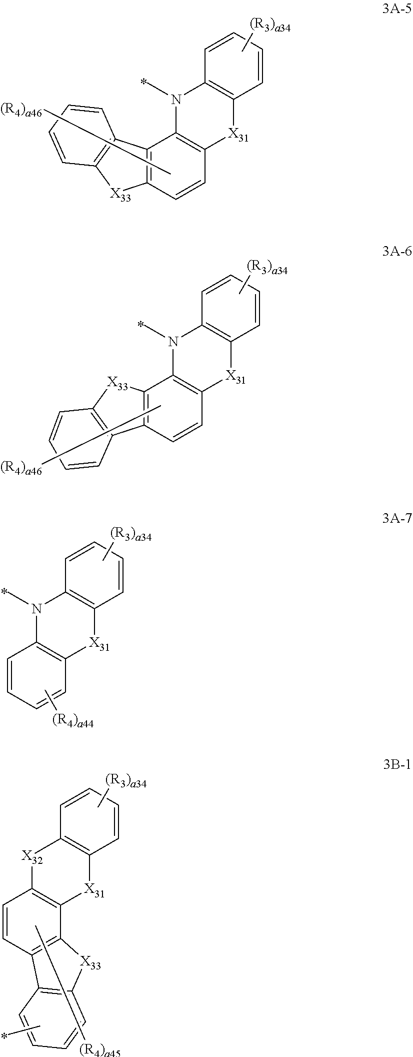

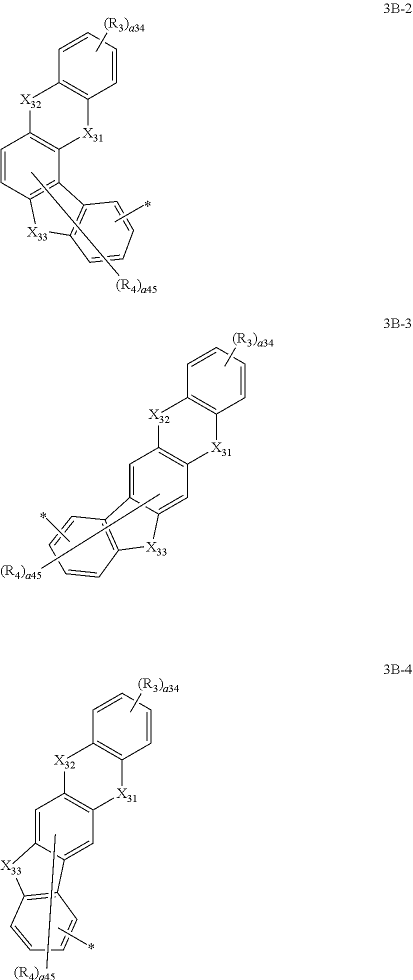

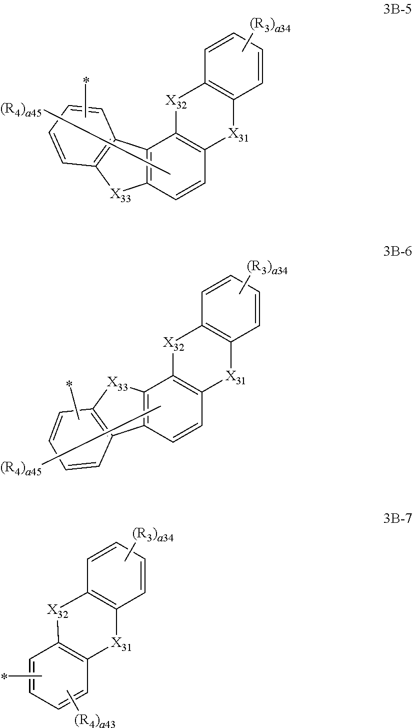

wherein Formulae 3A-1 to 3A-7 and 3B-1 to 3B-7,

X₃₃ may be selected from O, S, N(R₃₅), C(R₃₅)(R₃₆), and Si(R₃₅)(R₃₆),

X₃₁, X₃₂, R₃, and R₄ may respectively be understood by referring to the descriptions for those provided herein,

R₃₅ and R₃₆ may be understood by referring to the descriptions for R₃₁ provided herein,

a34 may be an integer from 0 to 4,

a45 may be an integer from 0 to 5,

a43 may be an integer from 0 to 3, and

* indicates a binding site to an adjacent atom.

In some embodiments, in Formulae 3A-1 to 3A-7, X₃₁ may be a single bond, but embodiments are not limited thereto.

In one or more embodiments, in Formula 1, Ar₁₂ may be selected from groups represented by Formulae 3A-1(1) to 3A-6(1), 3A-7(1) to 3A-7(3), and 3B-7(1) to 3B-7(14):

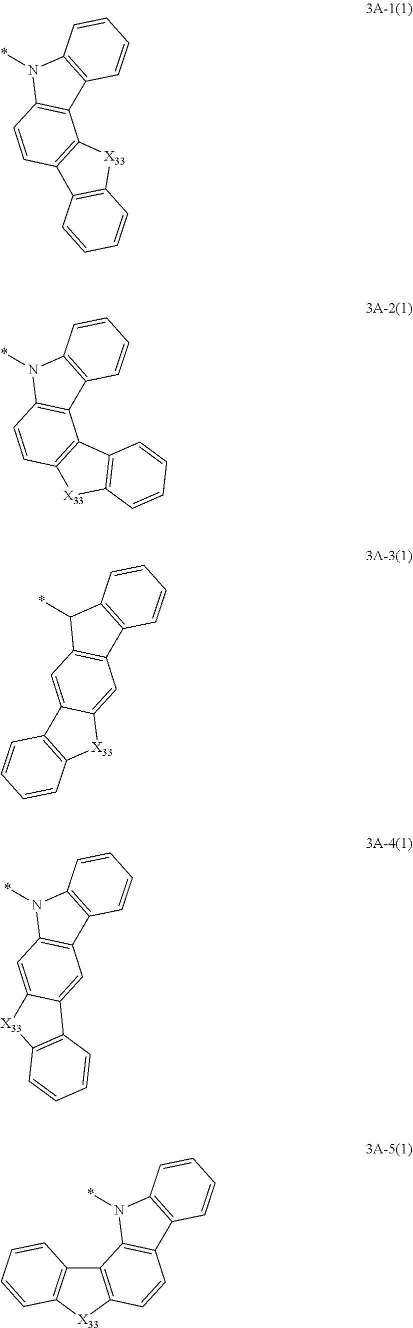

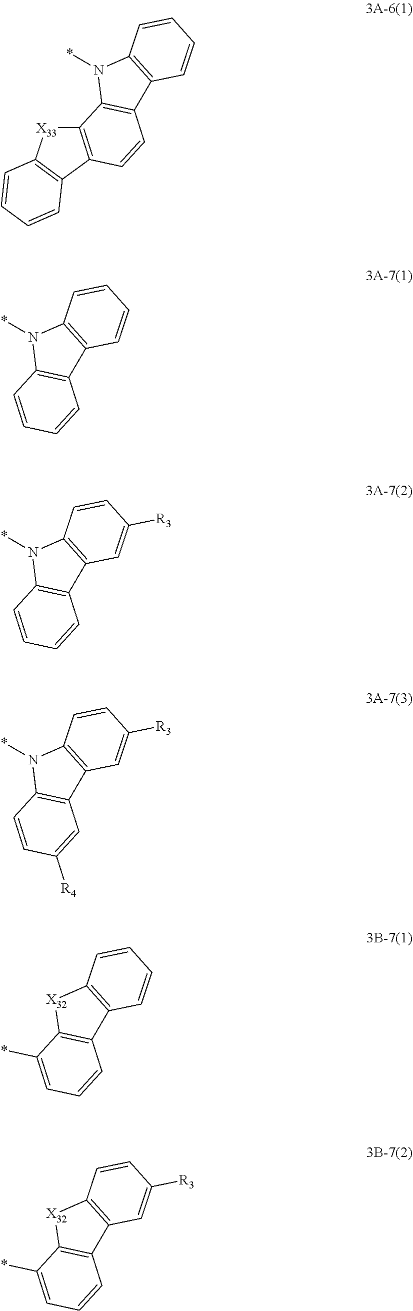

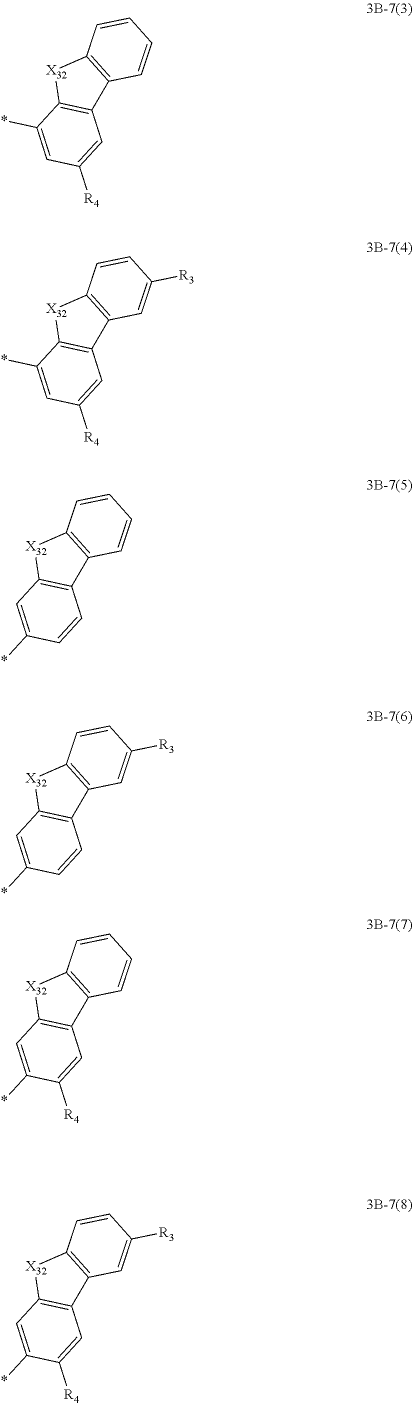

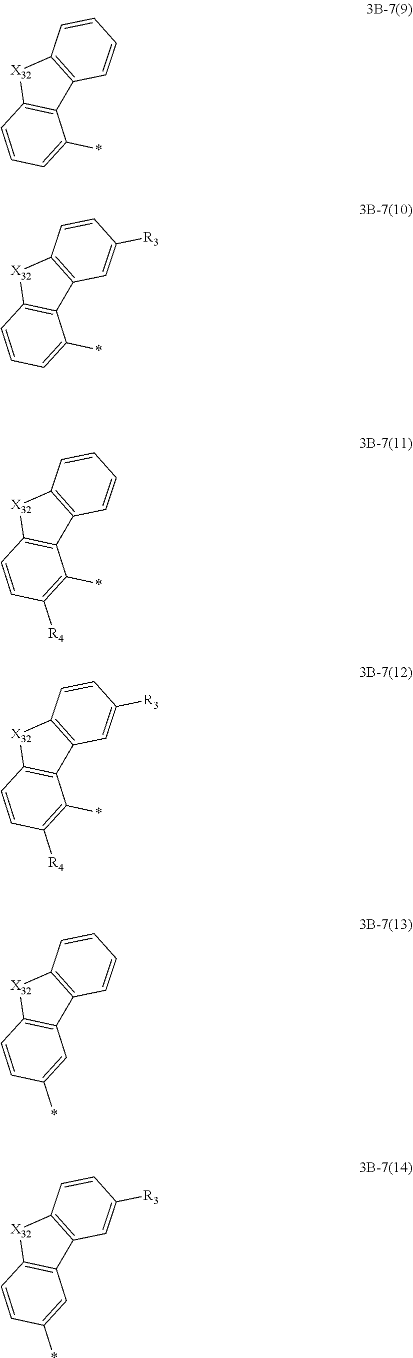

wherein, in Formulae 3A-1(1) to 3A-6(1), 3A-7(1) to 3A-7(3), and 3B-7(1) to 3B-7(14),

X₃₂ may be selected from O, S, N(R₃₃), C(R₃₃)(R₃₄), and Si(R₃₃)(R₃₄),

X₃₃ may be selected from O, S, N(R₃₅), C(R₃₅)(R₃₆), and Si(R₃₅)(R₃₆),

R₃ and R₄ may respectively be understood by referring to the descriptions for those provided herein, provided that each of R₃ and R₄ is not hydrogen,

R₃₅ and R₃₄ may respectively be understood by referring to the descriptions for those provided herein.

R₃₅ and R₃₆ may be understood by referring to the descriptions for R₃₁ provided herein, and

* indicates a binding site to an adjacent atom.

In some embodiments, at least one selected from R₃ and R₄ in Formulae 3A-7(2), 3A-7(3) and 3B-7(2) to 3B-7(4), 3B-7(6) to 3B-7(8), 3B-7(10) to 3B-7(12), and 3B-7(14) may be a cyano group, but embodiments are not limited thereto.

In Formula 1, R₁₁ to R₁₃ may each independently be selected from a phenyl group, a biphenyl group, a terphenyl group, and a tetraphenyl group, each unsubstituted or substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₁-C₆₀ alkoxy group, a phenyl group, a biphenyl group, a terphenyl group, and a tetraphenyl group, wherein at least one selected from R₁₁ to R₁₃ may independently be selected from a cyano group-containing phenyl group, a cyano group-containing biphenyl group, a cyano group-containing terphenyl group, and a cyano group-containing tetraphenyl group.

The term “biphenyl group” as used herein refers to a monovalent group having a backbone in which two phenyl groups are bound via a single bond. The term “terphenyl group” as used herein refers to a monovalent group having a backbone in which three phenyl groups are bound via a single bond. The term “tetraphenyl group” as used herein refers to a monovalent group having a backbone in which four phenyl groups are bound via a single bond.

In an embodiment, in Formula 1, R₁₁ to R₁₃ may each independently be selected from a phenyl group, a biphenyl group, a terphenyl group, and a tetraphenyl group, each unsubstituted or substituted with at least one selected from deuterium, a cyano group, a C₁-C₂₀ alkyl group, a phenyl group, a biphenyl group, a terphenyl group, and a tetraphenyl group, wherein at least one selected from R₁₁ to R₁₃ may independently be selected from groups represented by Formulae 4-1 to 4-4:

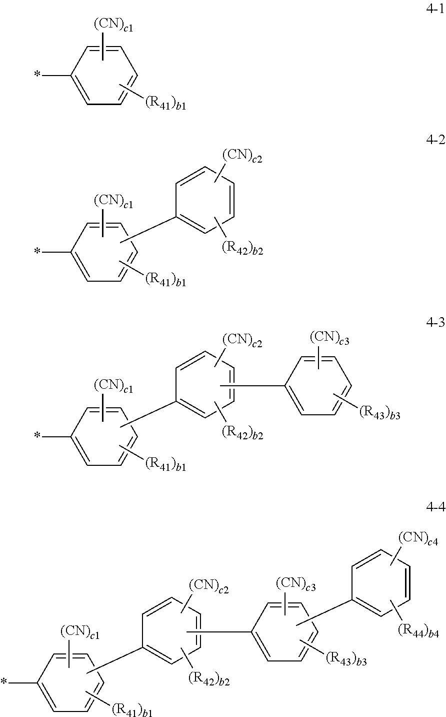

wherein, in Formulae 4-1 to 4-4,

R₄₁ to R₄₄ may each independently be selected from hydrogen, deuterium, a C₁-C₂₀ alkyl group, a phenyl group, a biphenyl group, and a terphenyl group,

b1 to b4 and c1 to c4 may each independently be an integer from 0 to 4,

* indicates a binding site to an adjacent atom, and

c1 in Formula 4-1, c1+c2 in Formula 4-2, c1+c2+c3 in Formula 4-3, and c1+c2+c3+c4 in Formula 4 may each be 1 or greater (for example, 1, 2, or 3).

In one or more embodiments, in Formula 1, at least one selected from R₁₁ to R₁₃ may independently be selected from groups represented by Formulae 4-2A to 4-2C, 4-3A to 4-3I, and 4-4A to 4-4AA:

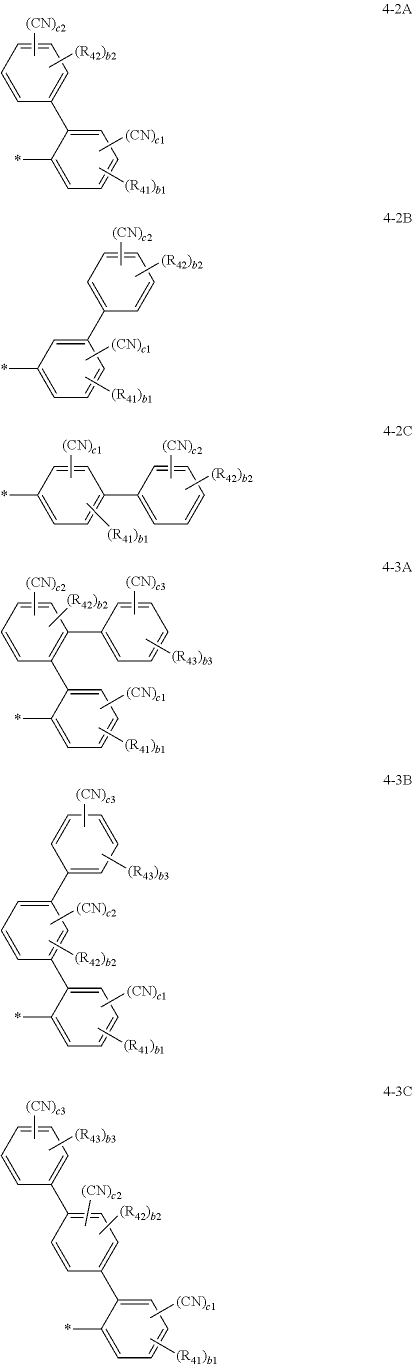

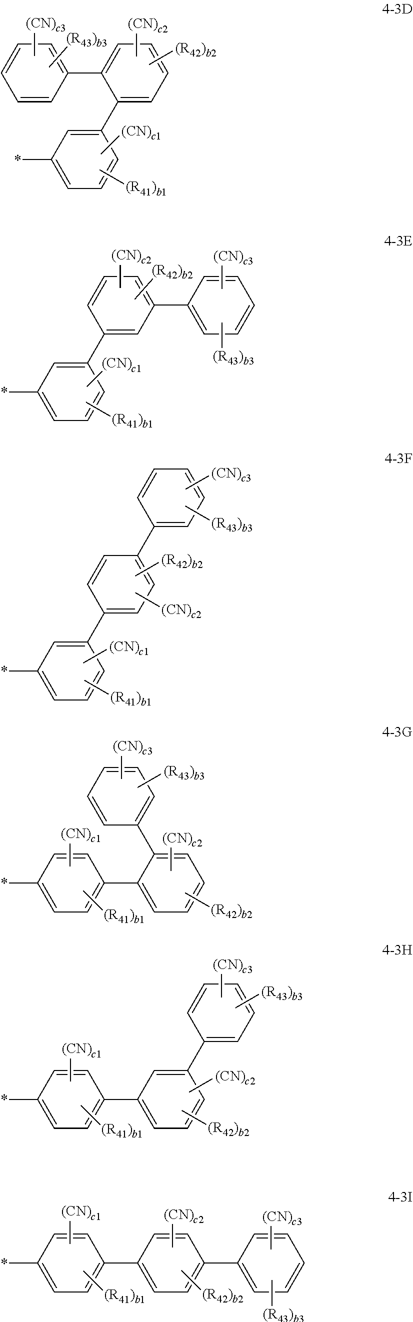

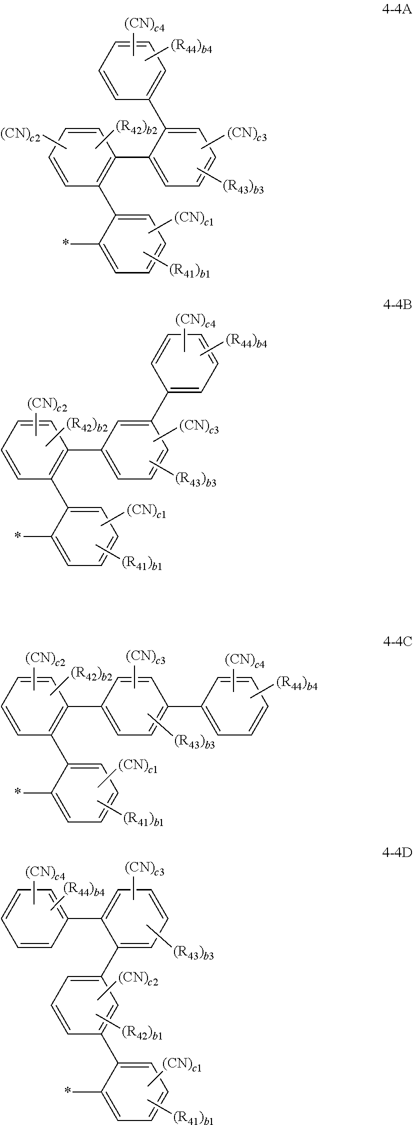

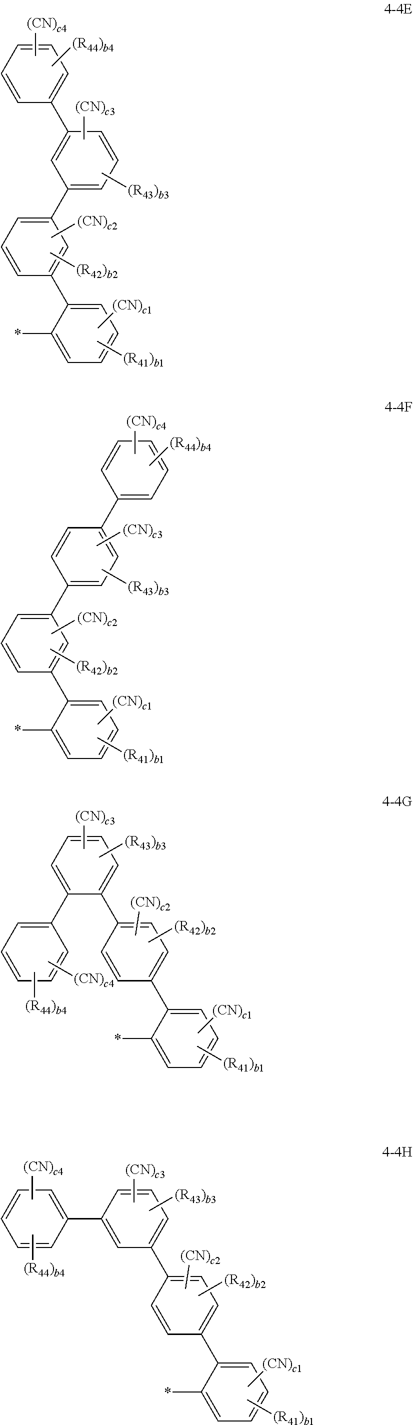

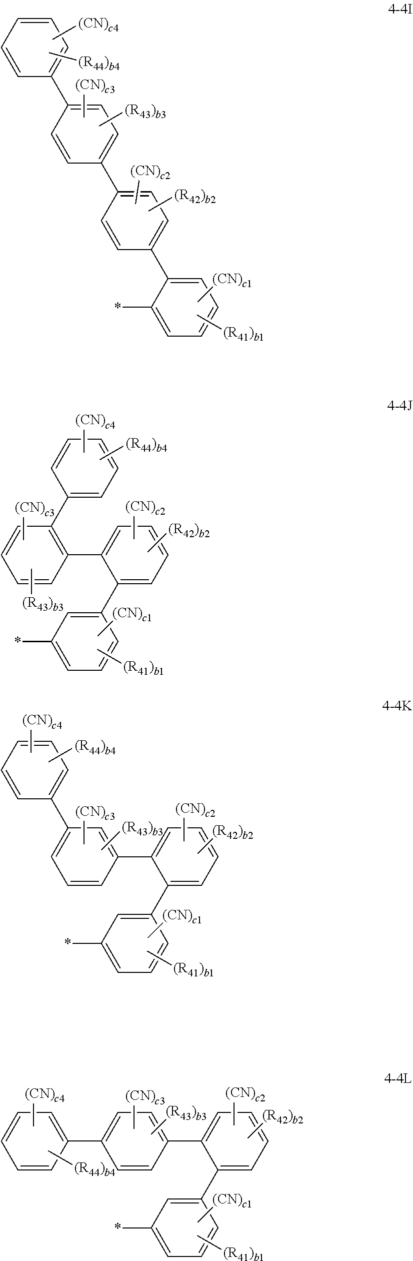

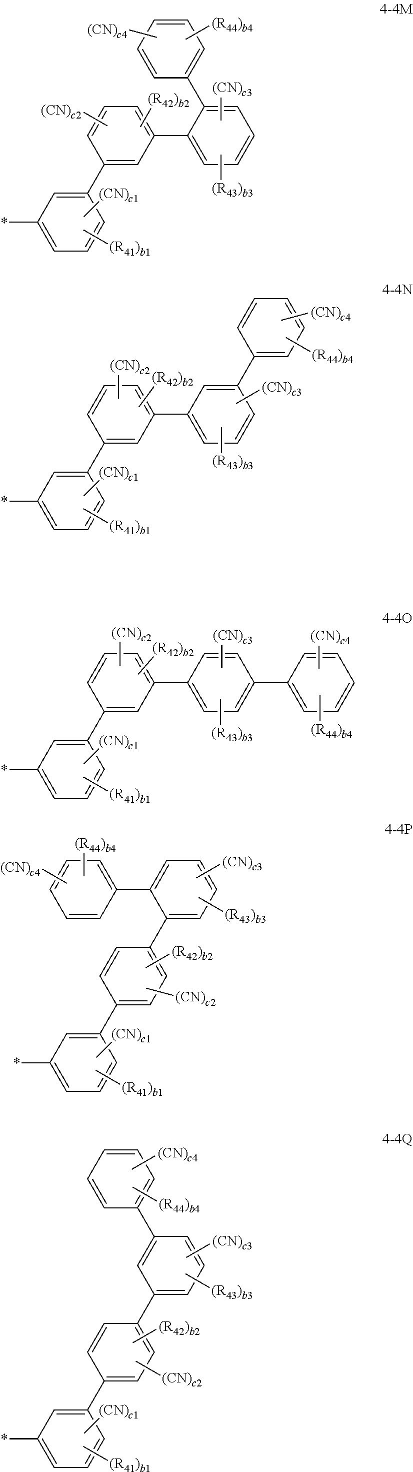

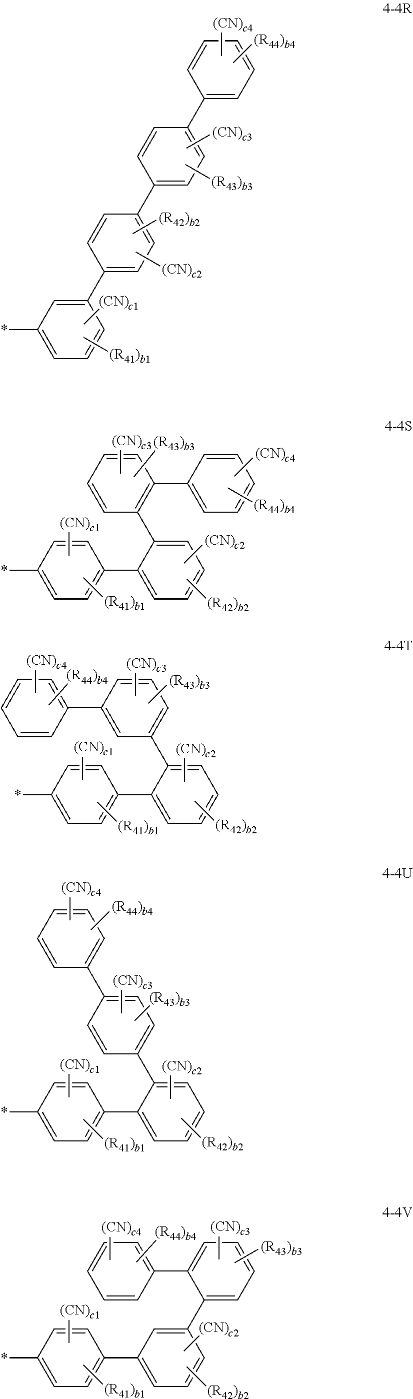

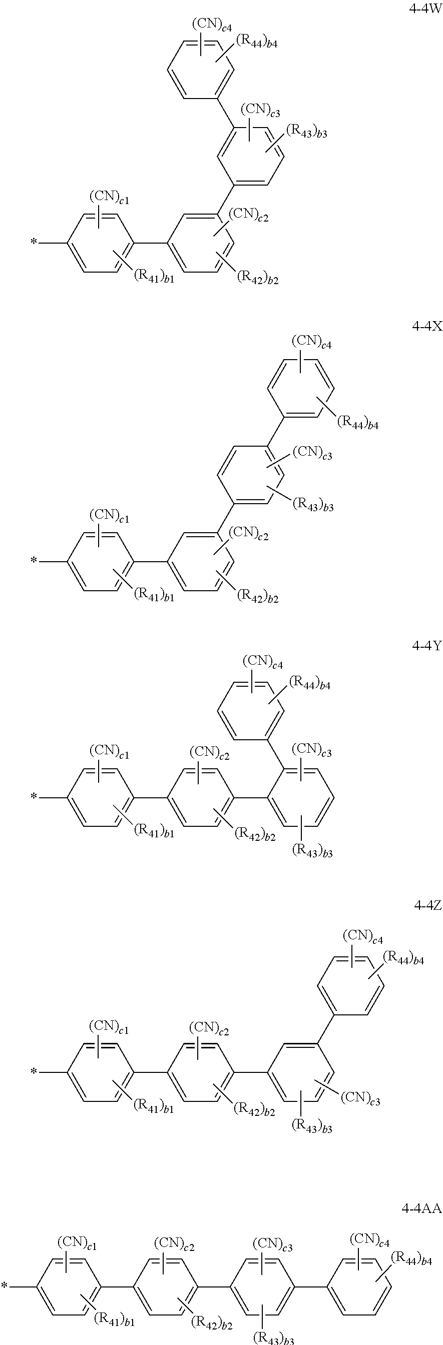

wherein, in Formulae 4-2A to 4-2C, 4-3A to 4-3I, and 4-4A to 4-4AA, R₄₁ to R₄₄, b1 to b4, and c1 to c4 may respectively be understood by referring to the descriptions for those provided herein,

* indicates a binding site to an adjacent atom, and

c1+c2 in Formulae 4-2A to 4-2C, c1+c2+c3 in Formulae 4-3A to 4-3I, and c1+c2+c3+c4 in Formulae 4-4A to 4-4AA may each be 1 or greater (for example, 1, 2, or 3).

In one or more embodiments, in Formula 1, at least one selected from R₁₁ to R₁₃ may independently be selected from groups represented by Formulae 4-1(1) to 4-1(8) and 4-2(1) to 4-2(78):

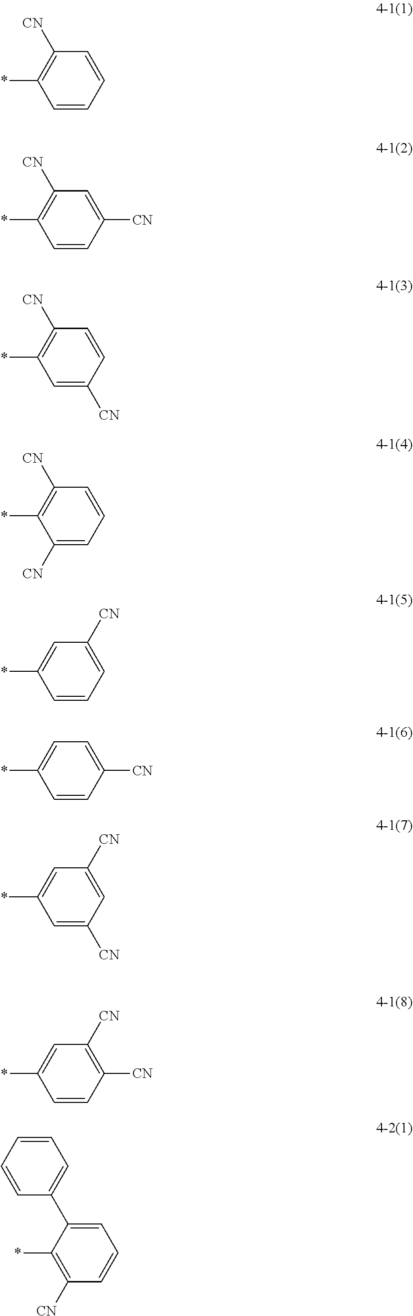

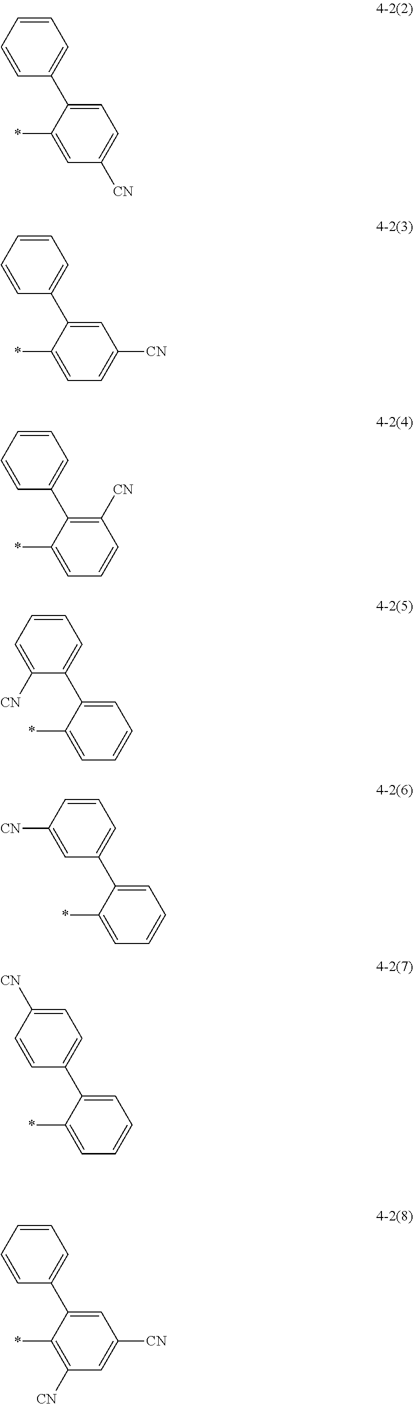

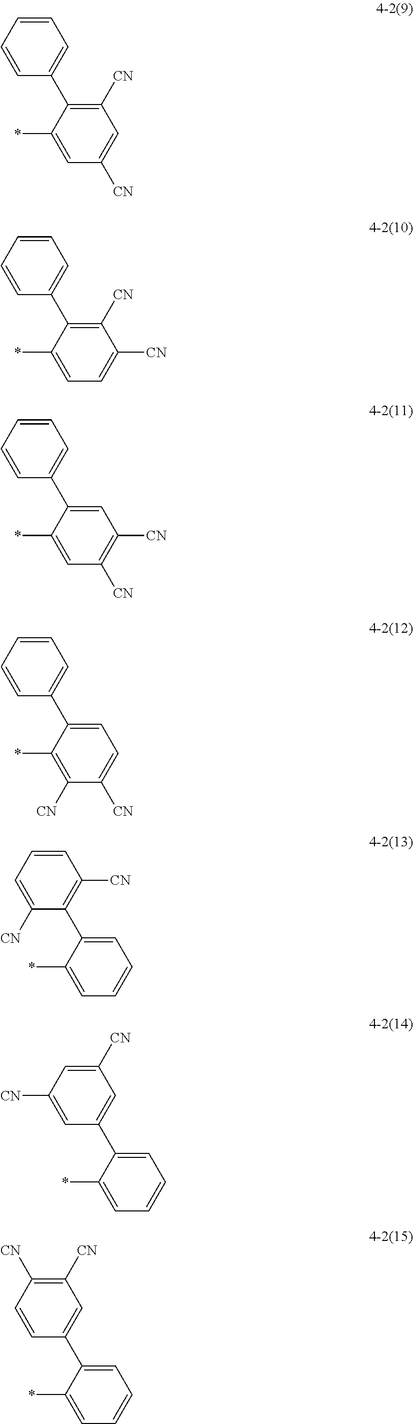

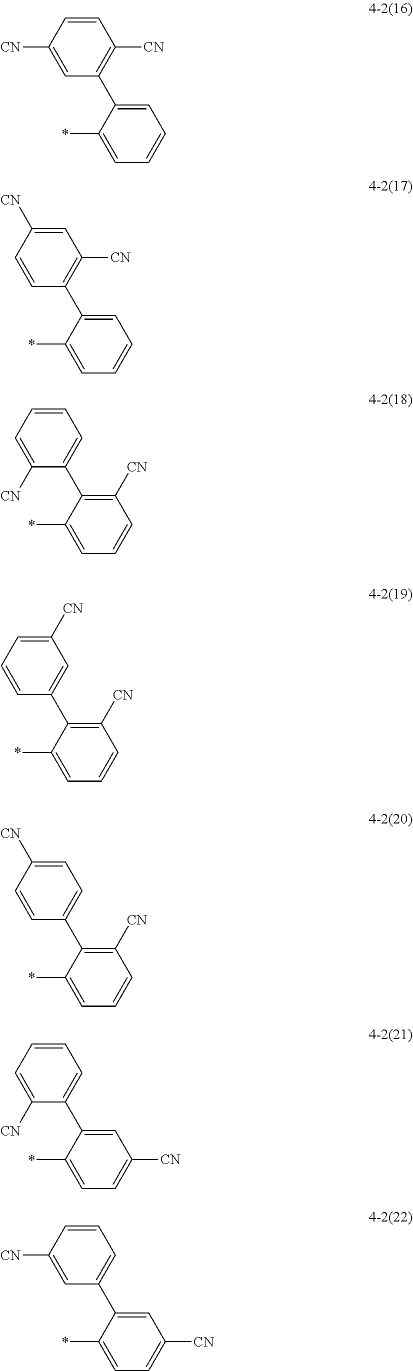

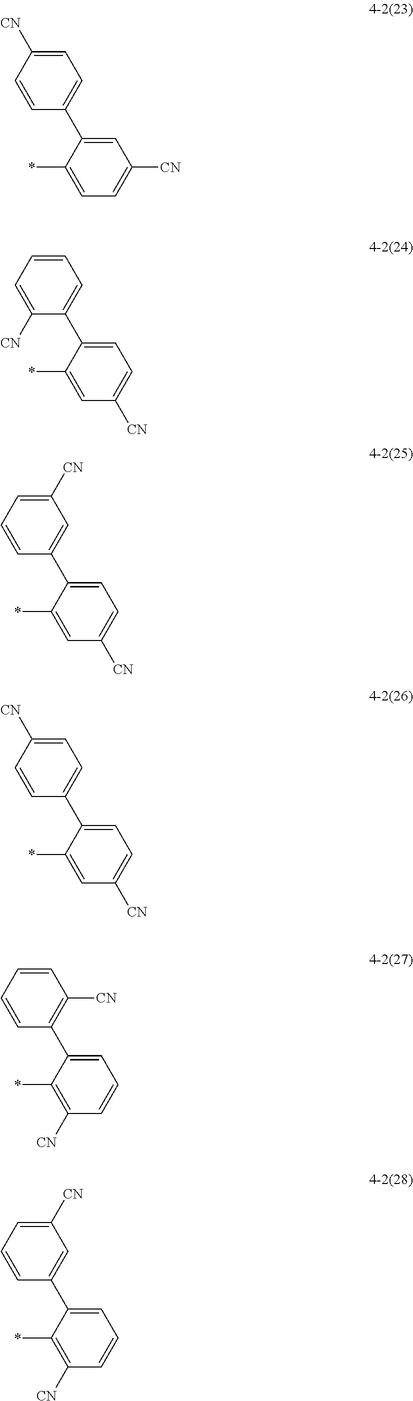

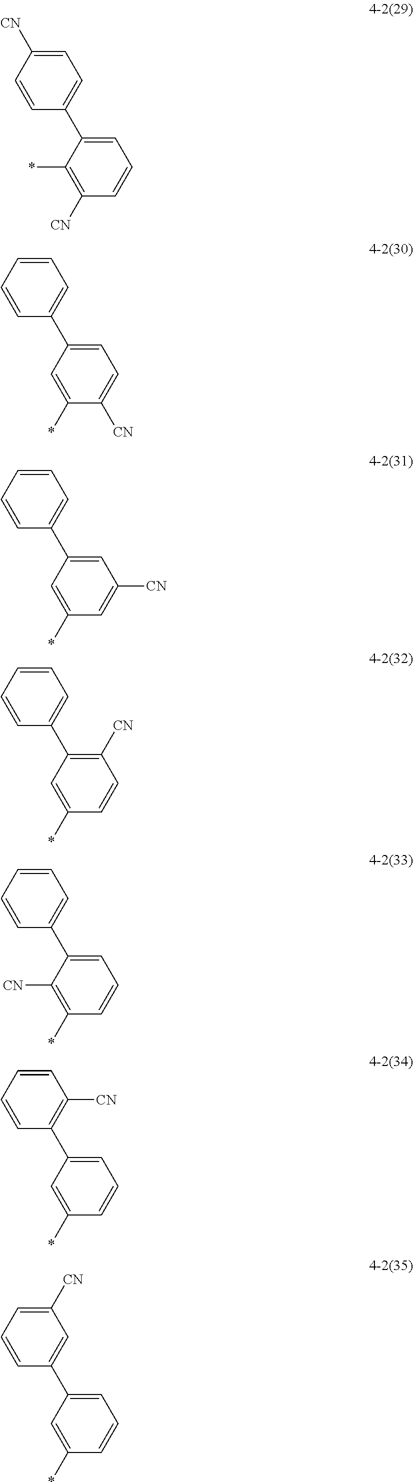

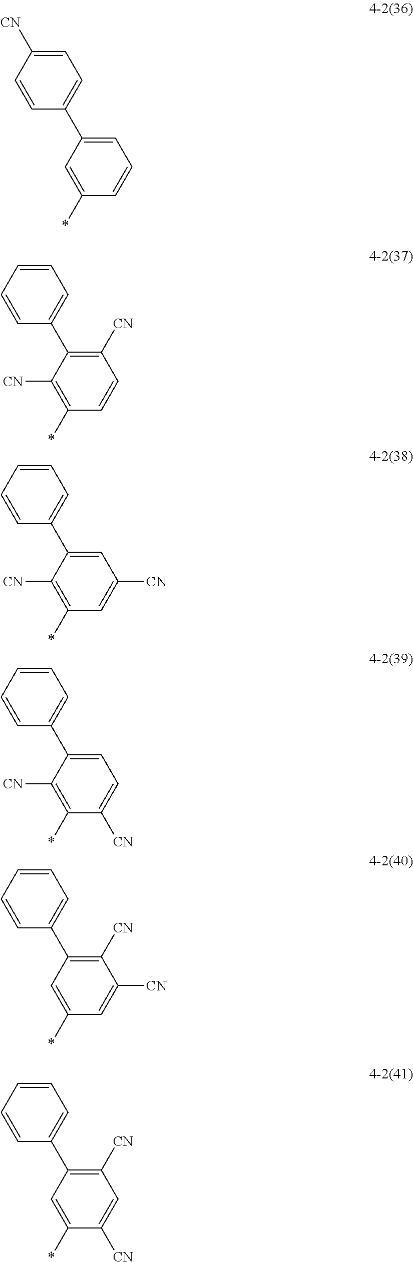

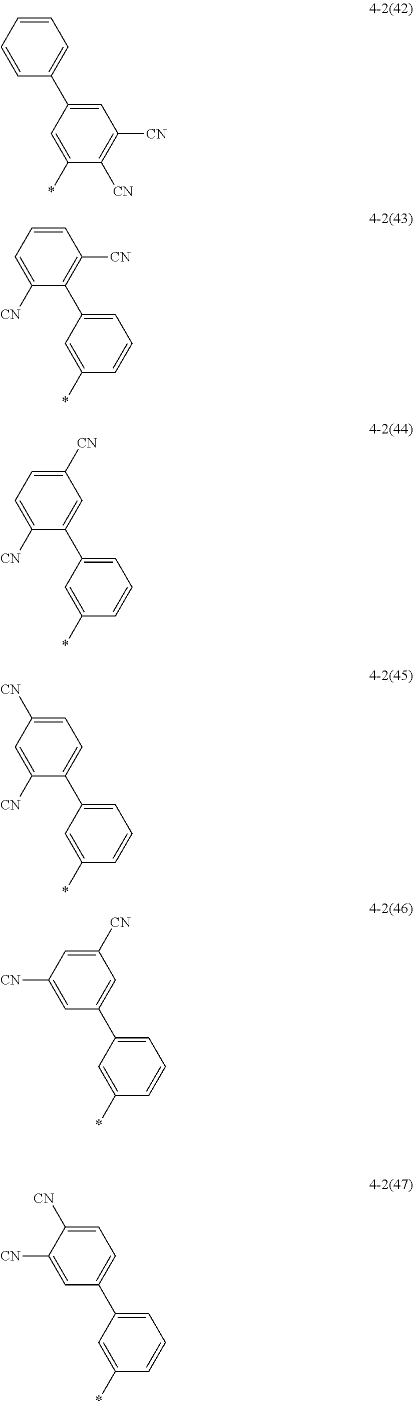

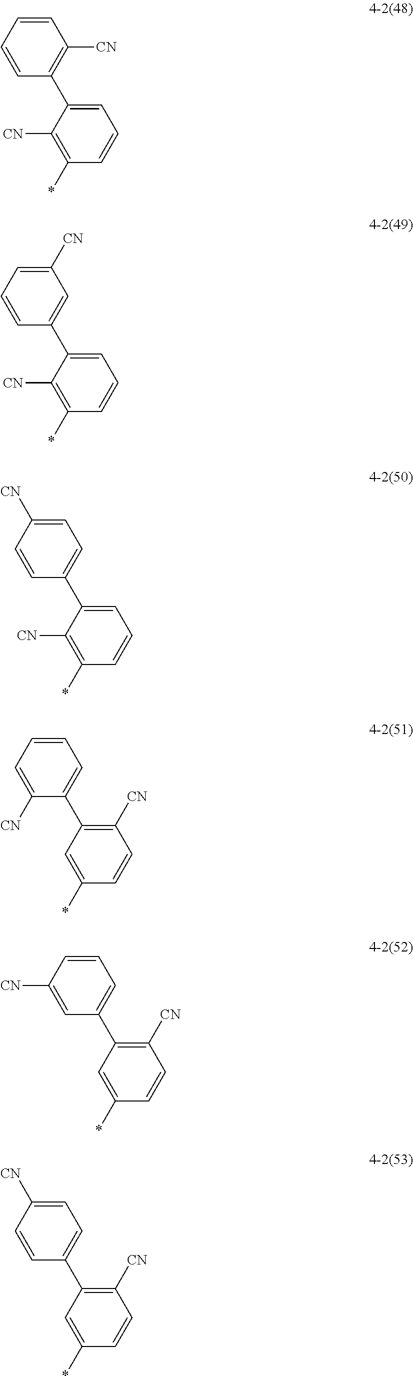

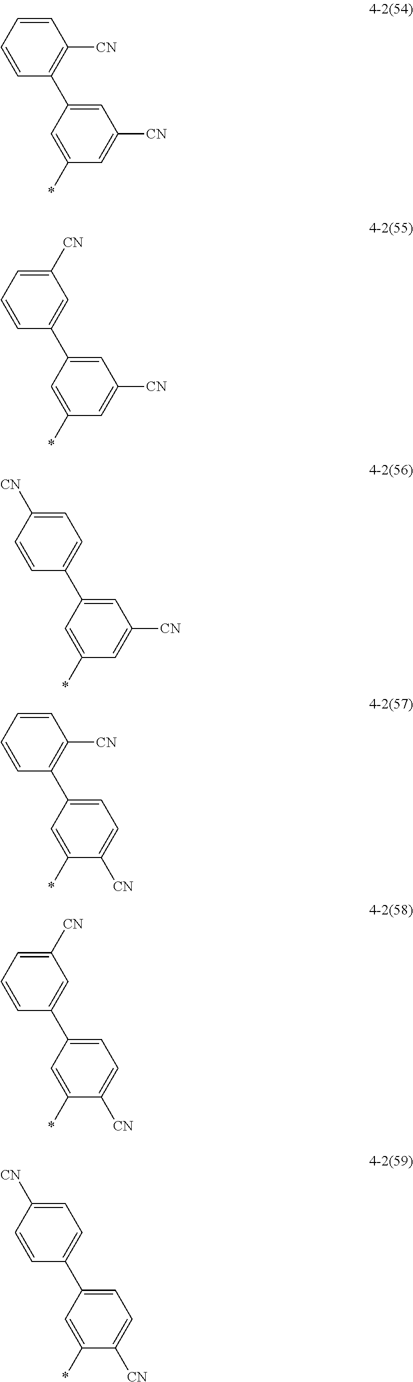

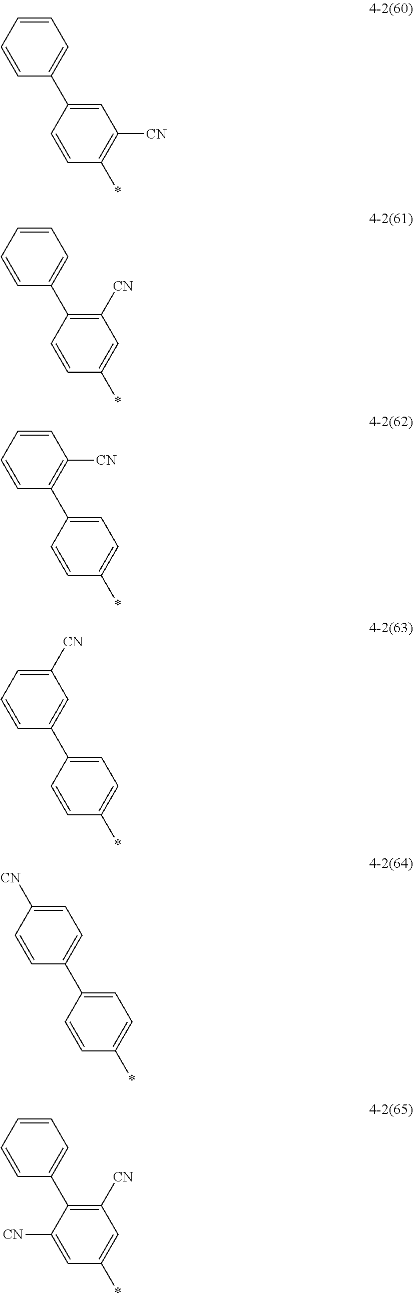

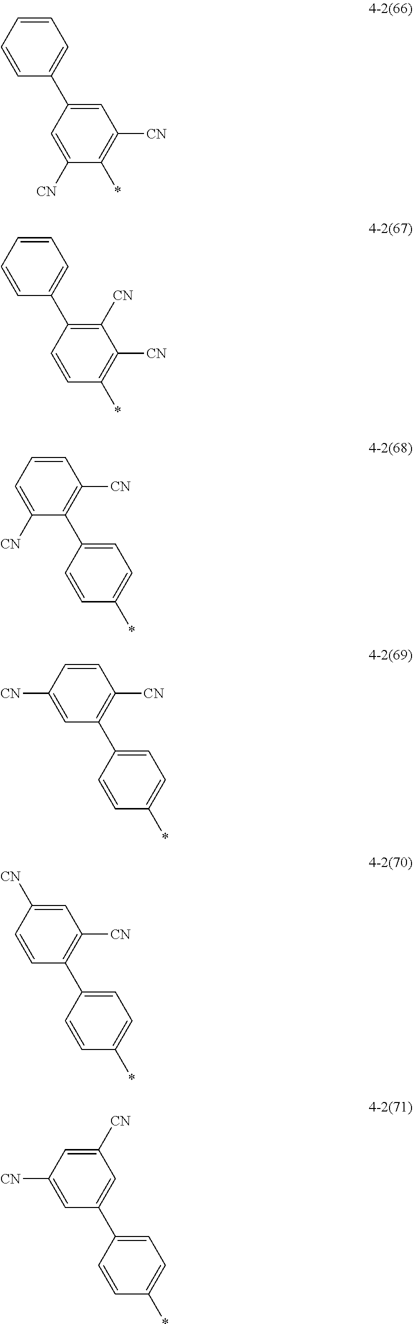

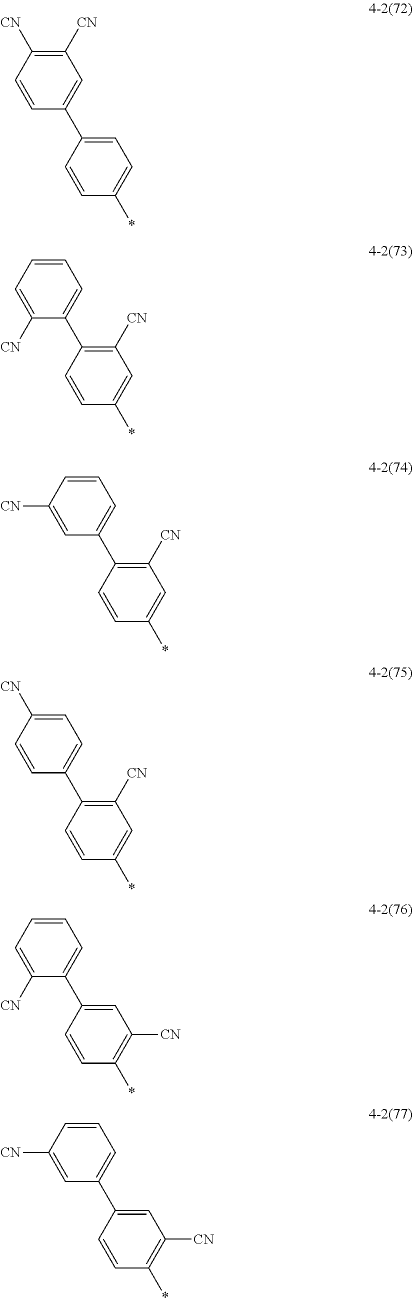

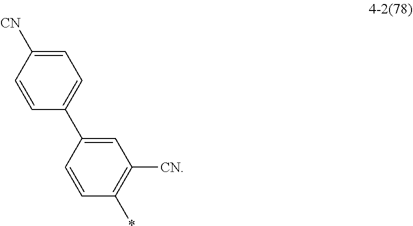

In Formulae 4-1(1) to 4-1(8) and 4-2(1) to 4-2(78), * indicates a binding site to an adjacent atom.

R₁ to R₄, R₁₄, R₂₁, R₂₂, and R₃₁ to R₃₄ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇).

In some embodiments, R₁ to R₄, R₁₄, R₂₁, R₂₂, and R₃₁ to R₃₄ may each independently be selected from

hydrogen, deuterium, a cyano group, a C₁-C₂₀ alkyl group, a phenyl group, a biphenyl group, a terphenyl group, a tetraphenyl group, a naphthyl group, a fluorenyl group, a carbazolyl group, a dibenzofuranyl group, a dibenzothiophenyl group, and a dibenzosilolyl group; and

a C₁-C₂₀ alkyl group, a phenyl group, a biphenyl group, a terphenyl group, a tetraphenyl group, a naphthyl group, a fluorenyl group, a carbazolyl group, a dibenzofuranyl group, a dibenzothiophenyl group, and a dibenzosilolyl group, each substituted with at least one selected from deuterium, a cyano group, a C₁-C₂₀ alkyl group, a phenyl group, a biphenyl group, a terphenyl group, a tetraphenyl group, a naphthyl group, a fluorenyl group, a carbazolyl group, a dibenzofuranyl group, a dibenzothiophenyl group, and a dibenzosilolyl group.

In Formula 1, a1 to a4 may each indicate the number of R₁ groups to R₄ groups, respectively. a1 to a4 may each independently be an integer from 0 to 10.

In an embodiment, in Formula 1,

R₁₁, R₁₃, and R₁₄ may each be hydrogen, and

R₁₂ may be a cyano group-containing phenyl group, a cyano group-containing biphenyl group, a cyano group-containing terphenyl group, or a cyano group-containing tetraphenyl group (for example, R₁₂ may be selected from groups represented by Formulae 4-1 to 4-4, groups represented by Formulae 4-2A to 4-2C, 4-3A to 4-3I, and 4-4A to 4-4AA, or groups represented by Formulae 4-1(1) to 4-1(8) and 4-2(1) to 4-2(9)), but embodiments are not limited thereto.

In an embodiment, in Formula 1, at least one selected from R₁ to R₄ may be (for example, at least one selected from R₃ and R₄) may be a cyano group.

In one or more embodiments, the number of cyano groups in Formula 1 may be 1, 2, 3, 4, or 5.

In one or more embodiments, the condensed cyclic compound may be selected from Compounds 1 to 428:

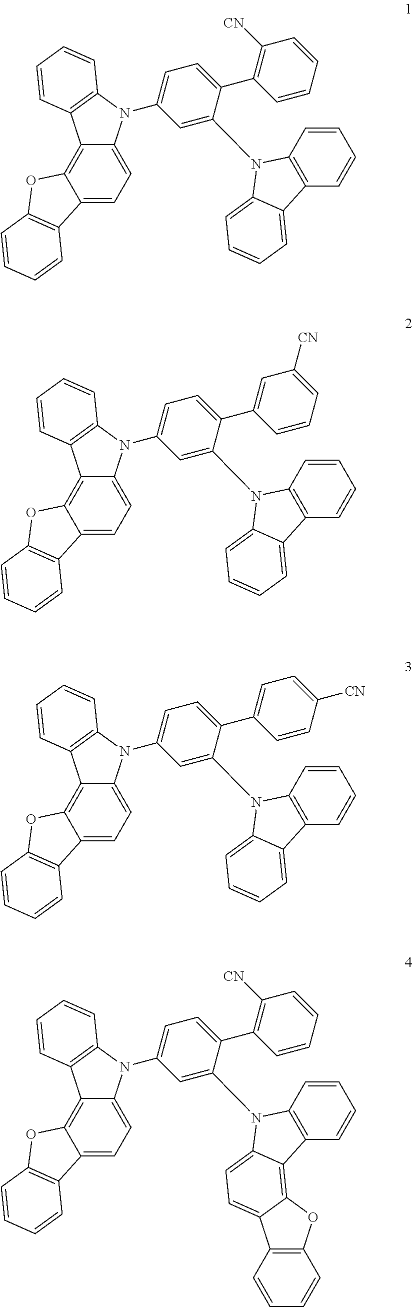

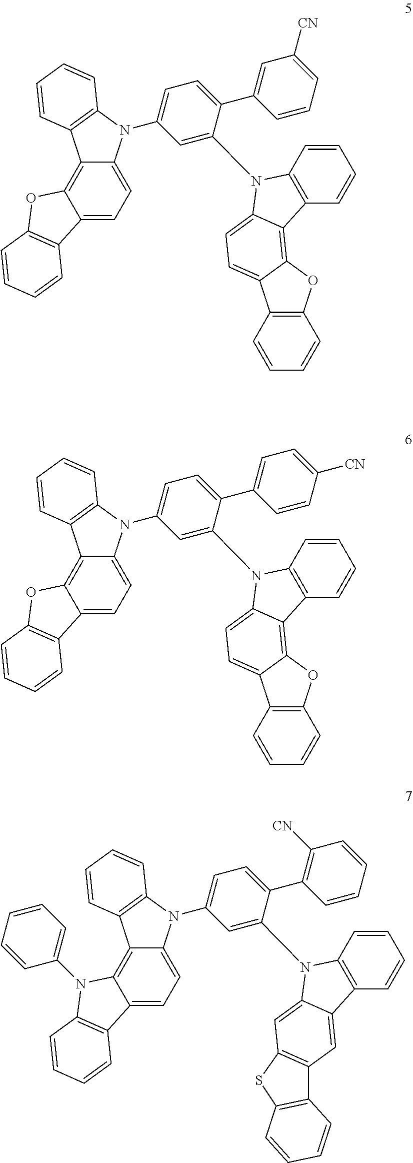

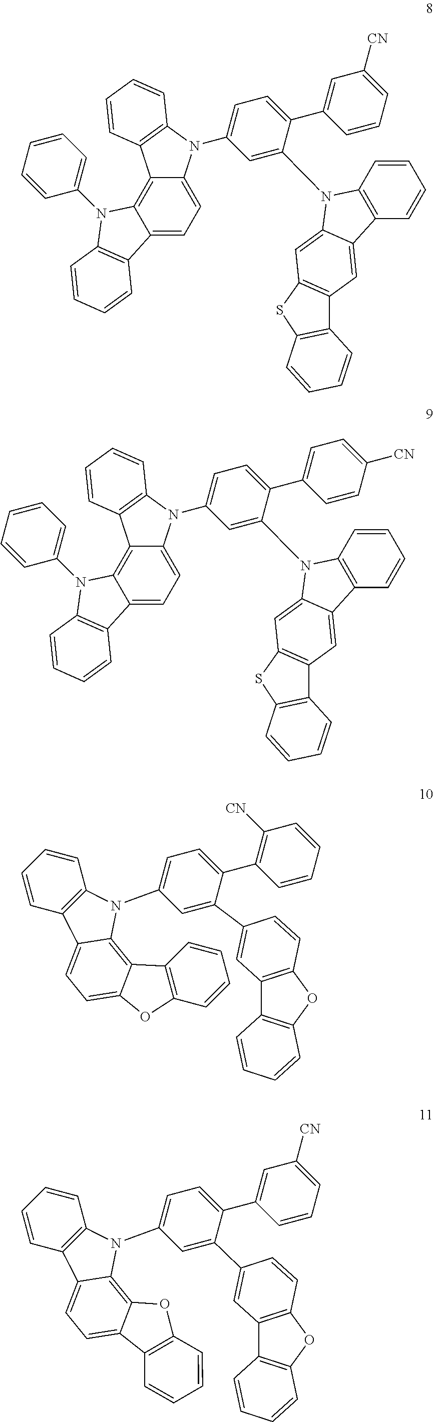

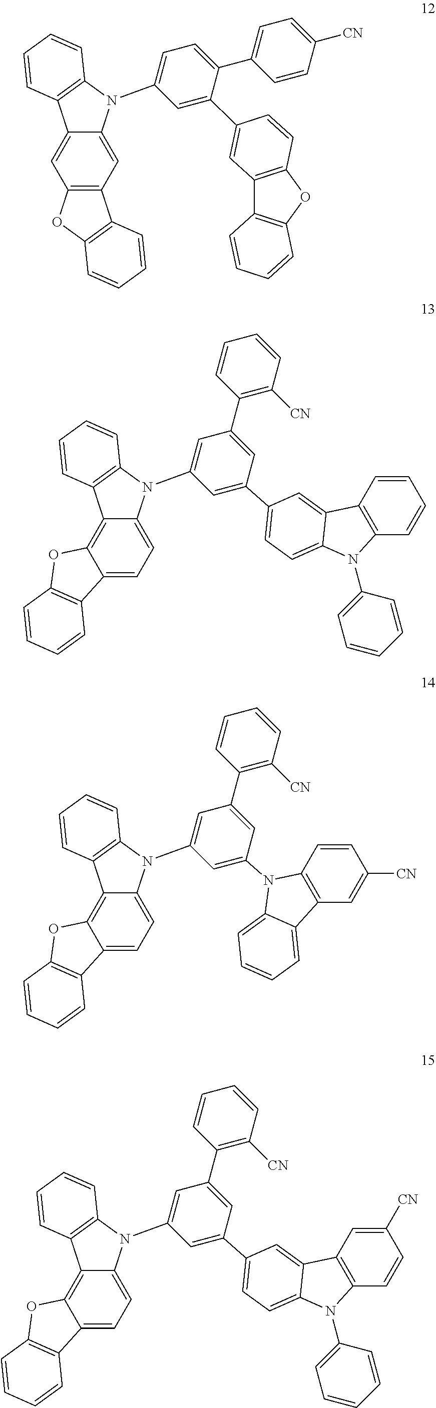 [SMILES: N#Cc1ccc(-c2ccc(-n3c4ccccc4c4cc5oc6ccccc6c5cc43)cc2-c2ccc3oc4ccccc4c3c2)cc1 ; N#Cc1ccccc1-c1cc(-c2ccc3c(c2)c2ccccc2n3-c2ccccc2)cc(-n2c3ccccc3c3c4oc5ccccc5c4ccc32)c1 ; N#Cc1ccc2c(c1)c1ccccc1n2-c1cc(-c2ccccc2C#N)cc(-n2c3ccccc3c3c4oc5ccccc5c4ccc32)c1 ; N#Cc1ccc2c(c1)c1cc(-c3cc(-c4ccccc4C#N)cc(-n4c5ccccc5c5c6oc7ccccc7c6ccc54)c3)ccc1n2-c1ccccc1]

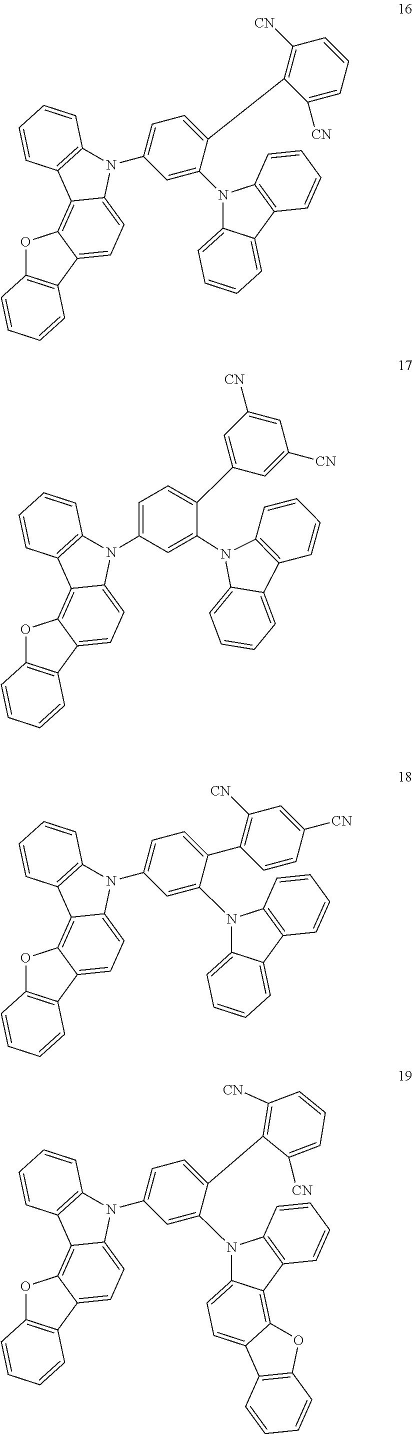

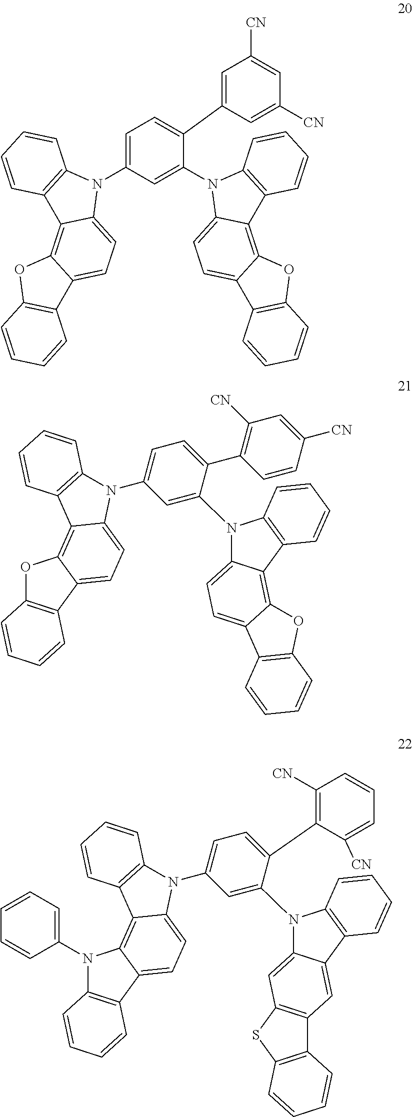

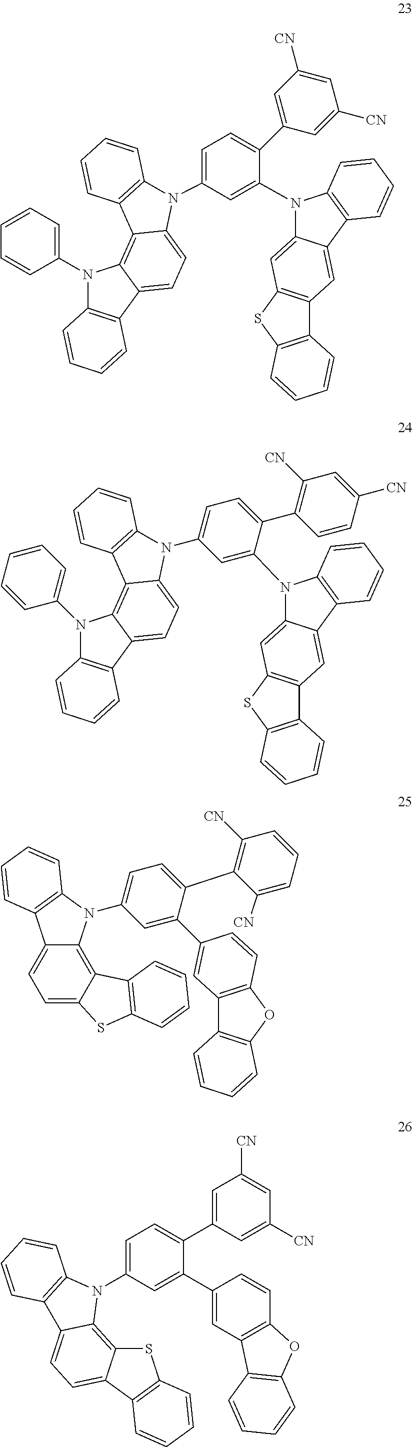

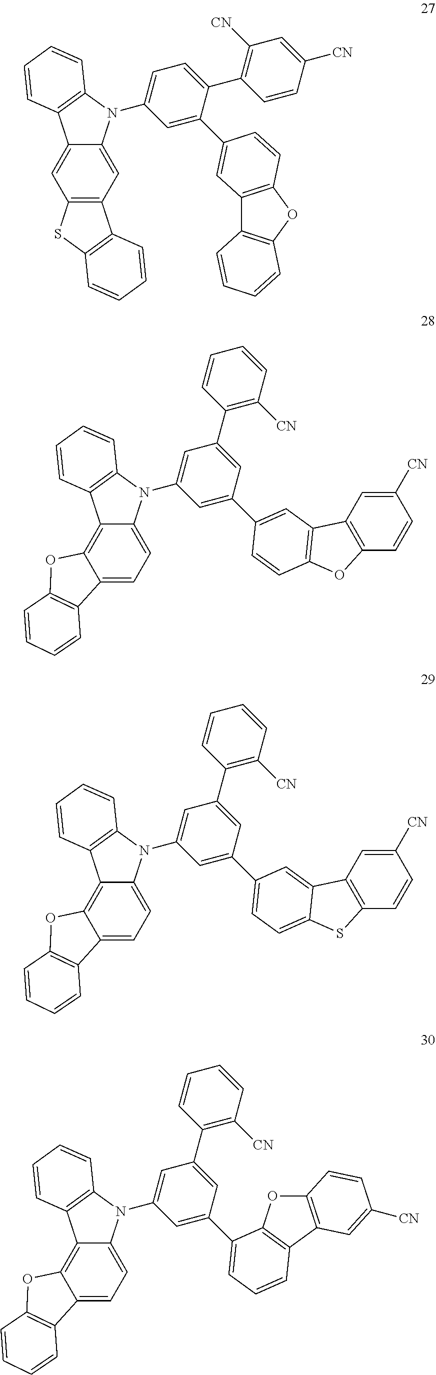

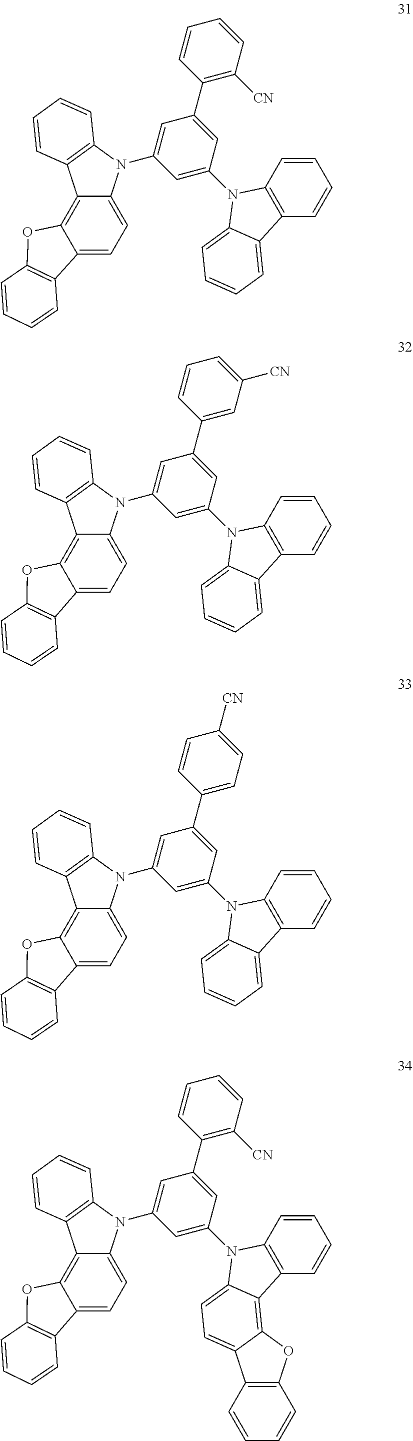 [SMILES: N#Cc1ccccc1-c1cc(-n2c3ccccc3c3ccccc32)cc(-n2c3ccccc3c3c4oc5ccccc5c4ccc32)c1 ; N#Cc1cccc(-c2cc(-n3c4ccccc4c4ccccc43)cc(-n3c4ccccc4c4c5oc6ccccc6c5ccc43)c2)c1 ; N#Cc1ccc(-c2cc(-n3c4ccccc4c4ccccc43)cc(-n3c4ccccc4c4c5oc6ccccc6c5ccc43)c2)cc1 ; N#Cc1ccccc1-c1cc(-n2c3ccccc3c3c4oc5ccccc5c4ccc32)cc(-n2c3ccccc3c3c4oc5ccccc5c4ccc32)c1]

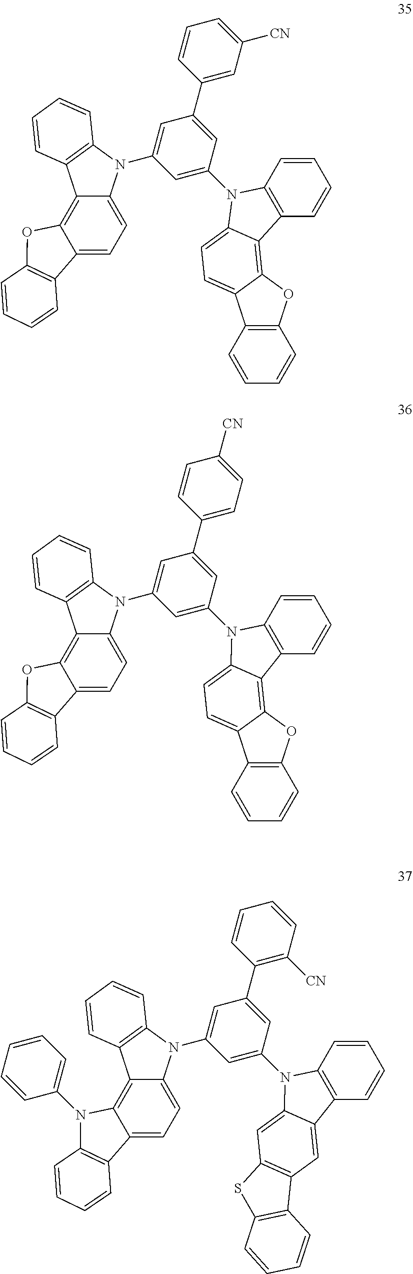

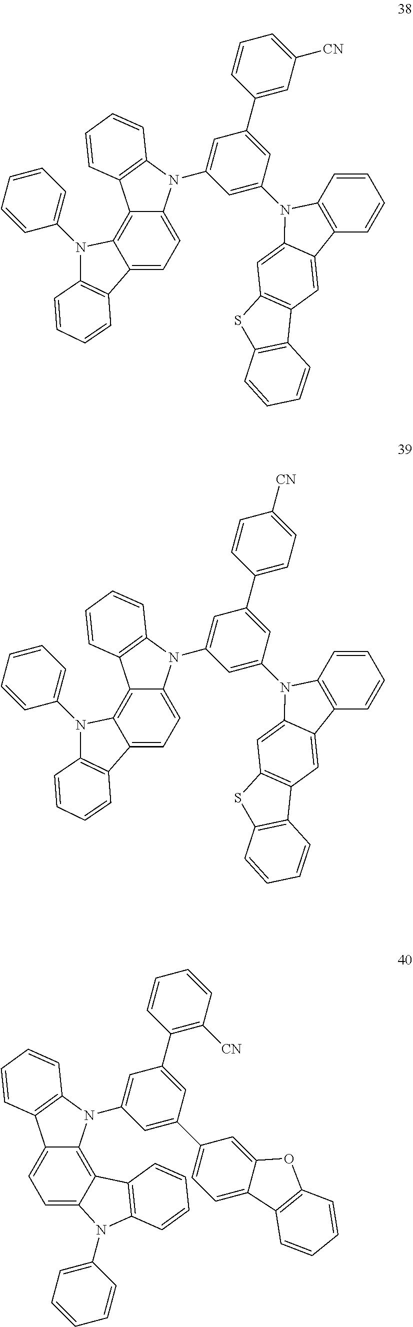 [SMILES: N#Cc1cccc(-c2cc(-n3c4ccccc4c4cc5c(cc43)sc3ccccc35)cc(-n3c4ccccc4c4c3ccc3c5ccccc5n(-c5ccccc5)c34)c2)c1 ; N#Cc1ccc(-c2cc(-n3c4ccccc4c4cc5c(cc43)sc3ccccc35)cc(-n3c4ccccc4c4c3ccc3c5ccccc5n(-c5ccccc5)c34)c2)cc1 ; N#Cc1ccccc1-c1cc(-c2ccc3c(c2)oc2ccccc23)cc(-n2c3ccccc3c3ccc4c(c5ccccc5n4-c4ccccc4)c32)c1]

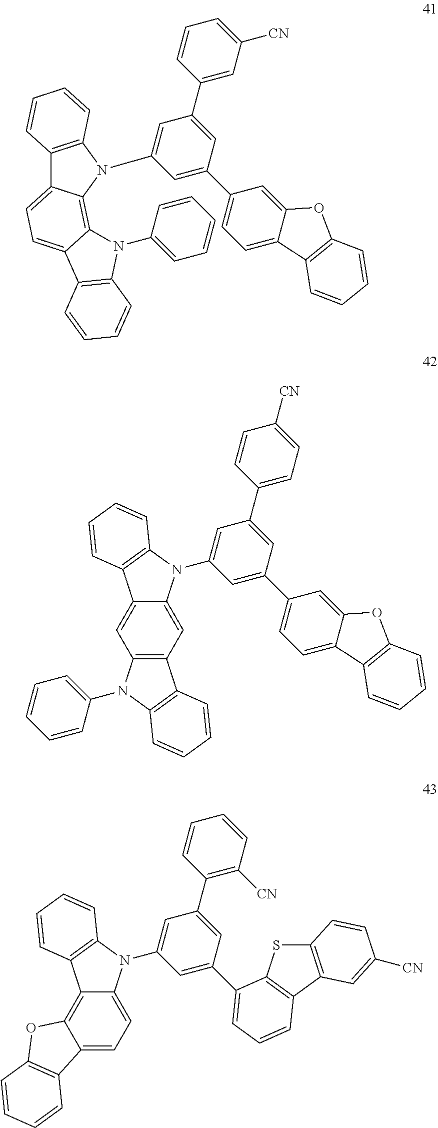 [SMILES: N#Cc1cccc(-c2cc(-c3ccc4c(c3)oc3ccccc34)cc(-n3c4ccccc4c4ccc5c6ccccc6n(-c6ccccc6)c5c43)c2)c1 ; N#Cc1ccc(-c2cc(-c3ccc4c(c3)oc3ccccc34)cc(-n3c4ccccc4c4cc5c(cc43)c3ccccc3n5-c3ccccc3)c2)cc1 ; N#Cc1ccc2sc3c(-c4cc(-c5ccccc5C#N)cc(-n5c6ccccc6c6c7oc8ccccc8c7ccc65)c4)cccc3c2c1]

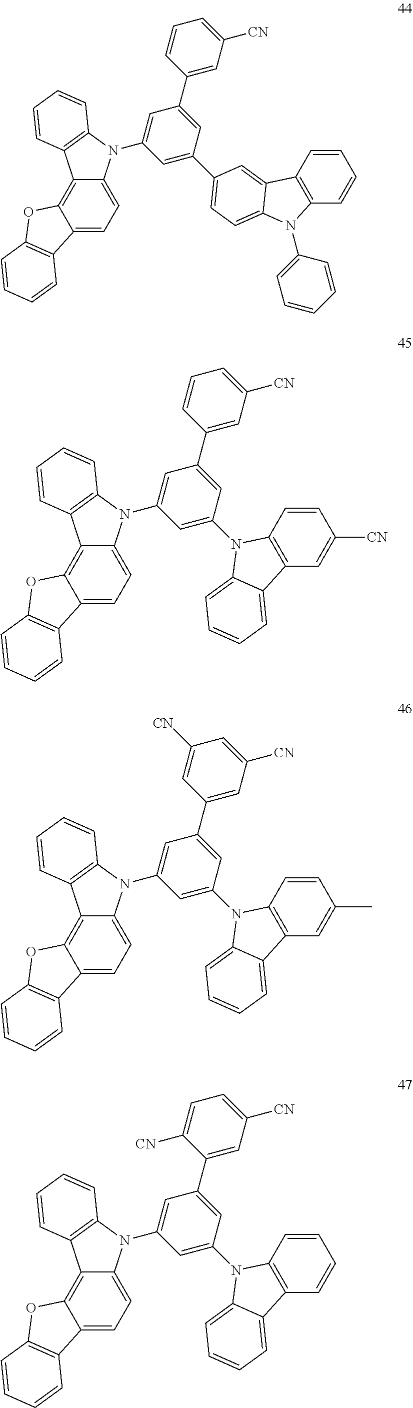

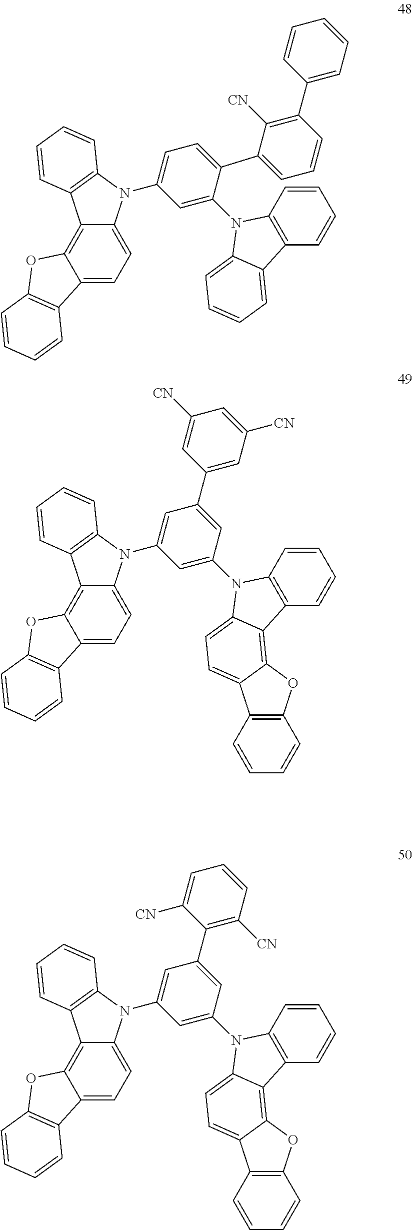

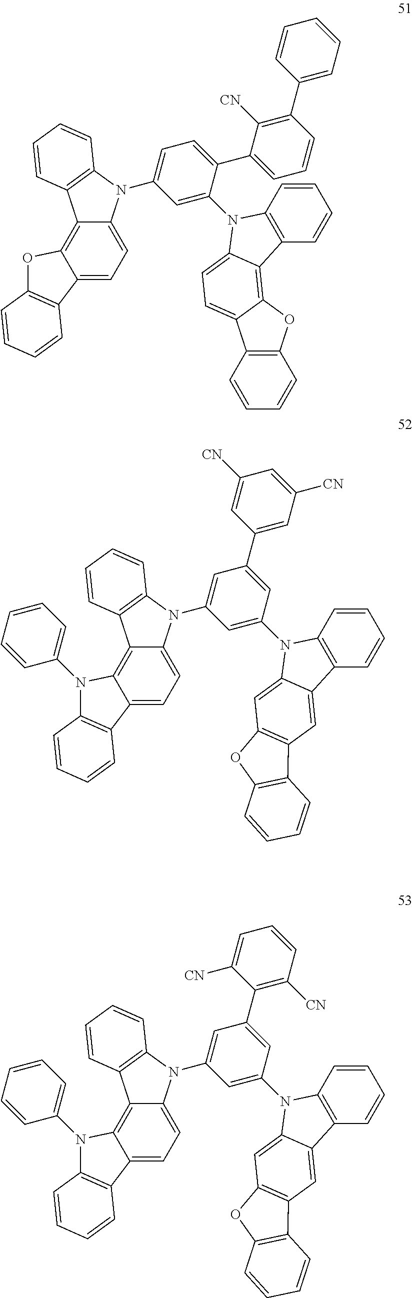

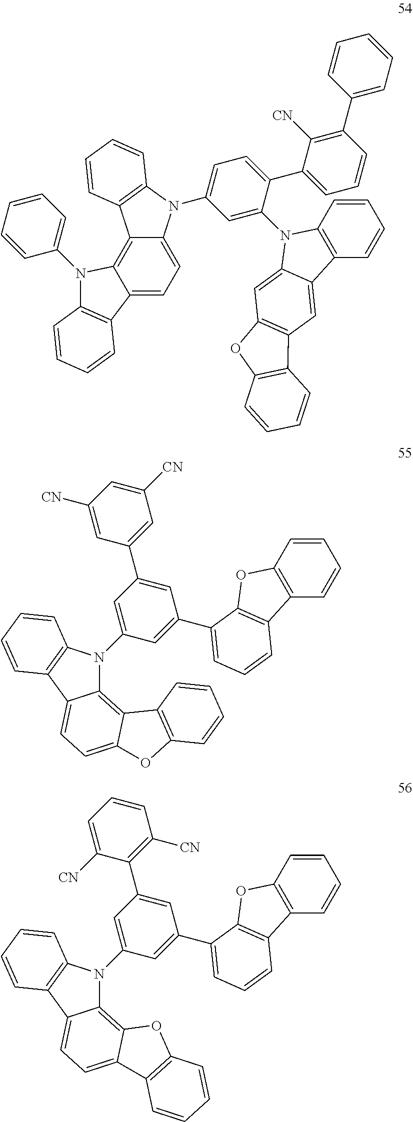

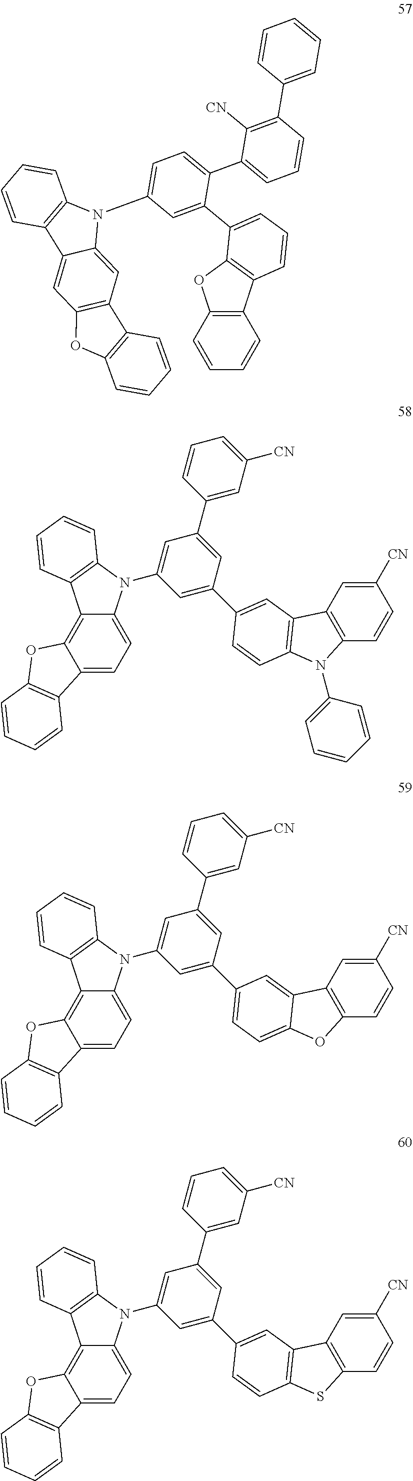

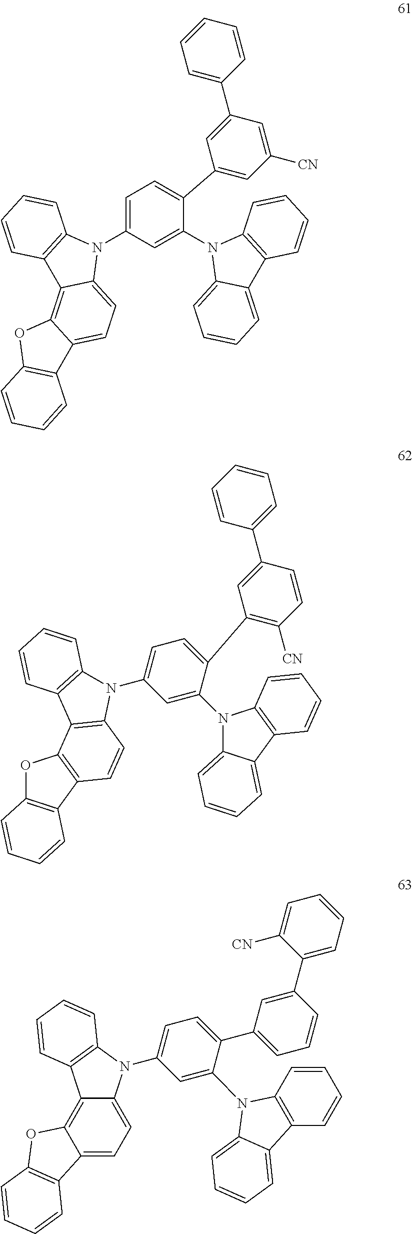

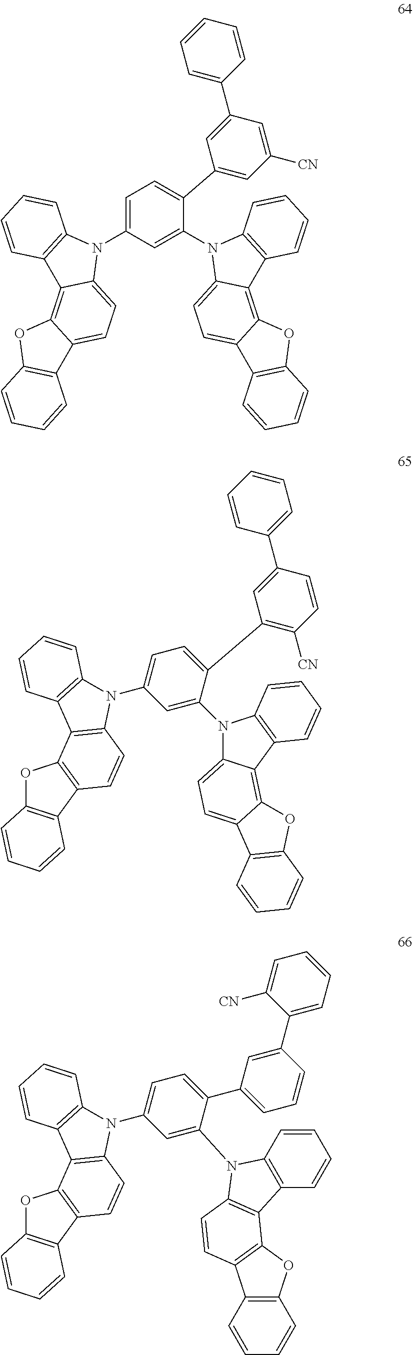

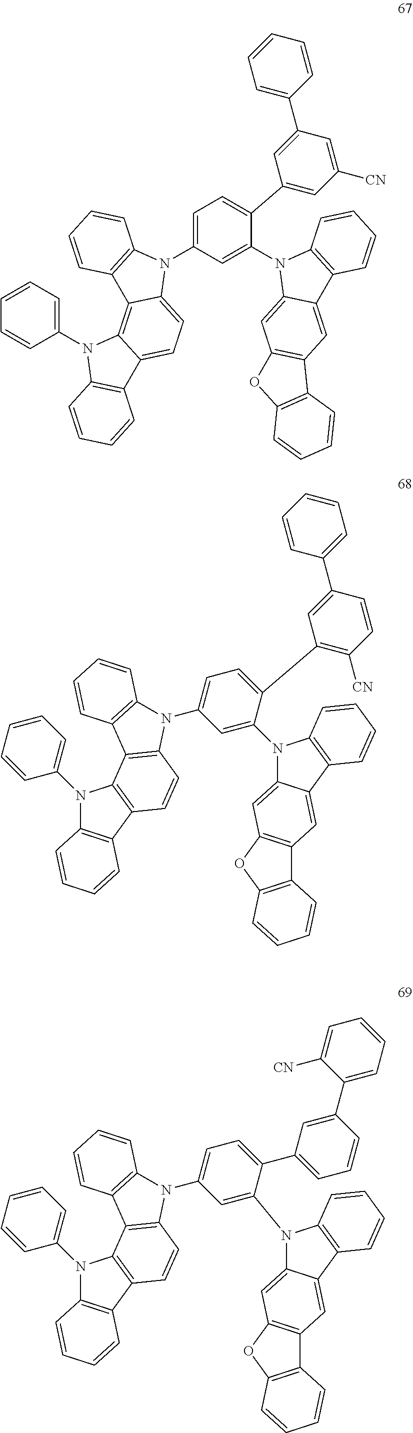

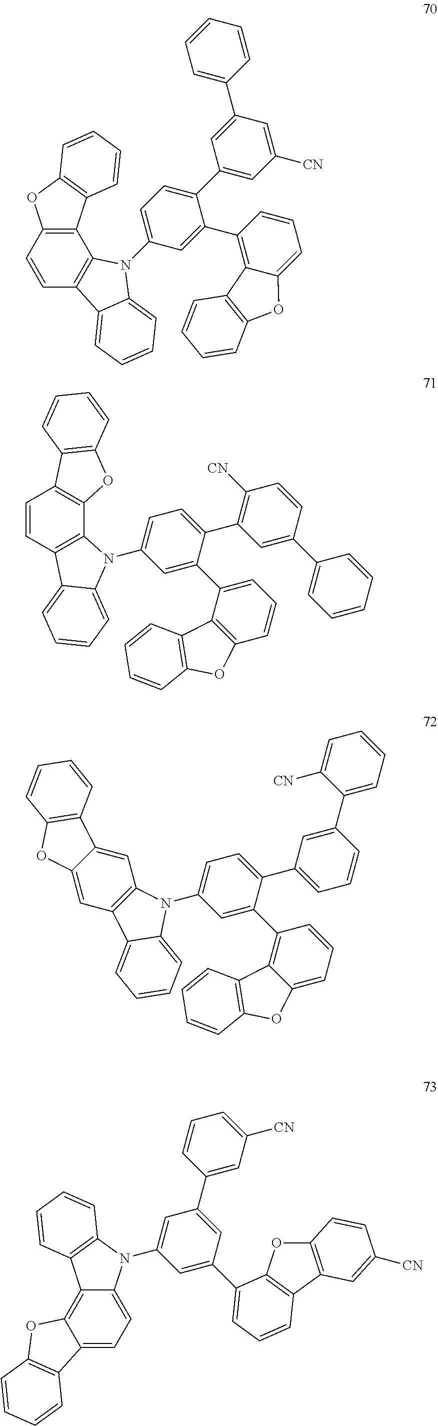

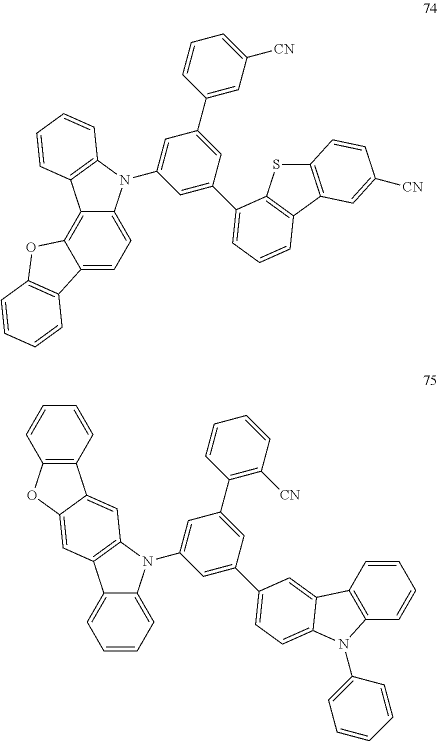 [SMILES: N#Cc1cccc(-c2cc(-c3cccc4c3sc3ccc(C#N)cc34)cc(-n3c4ccccc4c4c5oc6ccccc6c5ccc43)c2)c1 ; N#Cc1ccccc1-c1cc(-c2ccc3c(c2)c2ccccc2n3-c2ccccc2)cc(-n2c3ccccc3c3cc4oc5ccccc5c4cc32)c1]

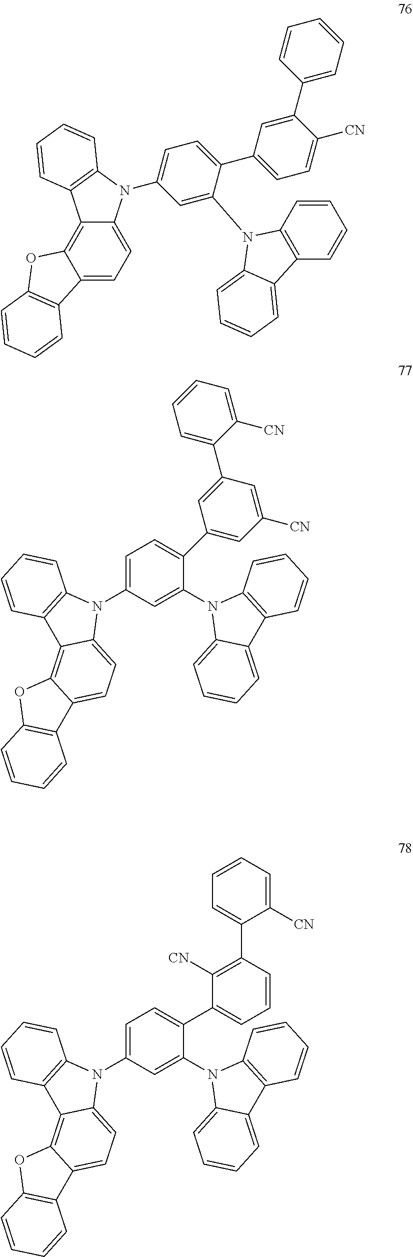

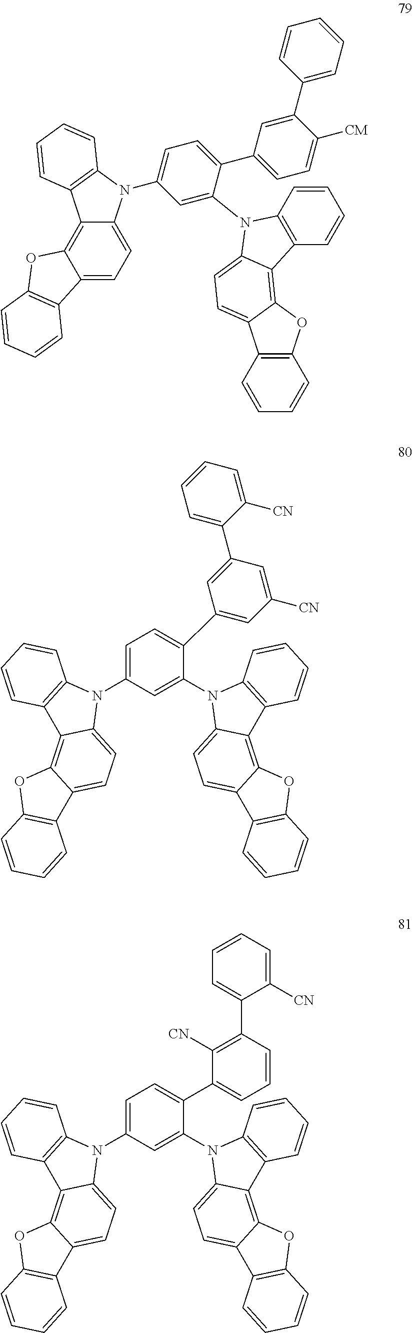

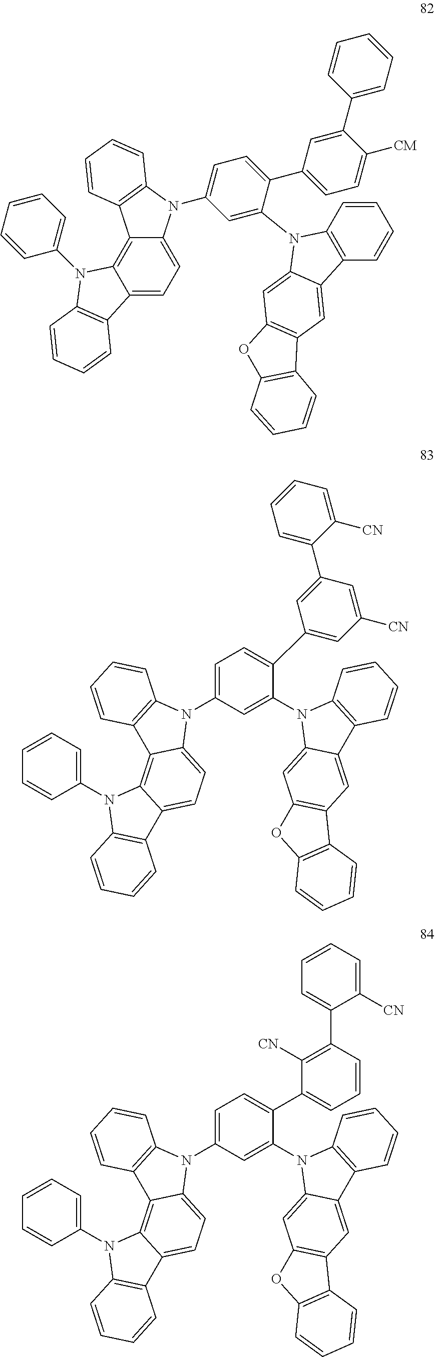

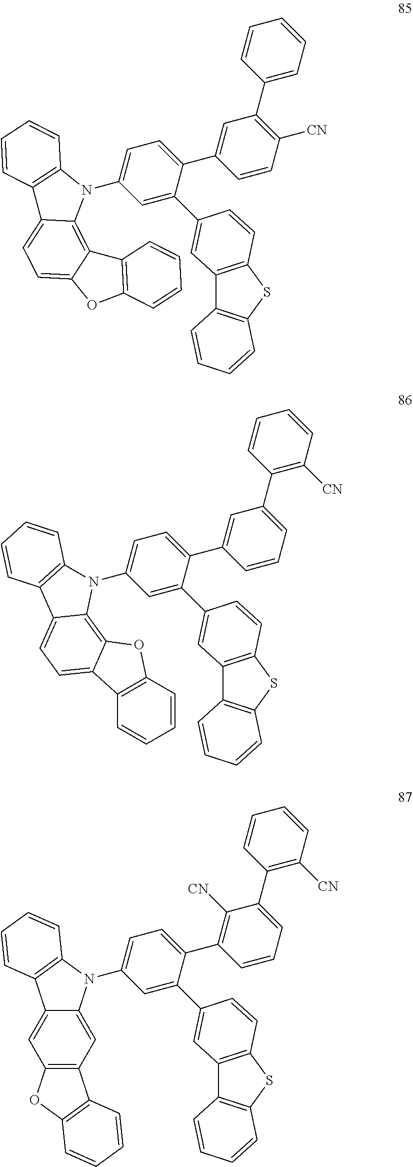

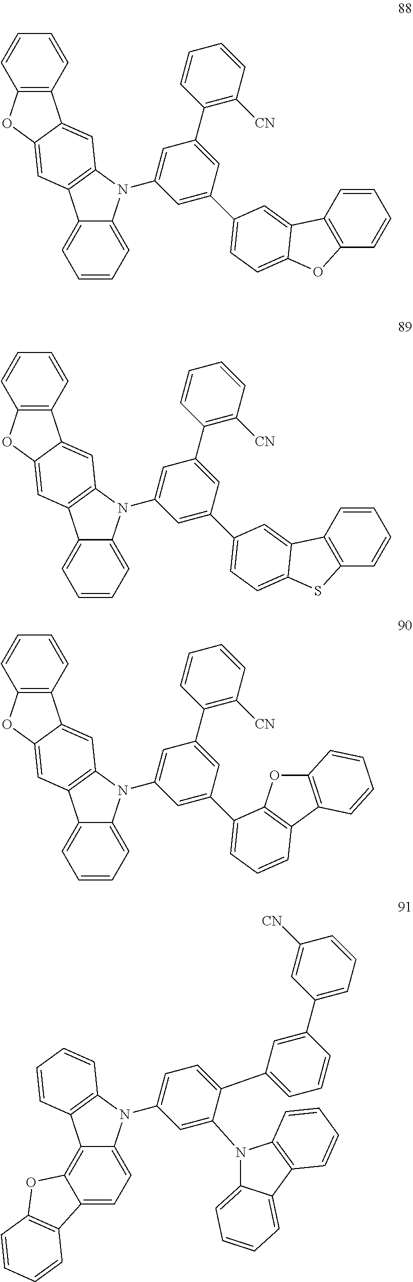

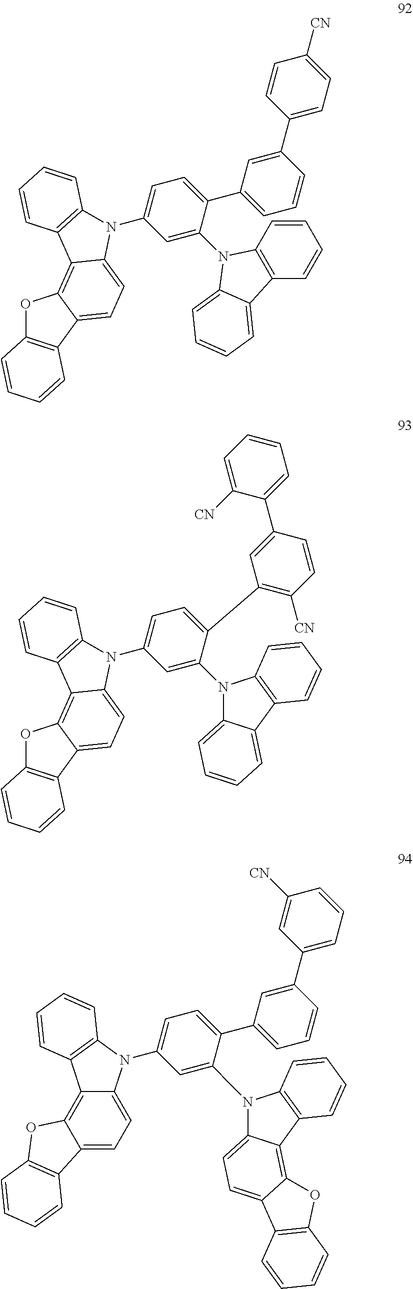

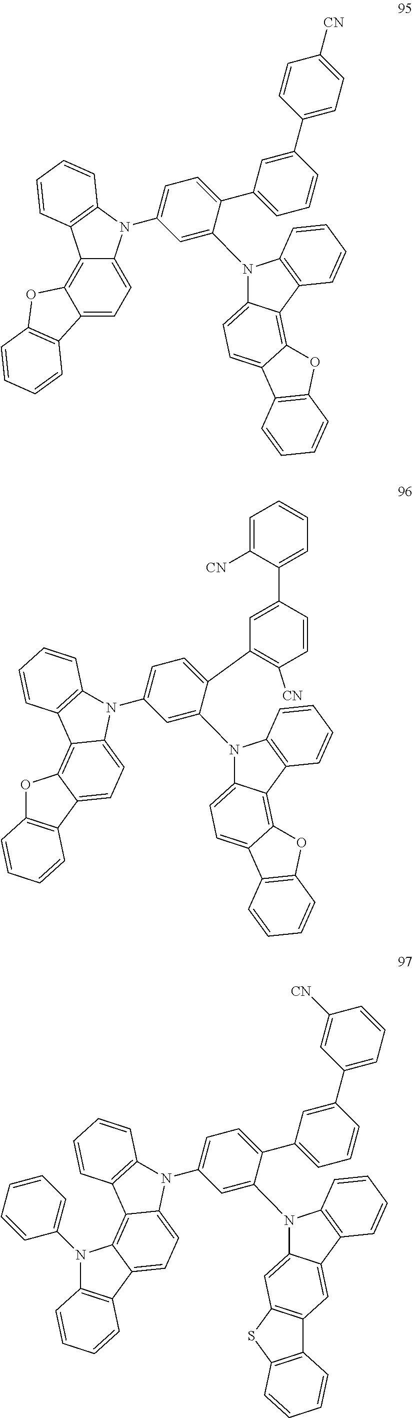

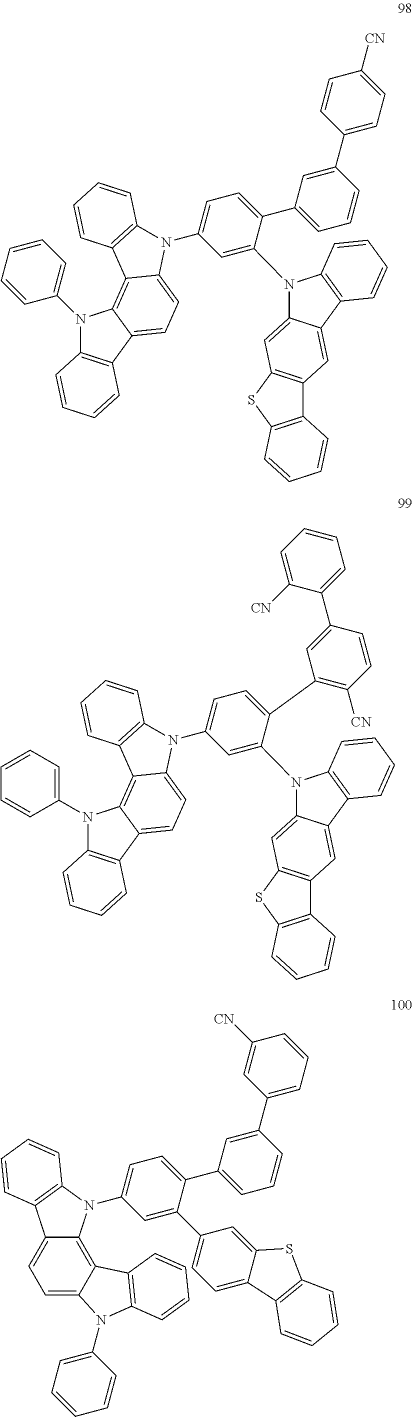

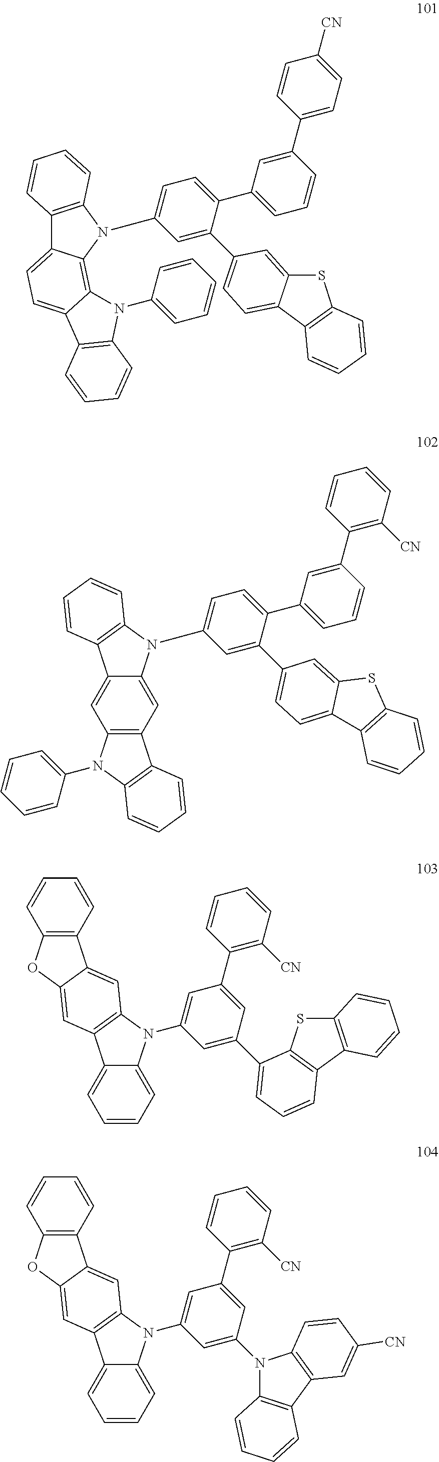 [SMILES: N#Cc1ccc(-c2cccc(-c3ccc(-n4c5ccccc5c5ccc6c7ccccc7n(-c7ccccc7)c6c54)cc3-c3ccc4c(c3)sc3ccccc34)c2)cc1 ; N#Cc1ccccc1-c1cccc(-c2ccc(-n3c4ccccc4c4cc5c(cc43)c3ccccc3n5-c3ccccc3)cc2-c2ccc3c(c2)sc2ccccc23)c1 ; N#Cc1ccccc1-c1cc(-c2cccc3c2sc2ccccc23)cc(-n2c3ccccc3c3cc4oc5ccccc5c4cc32)c1 ; N#Cc1ccc2c(c1)c1ccccc1n2-c1cc(-c2ccccc2C#N)cc(-n2c3ccccc3c3cc4oc5ccccc5c4cc32)c1]

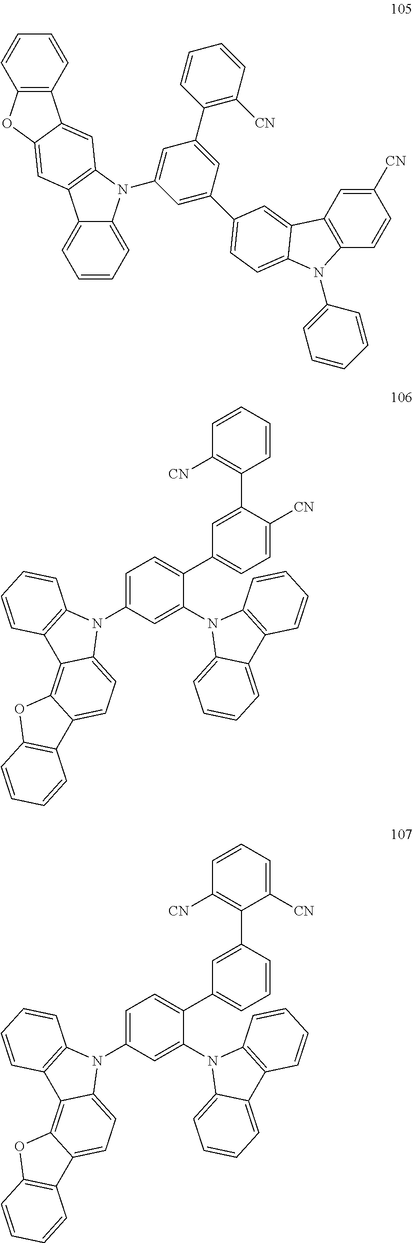

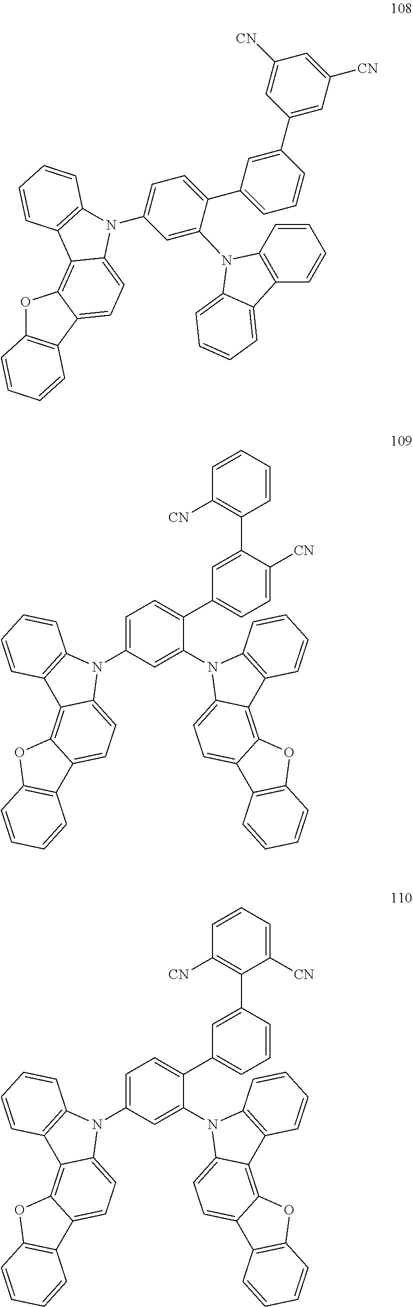

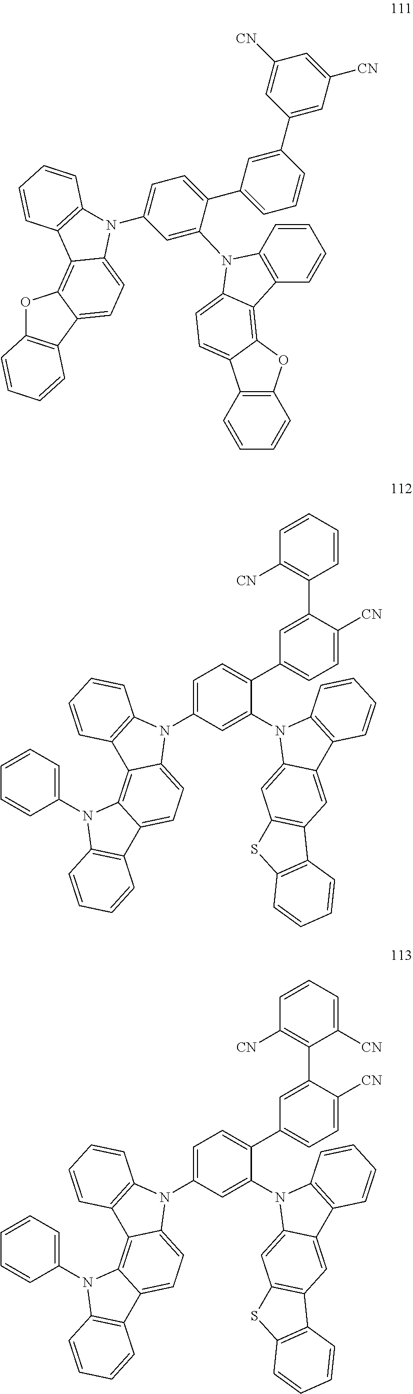

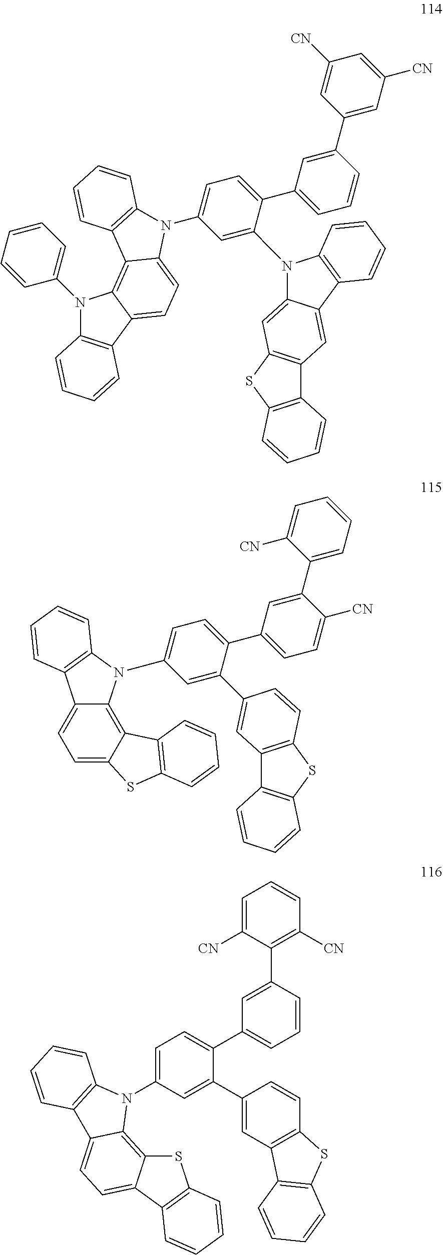

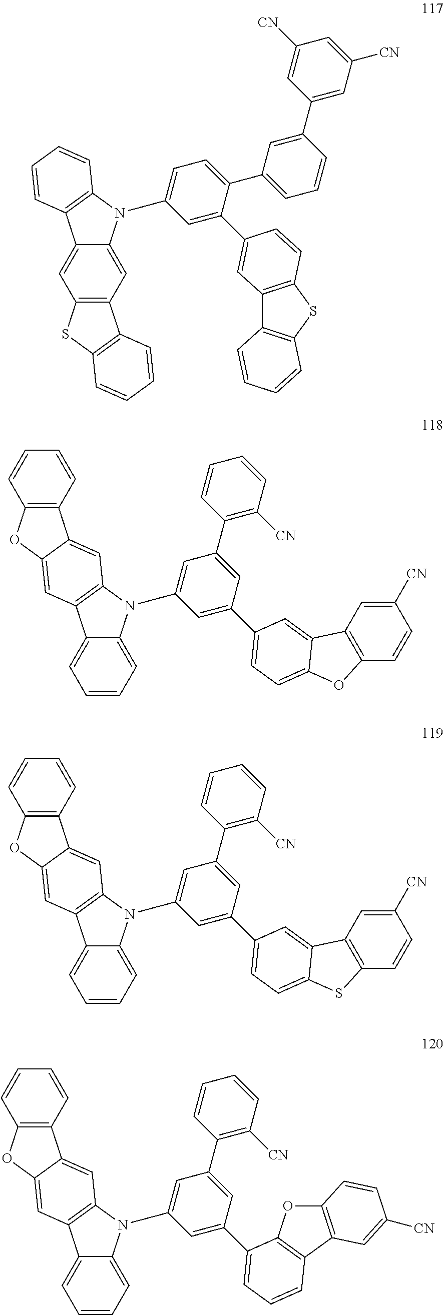

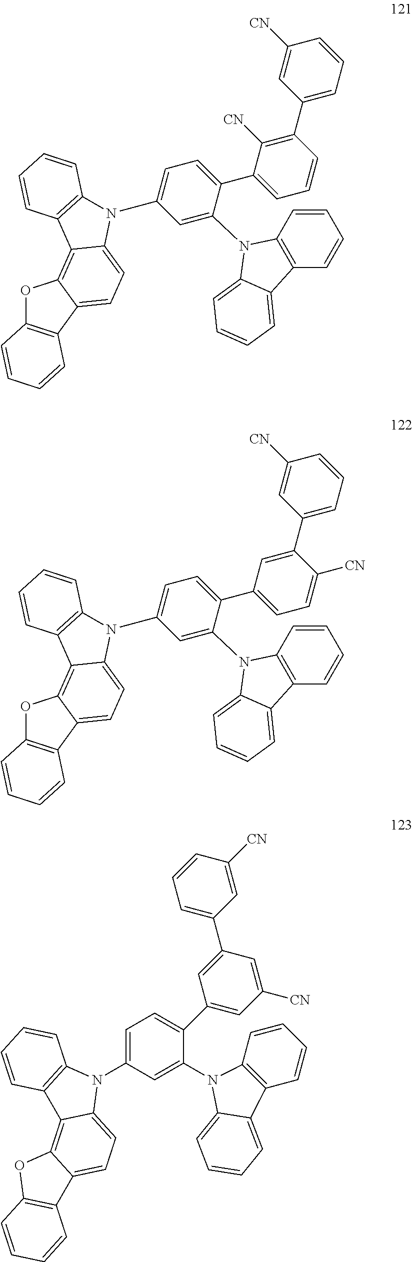

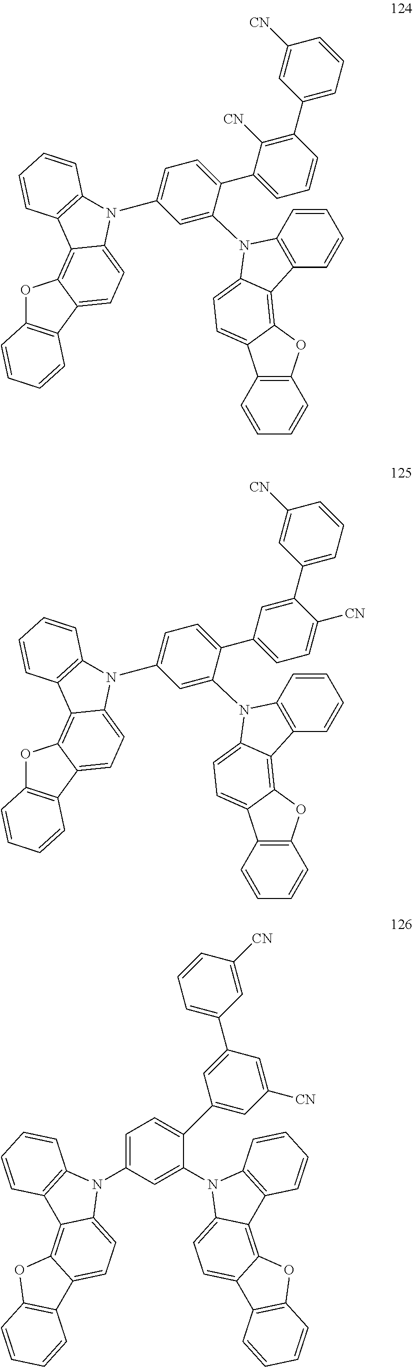

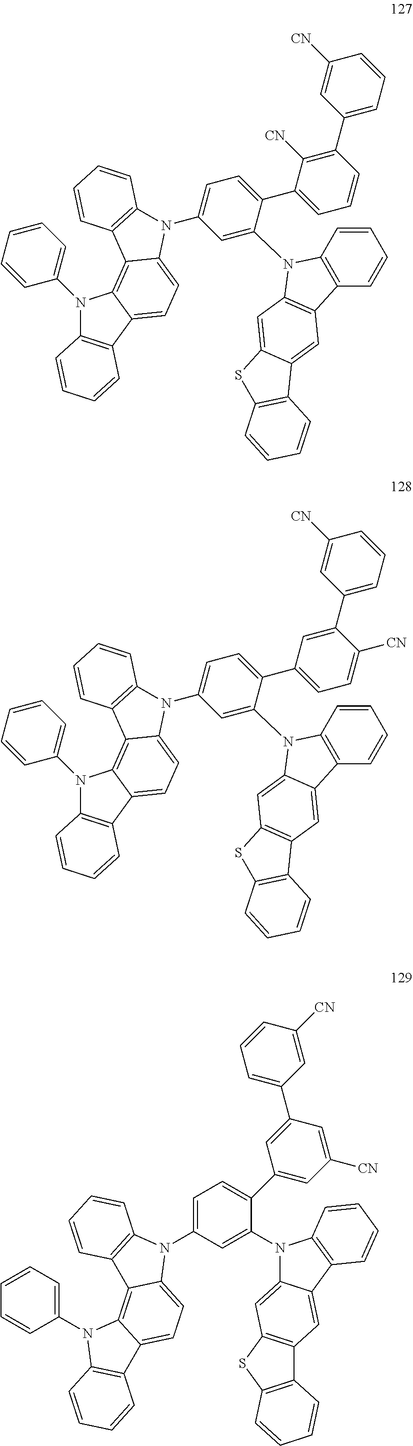

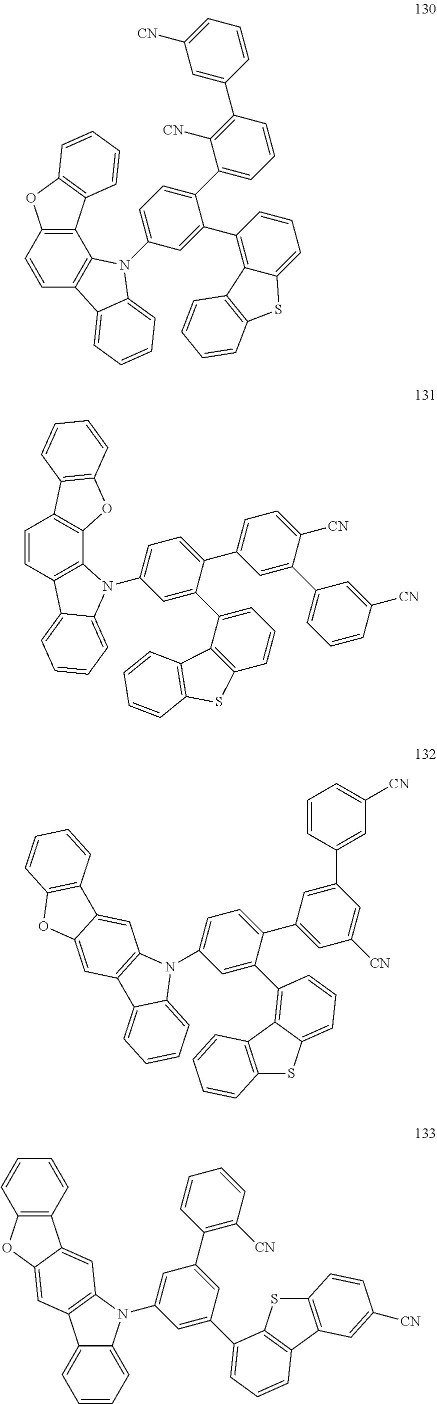

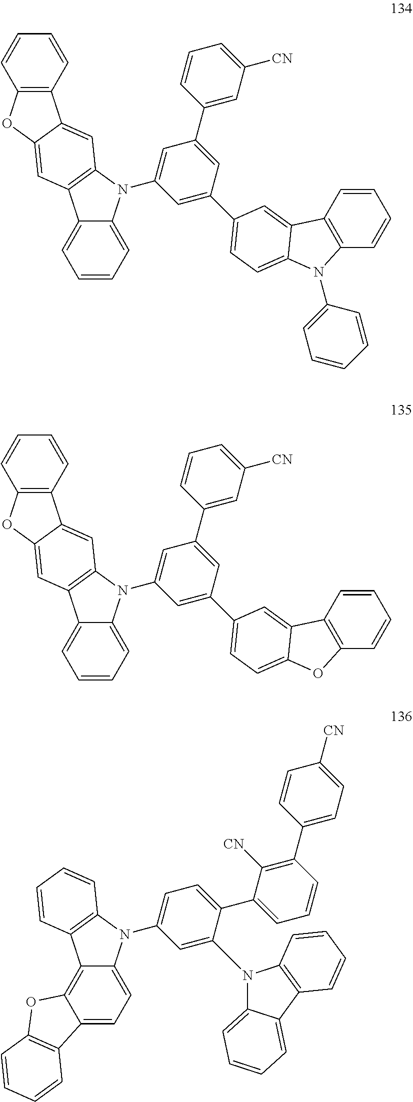

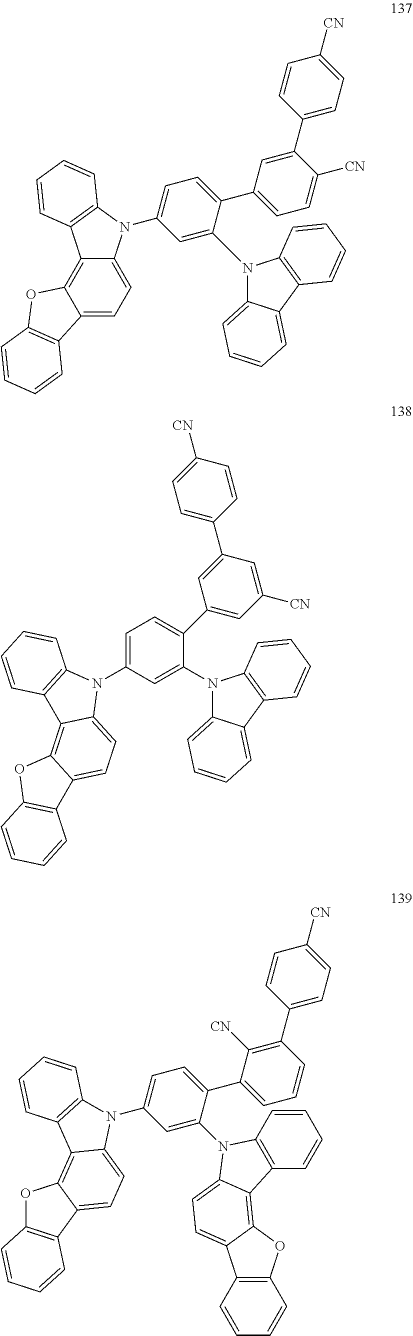

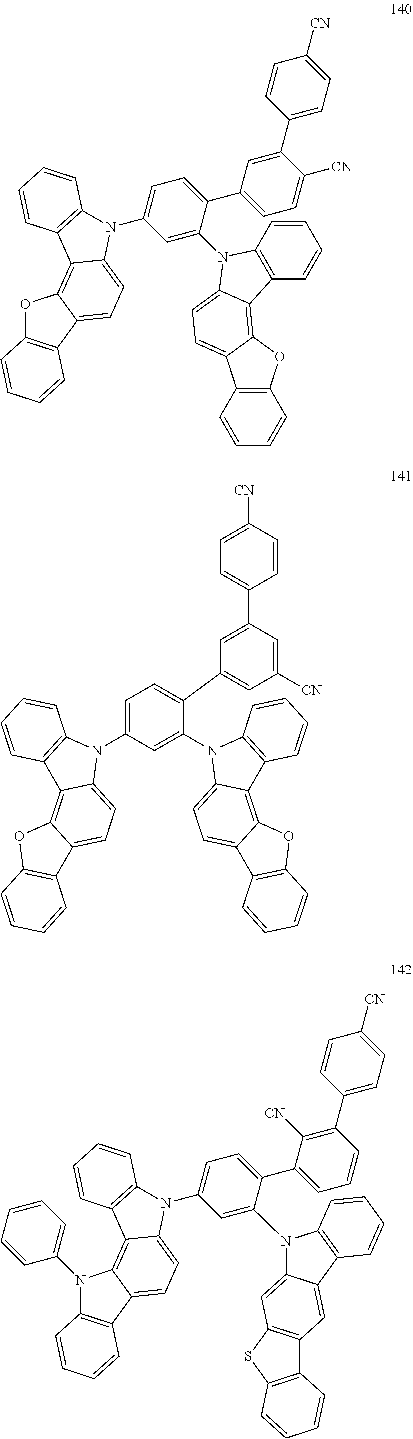

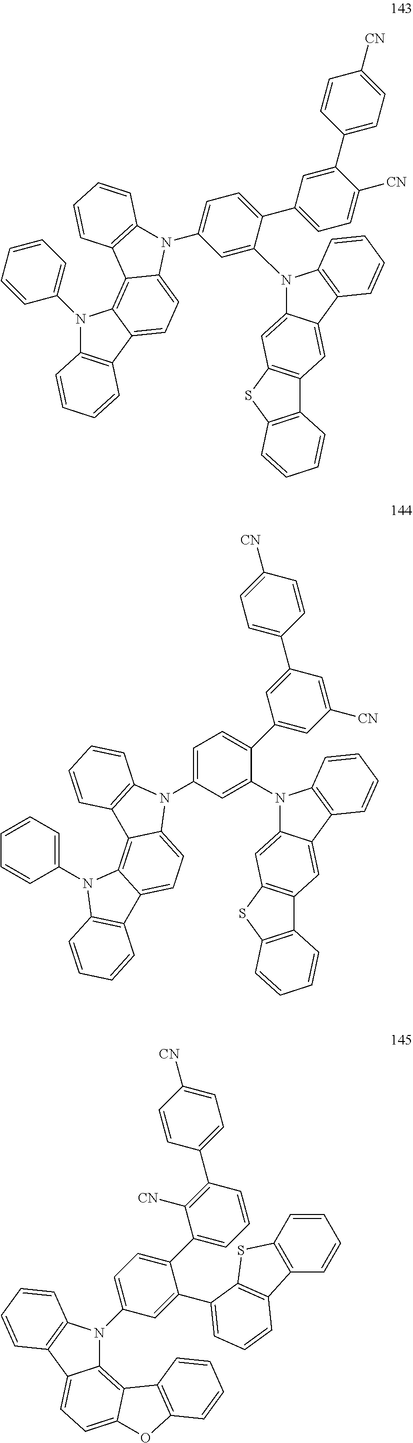

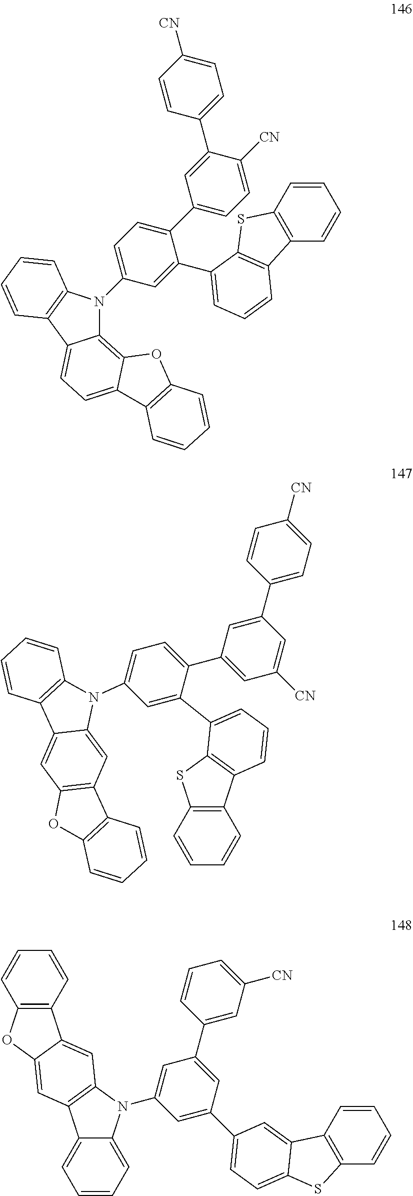

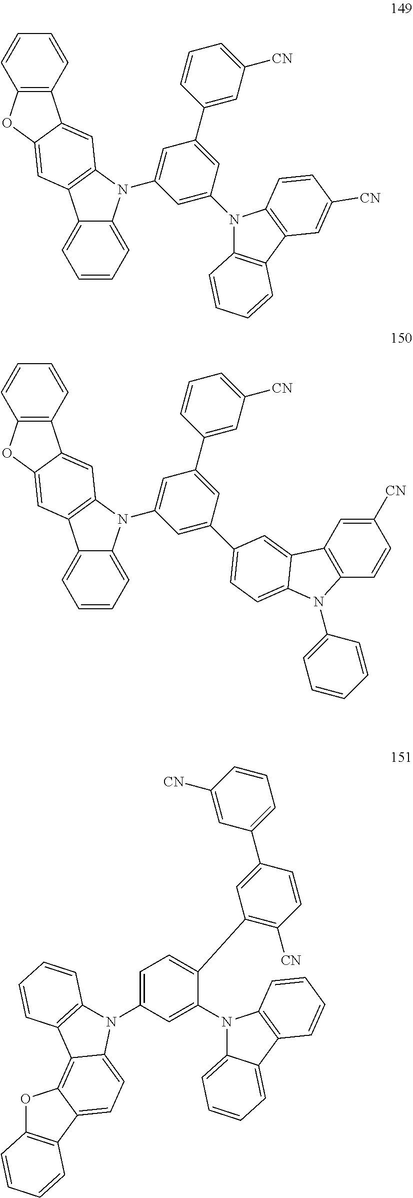

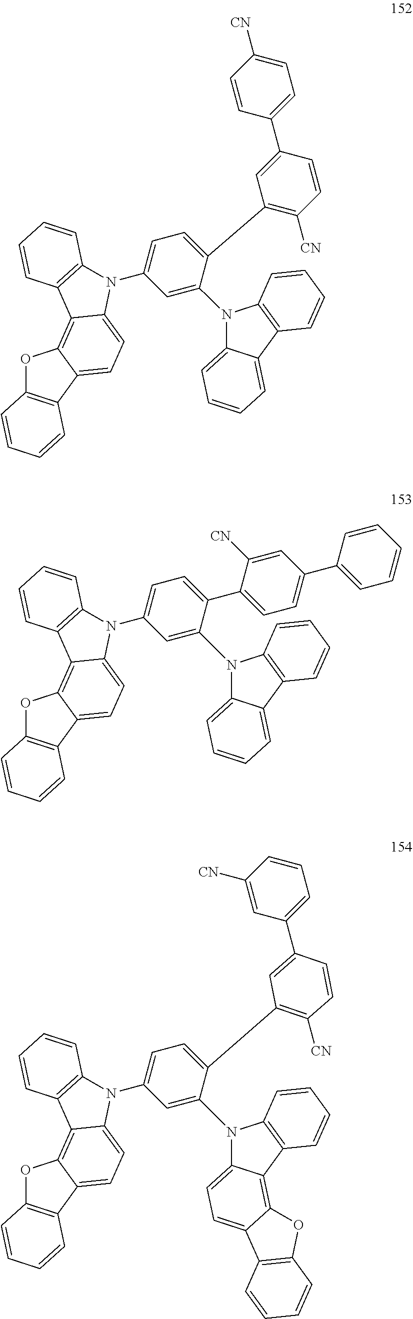

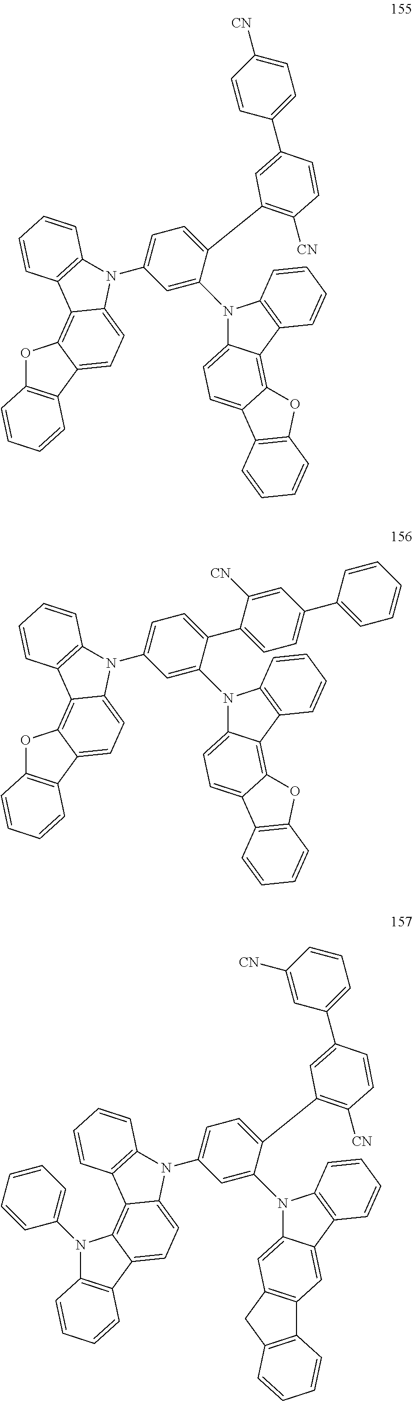

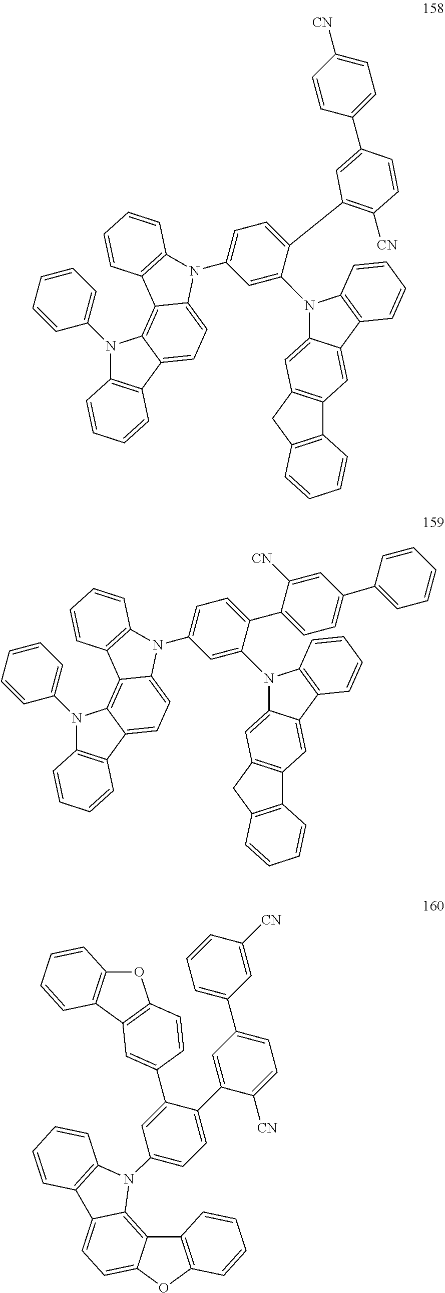

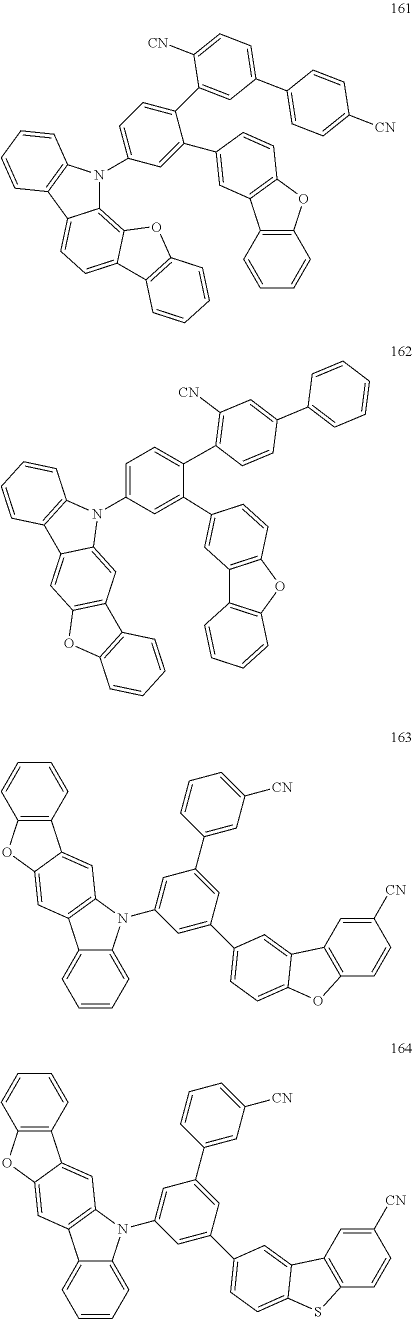

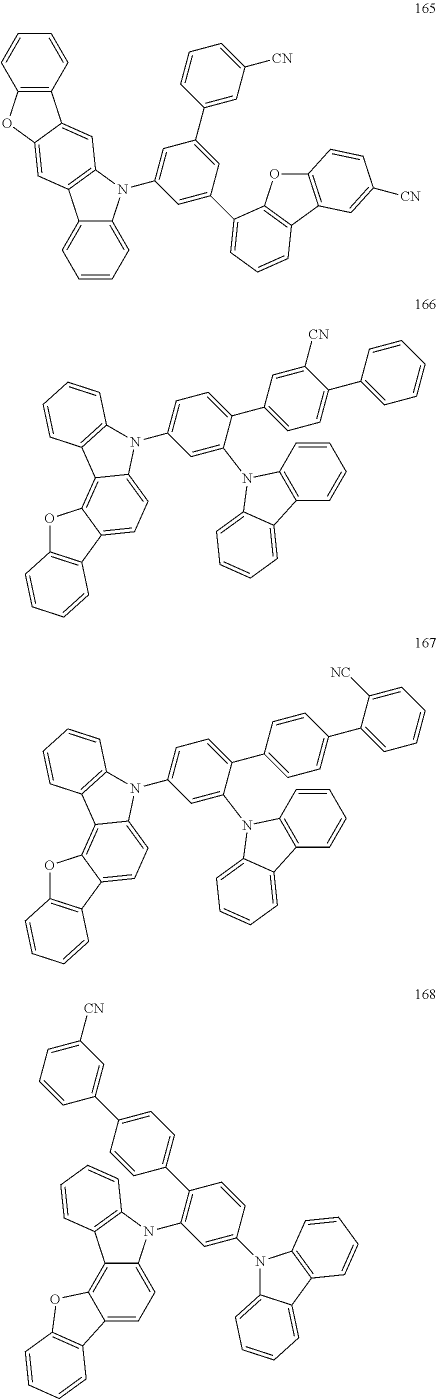 [SMILES: N#Cc1cccc(-c2cc(-c3cccc4c3oc3ccc(C#N)cc34)cc(-n3c4ccccc4c4cc5oc6ccccc6c5cc43)c2)c1 ; N#Cc1cc(-c2ccc(-n3c4ccccc4c4c5oc6ccccc6c5ccc43)cc2-n2c3ccccc3c3ccccc32)ccc1-c1ccccc1 ; N#Cc1ccccc1-c1ccc(-c2ccc(-n3c4ccccc4c4c5oc6ccccc6c5ccc43)cc2-n2c3ccccc3c3ccccc32)cc1 ; N#Cc1cccc(-c2ccc(-c3ccc(-n4c5ccccc5c5ccccc54)cc3-n3c4ccccc4c4c5oc6ccccc6c5ccc43)cc2)c1]

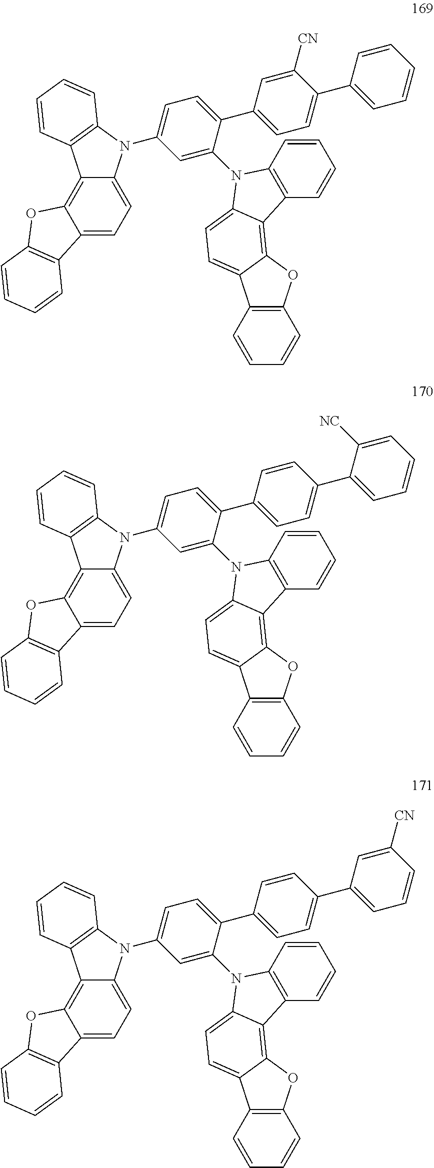 [SMILES: N#Cc1cc(-c2ccc(-n3c4ccccc4c4c5oc6ccccc6c5ccc43)cc2-n2c3ccccc3c3c4oc5ccccc5c4ccc32)ccc1-c1ccccc1 ; N#Cc1ccccc1-c1ccc(-c2ccc(-n3c4ccccc4c4c5oc6ccccc6c5ccc43)cc2-n2c3ccccc3c3c4oc5ccccc5c4ccc32)cc1 ; N#Cc1cccc(-c2ccc(-c3ccc(-n4c5ccccc5c5c6oc7ccccc7c6ccc54)cc3-n3c4ccccc4c4c5oc6ccccc6c5ccc43)cc2)c1]

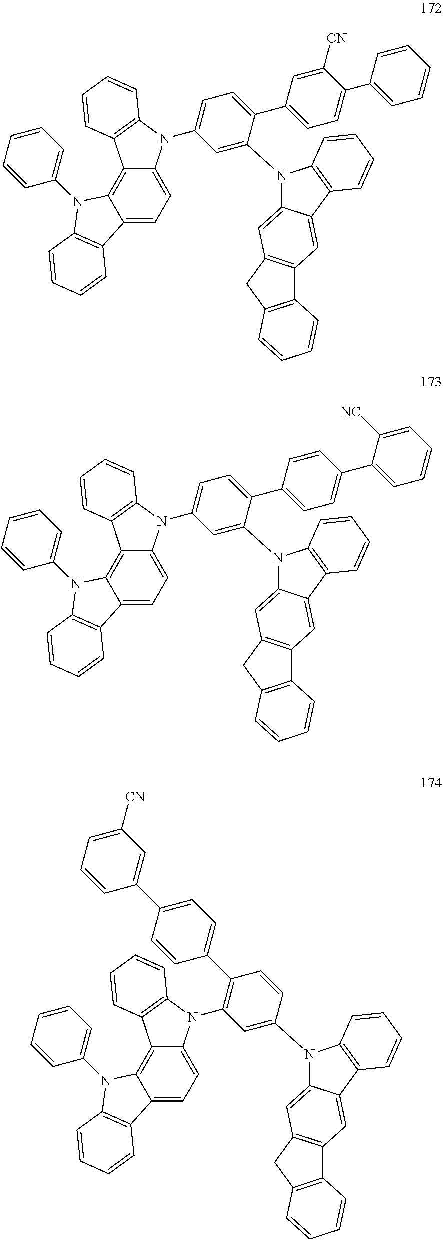 [SMILES: N#Cc1cc(-c2ccc(-n3c4ccccc4c4c3ccc3c5ccccc5n(-c5ccccc5)c34)cc2-n2c3ccccc3c3cc4c(cc32)Cc2ccccc2-4)ccc1-c1ccccc1 ; N#Cc1ccccc1-c1ccc(-c2ccc(-n3c4ccccc4c4c3ccc3c5ccccc5n(-c5ccccc5)c34)cc2-n2c3ccccc3c3cc4c(cc32)Cc2ccccc2-4)cc1 ; N#Cc1cccc(-c2ccc(-c3ccc(-n4c5ccccc5c5cc6c(cc54)Cc4ccccc4-6)cc3-n3c4ccccc4c4c3ccc3c5ccccc5n(-c5ccccc5)c34)cc2)c1]

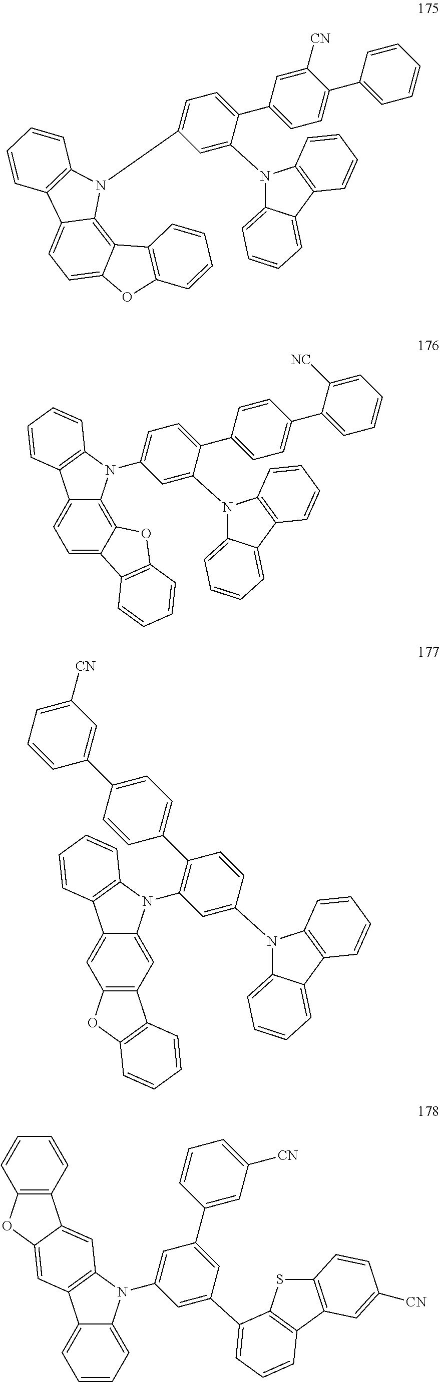 [SMILES: N#Cc1cc(-c2ccc(-n3c4ccccc4c4ccc5oc6ccccc6c5c43)cc2-n2c3ccccc3c3ccccc32)ccc1-c1ccccc1 ; N#Cc1ccccc1-c1ccc(-c2ccc(-n3c4ccccc4c4ccc5c6ccccc6oc5c43)cc2-n2c3ccccc3c3ccccc32)cc1 ; N#Cc1cccc(-c2ccc(-c3ccc(-n4c5ccccc5c5ccccc54)cc3-n3c4ccccc4c4cc5oc6ccccc6c5cc43)cc2)c1 ; N#Cc1cccc(-c2cc(-c3cccc4c3sc3ccc(C#N)cc34)cc(-n3c4ccccc4c4cc5oc6ccccc6c5cc43)c2)c1]

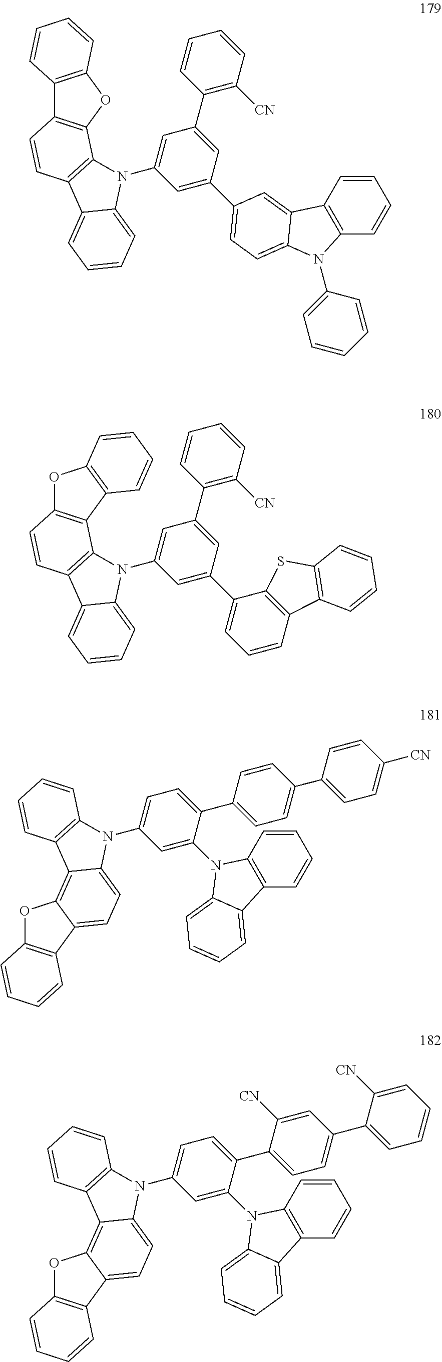

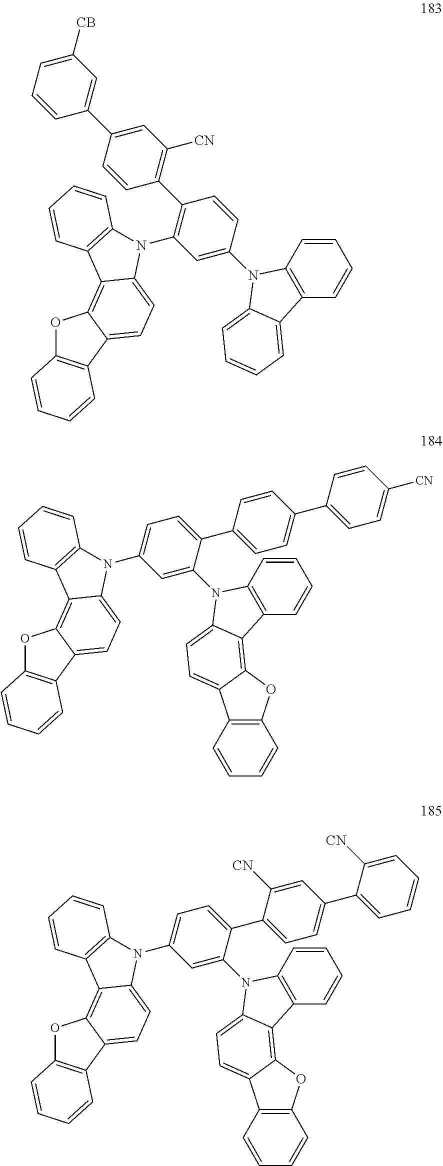

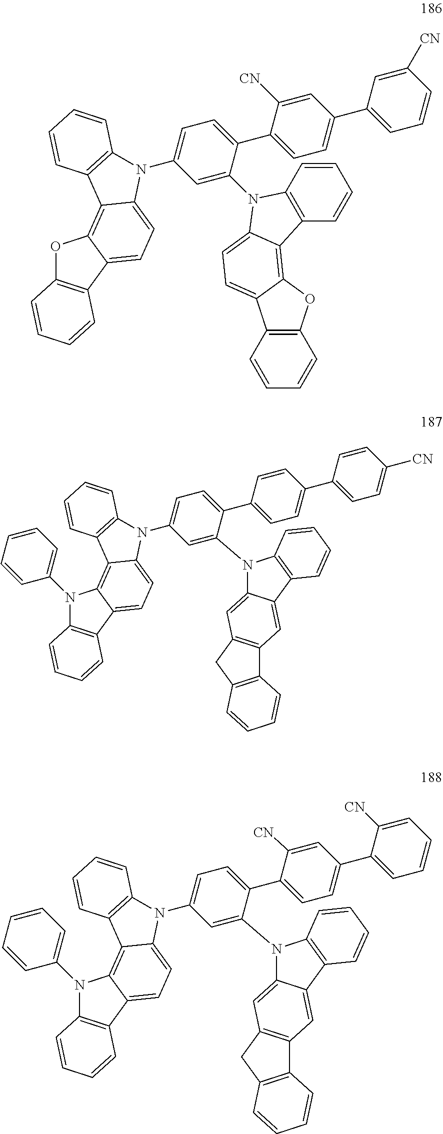

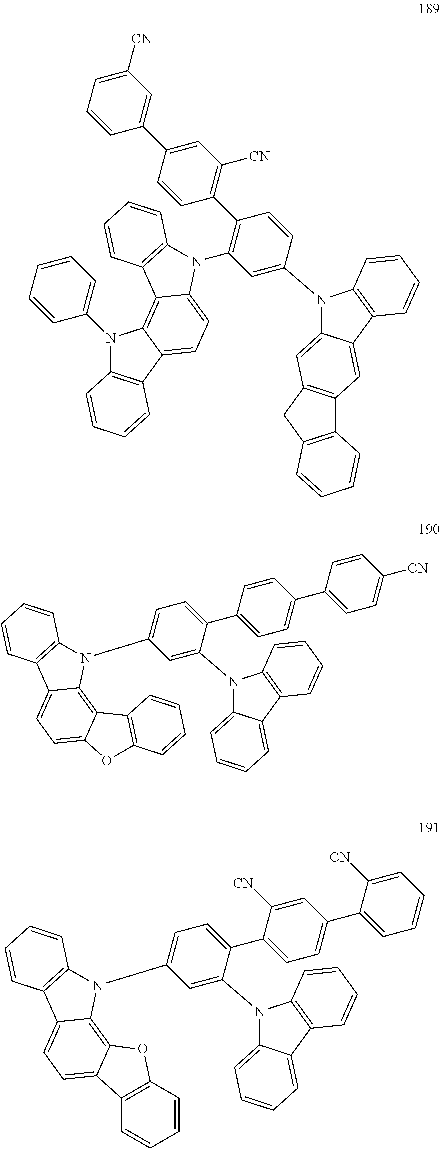

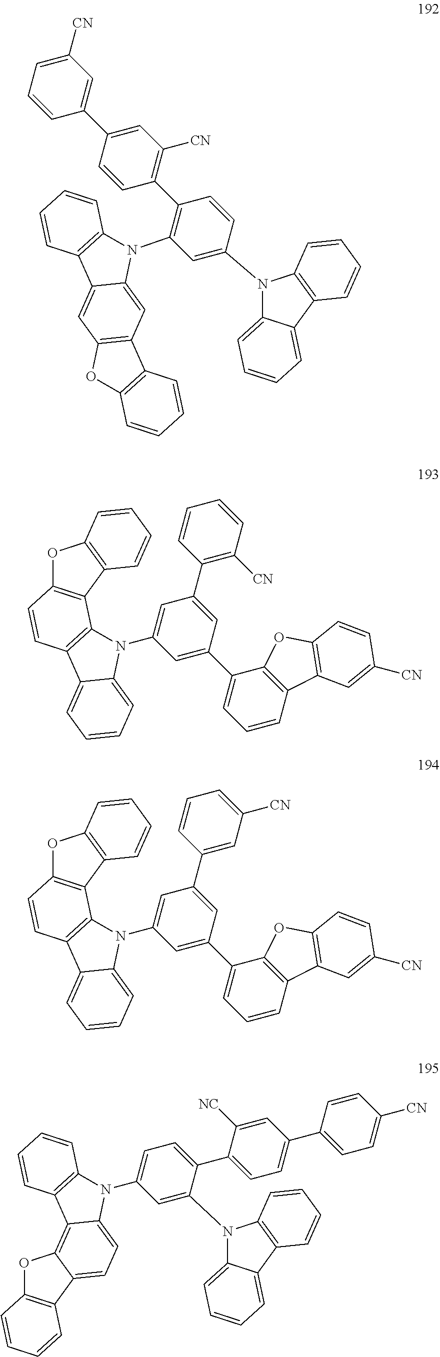 [SMILES: N#Cc1cccc(-c2ccc(-c3ccc(-n4c5ccccc5c5ccccc54)cc3-n3c4ccccc4c4cc5oc6ccccc6c5cc43)c(C#N)c2)c1 ; N#Cc1ccc2oc3c(-c4cc(-c5ccccc5C#N)cc(-n5c6ccccc6c6ccc7oc8ccccc8c7c65)c4)cccc3c2c1 ; N#Cc1cccc(-c2cc(-c3cccc4c3oc3ccc(C#N)cc34)cc(-n3c4ccccc4c4ccc5oc6ccccc6c5c43)c2)c1 ; N#Cc1ccc(-c2ccc(-c3ccc(-n4c5ccccc5c5c6oc7ccccc7c6ccc54)cc3-n3c4ccccc4c4ccccc43)c(C#N)c2)cc1]

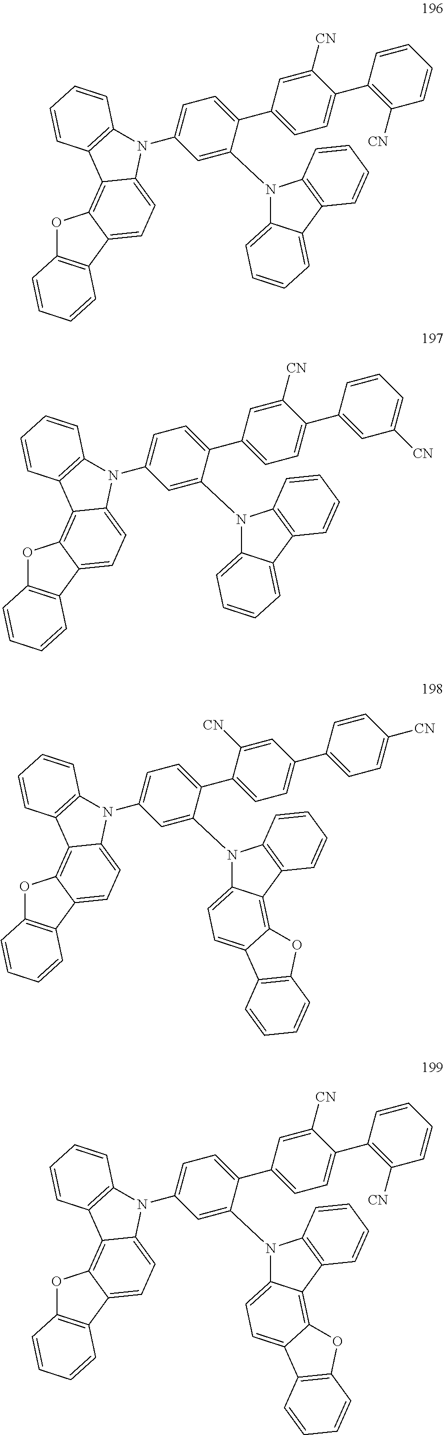

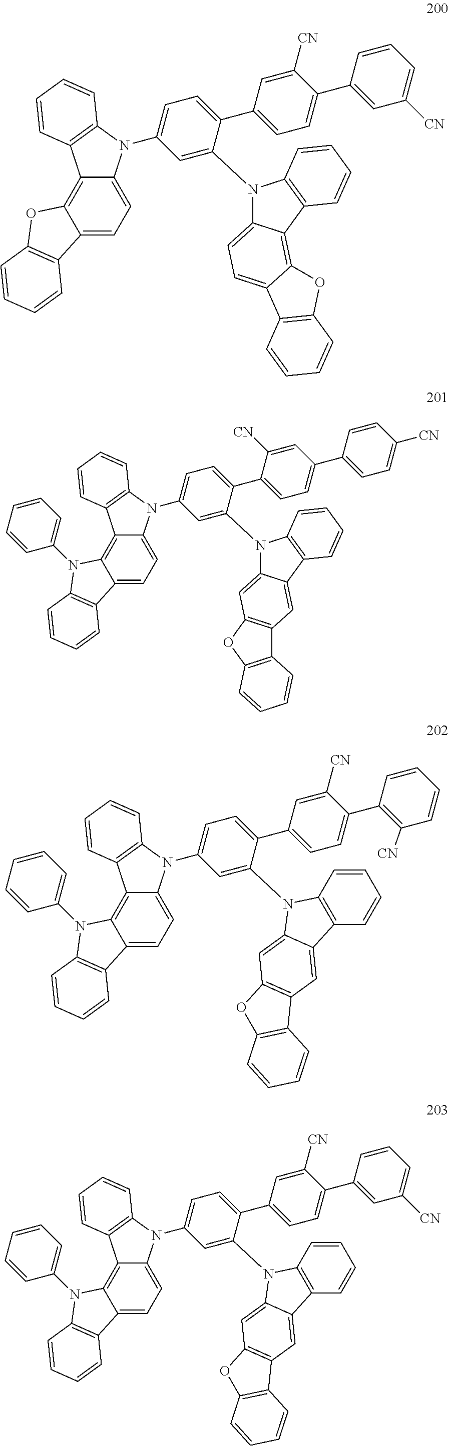

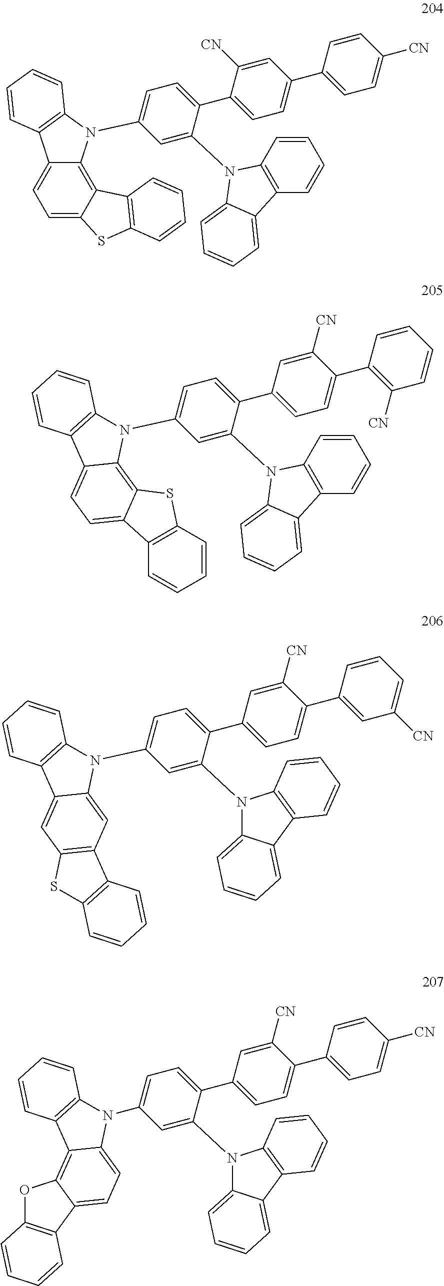

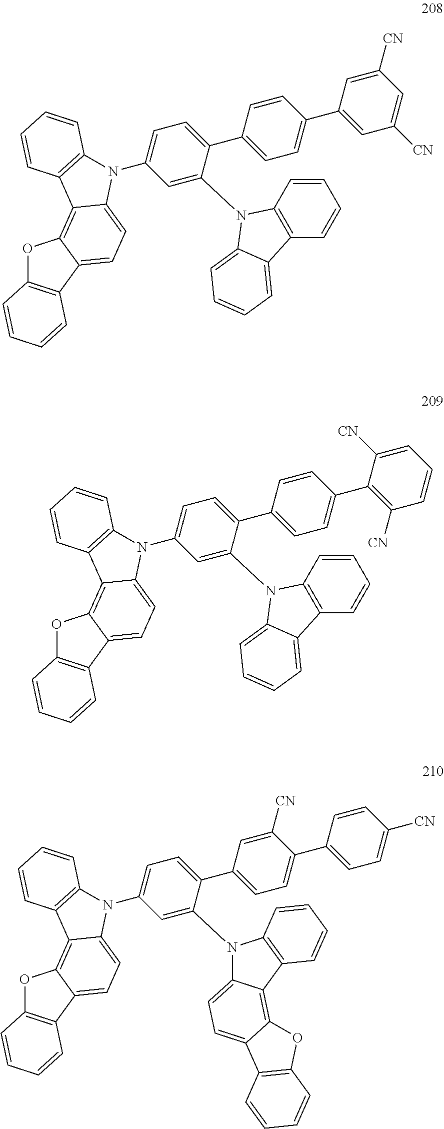

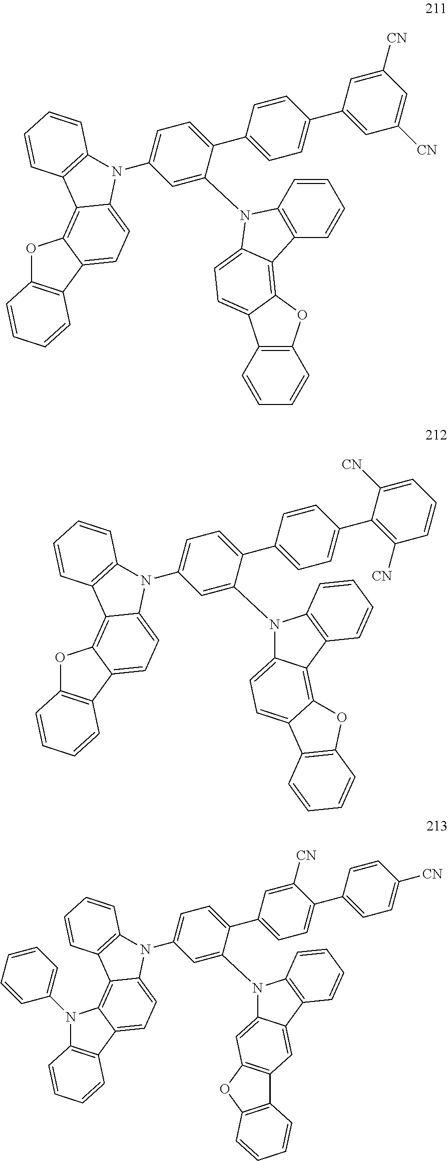

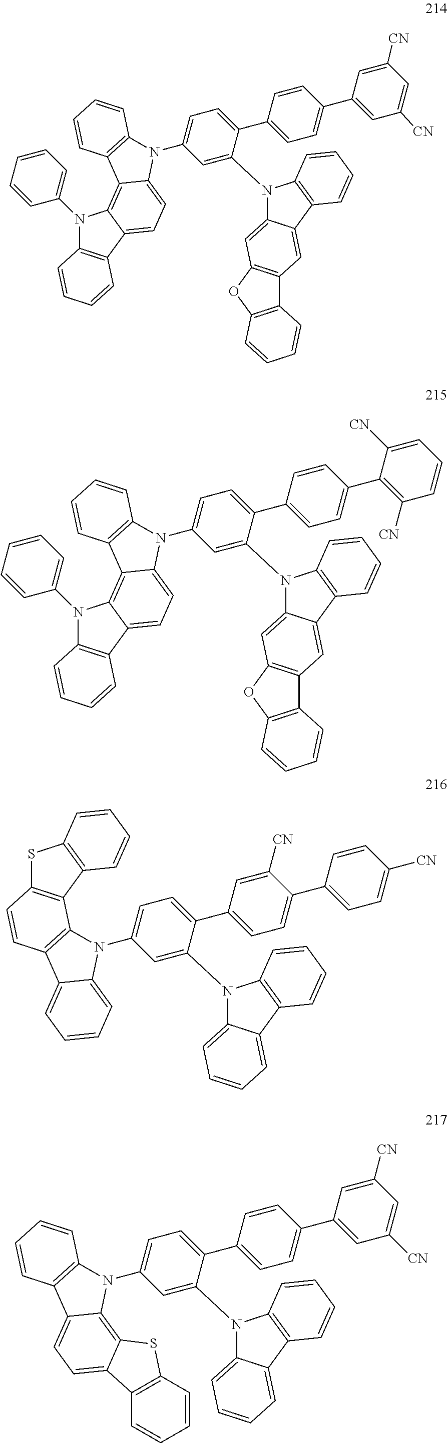

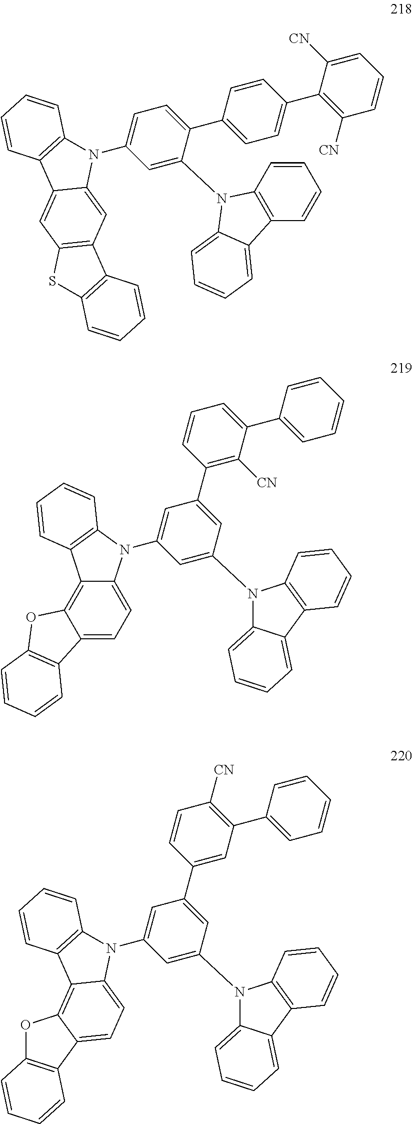

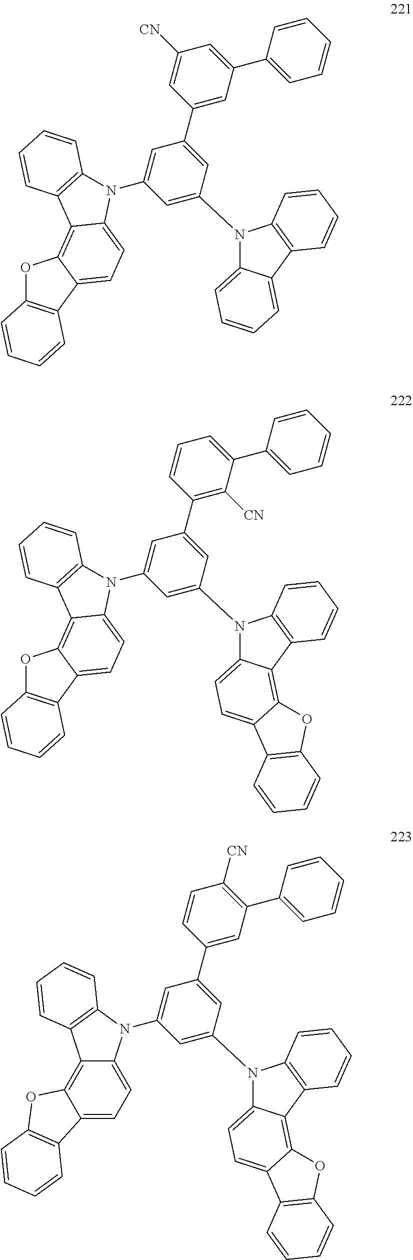

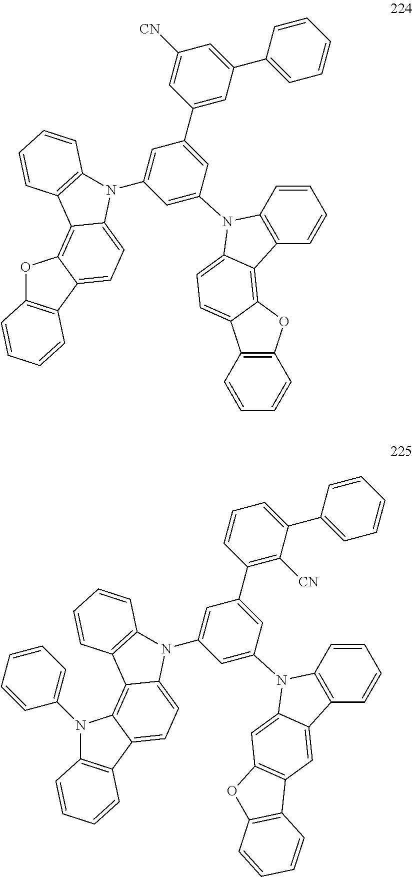

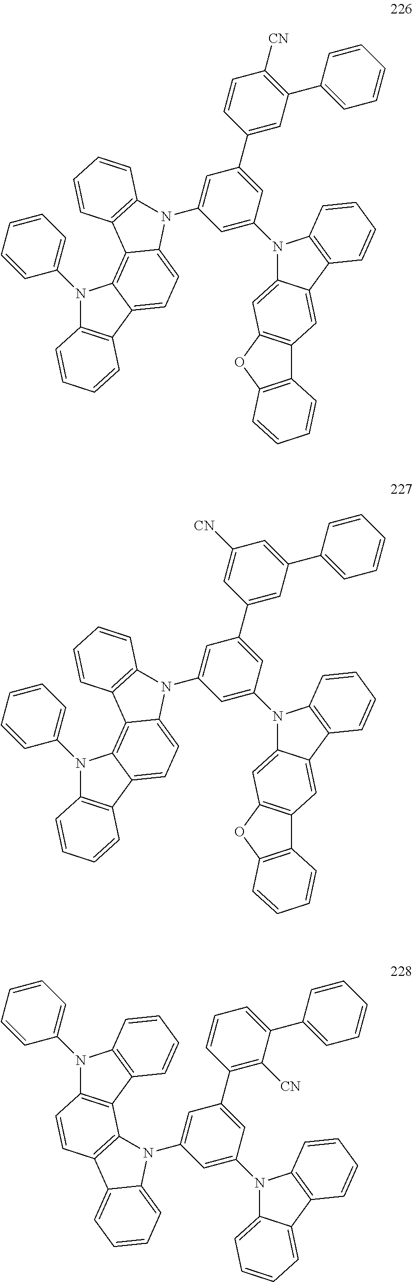

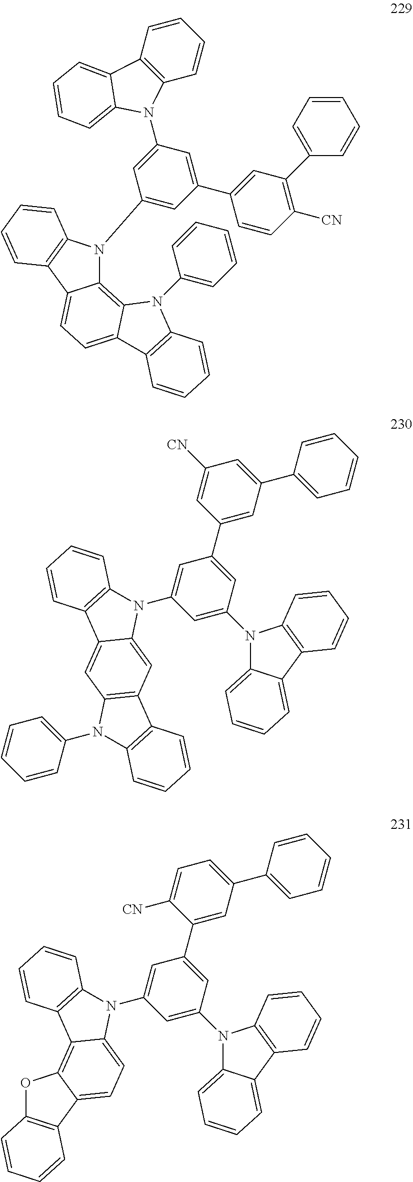

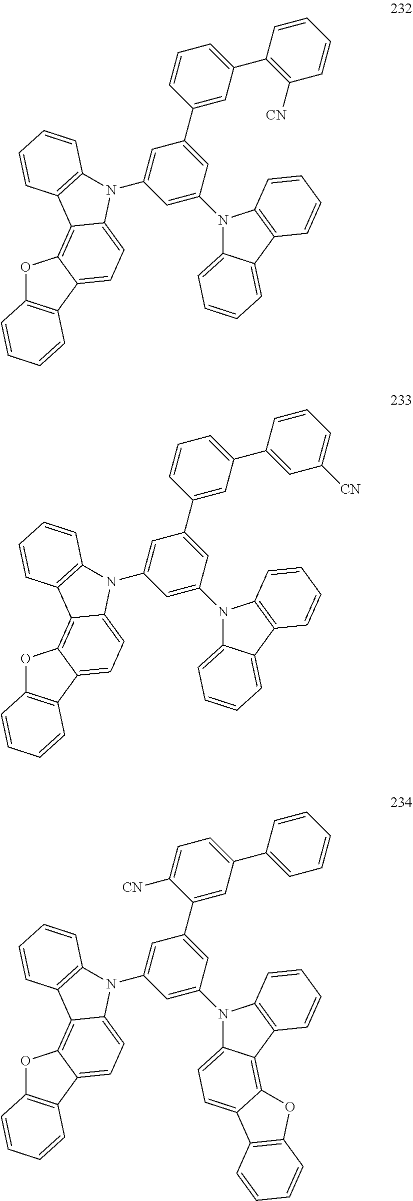

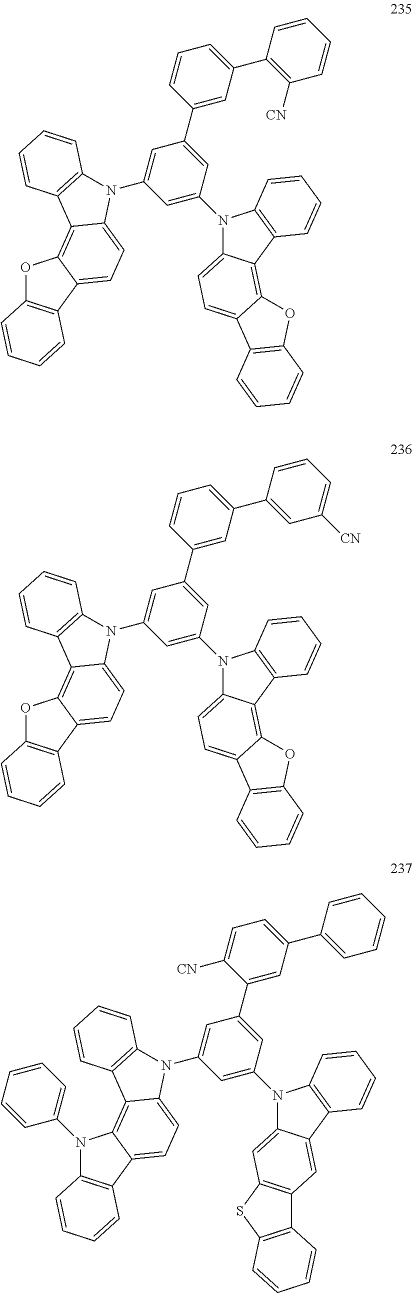

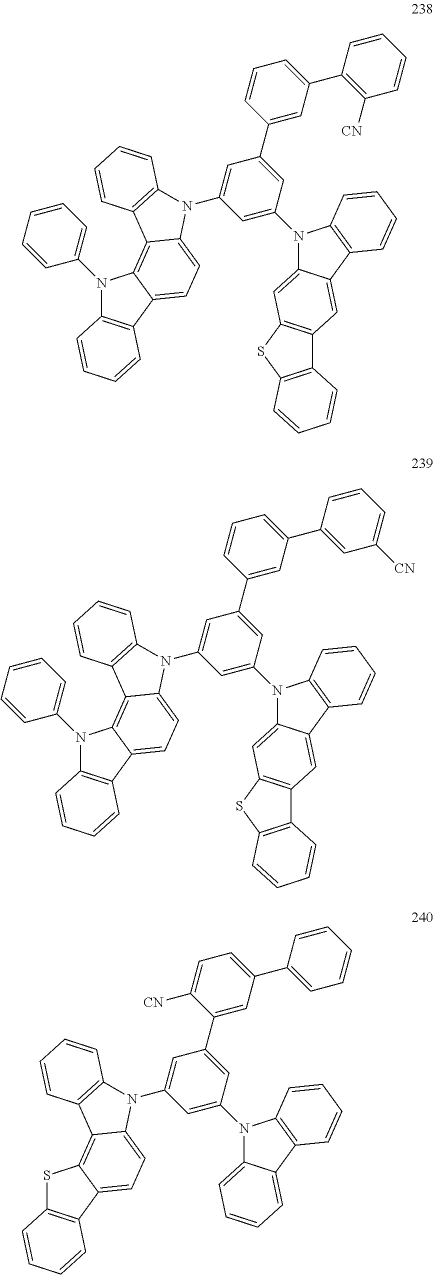

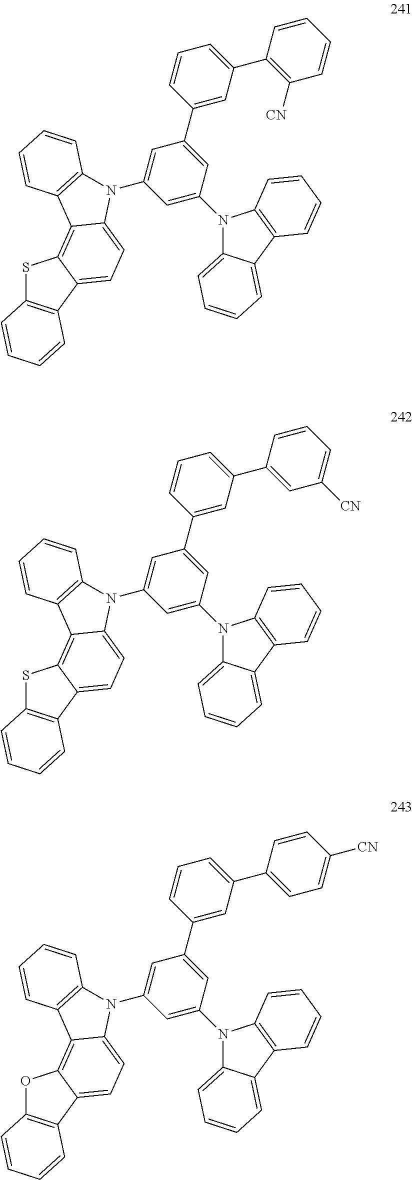

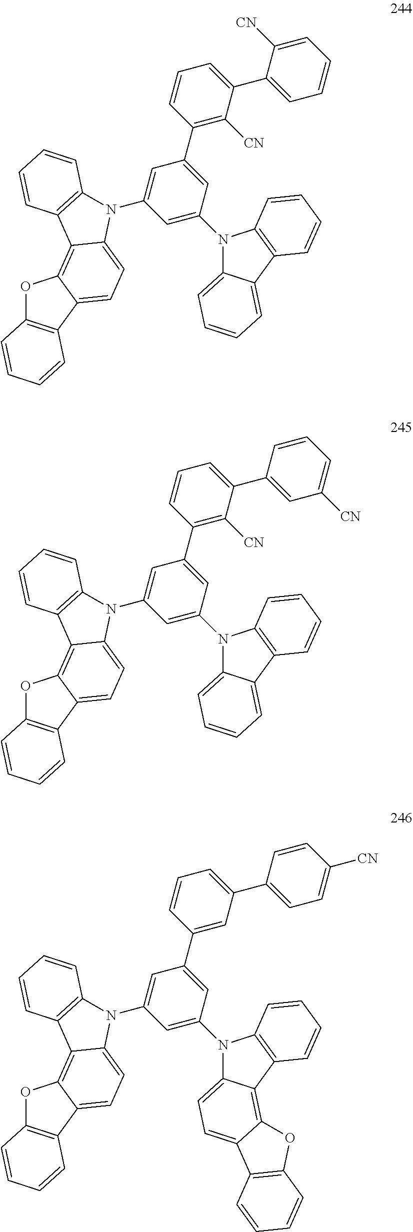

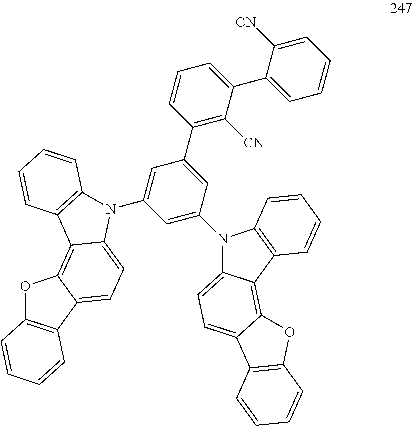

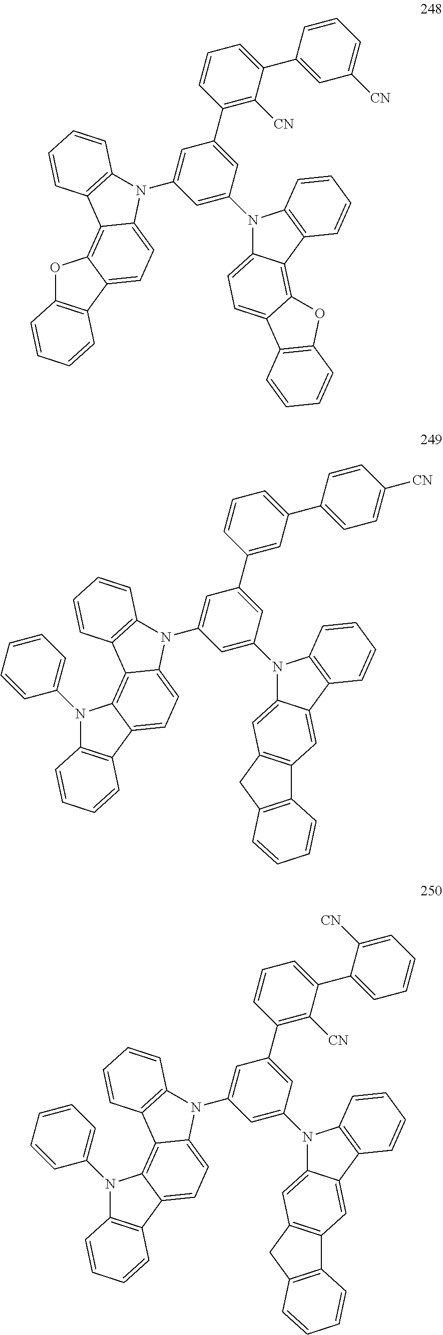

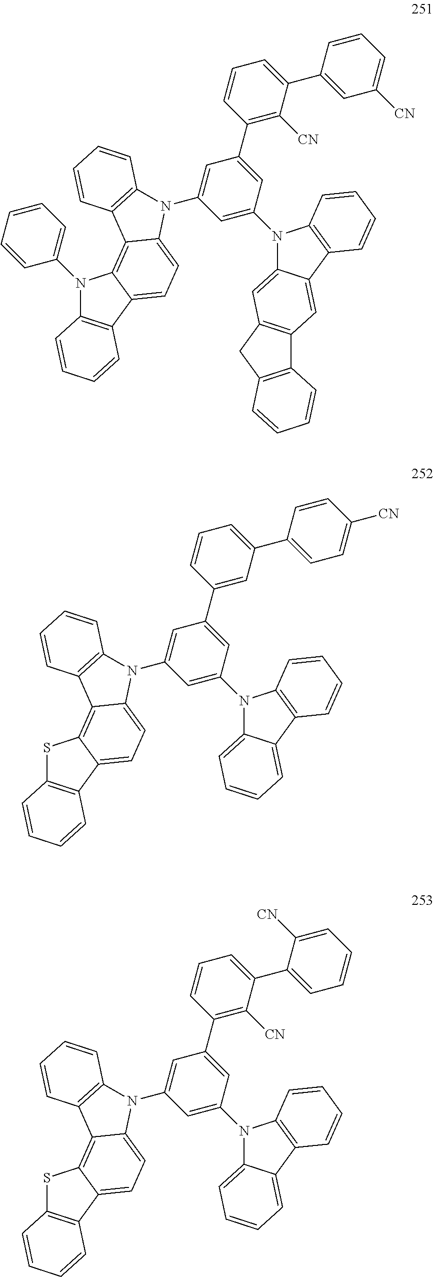

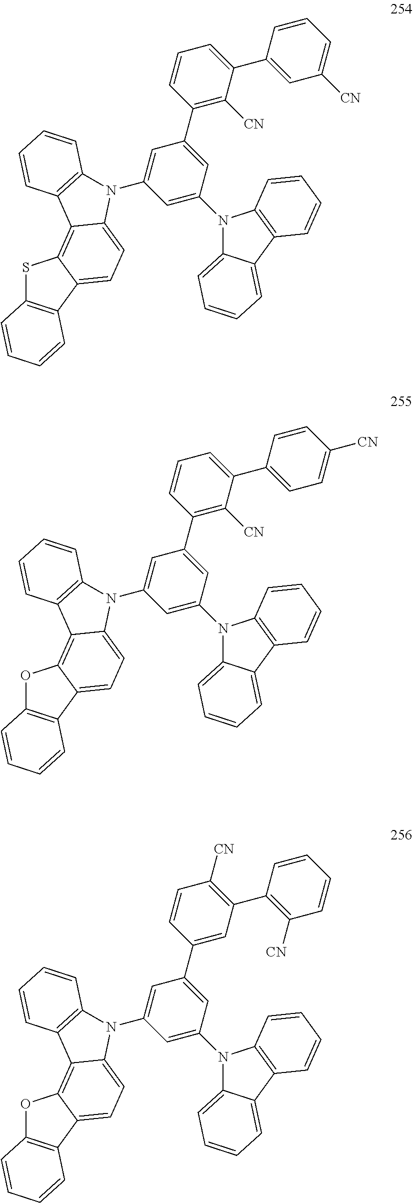

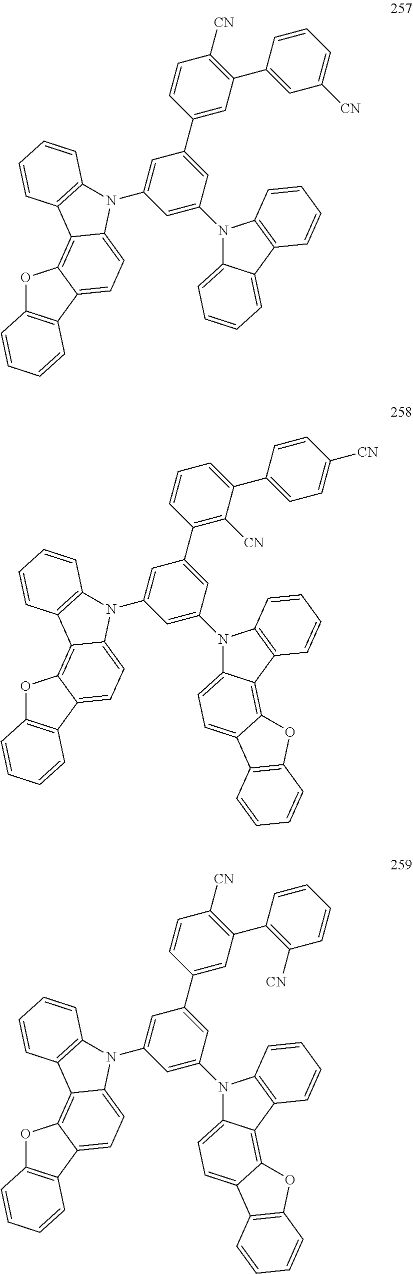

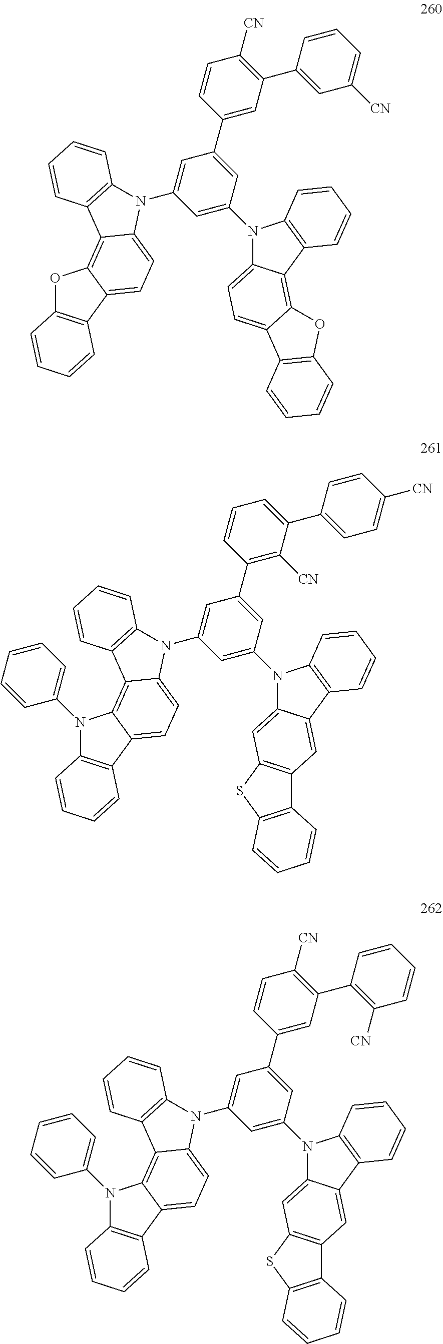

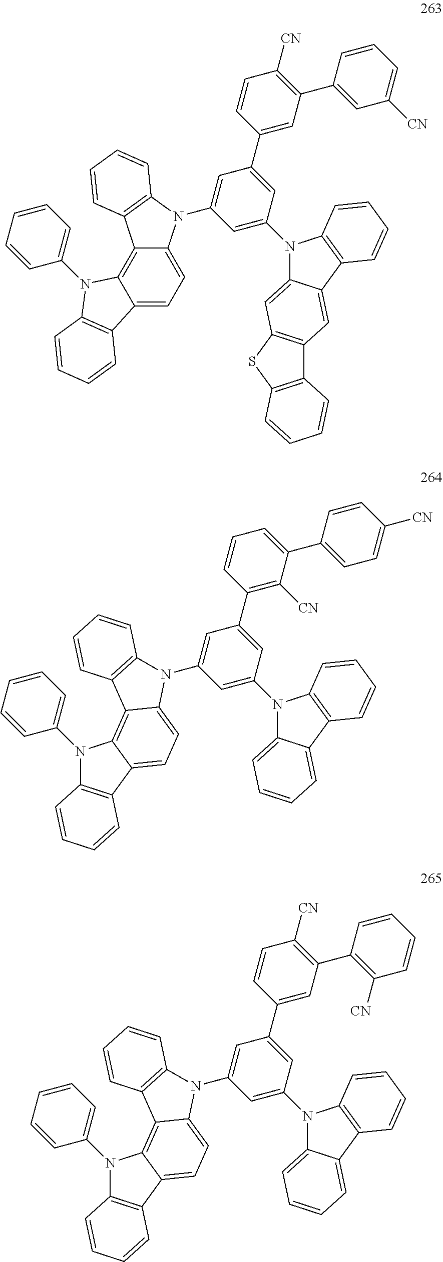

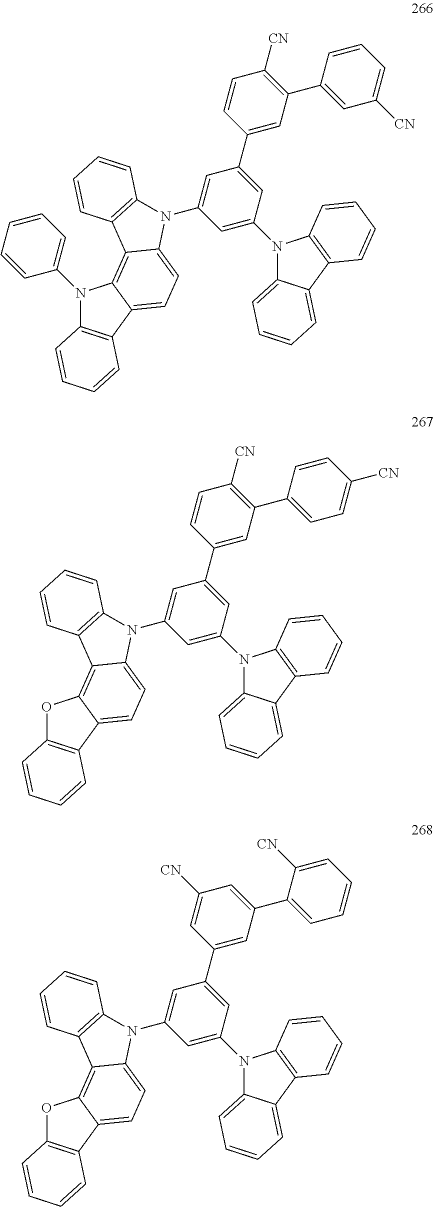

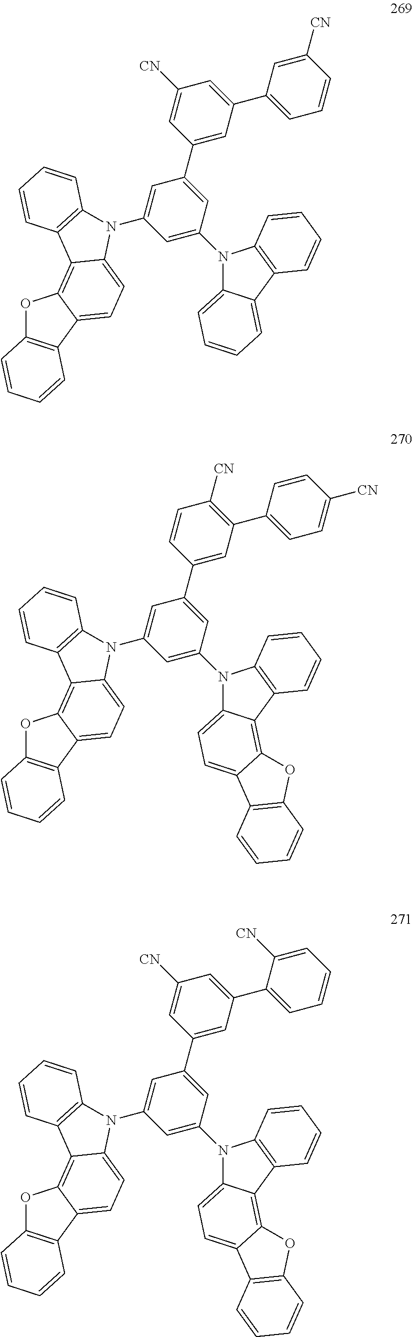

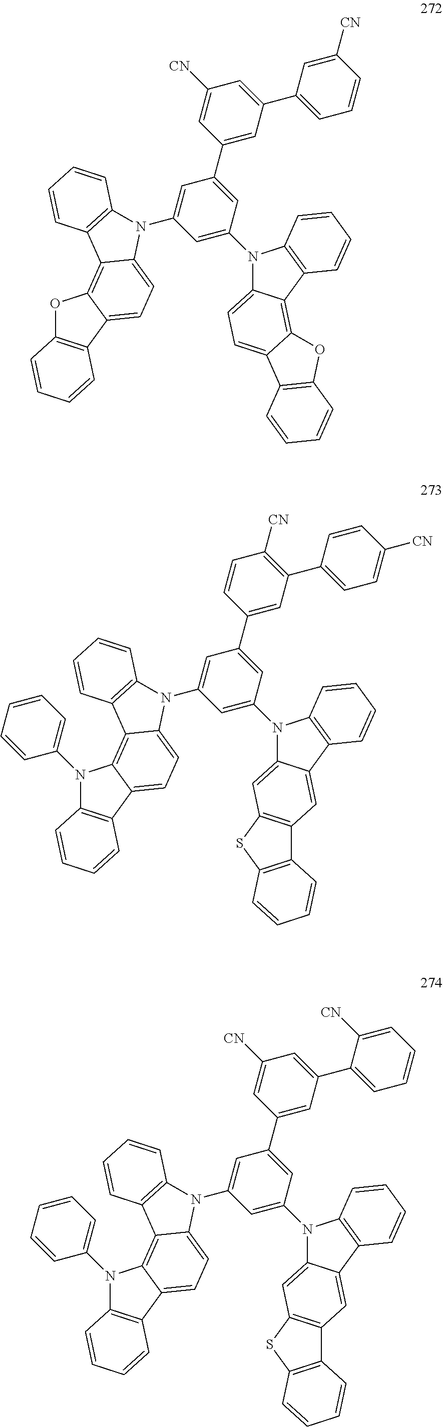

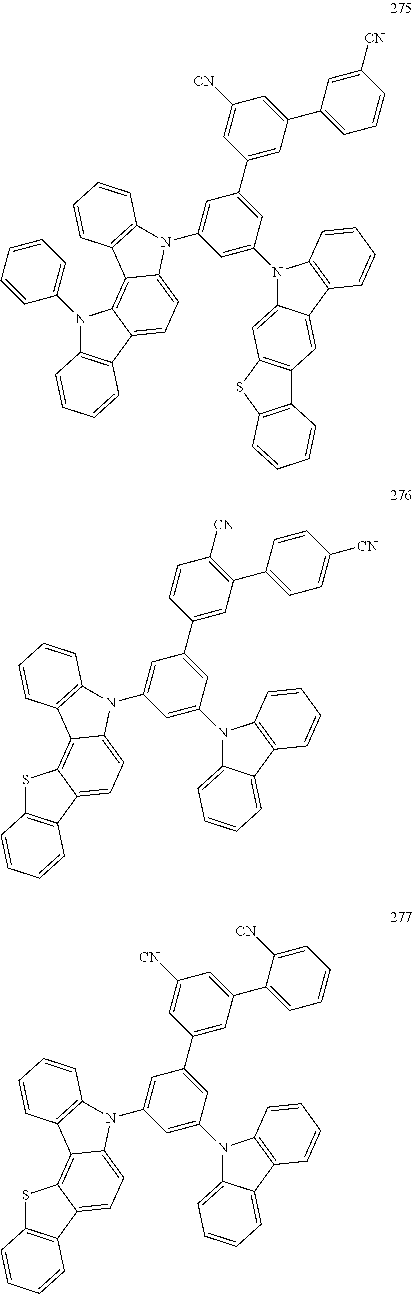

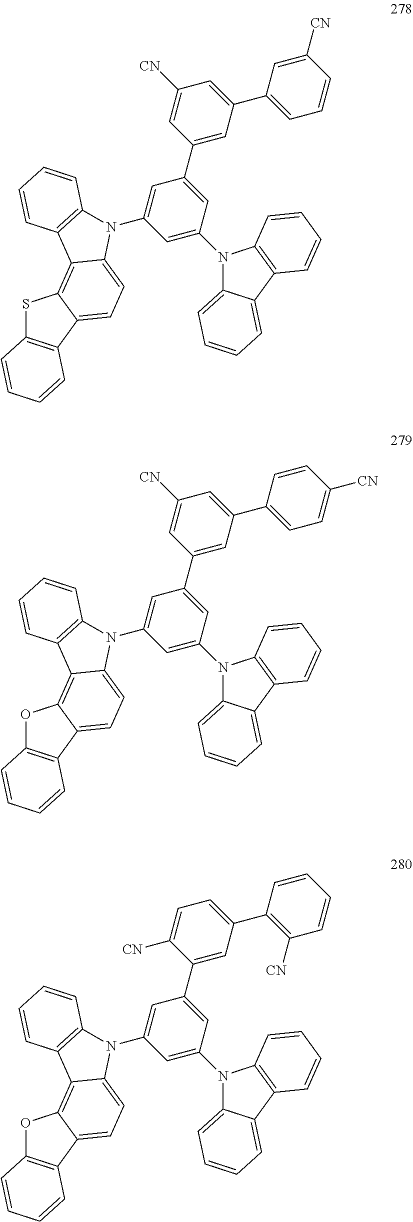

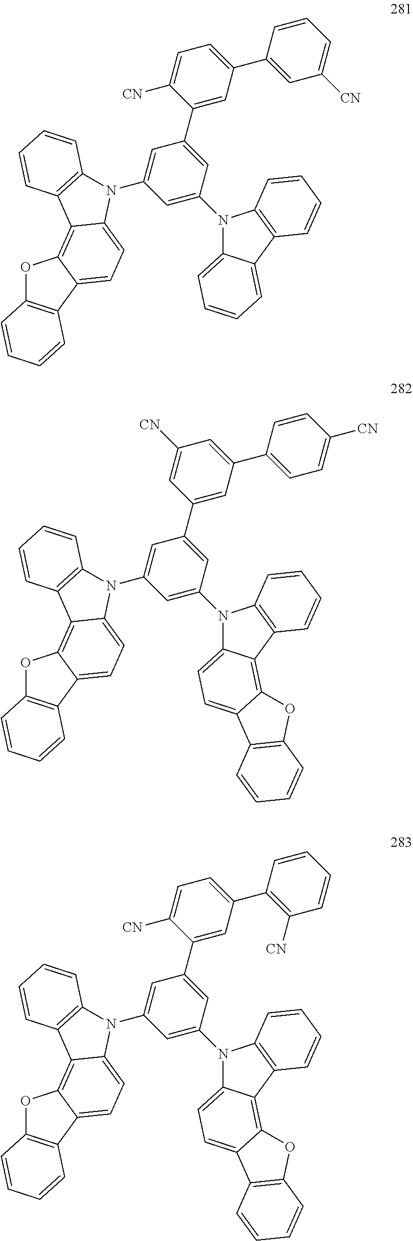

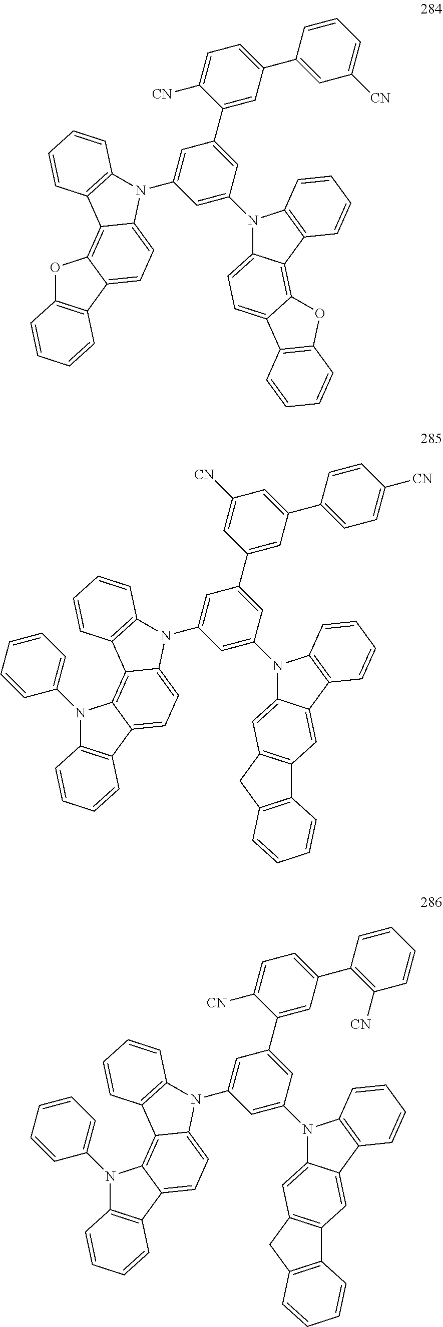

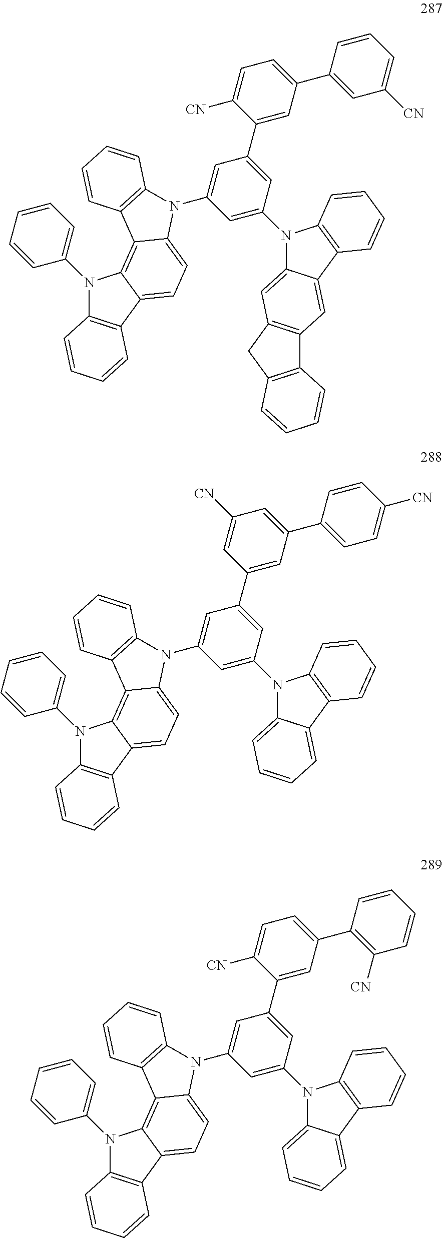

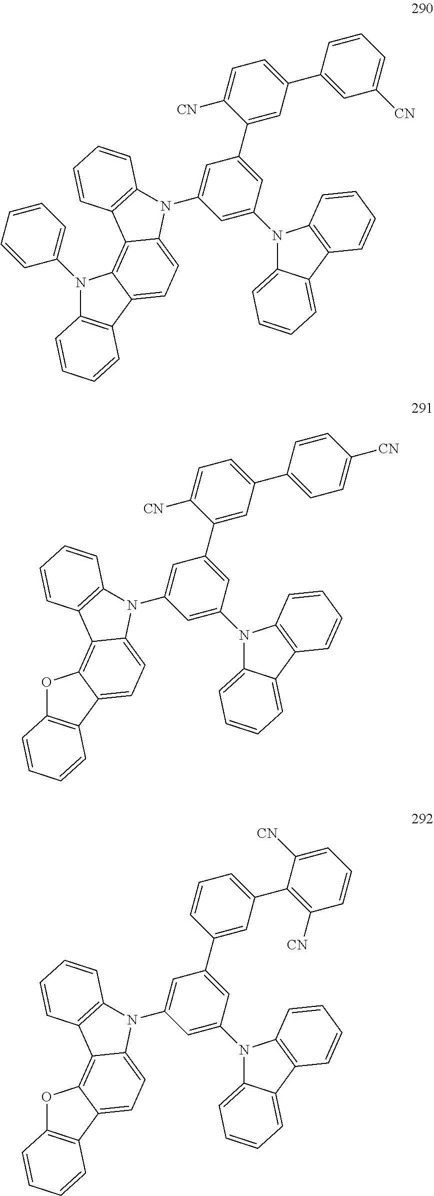

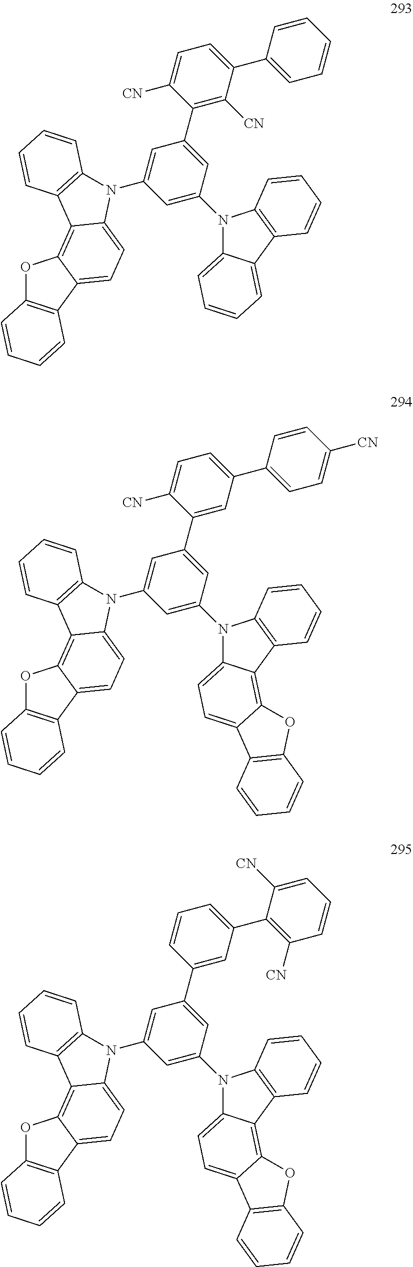

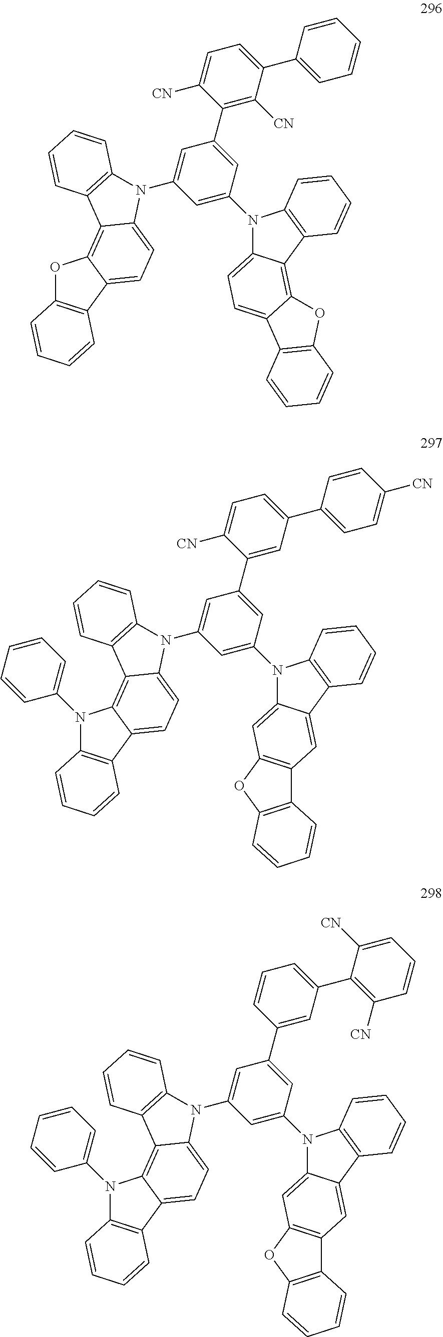

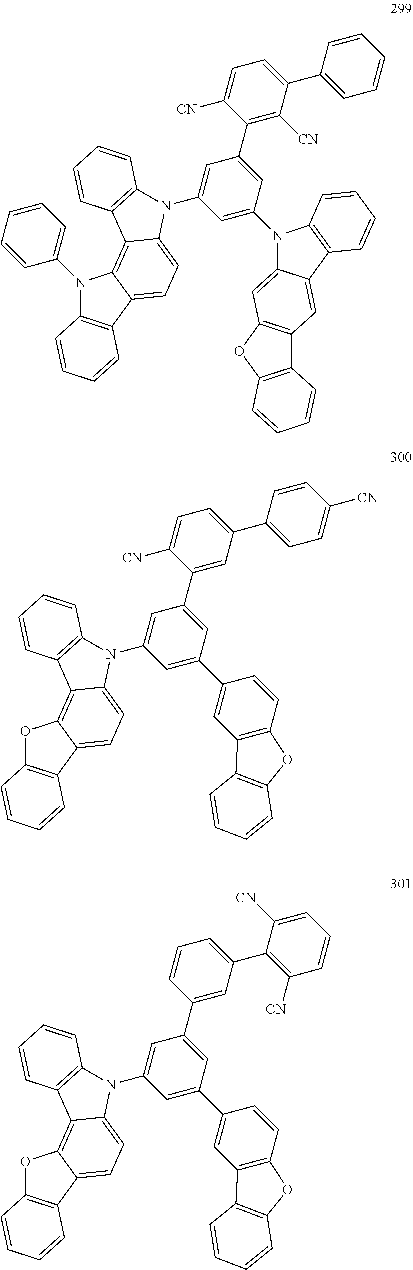

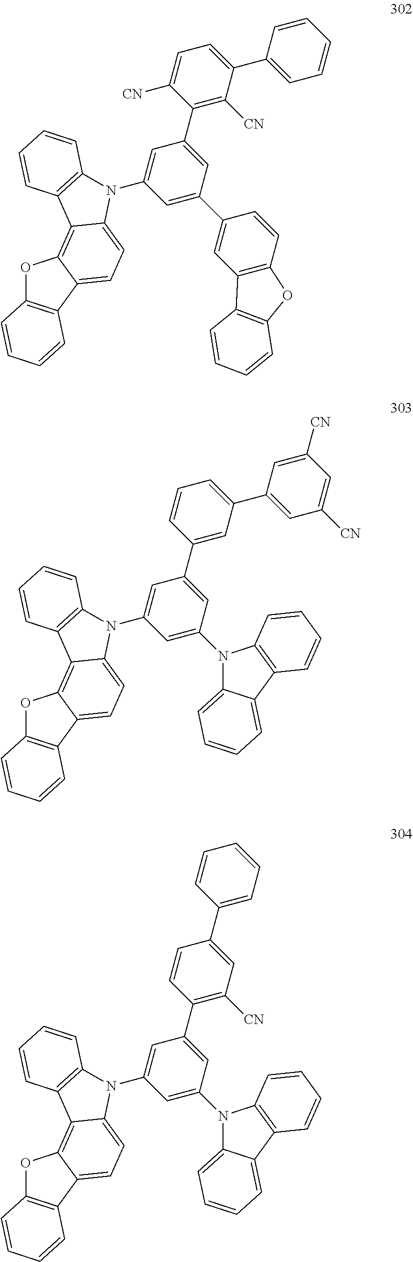

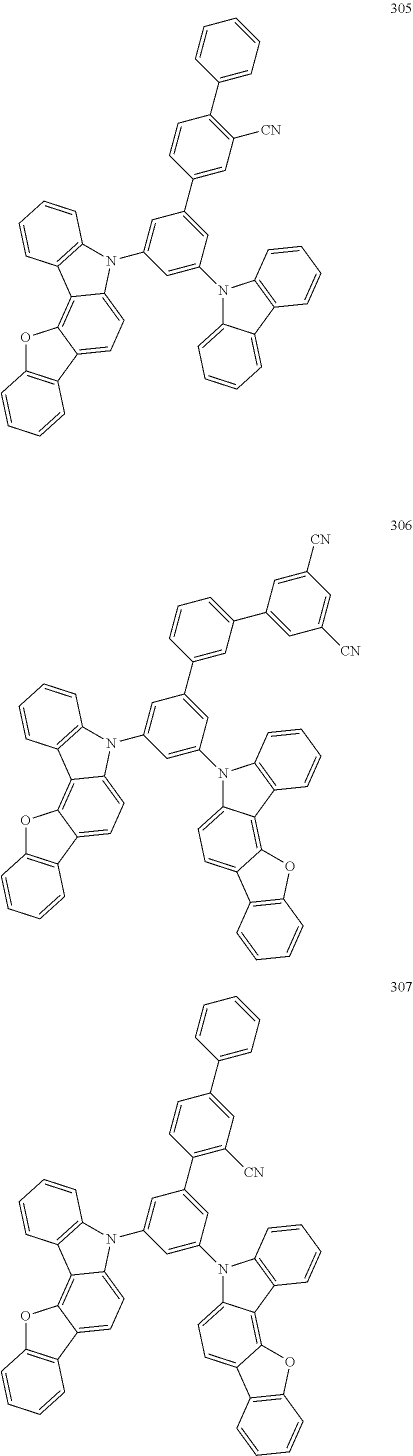

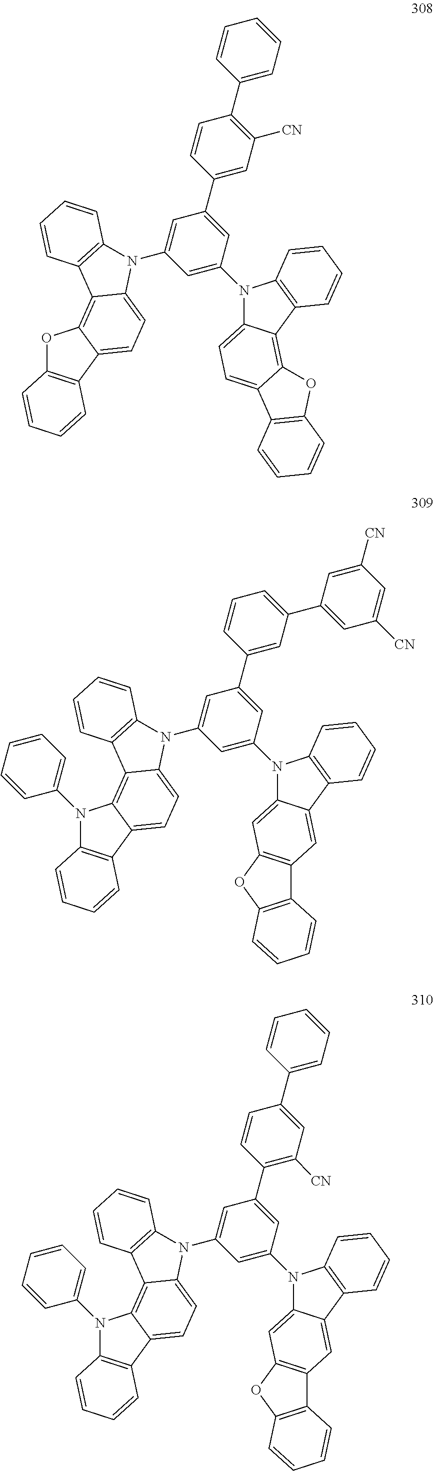 [SMILES: N#Cc1cc(-c2cc(-n3c4ccccc4c4c5oc6ccccc6c5ccc43)cc(-n3c4ccccc4c4c5oc6ccccc6c5ccc43)c2)ccc1-c1ccccc1 ; N#Cc1cc(C#N)cc(-c2cccc(-c3cc(-n4c5ccccc5c5cc6c(cc54)oc4ccccc46)cc(-n4c5ccccc5c5c4ccc4c6ccccc6n(-c6ccccc6)c45)c3)c2)c1 ; N#Cc1cc(-c2ccccc2)ccc1-c1cc(-n2c3ccccc3c3cc4c(cc32)oc2ccccc24)cc(-n2c3ccccc3c3c2ccc2c4ccccc4n(-c4ccccc4)c23)c1]

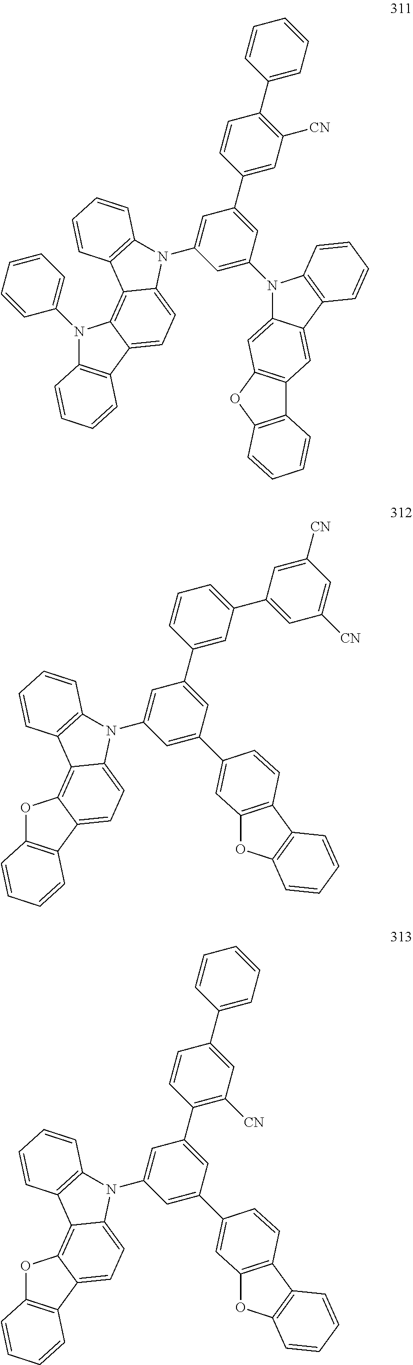 [SMILES: N#Cc1cc(-c2cc(-n3c4ccccc4c4cc5c(cc43)oc3ccccc35)cc(-n3c4ccccc4c4c3ccc3c5ccccc5n(-c5ccccc5)c34)c2)ccc1-c1ccccc1 ; N#Cc1cc(C#N)cc(-c2cccc(-c3cc(-c4ccc5c(c4)oc4ccccc45)cc(-n4c5ccccc5c5c6oc7ccccc7c6ccc54)c3)c2)c1 ; N#Cc1cc(-c2ccccc2)ccc1-c1cc(-c2ccc3c(c2)oc2ccccc23)cc(-n2c3ccccc3c3c4oc5ccccc5c4ccc32)c1]

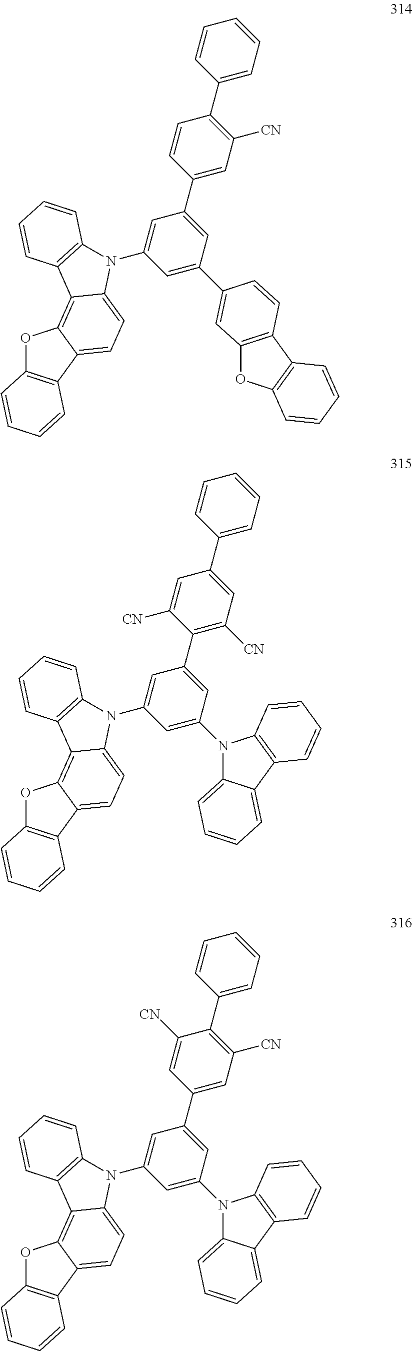

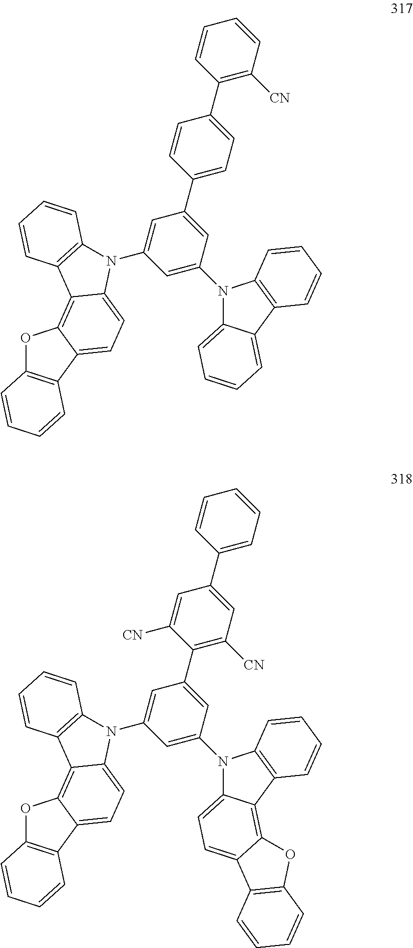

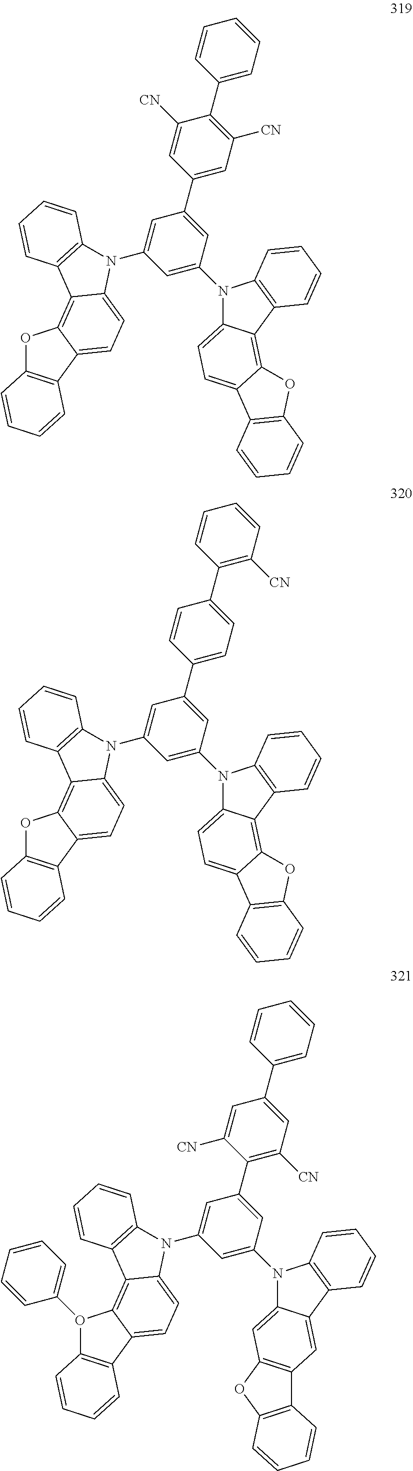

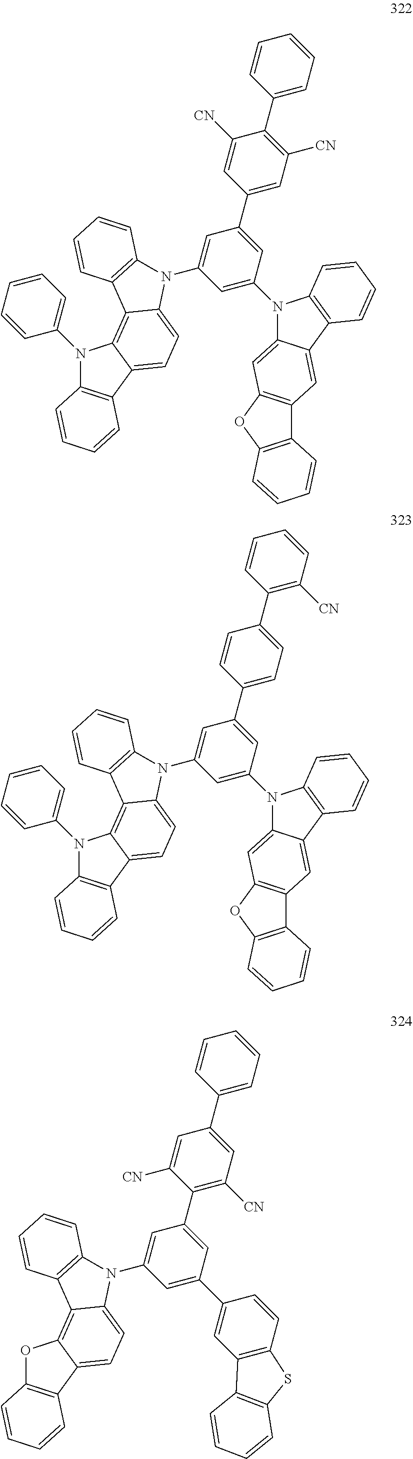

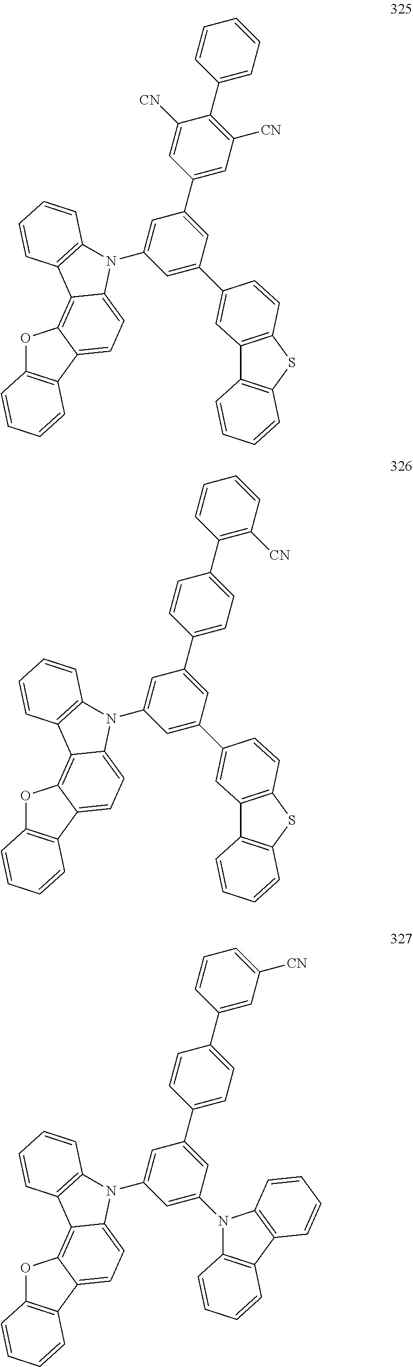

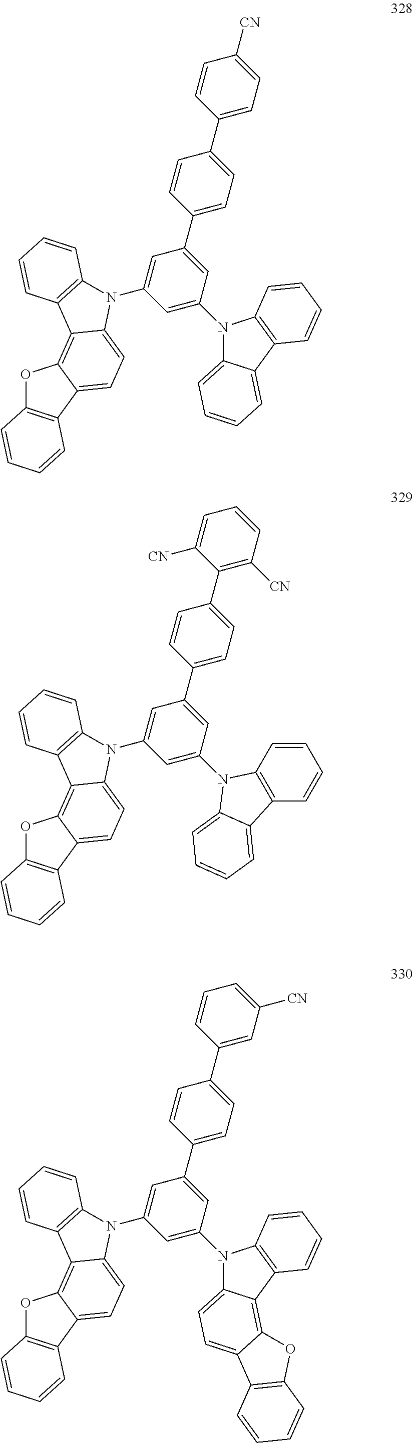

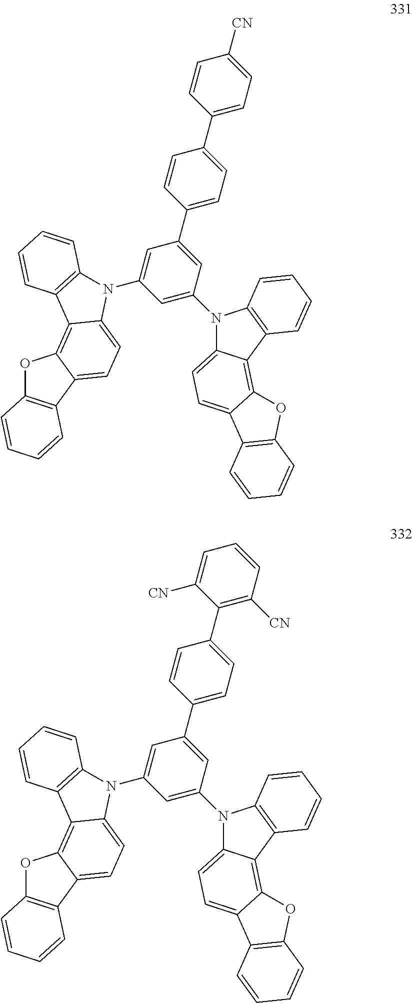

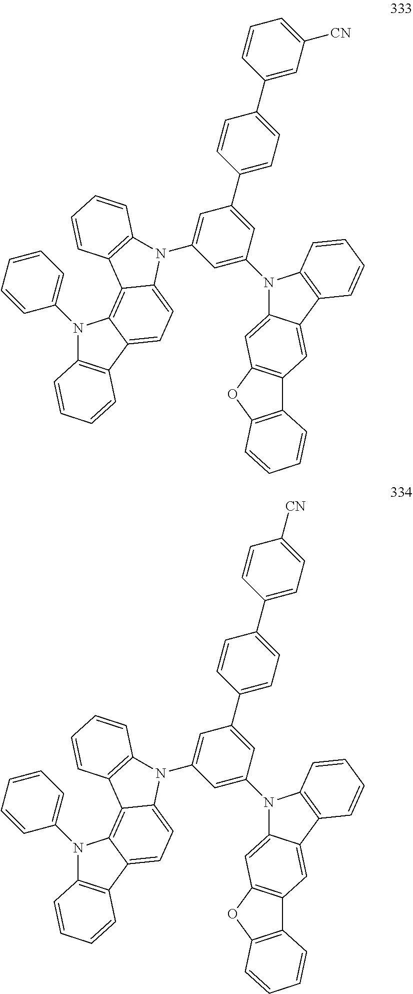

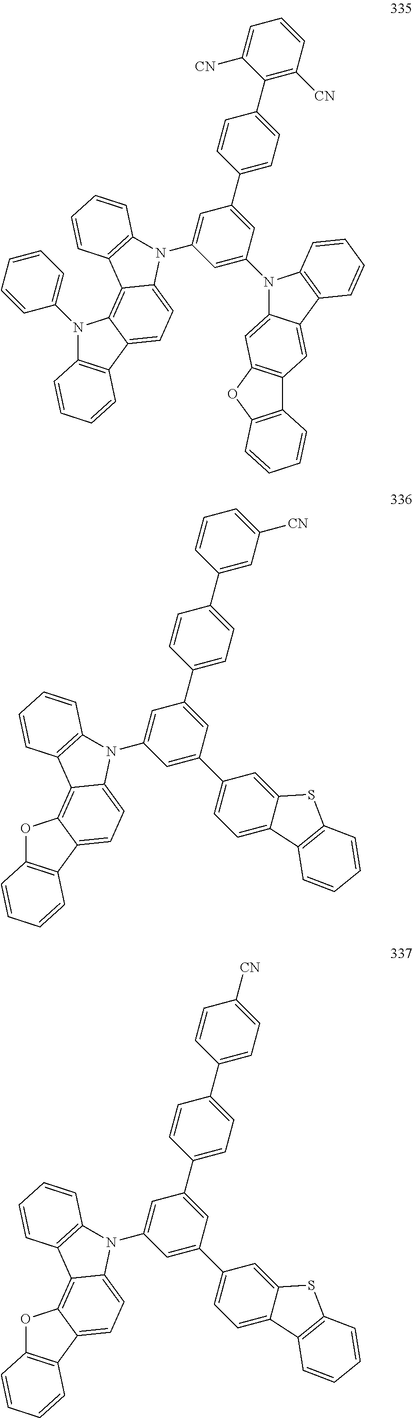

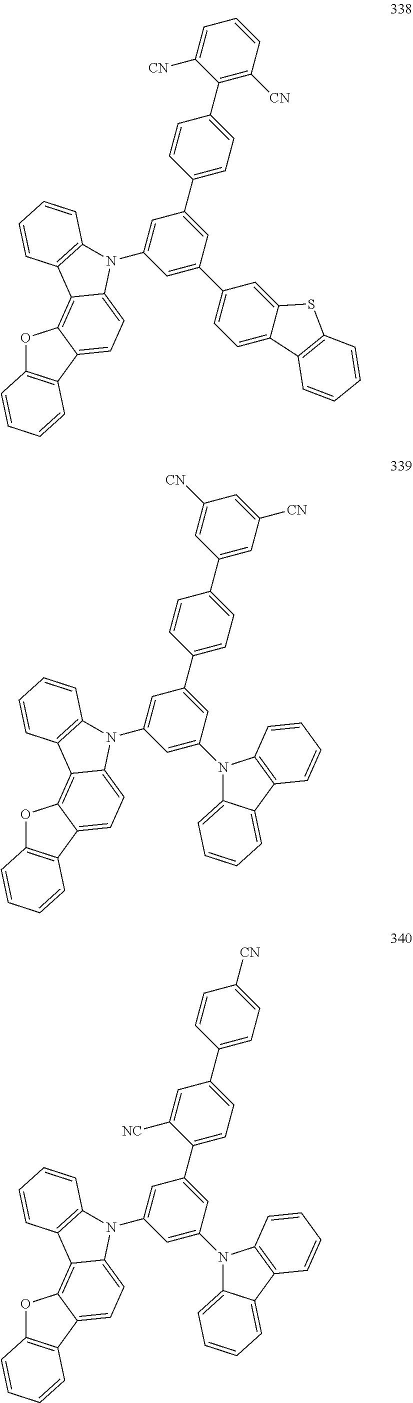

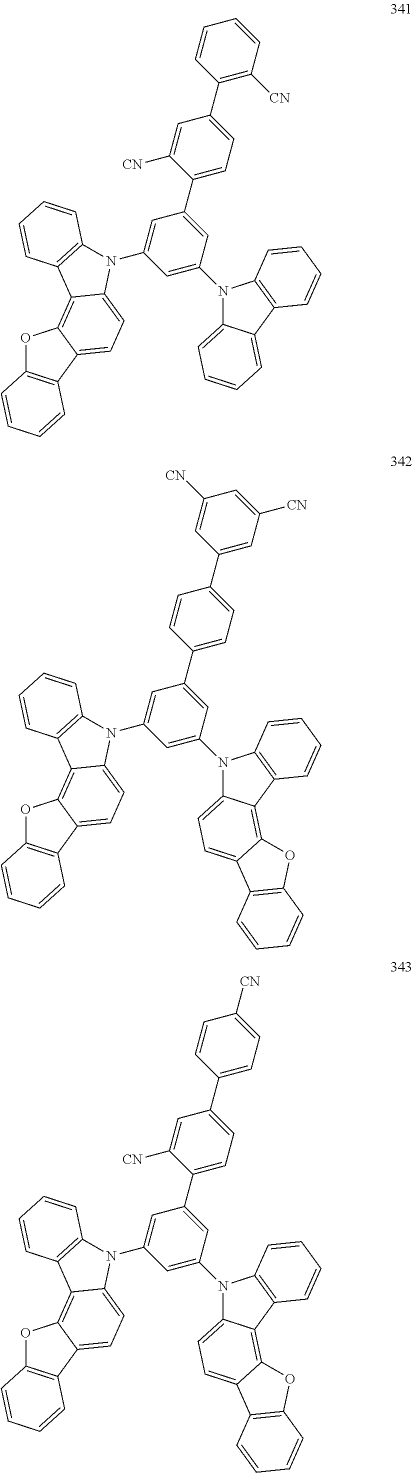

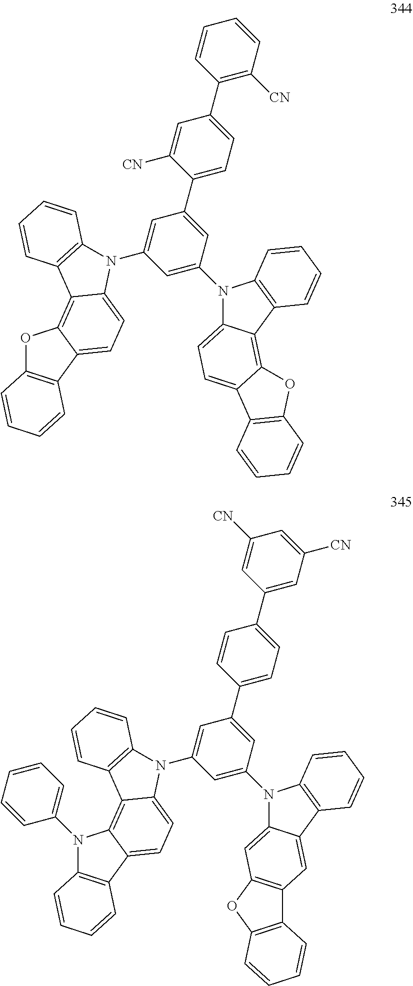

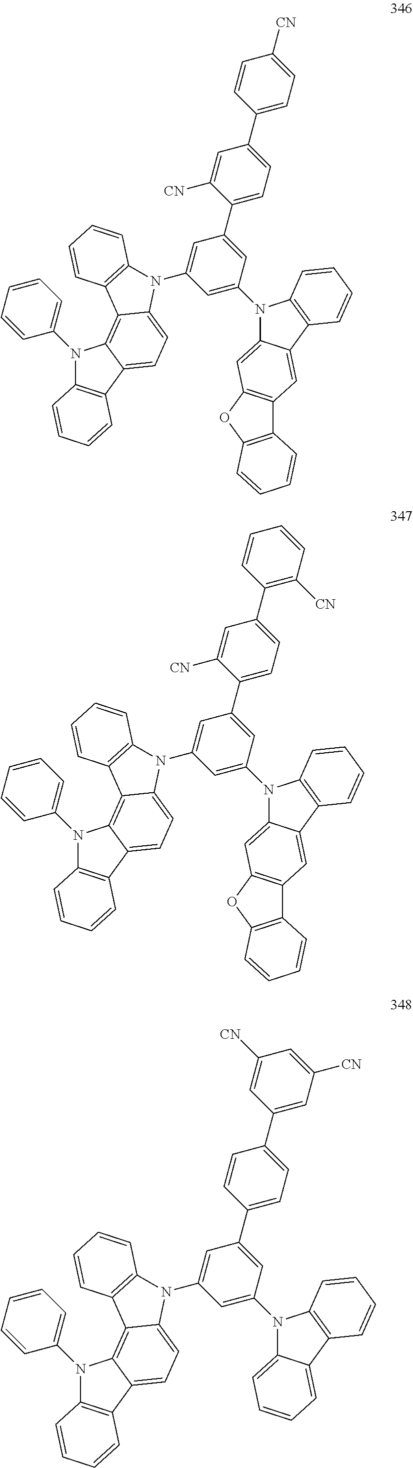

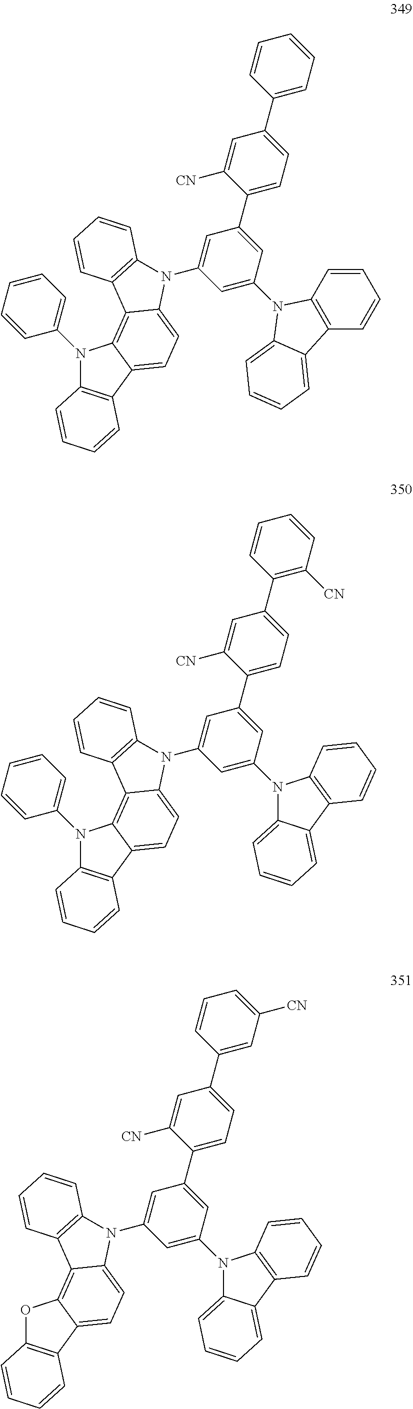

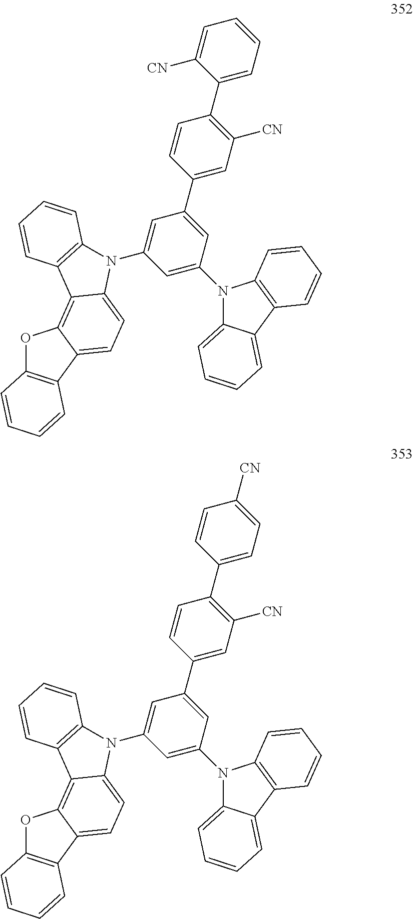

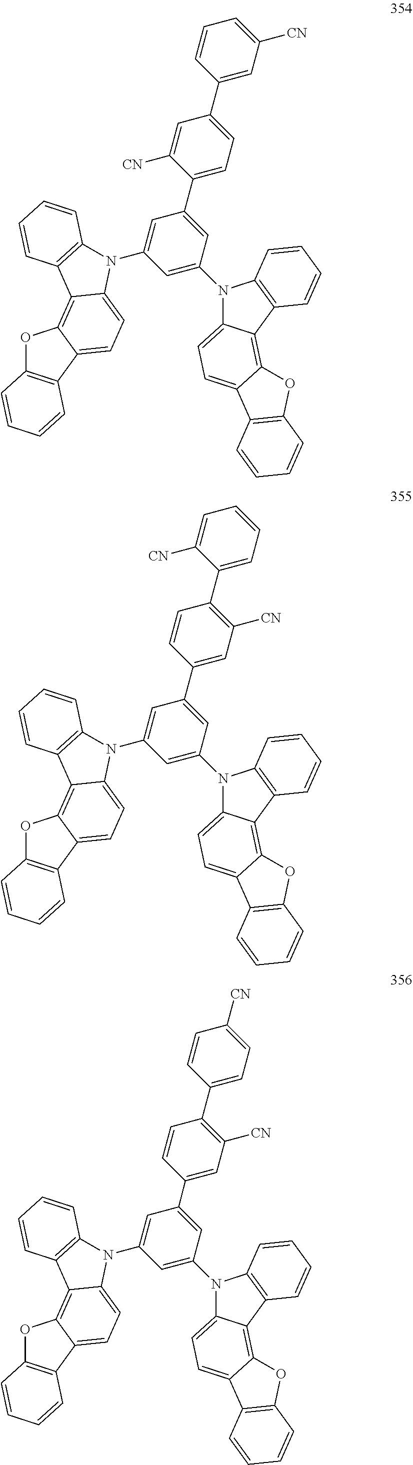

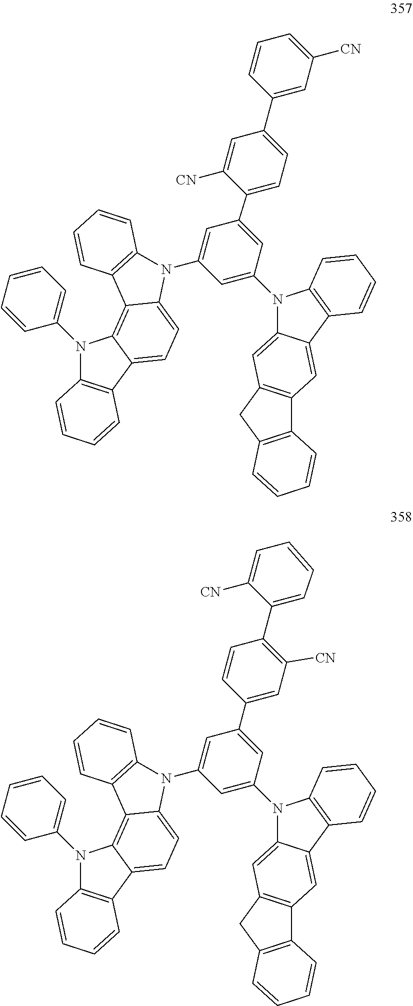

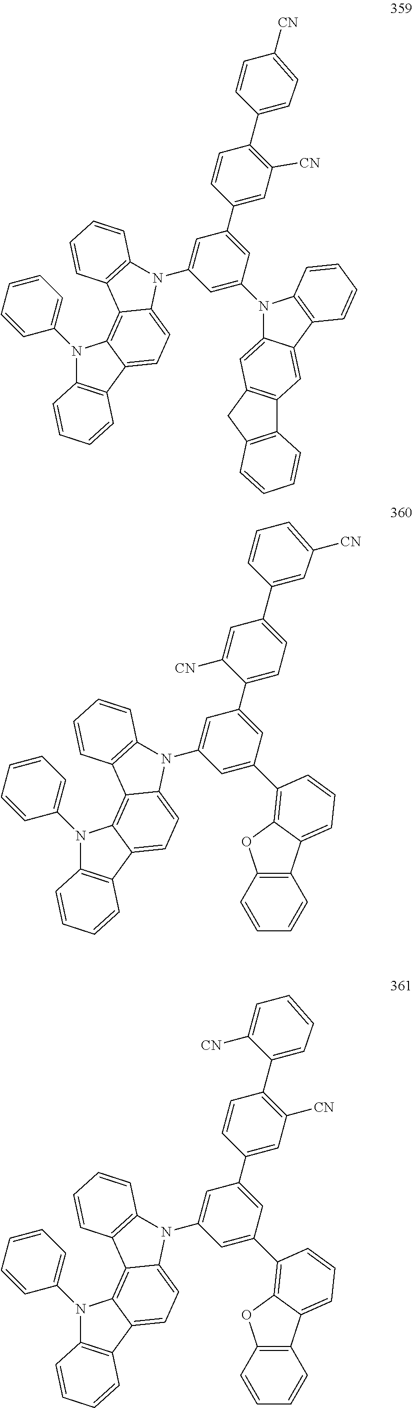

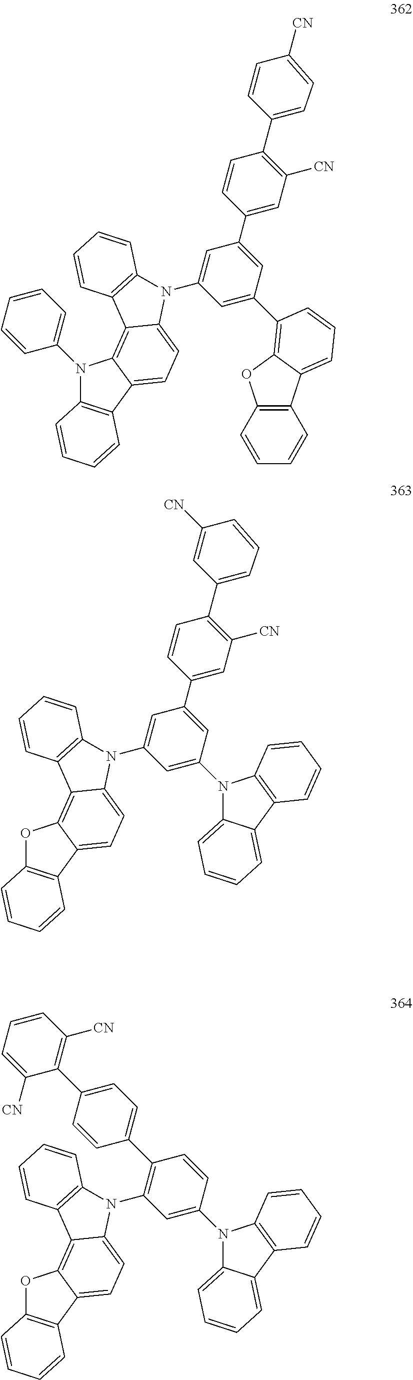

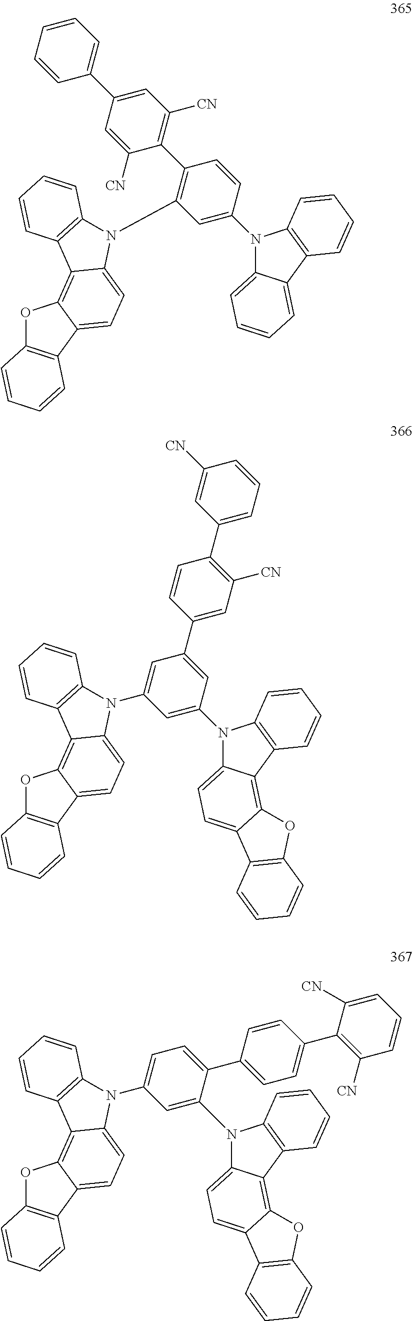

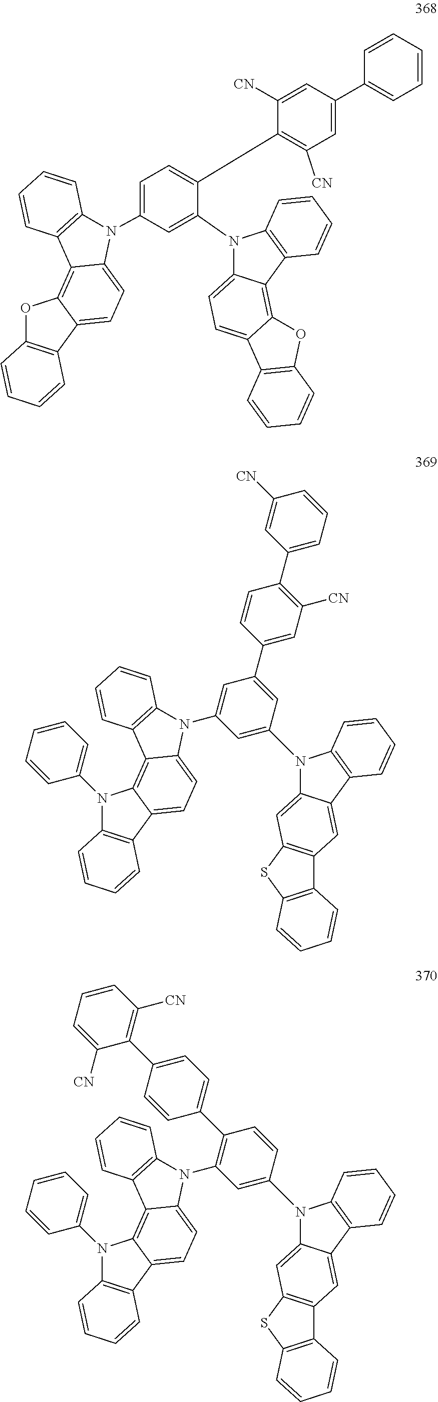

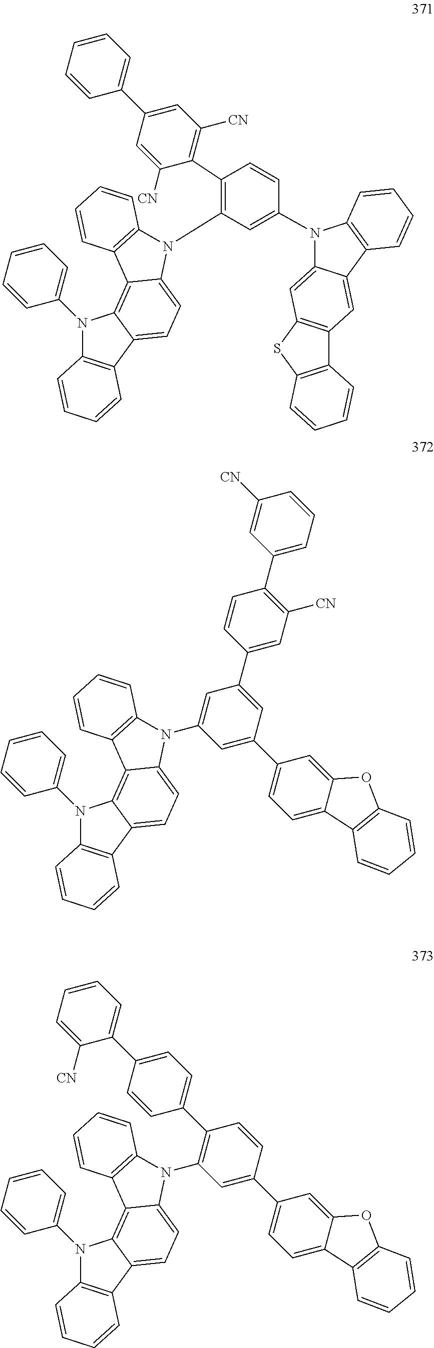

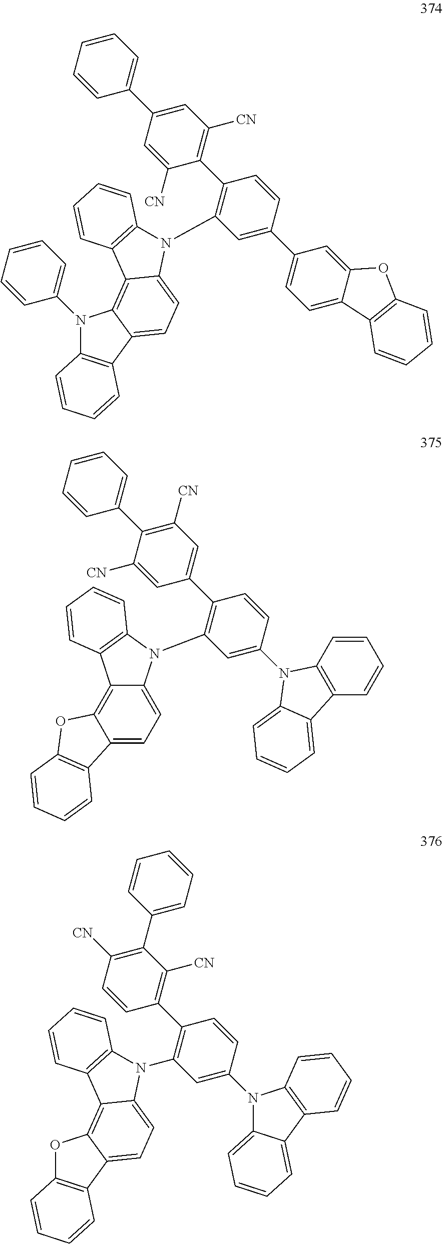

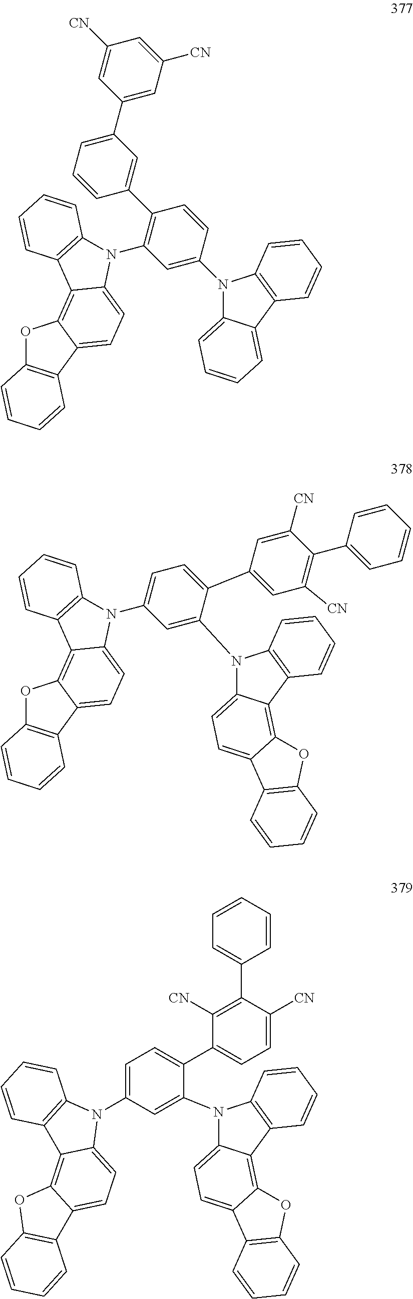

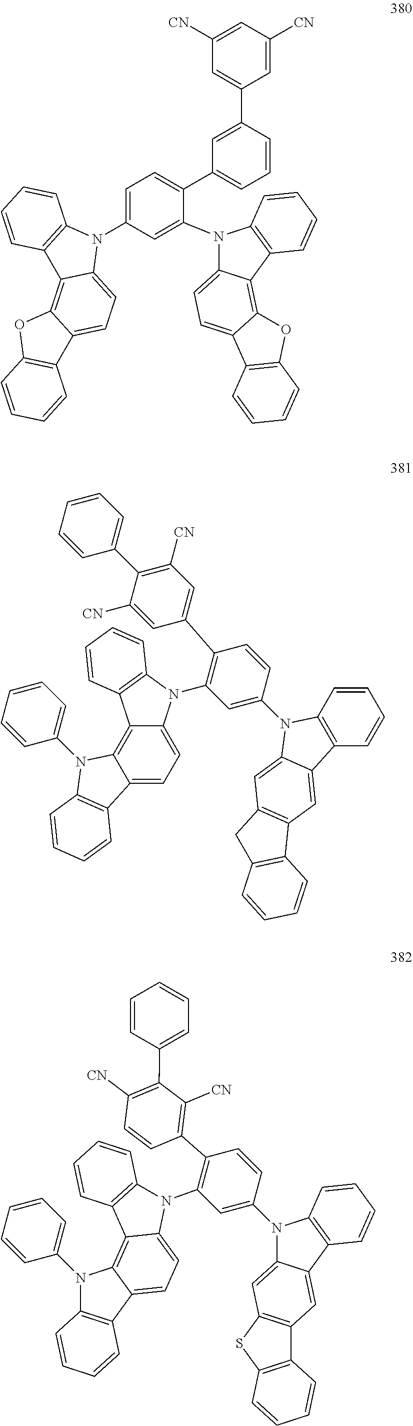

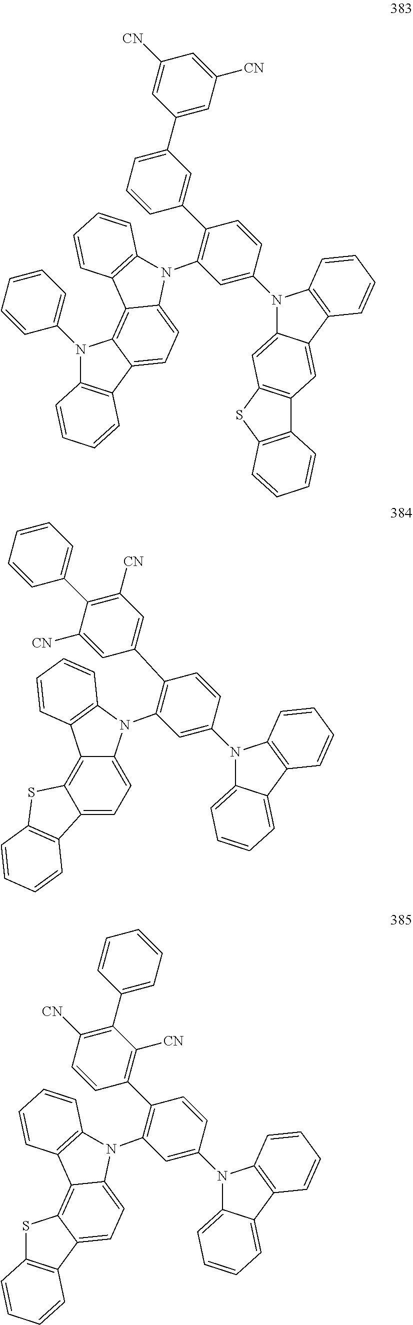

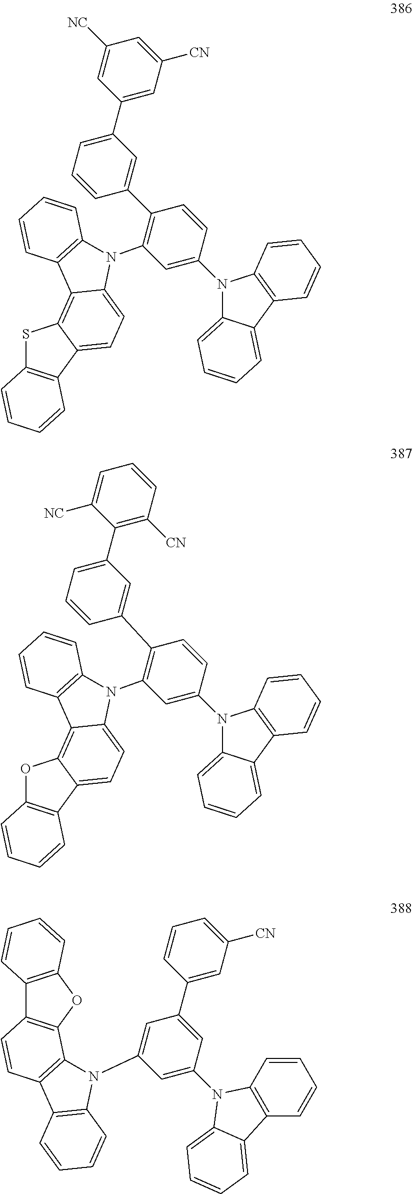 [SMILES: N#Cc1cc(C#N)cc(-c2cccc(-c3ccc(-n4c5ccccc5c5ccccc54)cc3-n3c4ccccc4c4c5sc6ccccc6c5ccc43)c2)c1 ; N#Cc1cccc(C#N)c1-c1cccc(-c2ccc(-n3c4ccccc4c4ccccc43)cc2-n2c3ccccc3c3c4oc5ccccc5c4ccc32)c1 ; N#Cc1cccc(-c2cc(-n3c4ccccc4c4ccccc43)cc(-n3c4ccccc4c4ccc5c6ccccc6oc5c43)c2)c1]

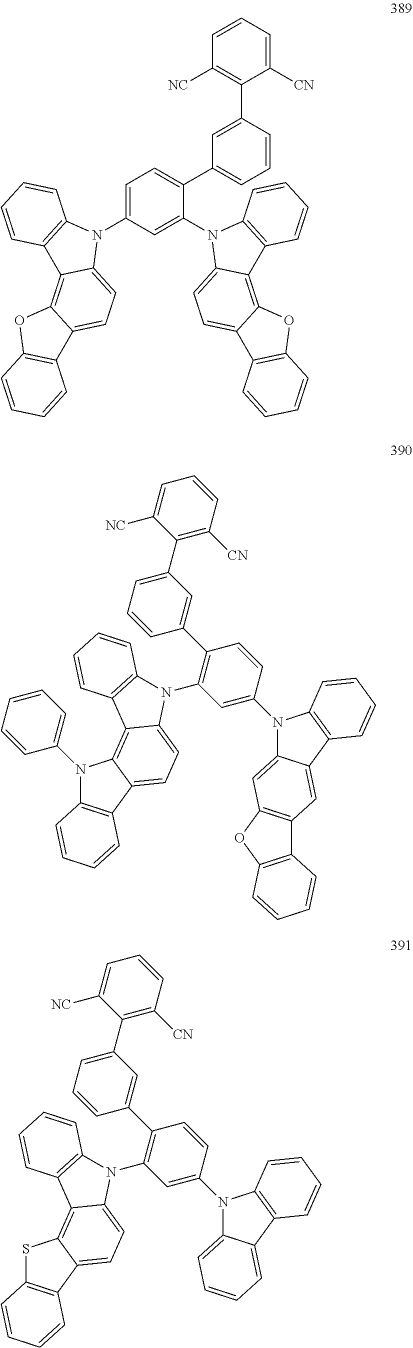 [SMILES: N#Cc1cccc(C#N)c1-c1cccc(-c2ccc(-n3c4ccccc4c4c5oc6ccccc6c5ccc43)cc2-n2c3ccccc3c3c4oc5ccccc5c4ccc32)c1 ; N#Cc1cccc(C#N)c1-c1cccc(-c2ccc(-n3c4ccccc4c4cc5c(cc43)oc3ccccc35)cc2-n2c3ccccc3c3c2ccc2c4ccccc4n(-c4ccccc4)c23)c1 ; N#Cc1cccc(C#N)c1-c1cccc(-c2ccc(-n3c4ccccc4c4ccccc43)cc2-n2c3ccccc3c3c4sc5ccccc5c4ccc32)c1]

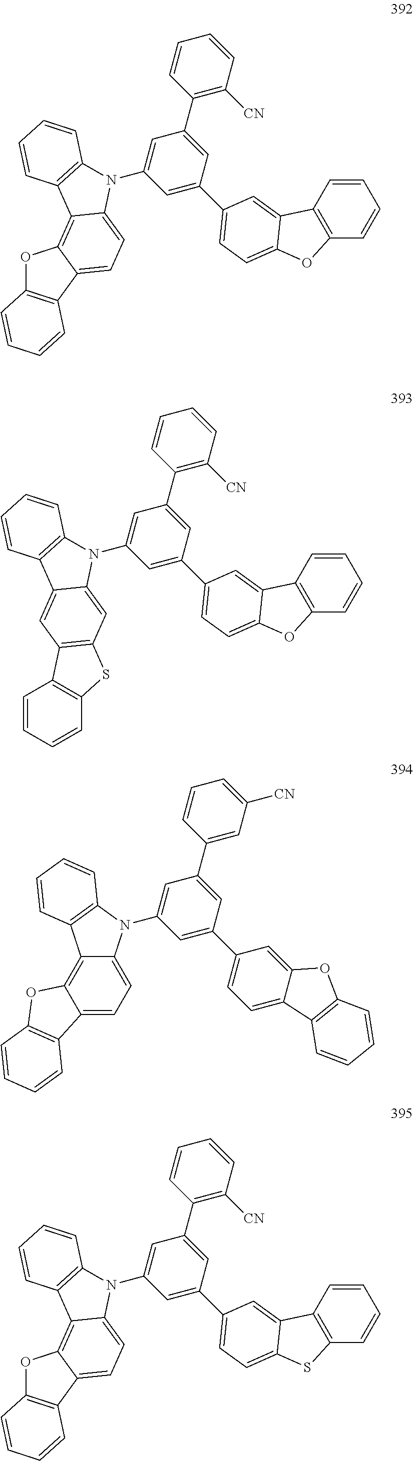 [SMILES: N#Cc1ccccc1-c1cc(-c2ccc3oc4ccccc4c3c2)cc(-n2c3ccccc3c3c4oc5ccccc5c4ccc32)c1 ; N#Cc1ccccc1-c1cc(-c2ccc3oc4ccccc4c3c2)cc(-n2c3ccccc3c3cc4c(cc32)sc2ccccc24)c1 ; N#Cc1cccc(-c2cc(-c3ccc4c(c3)oc3ccccc34)cc(-n3c4ccccc4c4c5oc6ccccc6c5ccc43)c2)c1 ; N#Cc1ccccc1-c1cc(-c2ccc3sc4ccccc4c3c2)cc(-n2c3ccccc3c3c4oc5ccccc5c4ccc32)c1]

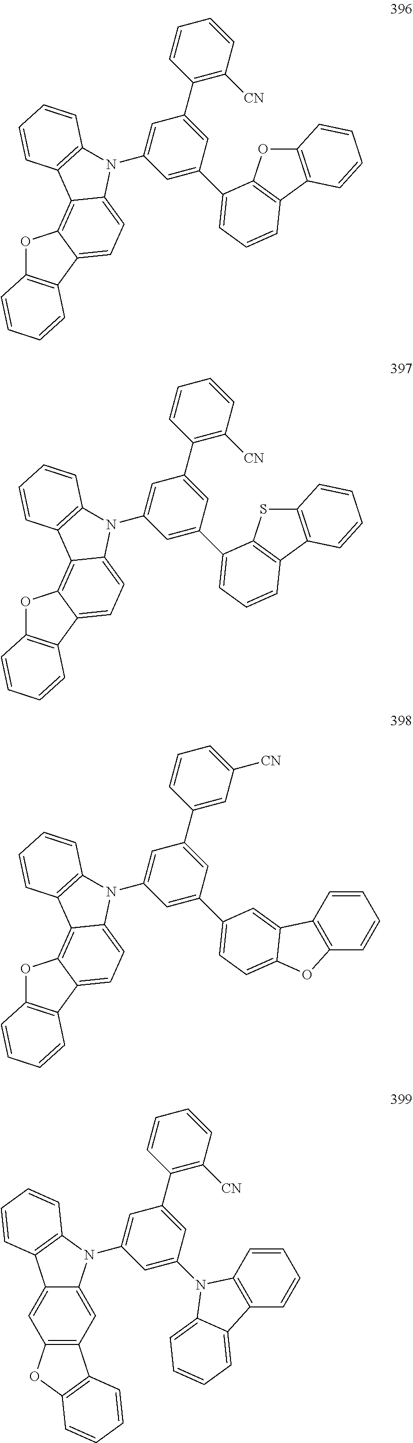 [SMILES: N#Cc1ccccc1-c1cc(-c2cccc3c2oc2ccccc23)cc(-n2c3ccccc3c3c4oc5ccccc5c4ccc32)c1 ; N#Cc1ccccc1-c1cc(-c2cccc3c2sc2ccccc23)cc(-n2c3ccccc3c3c4oc5ccccc5c4ccc32)c1 ; N#Cc1cccc(-c2cc(-c3ccc4oc5ccccc5c4c3)cc(-n3c4ccccc4c4c5oc6ccccc6c5ccc43)c2)c1 ; N#Cc1ccccc1-c1cc(-n2c3ccccc3c3ccccc32)cc(-n2c3ccccc3c3cc4oc5ccccc5c4cc32)c1]

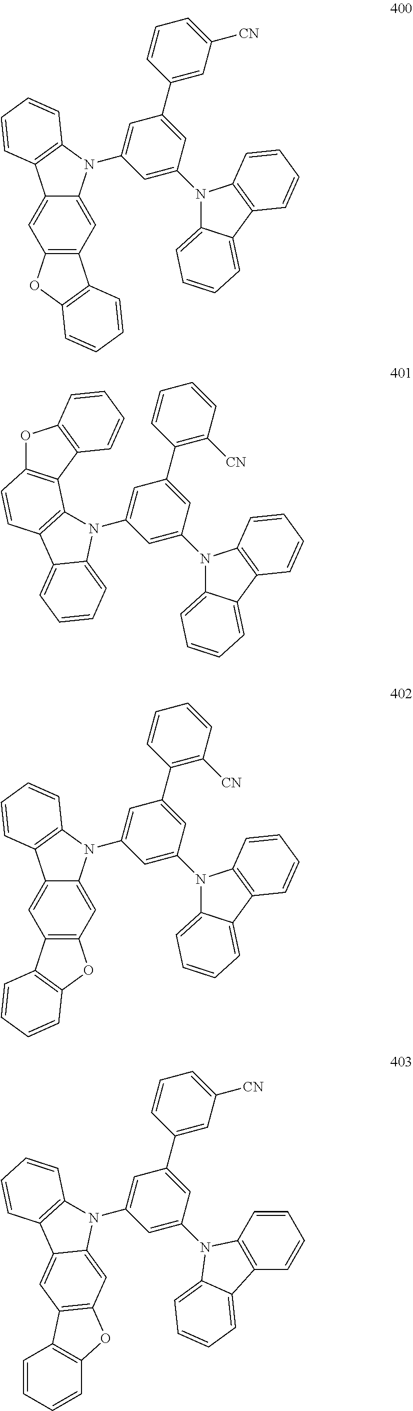

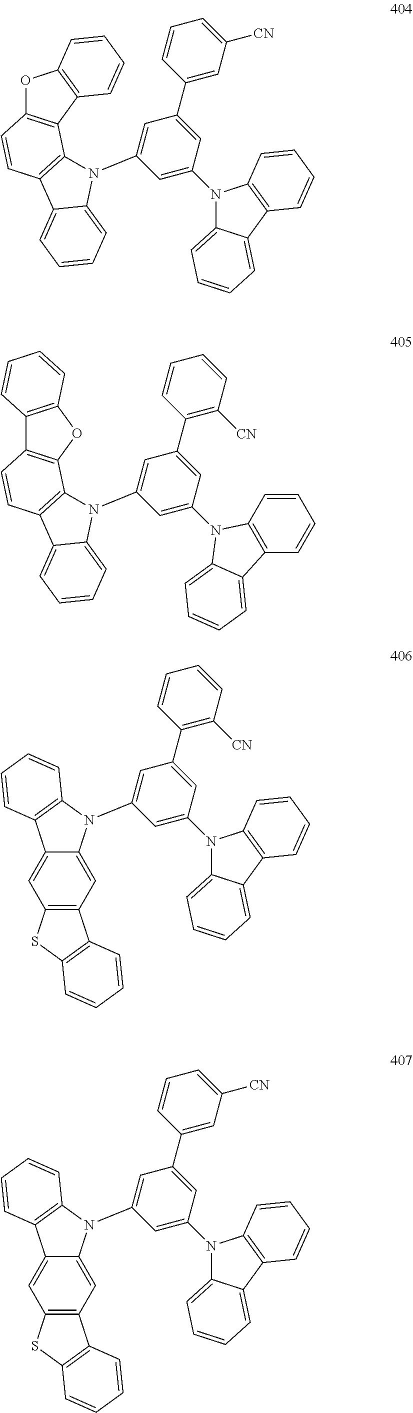

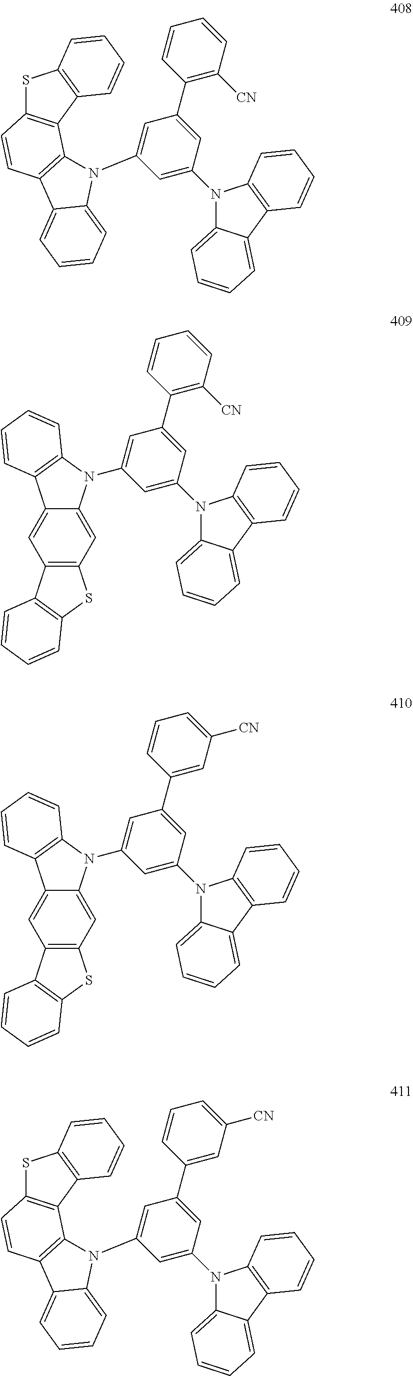 [SMILES: N#Cc1ccccc1-c1cc(-n2c3ccccc3c3ccccc32)cc(-n2c3ccccc3c3ccc4sc5ccccc5c4c32)c1 ; N#Cc1ccccc1-c1cc(-n2c3ccccc3c3ccccc32)cc(-n2c3ccccc3c3cc4c(cc32)sc2ccccc24)c1 ; N#Cc1cccc(-c2cc(-n3c4ccccc4c4ccccc43)cc(-n3c4ccccc4c4cc5c(cc43)sc3ccccc35)c2)c1 ; N#Cc1cccc(-c2cc(-n3c4ccccc4c4ccccc43)cc(-n3c4ccccc4c4ccc5sc6ccccc6c5c43)c2)c1]

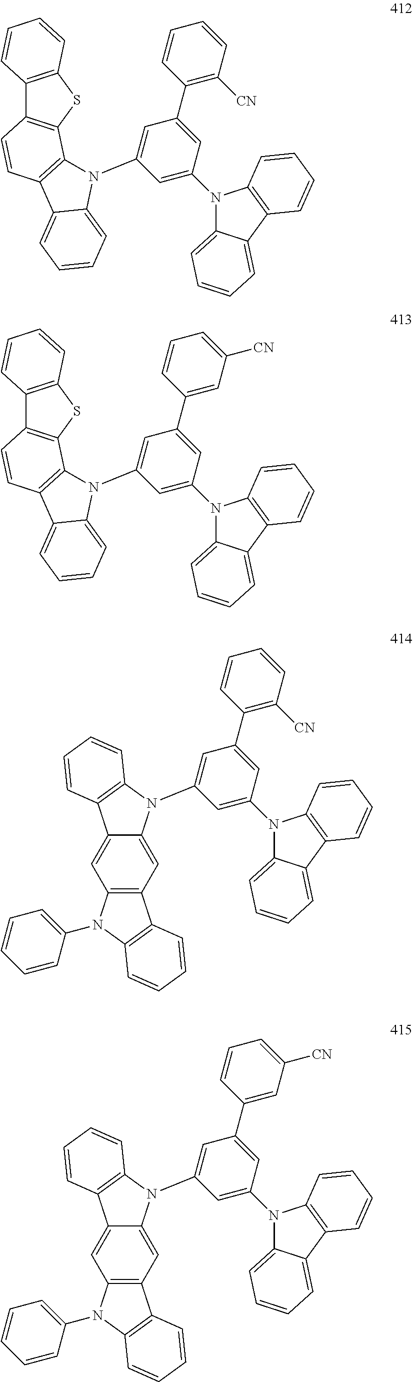

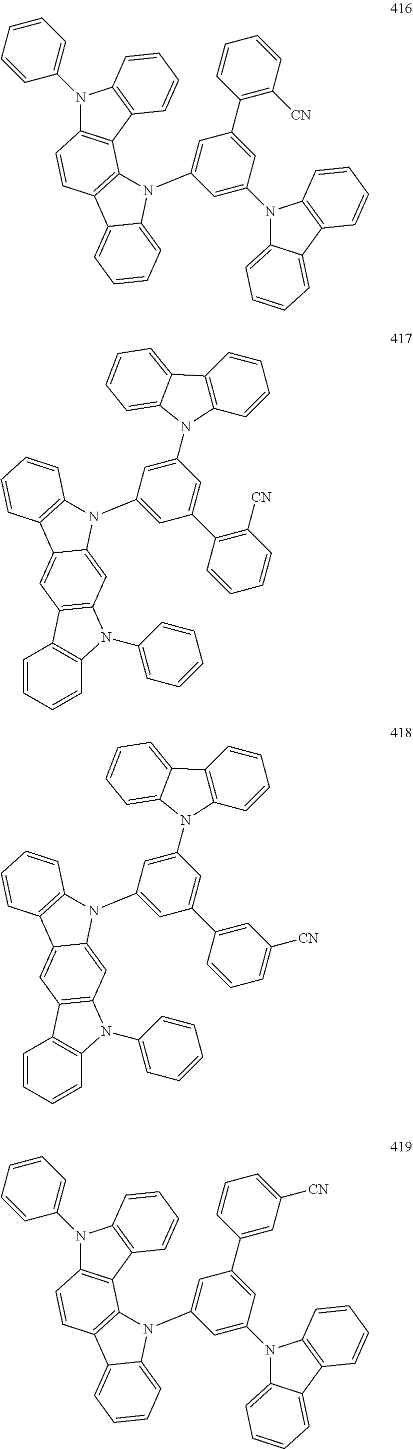 [SMILES: N#Cc1ccccc1-c1cc(-n2c3ccccc3c3ccccc32)cc(-n2c3ccccc3c3ccc4c(c5ccccc5n4-c4ccccc4)c32)c1 ; N#Cc1ccccc1-c1cc(-n2c3ccccc3c3ccccc32)cc(-n2c3ccccc3c3cc4c5ccccc5n(-c5ccccc5)c4cc32)c1 ; N#Cc1cccc(-c2cc(-n3c4ccccc4c4ccccc43)cc(-n3c4ccccc4c4cc5c6ccccc6n(-c6ccccc6)c5cc43)c2)c1 ; N#Cc1cccc(-c2cc(-n3c4ccccc4c4ccccc43)cc(-n3c4ccccc4c4ccc5c(c6ccccc6n5-c5ccccc5)c43)c2)c1]

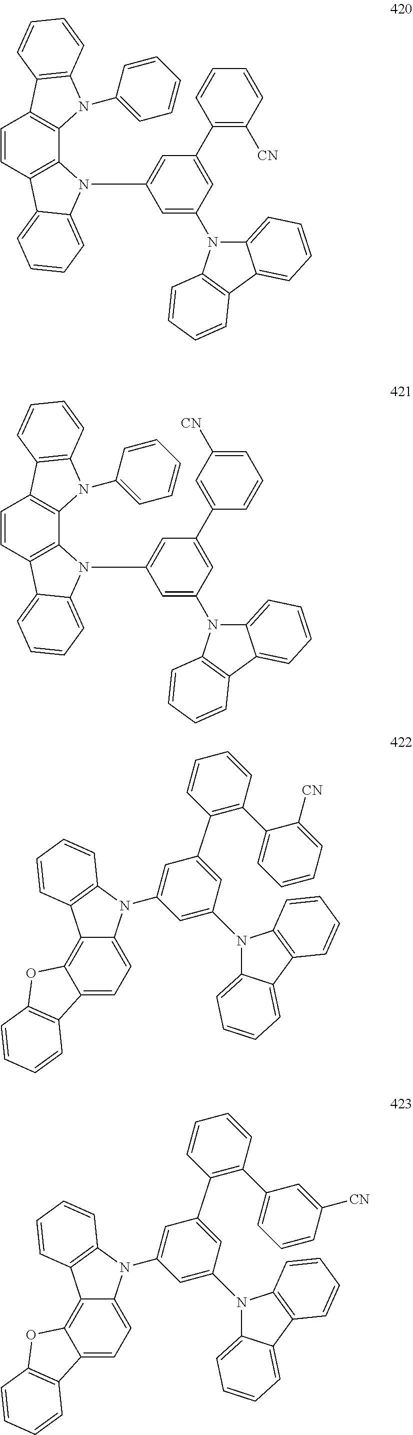

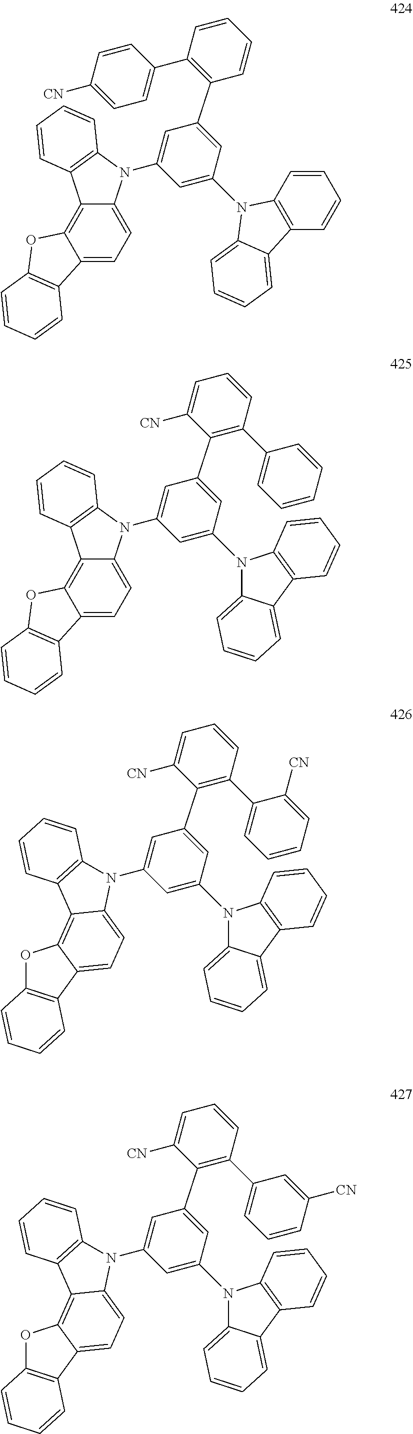

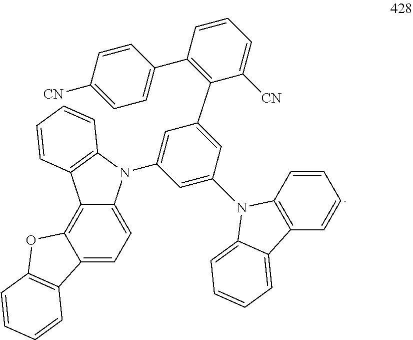

In Formula 1, Ar₁₁ may be represented by Formula 2, wherein A₁ in Formula 2 may be a dibenzofuran group, a dibenzothiophene group, a carbazole group, a fluorene group, a dibenzosilole group, an azadibenzofuran group, an azadibenzothiophene group, an azacarbazole group, an azafluorene group, or an azadibenzosilole group.

Accordingly, the condensed cyclic compound represented by Formula 1 may have excellent thermal stability due to high glass transition temperature (T_{g}) and thermal decomposition temperature (T_{d}), and high charge mobility. Thus, an electronic device, e.g., an organic light-emitting device, including the condensed cyclic compound represented by Formula 1 may have high luminescence efficiency and/or a long lifespan.

In addition, Ar₁₂ in Formula 1 may be a group represented by Formula 3A or Formula 3B, wherein at least one selected from R₁₁ to R₁₃ in Formula 1 may independently be selected from a cyano group-containing phenyl group, a cyano group-containing biphenyl group, a cyano group-containing terphenyl group, and a cyano group-containing tetraphenyl group. Accordingly, the condensed cyclic compound represented by Formula 1 may have a relatively high triplet energy (T₁) level. Therefore, an electronic device, e.g., an organic light-emitting device or an organic light-emitting device emitting blue light, including the condensed cyclic compound represented by Formula 1 may have high luminescence efficiency and/or a long lifespan.

As described above, the condensed cyclic compound represented by Formula 1 may have suitable electrical characteristics for a material for organic light-emitting devices, e.g., a host material in an emission layer, in particular, blue-light-emitting devices. Accordingly, an organic light-emitting device including the condensed cyclic compound may have high efficiency and/or a long lifespan.

For example, the highest occupied molecular orbital (HOMO), the lowest unoccupied molecular orbital (LUMO), triplet (T₁), and singlet (S₁) energy levels of some of the compounds described above and Compounds B and C were evaluated by using Gaussian according to a density functional theory (DFT) method (structure optimization is performed at a degree of B3LYP, and 6-31G(d,p)). The results thereof are shown in Table 1.

TABLE 1

| | HOMO | | | |
|---|---|---|---|---|
| Compound | (electron | | | |
| No. | volts, eV) | LUMO (eV) | T₁ (eV) | S₁ (eV) |
| 31 | −5.305 | −1.795 | 2.967 | 3.057 |
| 392 | −5.221 | −1.670 | 2.992 | 3.109 |
| 179 | −5.280 | −1.503 | 2.952 | 3.306 |
| 34 | −5.321 | −1.832 | 2.974 | 3.033 |
| 180 | −5.322 | −1.655 | 2.973 | 3.168 |
| 393 | −5.248 | −1.657 | 2.862 | 3.133 |
| 32 | −5.420 | −1.768 | 3.002 | 3.206 |
| 394 | −5.340 | −1.653 | 3.001 | 3.256 |
| 14 | −5.452 | −1.975 | 2.915 | 3.025 |
| 193 | −5.541 | −1.841 | 2.973 | 3.220 |
| 35 | −5.432 | −1.799 | 2.993 | 3.176 |
| 44 | −5.278 | −1.564 | 2.978 | 3.288 |
| 45 | −5.562 | −1.950 | 2.981 | 3.160 |
| 194 | −5.606 | −1.903 | 2.963 | 3.232 |
| B | −5.121 | −2.150 | 2.506 | 2.526 |
| C | −5.073 | −1.231 | 2.606 | 3.260 |

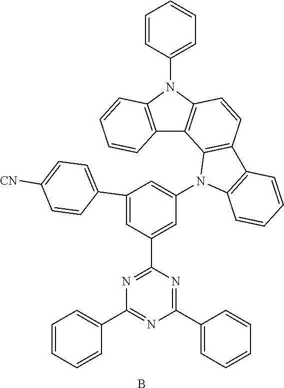

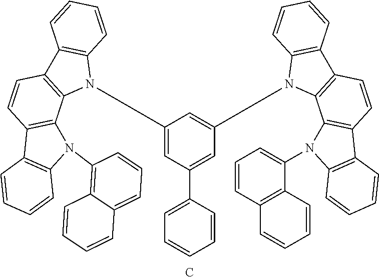

As it may be seen from Table 1, compounds fall within the condensed cyclic compound represented by Formula 1 have higher triplet energy (T₁) level than Compounds B and C. Therefore, the condensed cyclic compound represented by Formula 1 may be suitable for use as an emission layer material in an electronic device, e.g., an organic light-emitting device or an organic light-emitting device emitting blue light. In addition, the condensed cyclic compound has relatively small difference between S₁ energy level and T₁ energy level. Thus, the condensed cyclic compound may be a delayed fluorescence material.

A method of synthesizing the condensed cyclic compound represented by Formula 1 may be apparent to one of ordinary skill in the art by referring to Synthesis Examples provided herein.

According to one or more embodiments, a composition may include a first compound and a second compound,

wherein the first compound may be the condensed cyclic compound represented by Formula 1 provided herein, and

the second compound may include at least one selected from a carbazole group, a dibenzofuran group, a dibenzothiophene group, an indenocarbazole group, an indolocarbazole group, a benzofurocarbazole group, a benzothienocarbazole group, an acridine group, a dihydroacridine group, and a triindolobenzene group and may not include an electron withdrawing group,

wherein the electron withdrawing group may be selected from

—F, —CFH₂, —CF₂H, —CF₃, —CN, and —NO₂;

a C₁-C₆₀ alkyl group substituted with at least one selected from —F, —CFH₂, —CF₂H, —CF₃, —CN, and —NO₂;

a C₁-C₆₀ heteroaryl group and a monovalent non-aromatic condensed polycyclic heterocyclic group, each including *═N—*′ as a ring-forming moiety; and

a C₁-C₆₀ heteroaryl group and a monovalent non-aromatic condensed polycyclic heterocyclic group, each including *═N—*′ as a ring-forming moiety and each substituted with at least one selected from deuterium, —F, —CFH₂, —CF₂H, —CF₃, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₁₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

The composition may be, for example, used in the preparation of an organic layer in an electronic device (e.g., an organic light-emitting device).

The first compound in the composition may be an electron transport material, and the second compound may be a hole transport material.

In some embodiments, the composition may be consisting of the first compound and the second compound, but embodiments are not limited thereto.

The condensed cyclic compound represented by Formula 1 that may be the first compound in the composition may be understood by referring to the descriptions therefor provided herein.

In some embodiments, the second compound in the composition may be selected from Compound represented by Formula H-1:

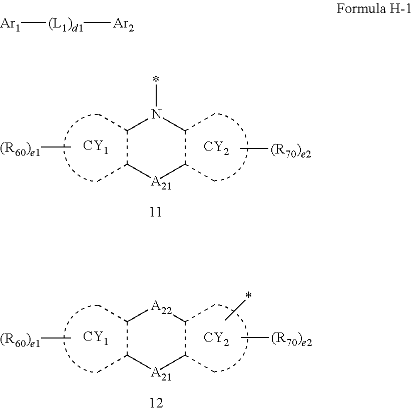

wherein, in Formulae H-1, 11, and 12,

L₁ may be selected from

a single bond, a phenylene group, a naphthylene group, a fluorenylene group, a carbazolylene group, a dibenzofuranylene group, and a dibenzothiophenylene group; and

a phenylene group, a naphthylene group, a fluorenylene group, a carbazolylene group, a dibenzofuranylene group, and a dibenzothiophenylene group, each substituted with at least one selected from deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a biphenyl group, and —Si(Q₁₁)(Q₁₂)(Q₁₃),

d1 may be an integer from 1 to 10; and when d1 is 2 or greater, at least two L₁ groups may be identical to or different from each other,

Ar₁ may be selected from groups represented by Formulae 11 and 12,

Ar₂ may be selected from

groups represented by Formulae 11 and 12, a phenyl group, and a naphthyl group; and

a phenyl group and a naphthyl group, each substituted with at least one selected from deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, and a biphenyl group,

CY₁ and CY₂ may each independently be selected from a benzene group, a naphthalene group, a fluorene group, a carbazole group, a benzocarbazole group, an indolocarbazole group, a dibenzofuran group, a dibenzothiophene group, and a dibenzosilole group,

A₂₁ may be selected from a single bond, O, S, N(R₅₁), C(R₅₁)(R₅₂), and Si(R₅₁)(R₅₂),

A₂₂ may be selected from a single bond, O, S, N(R₅₃), C(R₅₃)(R₄), and Si(R₅₃)(R₅₄),

at least one selected from A₂₁ and A₂₂ in Formula 12 may not be a single bond,

R₅₁ to R₅₄, R₆₀, and R₇₀ may each independently be selected from

hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;

a C₁-C₂₀ alkyl group and a C₁-C₂₀ alkoxy group, each substituted with at least one selected from deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, and a dibenzothiophenyl group;

a phenyl group, a naphthyl group, a fluorenyl group, a carbazolyl group, a dibenzofuranyl group, and a dibenzothiophenyl group;

a phenyl group, a naphthyl group, a fluorenyl group, a carbazolyl group, a dibenzofuranyl group, and a dibenzothiophenyl group, each substituted with at least one selected from deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, and a biphenyl group; and

—Si(Q₁)(Q₂)(Q₃),

e1 and e2 may each independently be an integer from 0 to 10,

wherein Q₁ to Q₃ and Q₁₁ to Q₁₃ may each independently be selected from hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, a dibenzothiophenyl group, and a biphenyl group, and

* indicates a binding site to an adjacent atom.

In some embodiments, in Formulae 11 and 12, at least one selected from CY₁ and CY₂ may be a benzene group, but embodiments are not limited thereto.

In an embodiment, in Formula H-1,

Ar₁ may be selected from groups represented by Formulae 11-1 to 11-8 and 12-1 to 12-8, and

Ar₂ may be selected from

groups represented by Formulae 11-1 to 11-8 and 12-1 to 12-8, a phenyl group, and a naphthyl group; and

a phenyl group and a naphthyl group, each substituted with at least one selected from deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, and a biphenyl group, but embodiments are not limited thereto:

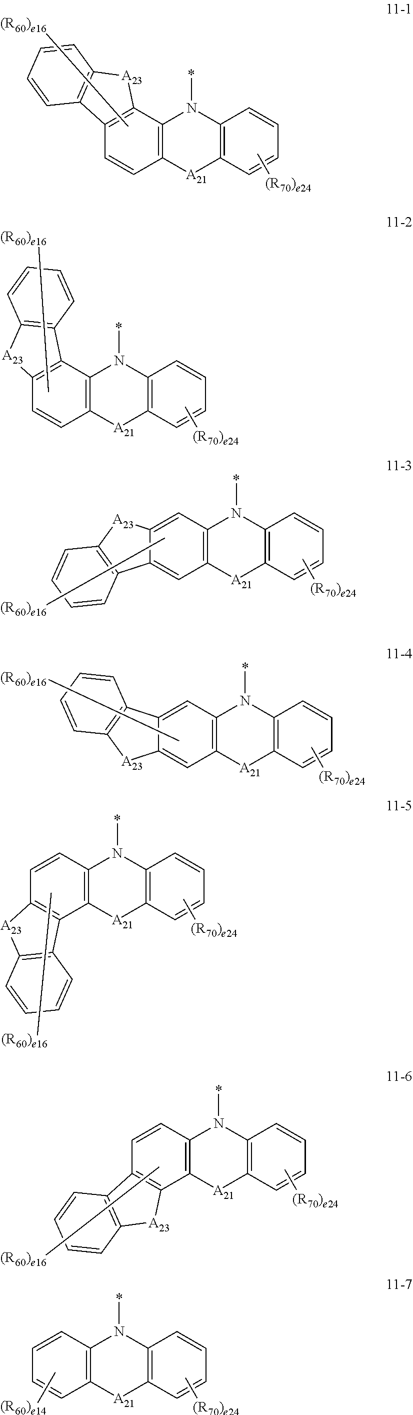

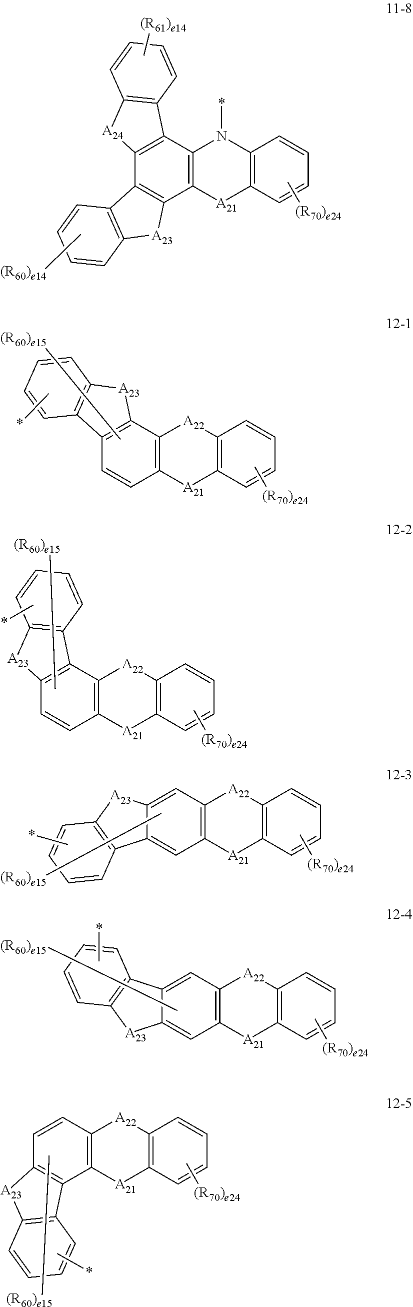

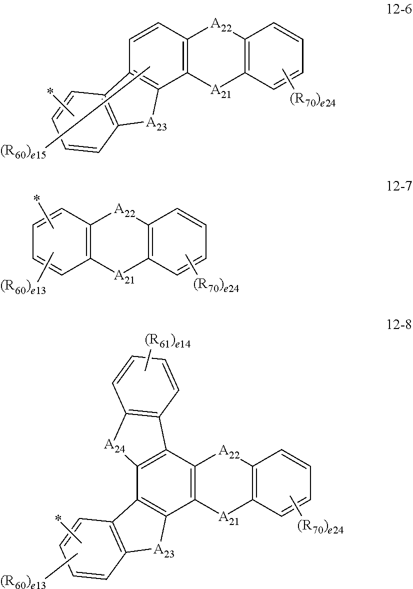

wherein, in Formulae 11-1 to 11-8 and 12-1 to 12-8,

A₂₃ may be selected from O, S, N(R₅₅), C(R₅₅)(R₅₆), and Si(R₅₅)(R₅₆),

A₂₄ may be selected from O, S, N(R₅₇), C(R₅₇)(R₅₈), and Si(R₅₇)(R₅₈),

A₂₁, A₂₂, R₆₀, and R₇₀ may respectively be understood by referring to the descriptions for those provided herein,

R₅₅ to R₅₈ may each be understood by referring to the descriptions for R₅₁ provided herein,

e16 may be an integer from 0 to 6,

e15 may be an integer from 0 to 5,

e14 may be an integer from 0 to 4,

e13 may be an integer from 0 to 3,

e24 may be an integer from 0 to 4, and

* indicates a binding site to an adjacent atom.

In one or more embodiments, in the composition,

i) the second compound may be represented by Formula H-1, provided that the second compound may be selected from compounds in which L₁ in Formula H-1 is a single bond; or

ii) the second compound may be selected from compounds represented by Formulae H-1(1) to H-1(52), but embodiments are not limited thereto:

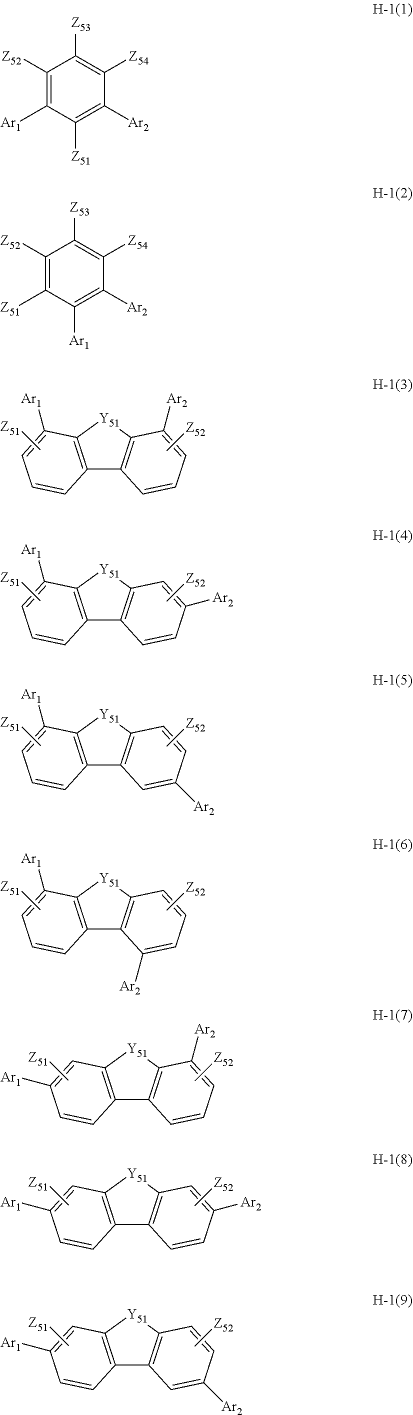

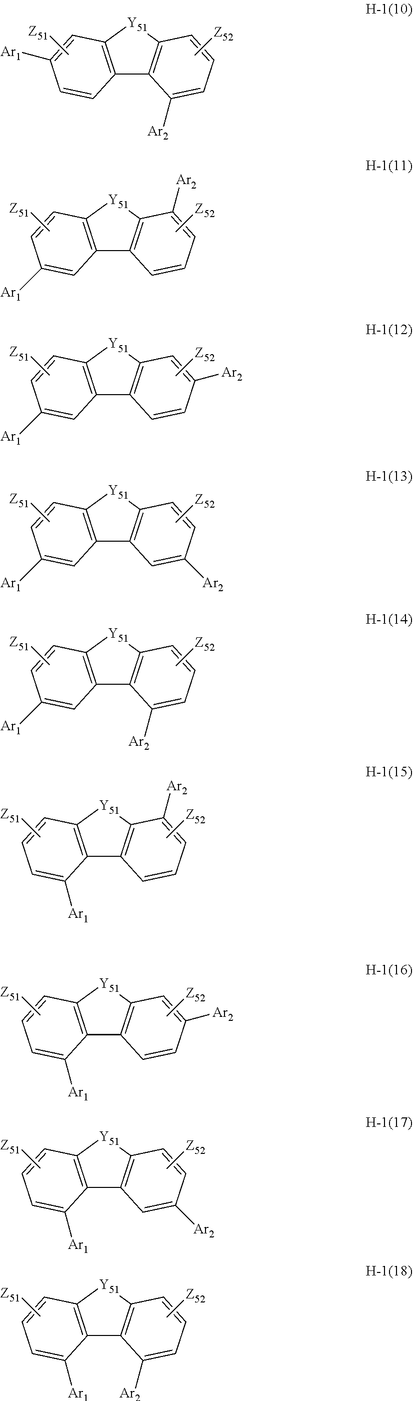

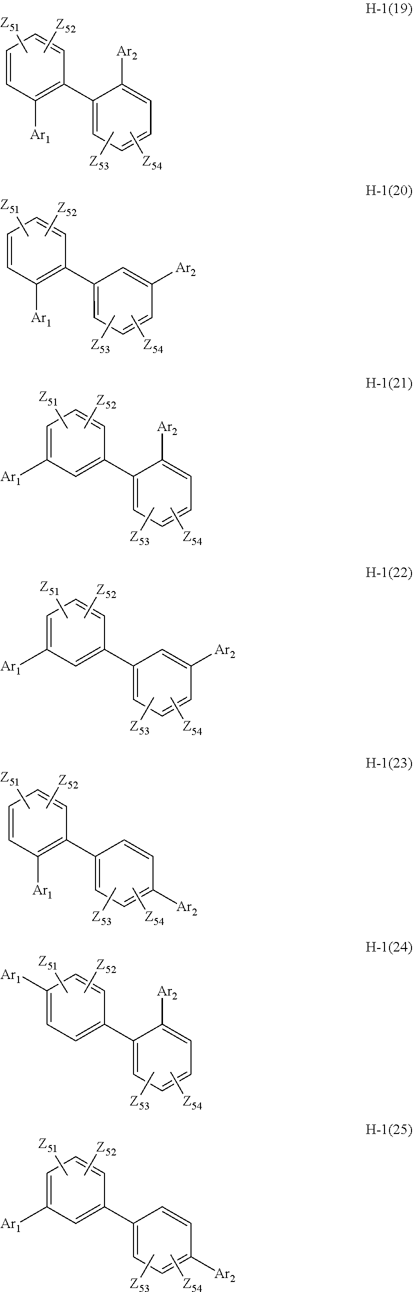

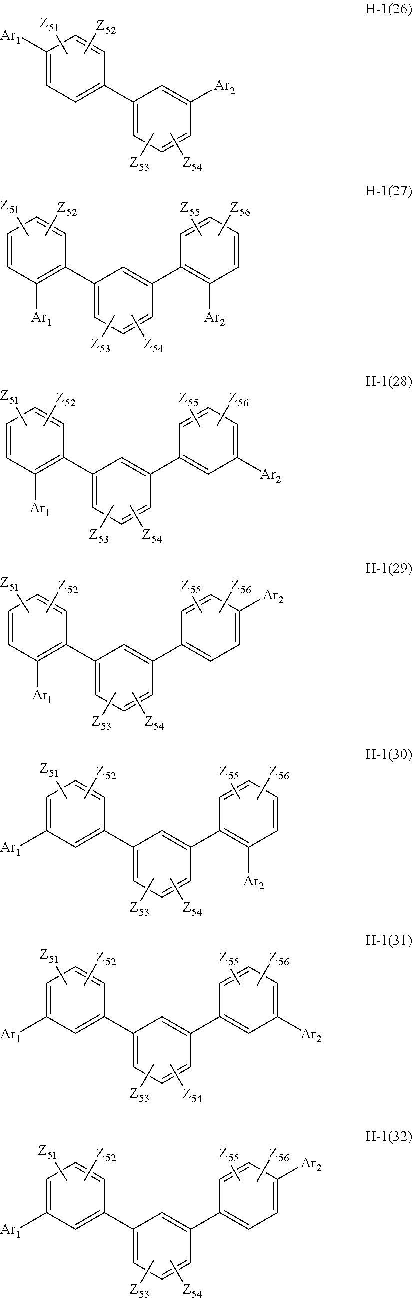

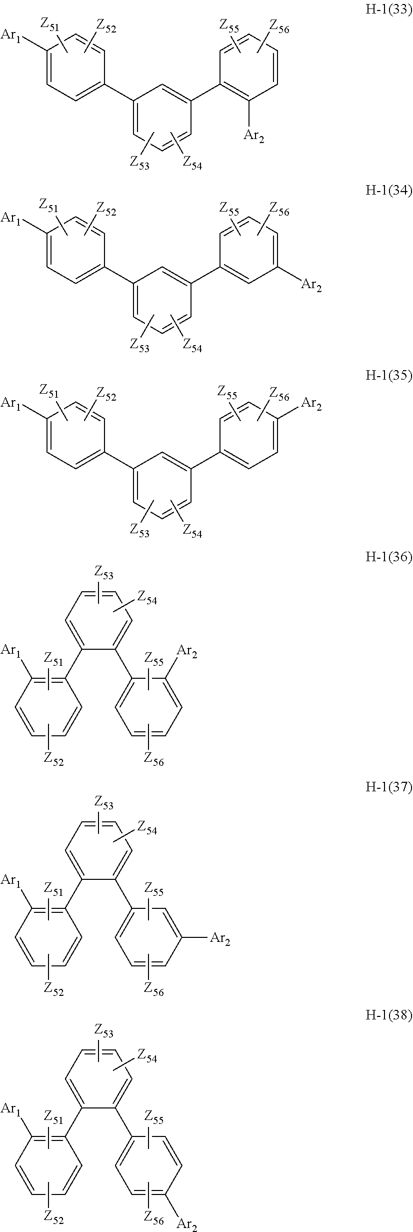

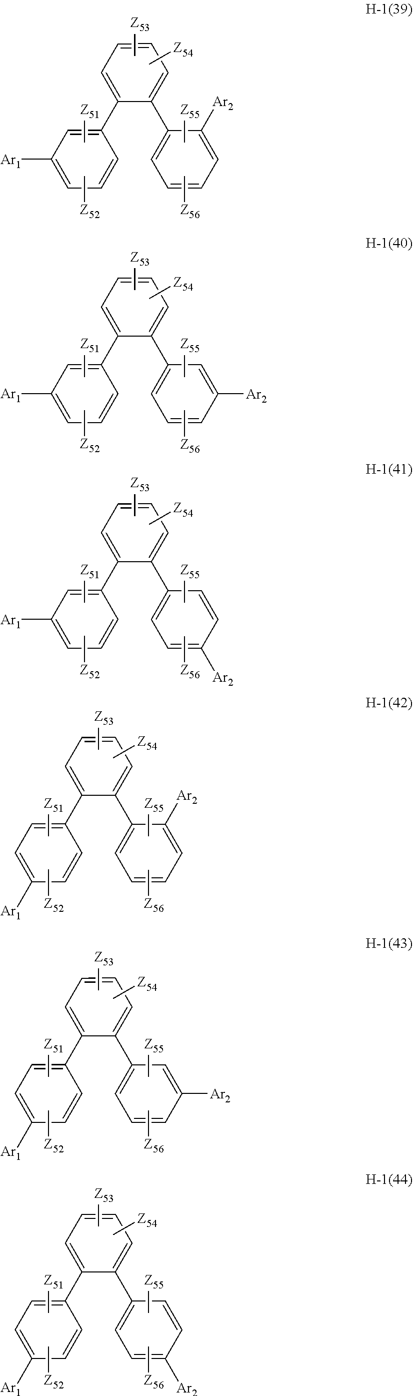

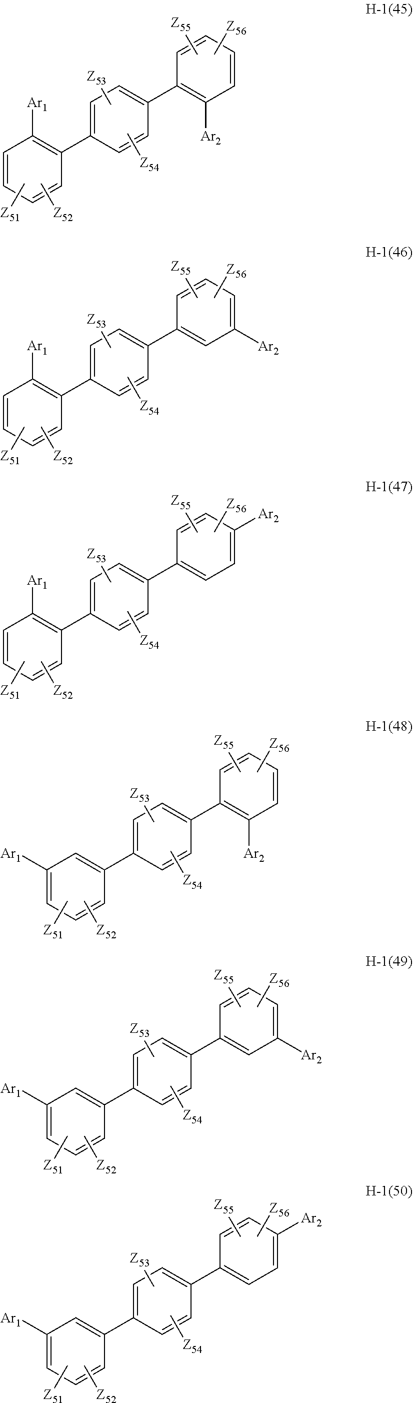

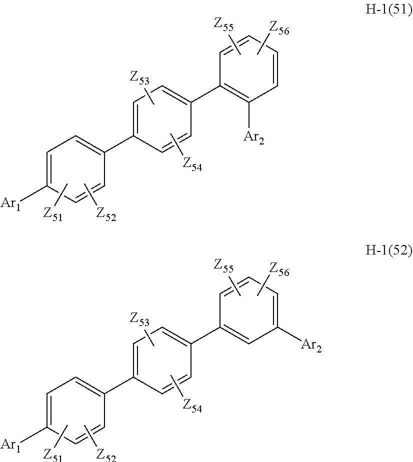

wherein, in Formulae H-1(1) to H-1(52),

Ar₁ and Ar₂ may respectively be understood by referring to the descriptions for those provided herein,

Y₅₁ may each independently be C(Z₅₃)(Z₅₄), Si(Z₅₃)(Z₅₄), N(Z₅₅), O, or S,

Z₅₁ to Z₅₆ may each independently be selected from hydrogen, deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a biphenyl group, and —Si(Q₁₁)(Q₁₂)(Q₁₃),

wherein Q₁₁ to Q₁₃ may each independently be selected from hydrogen, a C₁-C₁₀ alkyl group, a C₁-C₁₀ alkoxy group, a phenyl group, and a naphthyl group, but embodiments are not limited thereto.

In an embodiment, the second compound in the composition may be selected from Compounds H-1 to H-32, but embodiments are not limited thereto:

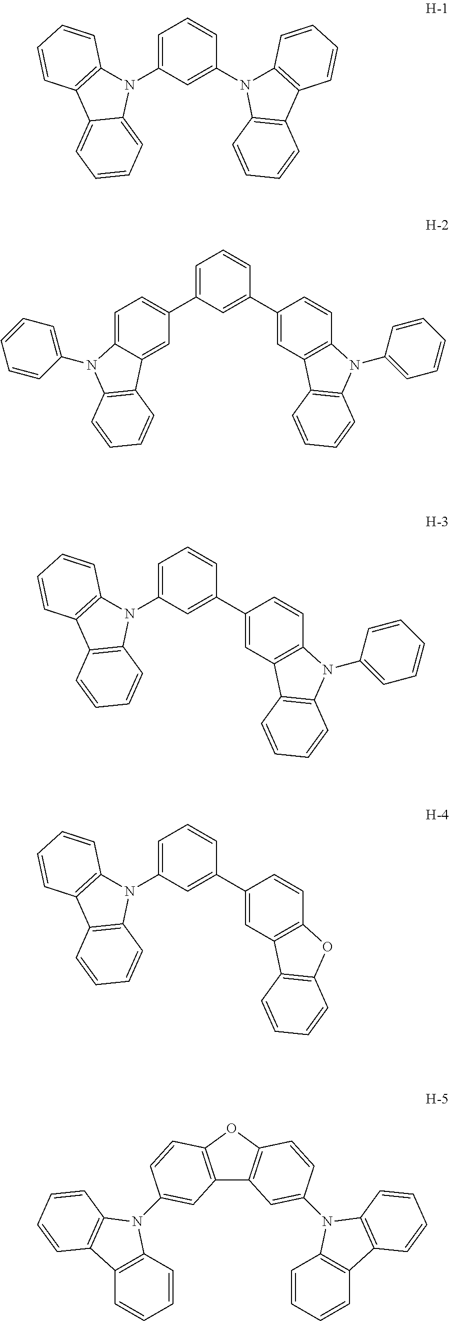 [SMILES: c1cc(-n2c3ccccc3c3ccccc32)cc(-n2c3ccccc3c3ccccc32)c1 ; c1ccc(-n2c3ccccc3c3cc(-c4cccc(-c5ccc6c(c5)c5ccccc5n6-c5ccccc5)c4)ccc32)cc1 ; c1ccc(-n2c3ccccc3c3cc(-c4cccc(-n5c6ccccc6c6ccccc65)c4)ccc32)cc1 ; c1cc(-c2ccc3oc4ccccc4c3c2)cc(-n2c3ccccc3c3ccccc32)c1 ; c1ccc2c(c1)c1ccccc1n2-c1ccc2oc3ccc(-n4c5ccccc5c5ccccc54)cc3c2c1]

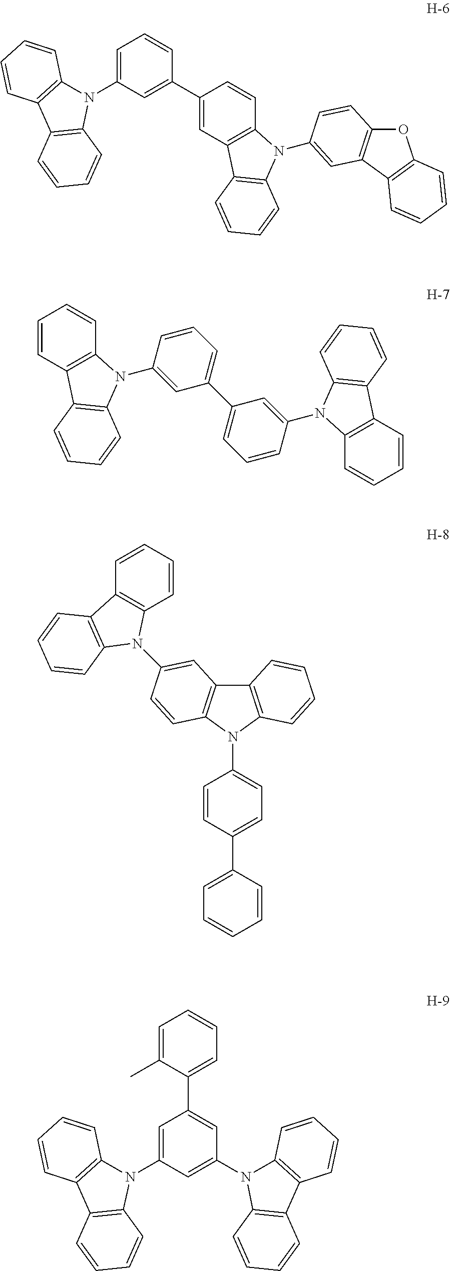 [SMILES: c1cc(-c2ccc3c(c2)c2ccccc2n3-c2ccc3oc4ccccc4c3c2)cc(-n2c3ccccc3c3ccccc32)c1 ; c1cc(-c2cccc(-n3c4ccccc4c4ccccc43)c2)cc(-n2c3ccccc3c3ccccc32)c1 ; c1ccc(-c2ccc(-n3c4ccccc4c4cc(-n5c6ccccc6c6ccccc65)ccc43)cc2)cc1 ; Cc1ccccc1-c1cc(-n2c3ccccc3c3ccccc32)cc(-n2c3ccccc3c3ccccc32)c1]

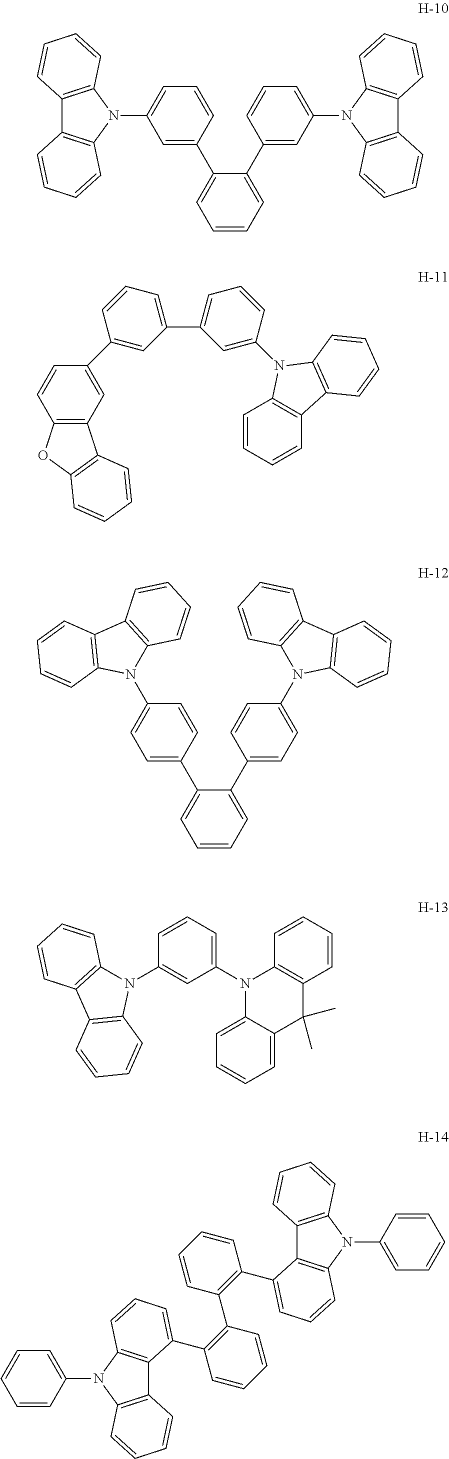 [SMILES: c1cc(-c2ccccc2-c2cccc(-n3c4ccccc4c4ccccc43)c2)cc(-n2c3ccccc3c3ccccc32)c1 ; c1cc(-c2cccc(-n3c4ccccc4c4ccccc43)c2)cc(-c2ccc3oc4ccccc4c3c2)c1 ; c1ccc(-c2ccc(-n3c4ccccc4c4ccccc43)cc2)c(-c2ccc(-n3c4ccccc4c4ccccc43)cc2)c1 ; CC1(C)c2ccccc2N(c2cccc(-n3c4ccccc4c4ccccc43)c2)c2ccccc21 ; c1ccc(-n2c3ccccc3c3c(-c4ccccc4-c4ccccc4-c4cccc5c4c4ccccc4n5-c4ccccc4)cccc32)cc1]

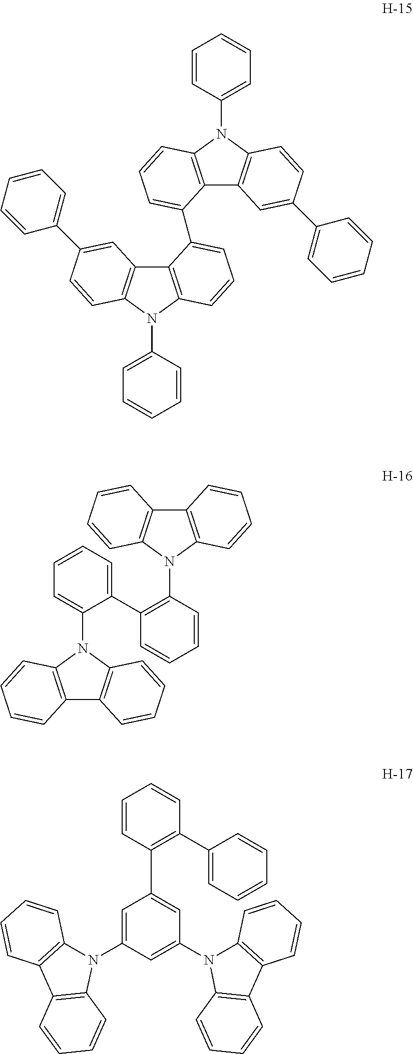 [SMILES: c1ccc(-c2ccc3c(c2)c2c(-c4cccc5c4c4cc(-c6ccccc6)ccc4n5-c4ccccc4)cccc2n3-c2ccccc2)cc1 ; c1ccc(-n2c3ccccc3c3ccccc32)c(-c2ccccc2-n2c3ccccc3c3ccccc32)c1 ; c1ccc(-c2ccccc2-c2cc(-n3c4ccccc4c4ccccc43)cc(-n3c4ccccc4c4ccccc43)c2)cc1]

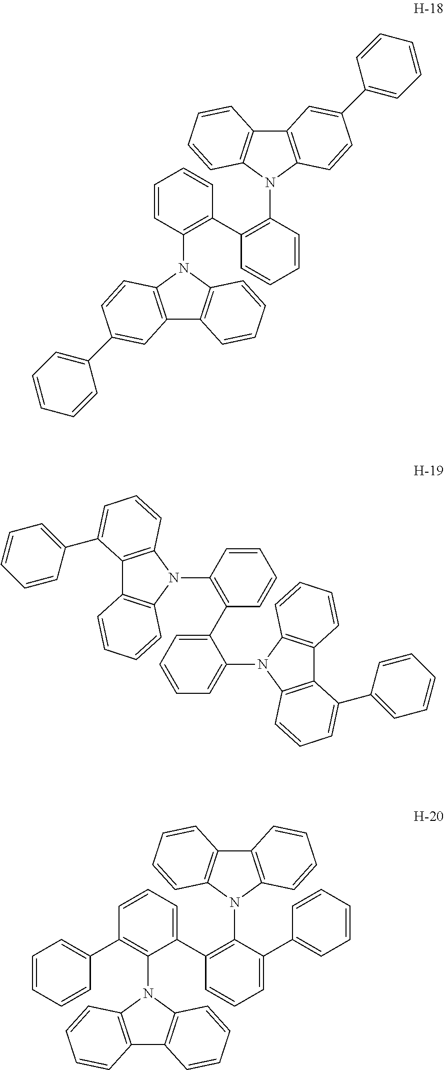 [SMILES: c1ccc(-c2ccc3c(c2)c2ccccc2n3-c2ccccc2-c2ccccc2-n2c3ccccc3c3cc(-c4ccccc4)ccc32)cc1 ; c1ccc(-c2cccc3c2c2ccccc2n3-c2ccccc2-c2ccccc2-n2c3ccccc3c3c(-c4ccccc4)cccc32)cc1 ; c1ccc(-c2cccc(-c3cccc(-c4ccccc4)c3-n3c4ccccc4c4ccccc43)c2-n2c3ccccc3c3ccccc32)cc1]

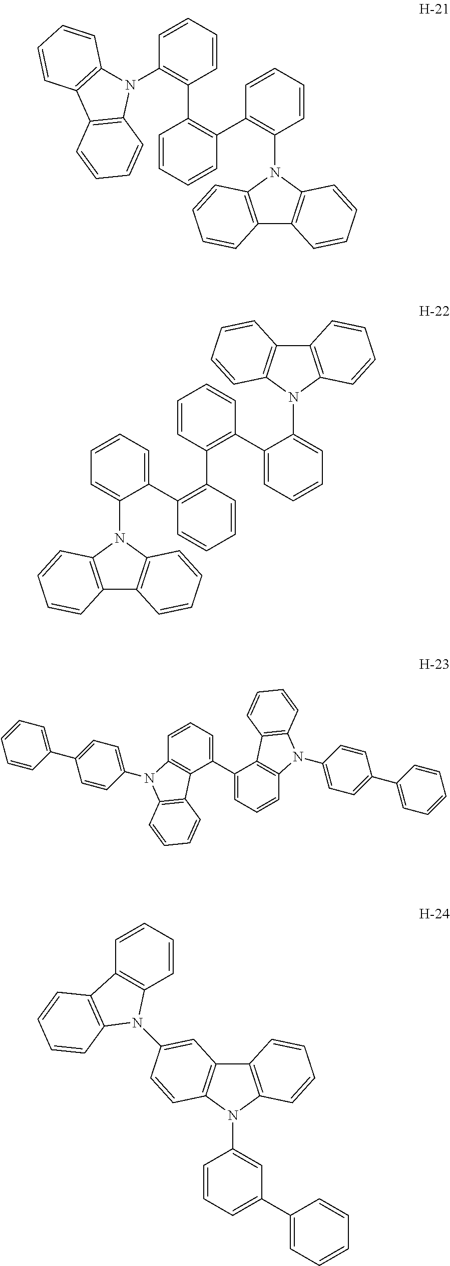 [SMILES: c1ccc(-c2ccccc2-n2c3ccccc3c3ccccc32)c(-c2ccccc2-n2c3ccccc3c3ccccc32)c1 ; c1ccc(-c2ccccc2-n2c3ccccc3c3ccccc32)c(-c2ccccc2-c2ccccc2-n2c3ccccc3c3ccccc32)c1 ; c1ccc(-c2ccc(-n3c4ccccc4c4c(-c5cccc6c5c5ccccc5n6-c5ccc(-c6ccccc6)cc5)cccc43)cc2)cc1 ; c1ccc(-c2cccc(-n3c4ccccc4c4cc(-n5c6ccccc6c6ccccc65)ccc43)c2)cc1]

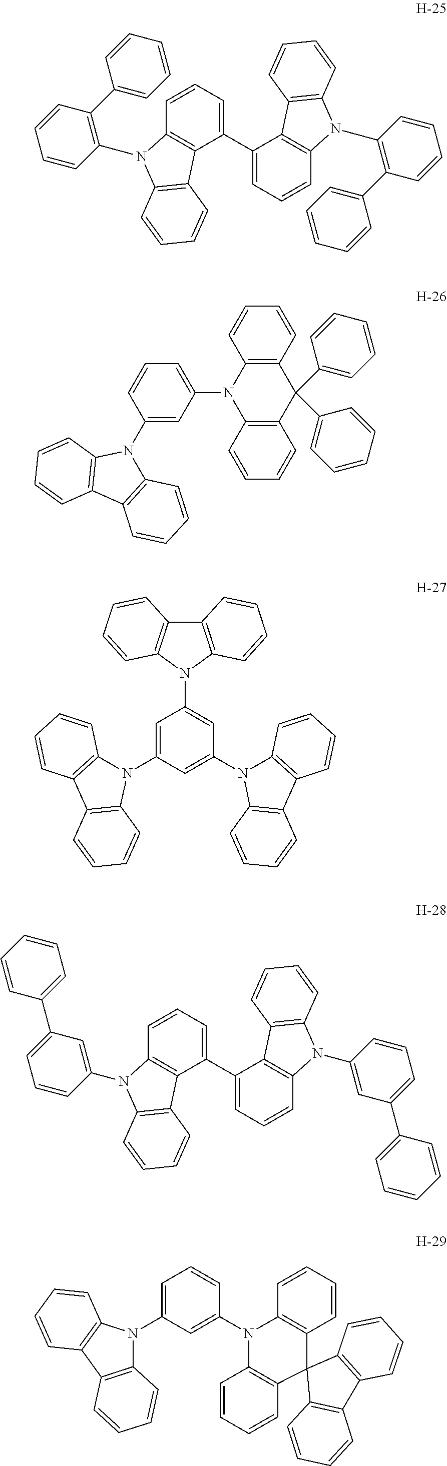 [SMILES: c1ccc(-c2ccccc2-n2c3ccccc3c3c(-c4cccc5c4c4ccccc4n5-c4ccccc4-c4ccccc4)cccc32)cc1 ; c1ccc(C2(c3ccccc3)c3ccccc3N(c3cccc(-n4c5ccccc5c5ccccc54)c3)c3ccccc32)cc1 ; c1ccc2c(c1)c1ccccc1n2-c1cc(-n2c3ccccc3c3ccccc32)cc(-n2c3ccccc3c3ccccc32)c1 ; c1ccc(-c2cccc(-n3c4ccccc4c4c(-c5cccc6c5c5ccccc5n6-c5cccc(-c6ccccc6)c5)cccc43)c2)cc1 ; c1cc(N2c3ccccc3C3(c4ccccc4-c4ccccc43)c3ccccc32)cc(-n2c3ccccc3c3ccccc32)c1]

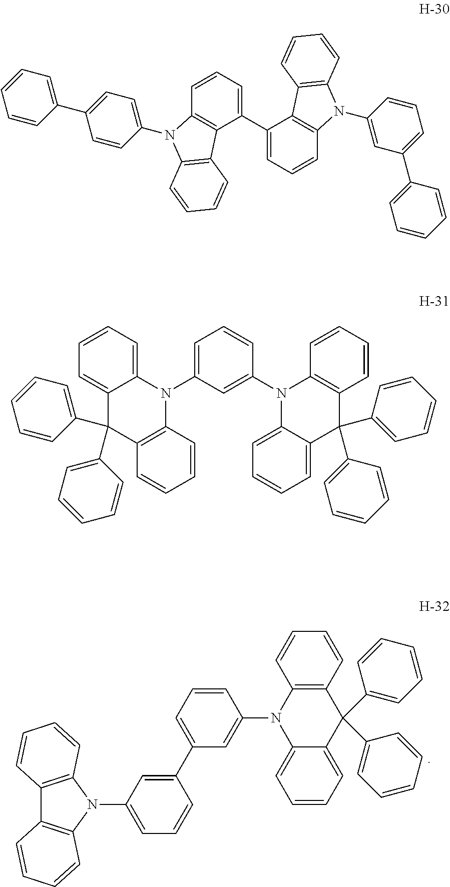 [SMILES: c1ccc(-c2ccc(-n3c4ccccc4c4c(-c5cccc6c5c5ccccc5n6-c5cccc(-c6ccccc6)c5)cccc43)cc2)cc1 ; c1ccc(C2(c3ccccc3)c3ccccc3N(c3cccc(N4c5ccccc5C(c5ccccc5)(c5ccccc5)c5ccccc54)c3)c3ccccc32)cc1 ; c1ccc(C2(c3ccccc3)c3ccccc3N(c3cccc(-c4cccc(-n5c6ccccc6c6ccccc65)c4)c3)c3ccccc32)cc1]

In the composition, a weight ratio of the first compound to the second compound may be in a range of about 1:99 to about 99:1, and in some embodiments, about 70:30 to about 30:30. In some embodiments, in the composition, a weight ratio of the first compound to the second compound may be selected from a range of about 40:60 to about 60:40, but embodiments are not limited thereto. While not wishing to be bound by theory, it is understood that when a weight ratio of the first compound to the second compound in the composition is within any of these ranges, the composition may provide excellent charge transport balance.

The condensed cyclic compound represented by Formula 1 may be suitable for use in an organic layer of an organic light-emitting device, for example, in an emission layer and/or an electron transport region of the organic layer. According to an aspect of another embodiment, an organic light-emitting device may include: a first electrode, a second electrode, and an organic layer disposed between the first electrode and the second electrode, wherein the organic layer includes an emission layer, and wherein the organic layer may include at least one condensed cyclic compound represented by Formula 1 or the composition described above.

The organic light-emitting device including the organic layer including the condensed cyclic compound represented by Formula 1 or the composition including the first and second compounds may exhibit low driving voltage, high efficiency, high luminance, high quantum emission efficiency, and long lifespan.

In an embodiment, in the organic light-emitting device,

the first electrode may be an anode, and the second electrode may be a cathode,

the organic layer may include a hole transport region disposed between the first electrode and the emission layer and an electron transport region disposed between the emission layer and the second electrode,

wherein the hole transport region may include a hole injection layer, a hole transport layer, an electron blocking layer, a buffer layer, or any combination thereof, and

the electron transport region may include a hole blocking layer, an electron transport layer, an electron injection layer, or a combination thereof, but embodiments are not limited thereto.

In some embodiments, the emission layer in the organic light-emitting device may include at least one condensed cyclic compound represented by Formula 1 or the composition including the first and second compounds.

In an embodiment, the emission layer in the organic light-emitting device may include a host and a dopant, wherein the host may include at least one condensed cyclic compound represented by Formula 1 or the composition including the first and second compounds, and the dopant may include a phosphorescent dopant or a fluorescent dopant. In some embodiments, the dopant may include a phosphorescent dopant (e.g., an organometallic compound represented by Formula 81 provided herein). The condensed cyclic compound contained in the host may deliver energy to the dopant according to the delayed fluorescence mechanism. A content of the host in the emission layer may be greater than that of the dopant in the emission layer. The host may further include, in addition to the condensed cyclic compound represented by Formula 1 or the composition including the first and second compounds, any suitable host.

In an embodiment, the emission layer in the organic light-emitting device may include a host and a dopant, wherein the dopant may include at least one condensed cyclic compound represented by Formula 1. The condensed cyclic compound contained in the dopant may serve as an emitter for emitting delayed fluorescence according to the delayed fluorescence mechanism. In some embodiments, the dopant may further include any known emitting dopant, the condensed cyclic compound may serve as a dopant assisting energy deliverance to the emitting dopant according to the delayed fluorescence mechanism. A content of the host in the emission layer may be greater than that of the dopant in the emission layer. The host may include any suitable host.

The emission layer may emit red light, green light, or blue light.

In an embodiment, the emission layer may be a blue emission layer including a phosphorescent dopant, but embodiments are not limited thereto.

In some embodiments, the condensed cyclic compound represented by Formula 1 may be included in an electron transport region of the organic light-emitting device.

In some embodiments, an electron transport region of the organic light-emitting device may include at least one selected from a hole blocking layer and an electron transport layer, wherein at least one selected from the hole blocking layer and the electron transport layer may include the condensed cyclic compound represented by Formula 1.

In an embodiment, an electron transport region of the organic light-emitting device may include a hole blocking layer, wherein the hole blocking layer may include the condensed cyclic compound represented by Formula 1. The hole blocking layer may be in a direct contact with the emission layer.

In one or more embodiments, an organic layer of the organic light-emitting device may further include, in addition to the condensed cyclic compound represented by Formula 1,

i) the second compound;

ii) an organometallic compound represented by Formula 81; or

iii) any combination of i) and ii).

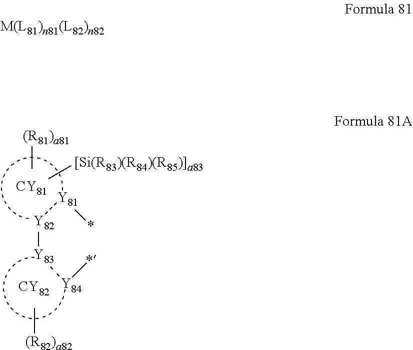

wherein, in Formulae 81 and 81A,

M may be selected from iridium (Ir), platinum (Pt), osmium (Os), titanium (Ti), zirconium (Zr), hafnium (Hf), europium (Eu), terbium (Tb), thulium (Tm), and rhodium (Rh),

L₈₁ may be a ligand represented by Formula 81A, n81 may be an integer from 1 to 3; and when n81 is 2 or greater, at least two L₈₁ groups may be identical to or different from each other,

L₈₂ may be an organic ligand, n82 may be an integer from 0 to 4; and when n82 is 2 or greater, at least two L₈₂ groups may be identical to or different from each other,

Y₈₁ to Y₈₄ may each independently be C or N,

Y₈₁ and Y₈₂ may be bound via a single bond or a double bond, Y₈₃ and Y₈₄ may be bound via a single bond or a double bond,

CY₈₁ and CY₈₂ may each independently be selected from a C₅-C₃₀ carbocyclic group and a C₁-C₃₀ heterocarbocyclic group,

CY₈₁ and CY₈₂ may optionally or additionally be bound via an organic linking group,

R₈₁ to R₈₅ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₆₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₈₁)(Q₈₂)(Q₈₃), —N(Q₈₄)(Q₈₅), —B(Q₈₆)(Q₈₇), and —P(═O)(Q₈₈)(Q₈₉),

a81 to a83 may each independently be an integer from 0 to 5,

when a81 is 2 or greater, at least two R₈₁ groups may be identical to or different from each other,

when a82 is 2 or greater, at least two R₈₂ groups may be identical to or different from each other,

when a81 is 2 or greater, two adjacent R₈₁ may be optionally bound to form a saturated or unsaturated C₂-C₃₀ ring (e.g., a benzene ring, a cyclopentane ring, a cyclohexane ring, a cyclopentene ring, a cyclohexene ring, a norbomane ring, a (bicyclo[2.2.1]heptanes) ring, a naphthalene ring, a benzoindene ring, a benzoindole ring, a benzofuran ring, a benzothiophene ring, a pyridine ring, a pyrimidine ring, or a pyrazine ring), or a saturated or unsaturated C₂-C₃₀ ring substituted with at least one R₈₈ (e.g., a benzene ring, a cyclopentane ring, a cyclohexane ring, a cyclopentene ring, a cyclohexene ring, a norbomane ring, a (bicyclo[2.2.1]heptanes) ring, a naphthalene ring, a benzoindene ring, a benzoindole ring, a benzofuran ring, a benzothiophene ring, a pyridine ring, a pyrimidine ring, or a pyrazine ring, each substituted with at least one R₈₈),

when a82 is 2 or greater, two adjacent R₈₂ may be optionally bound to form a saturated or unsaturated C₂-C₃₀ ring (e.g., a benzene ring, a cyclopentane ring, a cyclohexane ring, a cyclopentene ring, a cyclohexene ring, a norbomane ring, a (bicyclo[2.2.1]heptanes) ring, a naphthalene ring, a benzoindene ring, a benzoindole ring, a benzofuran ring, a benzothiophene ring, a pyridine ring, a pyrimidine ring, or a pyrazine ring), or a saturated or unsaturated C₂-C₃₀ ring substituted with at least one R₈₉ (e.g., a benzene ring, a cyclopentane ring, a cyclohexane ring, a cyclopentene ring, a cyclohexene ring, a norbornmane ring, a (bicyclo[2.2.1]heptanes) ring, a naphthalene ring, a benzoindene ring, a benzoindole ring, a benzofuran ring, a benzothiophene ring, a pyridine ring, a pyrimidine ring, or a pyrazine ring, each substituted with at least one R₈₉),

R₈₈ may be understood by referring to the descriptions for R₈₁ provided herein,

R₈₉ may be understood by referring to the descriptions for R₈₂ provided herein,

in Formula 81A, * and *′ each indicate a binding site to M in Formula 81, and

at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₉₁)(Q₉₂)(Q₉₃),

wherein Q₈₁ to Q₈₉ and Q₉₁ to Q₉₃ may each independently be selected from hydrogen, deuterium, a C₁-C₆₀ alkyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

In an embodiment, in Formula 81A,

a83 may be 1 or 2, and

R₈₃ to R₈₅ may each independently be selected from

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CH₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group, each substituted with at least one selected from deuterium, a C₁-C₁₀ alkyl group, and a phenyl group, but embodiments are not limited thereto.

In one or more embodiments, in Formula 81A,

Y₈₁ may be N, Y₈₂ and Y₈₃ may each be C, Y₈₄ may be N or C, and

CY₈₁ and CY₈₂ may each independently be selected from a cyclopentadiene group, a benzene group, a heptalene group, an indene group, a naphthalene group, an azulene group, a heptalene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a coronene group, an ovalene group, a pyrrole group, an isoindole group, an indole group, an indazole group, a pyrazole group, an imidazole group, a triazole group, an oxazole group, an isoxazole group, an oxadiazole group, a thiazole group, an isothiazole group, a thiadiazole group, a purine group, a furan group, a thiophene group, a pyridine group, a pyrimidine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, a benzofuran group, a benzothiophene group, an iso-benzothiazole group, a benzoxazole group, an isobenzoxazole group, a benzocarbazole group, a dibenzocarbazole group, an imidazopyridine group, an imidazopyrimidine group, a dibenzofuran group, a dibenzothiophene group, a dibenzothiophene sulfone group, a carbazole group, a dibenzosilole group, and a 2,3-dihydro-1H-imidazole group.

In one or more embodiments, in Formula 81A, Y₈₁ may be N, Y₈₂ to Y₈₄ may each be C, CY₈₁ may be selected from 5-membered rings including two N atoms as ring-forming atoms, and CY₈₂ may be selected from a benzene group, a naphthalene group a fluorene group, a dibenzofuran group, and a dibenzothiophene group, but embodiments are not limited thereto.

In one or more embodiments, in Formula 81A, Y₈₁ may be N, Y₈₂ to Y₈₄ may each be C, CY₈₁ may be an imidazole group or a 2,3-dihydro-1H-imidazole group, and CY₈₂ may be selected from a benzene group, a naphthalene group a fluorene group, a dibenzofuran group, and a dibenzothiophene group, but embodiments are not limited thereto.

In one or more embodiments, in Formula 81A,

Y₈₁ may be N, Y₈₂ to Y₈₄ may each be C,

CY₈₁ may be selected from a pyrrole group, a pyrazole group, an imidazole group, a triazole group, an oxazole group, an isoxazole group, an oxadiazole group, a thiazole group, an isothiazole group, a thiadiazole group, a pyridine group, a pyrimidine group, a quinoline group, an isoquinoline group, a benzoquinoline group, a phthalazine group, a naphthyridine group, a quinoxaline group, a quinazoline group, a cinnoline group, a benzimidazole group, an iso-benzothiazole group, a benzoxazole group, and an isobenzoxazole group, and

CY₈₂ may be selected from a cyclopentadiene group, a benzene group, a naphthalene group, a fluorene group, a benzofluorene group, a dibenzofluorene group, a phenanthrene group, an anthracene group, a triphenylene group, a pyrene group, a chrysene group, a perylene group, a benzofuran group, a benzothiophene group, a benzocarbazole group, a dibenzocarbazole group, a dibenzofuran group, a dibenzothiophene group, a dibenzothiophene sulfone group, a carbazole group, and a dibenzosilole group.

In one or more embodiments, in Formula 81A, R₈₁ and R₈₂ may each independently be selected from

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbomrnanyl group, a norbomenyl group, a cyclopentenyl group, a cyclohexenyl group, a cycloheptenyl group, a phenyl group, a naphthyl group, a pyridinyl group, and a pyrimidinyl group;

a cyclopentyl group, a cyclohexyl group, a cycloheptyl group, a cyclooctyl group, an adamantanyl group, a norbomrnanyl group, a norbomenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;

a cyclopentyl group, a cyclohexyl group, a cycloheptyl group, a cyclooctyl group, an adamantanyl group, a norbomrnanyl group, a norbomenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbomanyl group, a norbomenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and

—B(Q₈₆)(Q₈₇) and —P(═O)(Q₈₈)(Q₈₉),

wherein Q₈₆ to Q₈₉ may each independently be selected from

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CH₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group, each substituted with at least one selected from deuterium, a C₁-C₁₀ alkyl group, and a phenyl group.

In one or more embodiments, in Formula 81A, at least one of i) R₈₁(s) in the number of a81 and ii) R₈₂(s) in the number of a82 may be a cyano group.

In one or more embodiments, in Formula 81A, at least one of R₈₂(s) group in the number of a82 may be a cyano group.

In one or more embodiments, in Formula 81A, at least one of i) R₈₁(s) in the number of a81 and ii) R₈₂(s) in the number of a82 may be deuterium.

In one or more embodiments, in Formula 81, L₈₂ may be selected from ligands represented by Formulae 3-1(1) to 3-1(60), 3-1(61) to 3-1(69), 3-1(71) to 3-1(79), 3-1(81) to 3-1(88), 3-1(91) to 3-1(98), and 3-1(101) to 3-1(114):

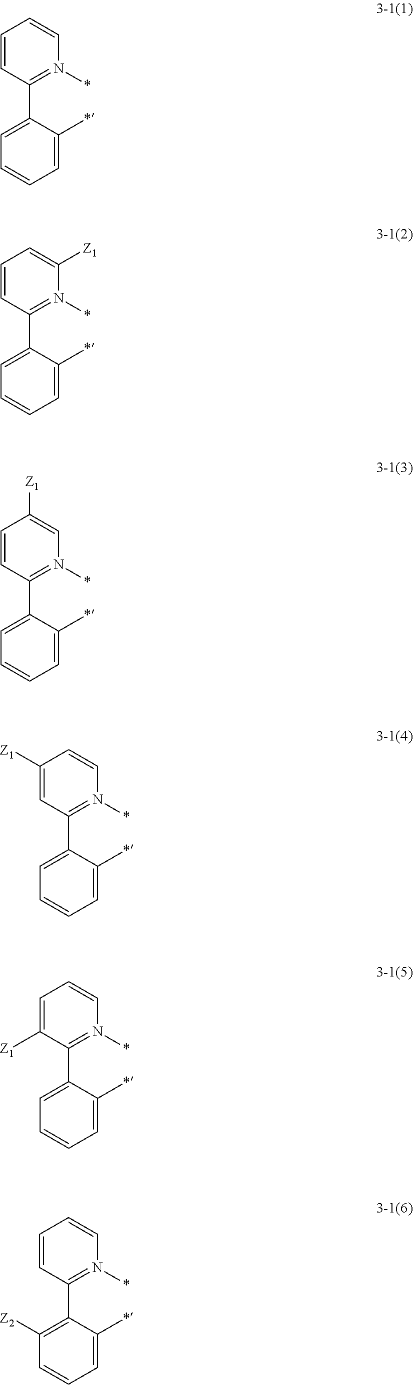

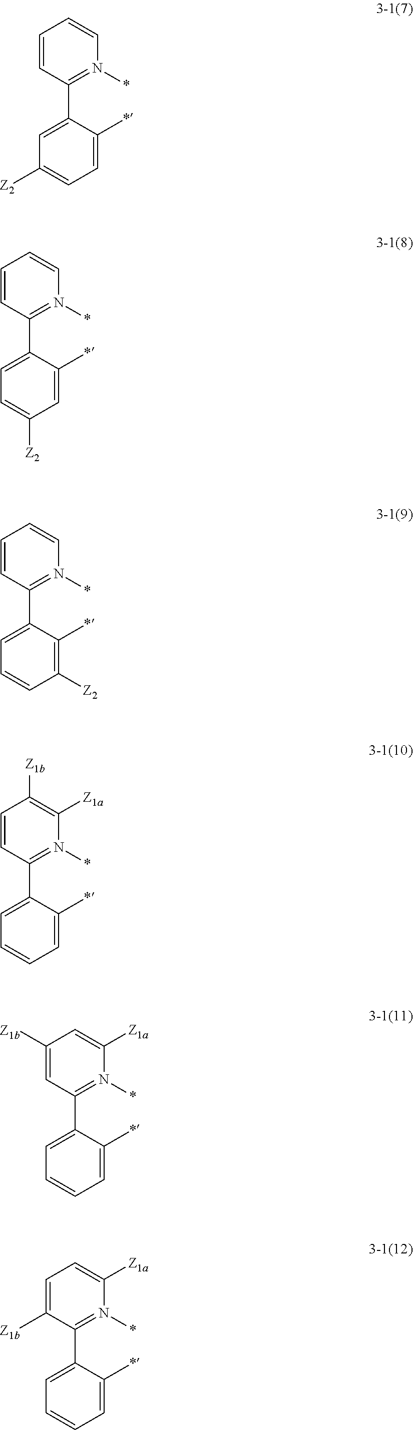

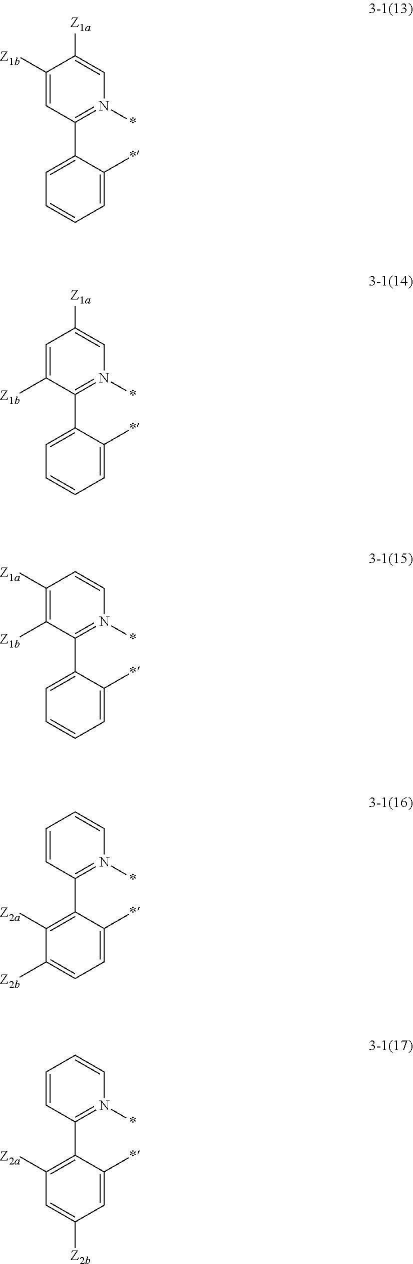

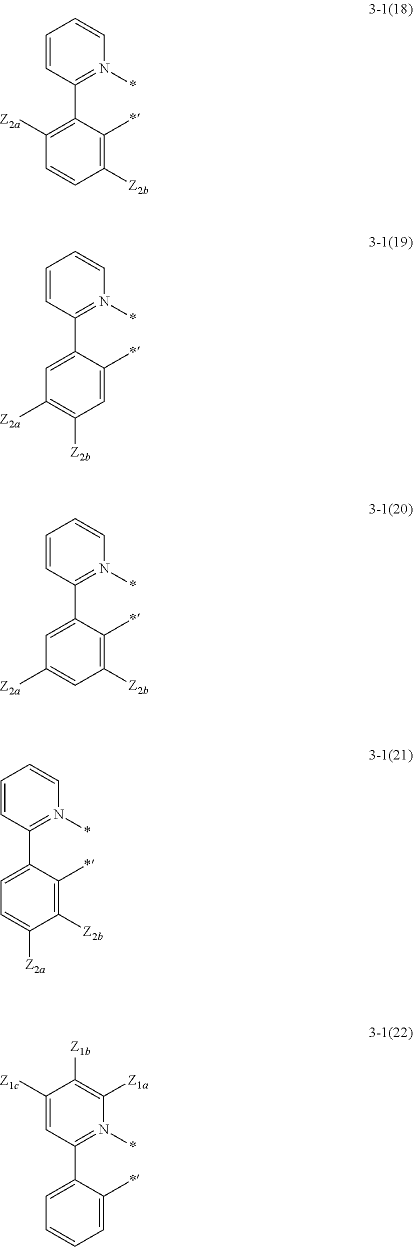

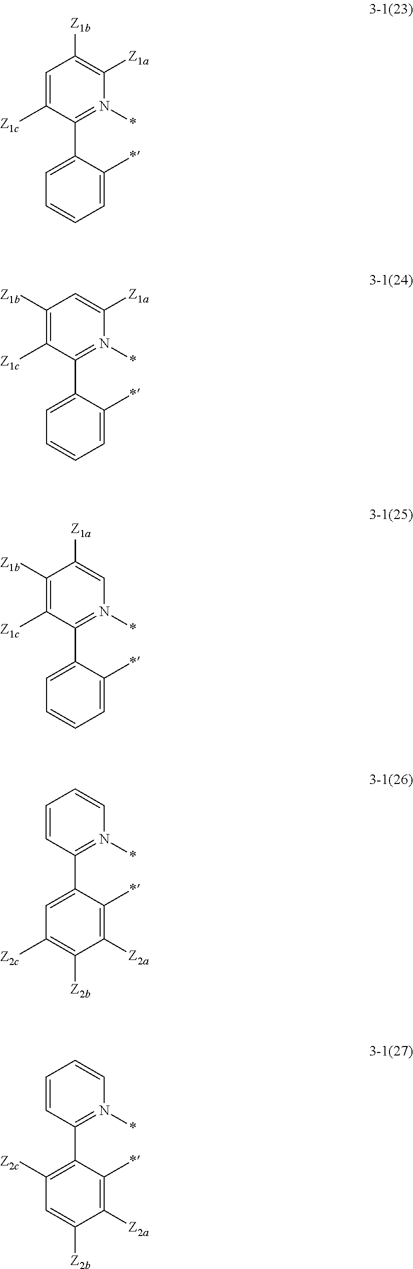

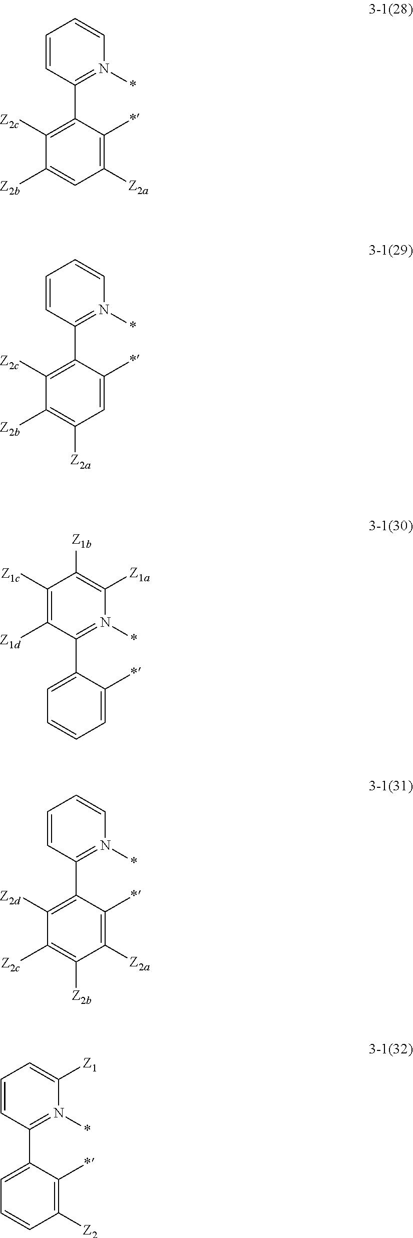

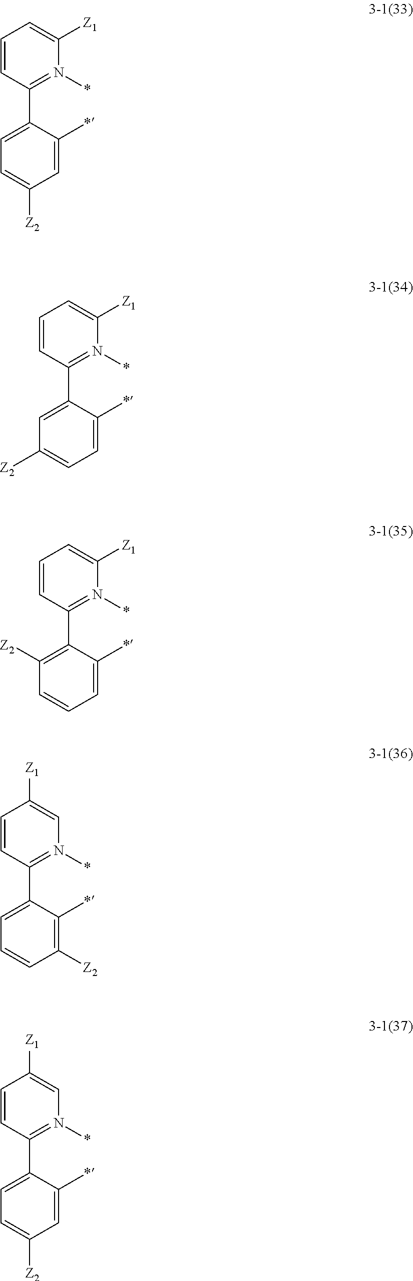

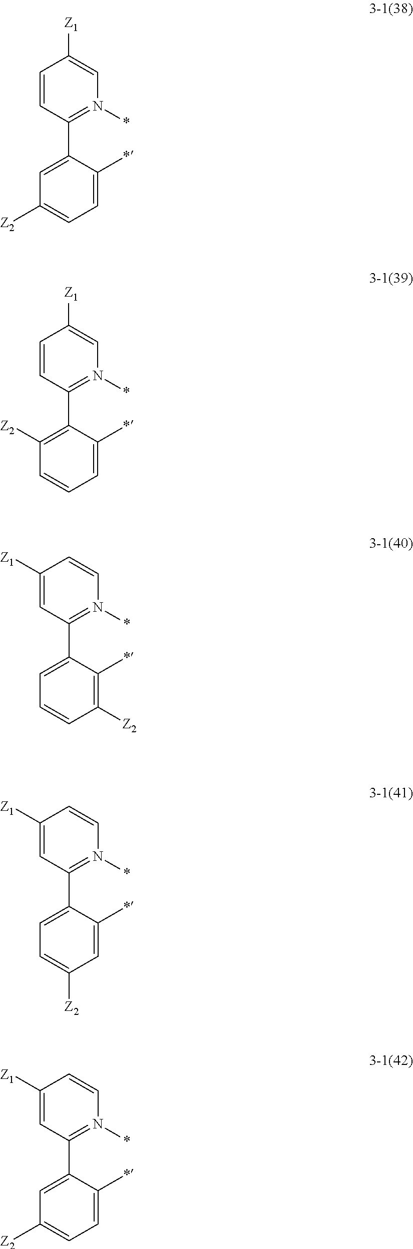

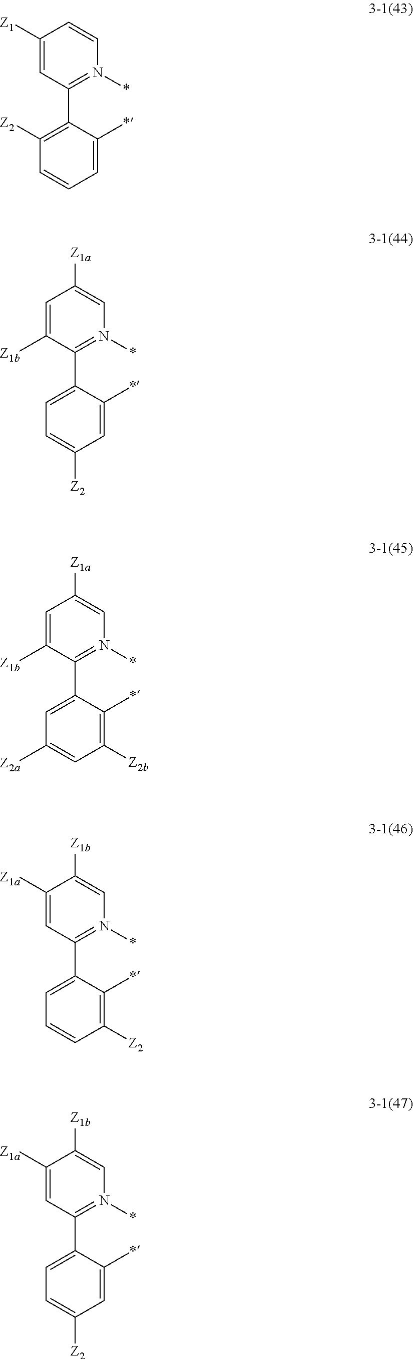

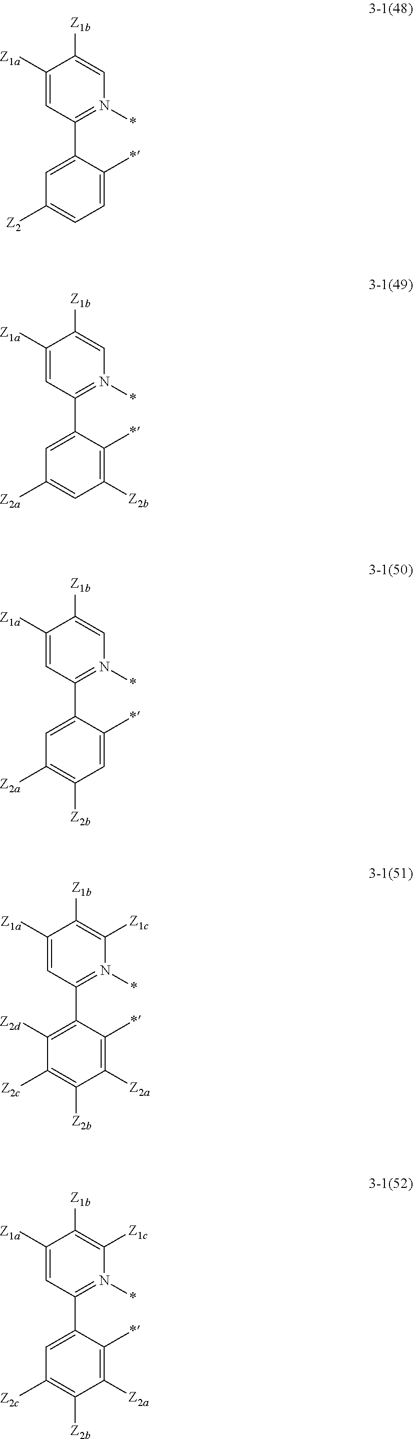

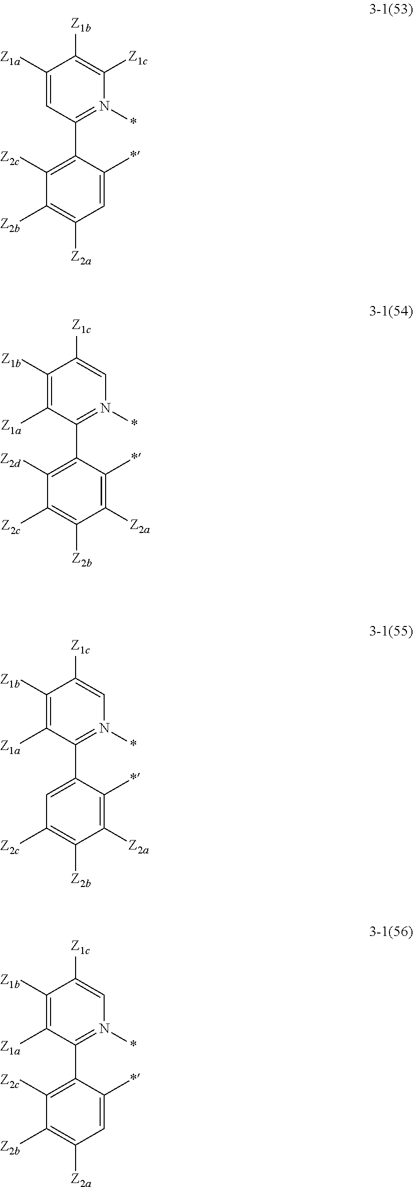

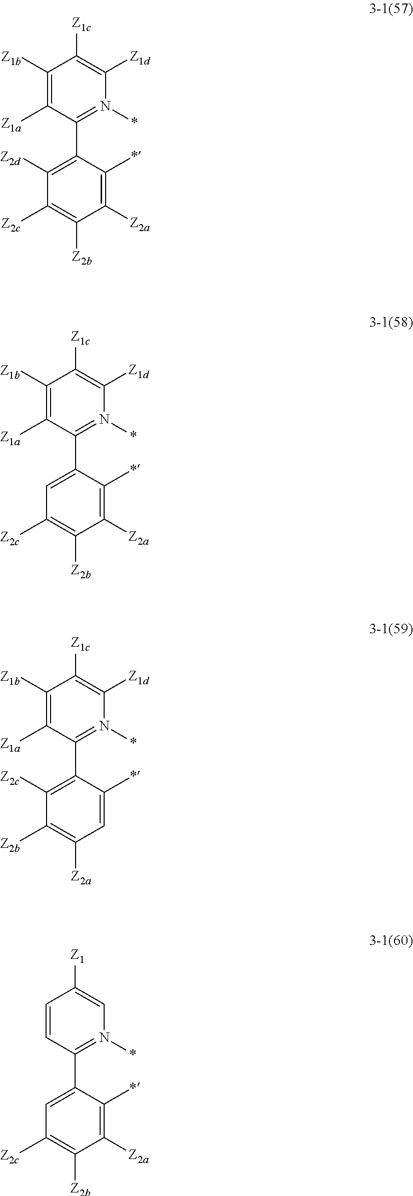

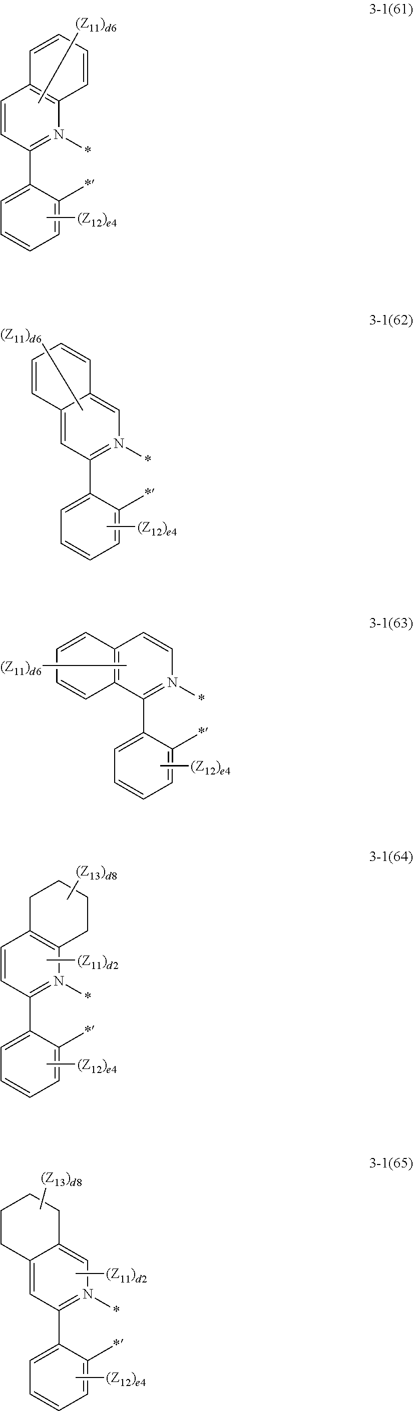

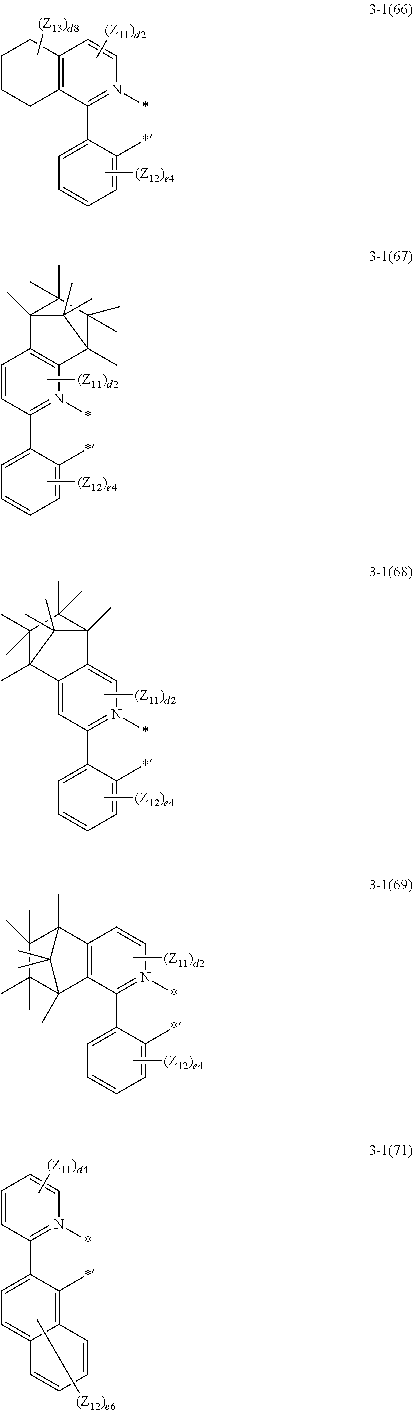

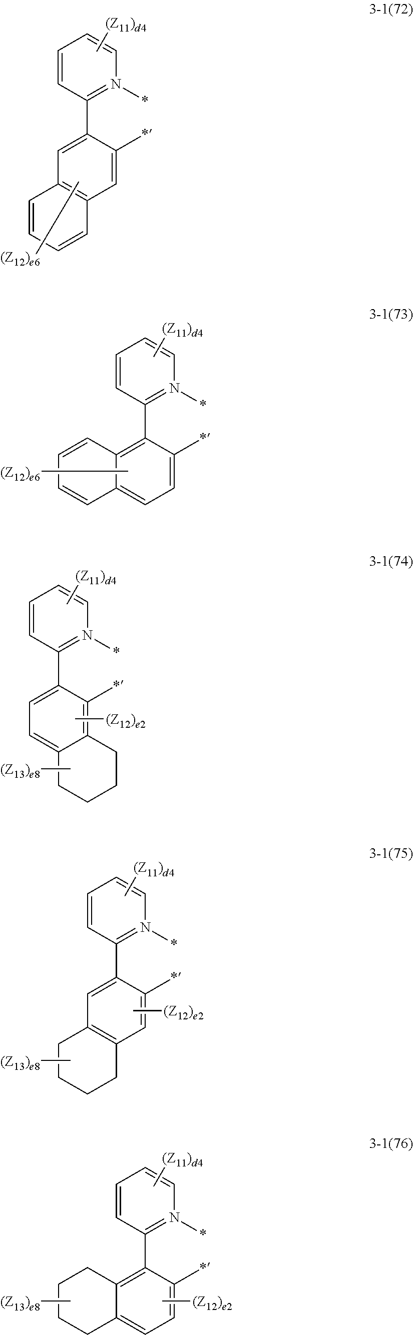

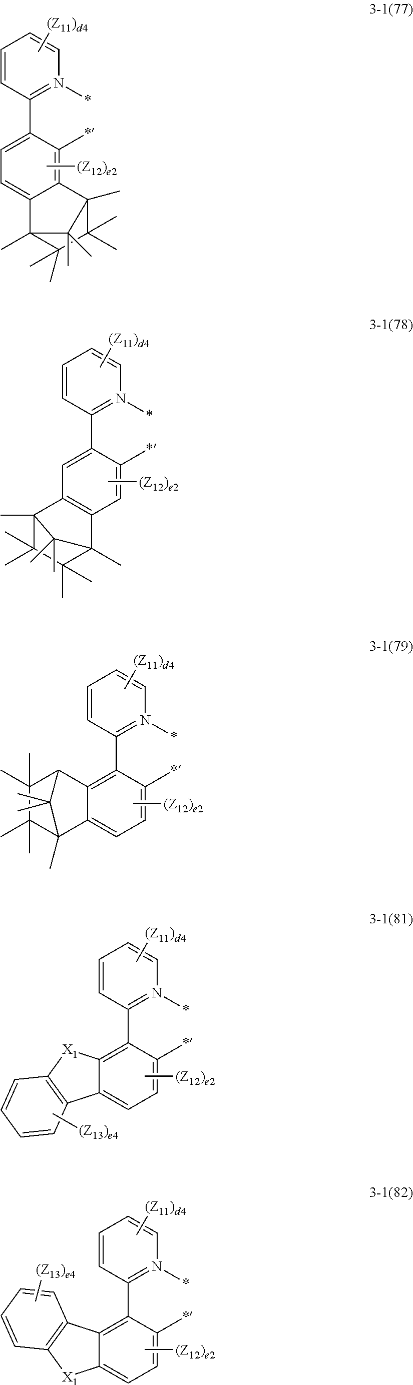

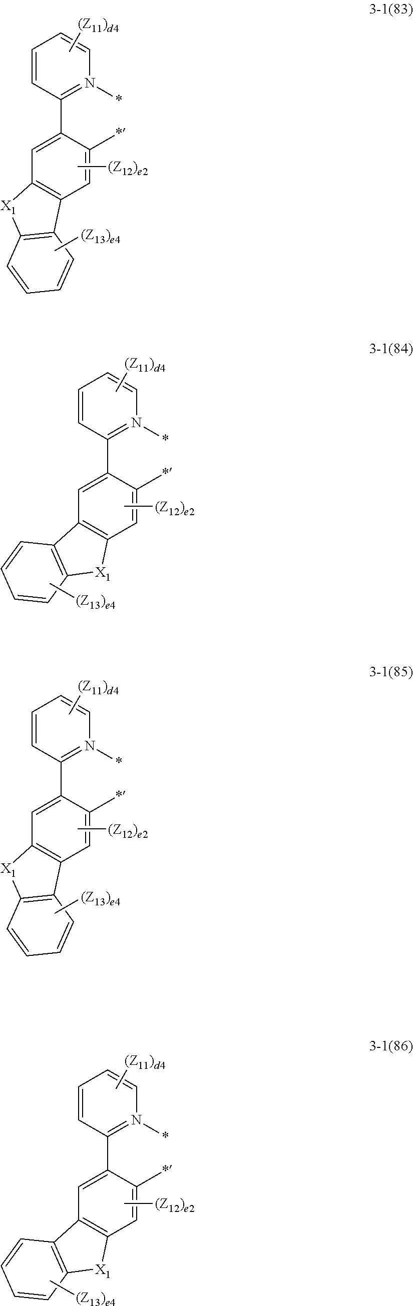

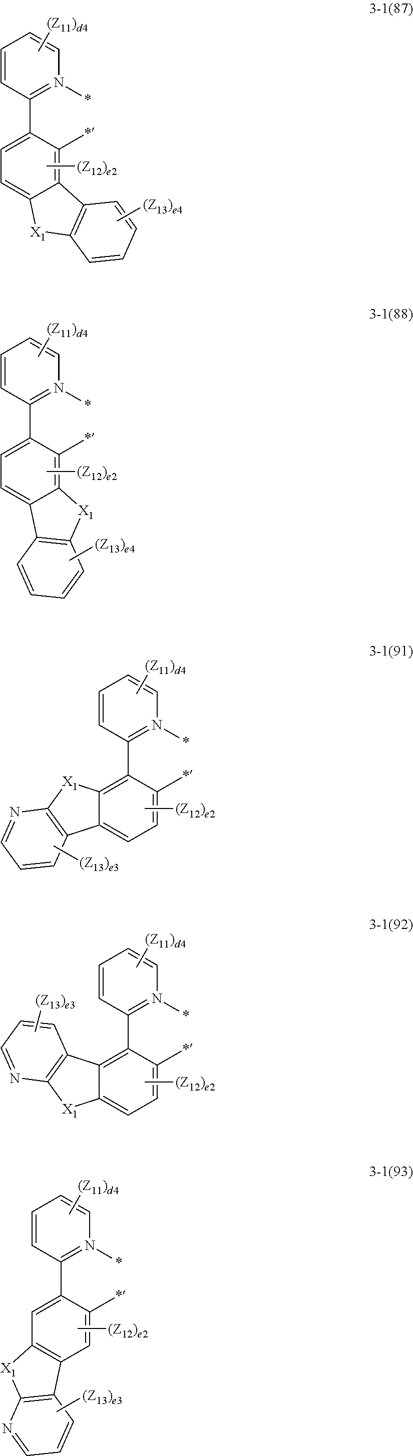

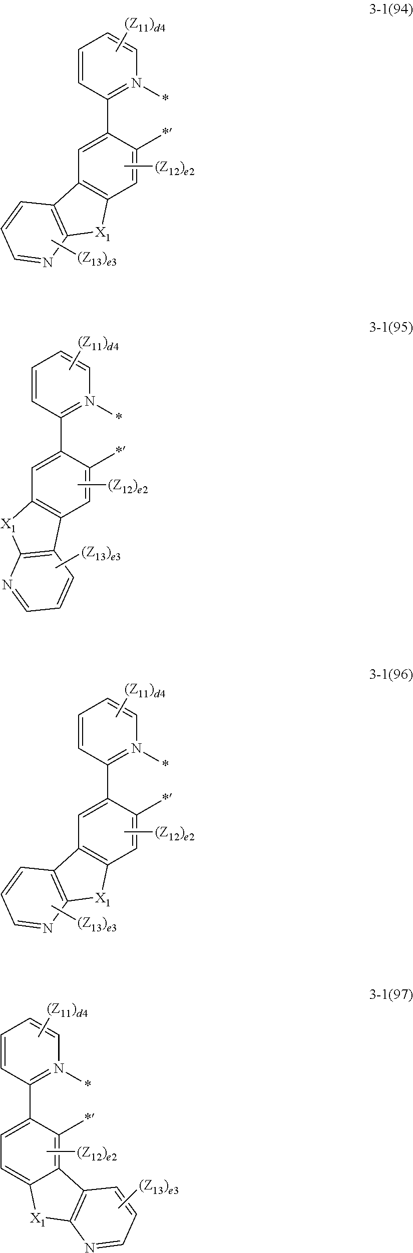

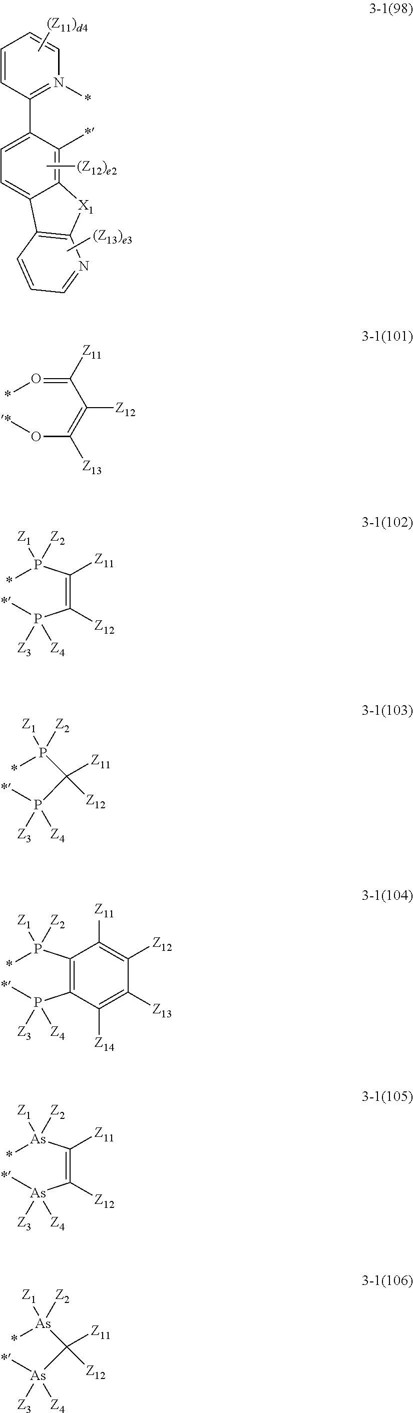

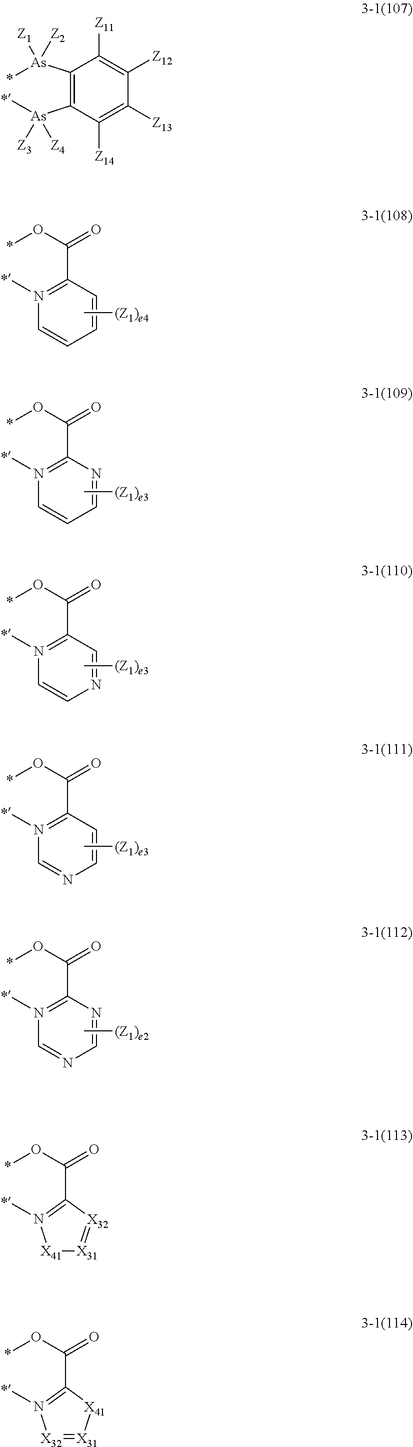

wherein, in Formulae 3-1(1) to 3-1(60), 3-1(61) to 3-1(69), 3-1(71) to 3-1(79), 3-1(81) to 3-1(88), 3-1(91) to 3-1(98), and 3-1(101) to 3-1(114),

X₁ may be O, S, C(Z₂₁)(Z₂₂), or N(Z₂₃),

X₃₁ may be N or C(Z₁ₐ), X₃₂ may be N or C(Z_{1b}),

X₄₁ may be O, S, N(Z₁ₐ), or C(Z₁ₐ)(Z_{1b}),

Z₁ to Z₄, Z₁ₐ, Z_{1b}, Z_{1c}, Z_{1d}, Z₂ₐ, Z_{2b}, Z_{2c}, Z_{2d}, Z₁₁ to Z₁₄, and Z₂₁ to Z₂₃ may each independently be selected from

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbomanyl group, a norbomenyl group, a cyclopentenyl group, a cyclohexenyl group, a cycloheptenyl group, a phenyl group, a naphthyl group, a pyridinyl group, and a pyrimidinyl group;

a cyclopentyl group, a cyclohexyl group, a cycloheptyl group, a cyclooctyl group, an adamantanyl group, a norbomanyl group, a norbomenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;

a cyclopentyl group, a cyclohexyl group, a cycloheptyl group, a cyclooctyl group, an adamantanyl group, a norbomanyl group, a norbomenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbomanyl group, a norbomenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and

—B(Q₈₆)(Q₈₇) and —P(═O)(Q₈₈)(Q₈₉),

wherein Q₈₆ to Q₈₉ may each independently be selected from

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CH₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group, each substituted with at least one selected from deuterium, a C₁-C₁₀ alkyl group, and a phenyl group.

d2 and e2 may each independently be 0 or 2,

e3 may be an integer from 0 to 3,

d4 and e4 may each independently be an integer from 0 to 4,

d6 and e6 may each independently be an integer from 0 to 6,

d8 and e8 may each independently be an integer from 0 to 8, and

* and *′ each indicate a binding site to M in Formula 1.

In one or more embodiments, in Formula 81, M may be Ir, and a sum of n81 and n82 may be 3. In one or more embodiments, in Formula 81, M may be Pt, and a sum of n81 and n82 may be 2.

In one or more embodiments, the organometallic compound represented by Formula 81 may be neutral, and may not include ion pairs of cations and anions.

In one or more embodiments, the organometallic compound represented by Formula 81 may include at least one selected from Compounds PD1 to PD78 and FIr₆, but embodiments are not limited thereto:

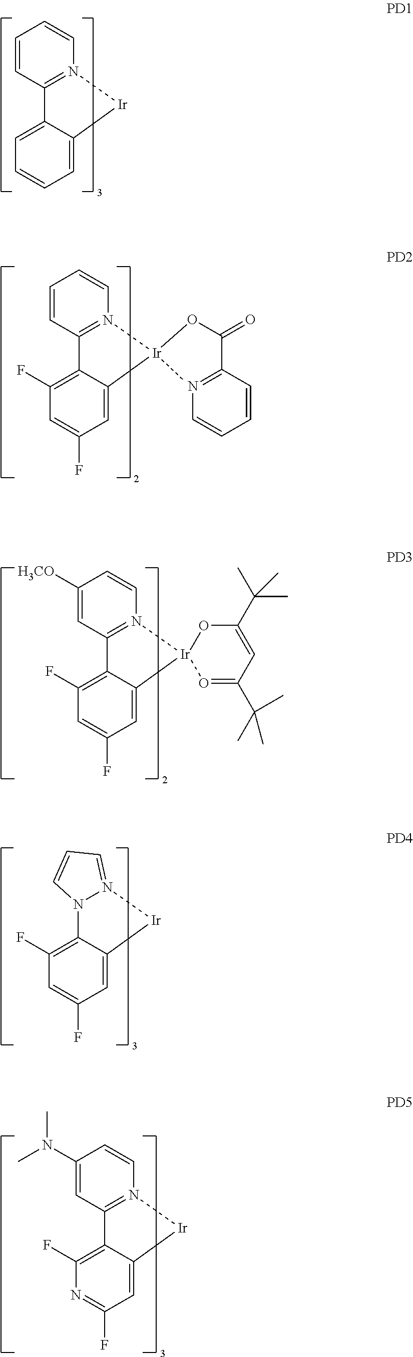

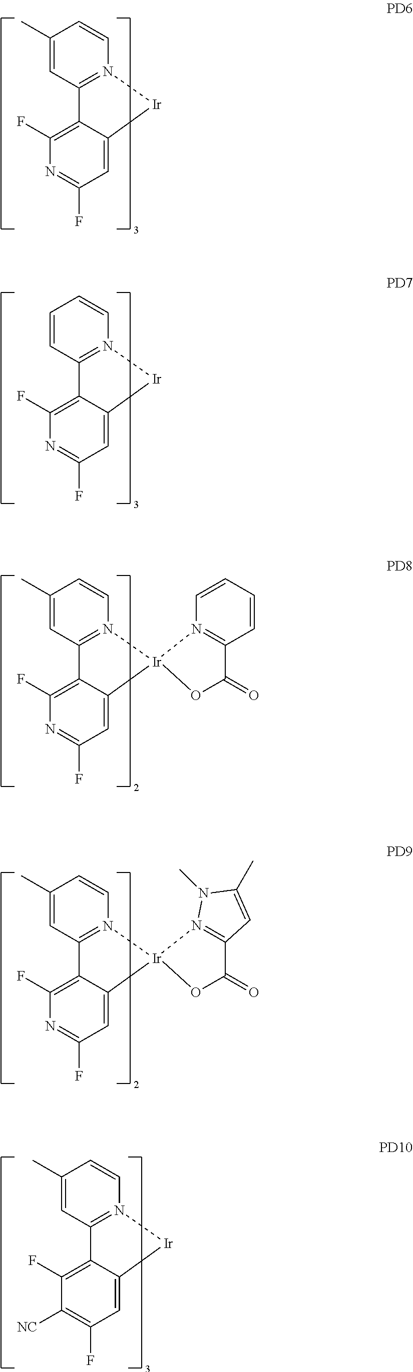

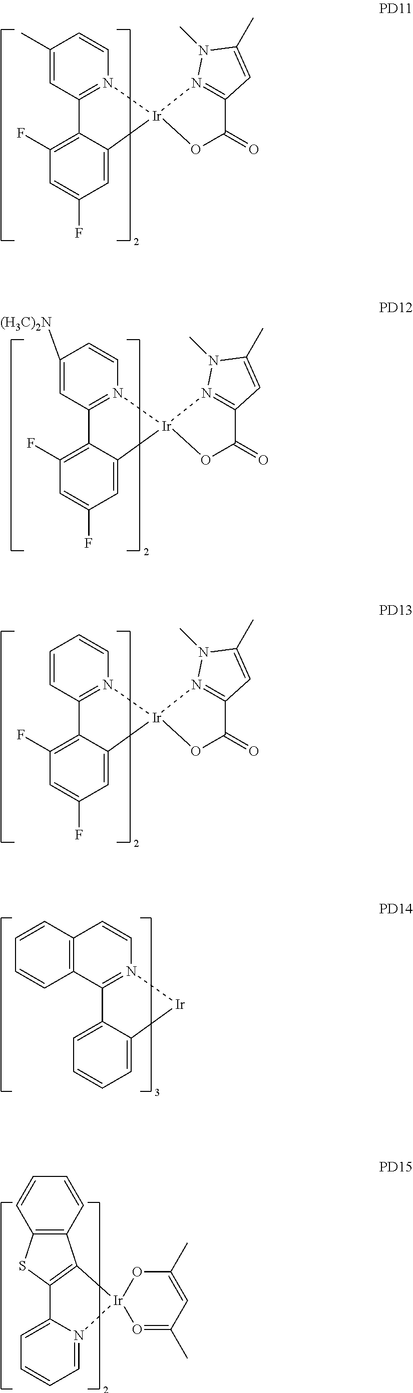

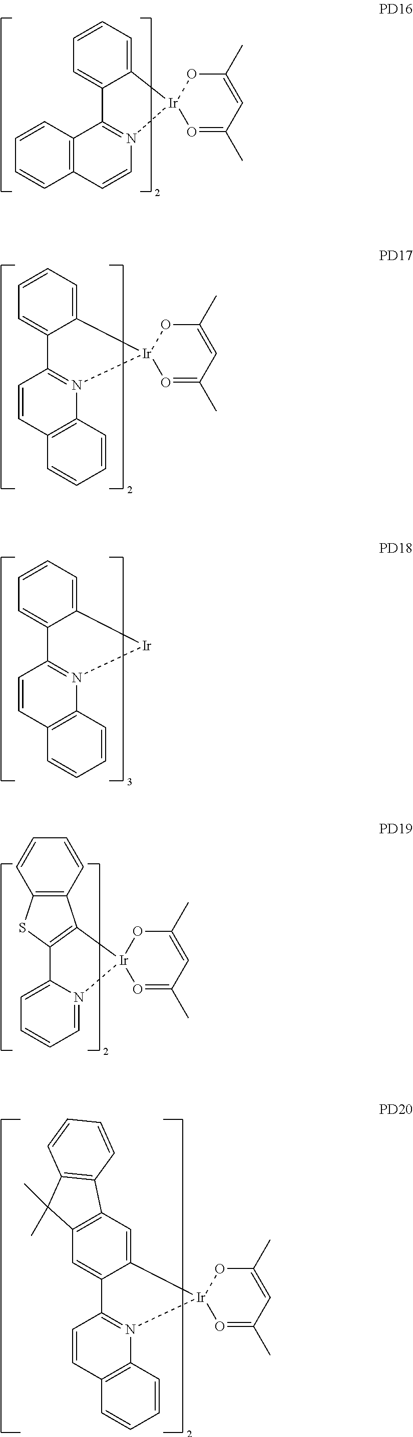

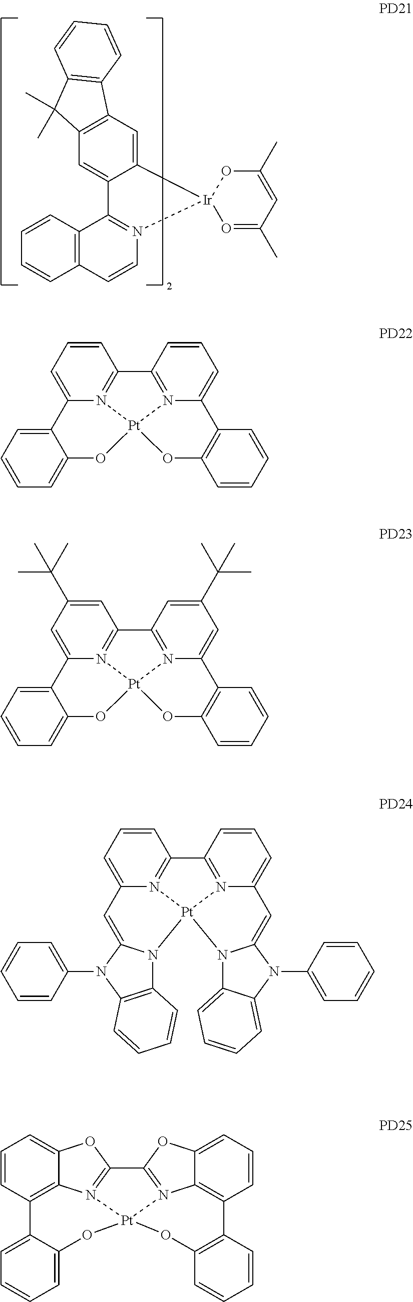

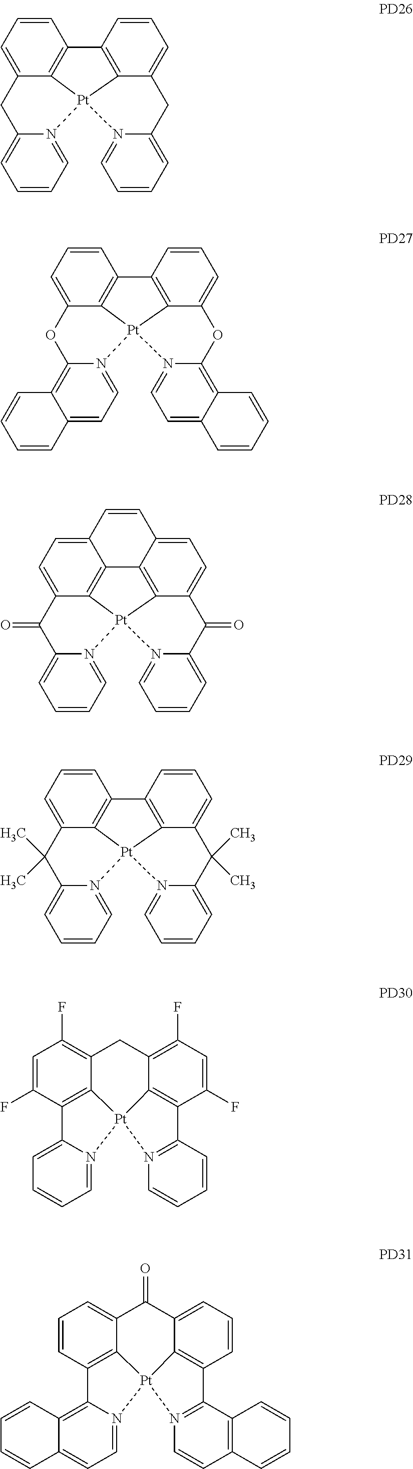

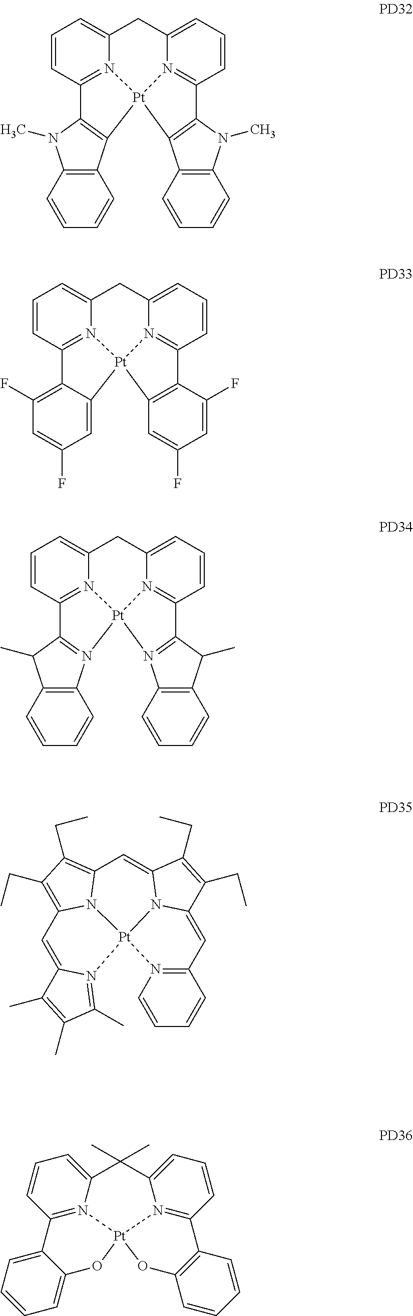

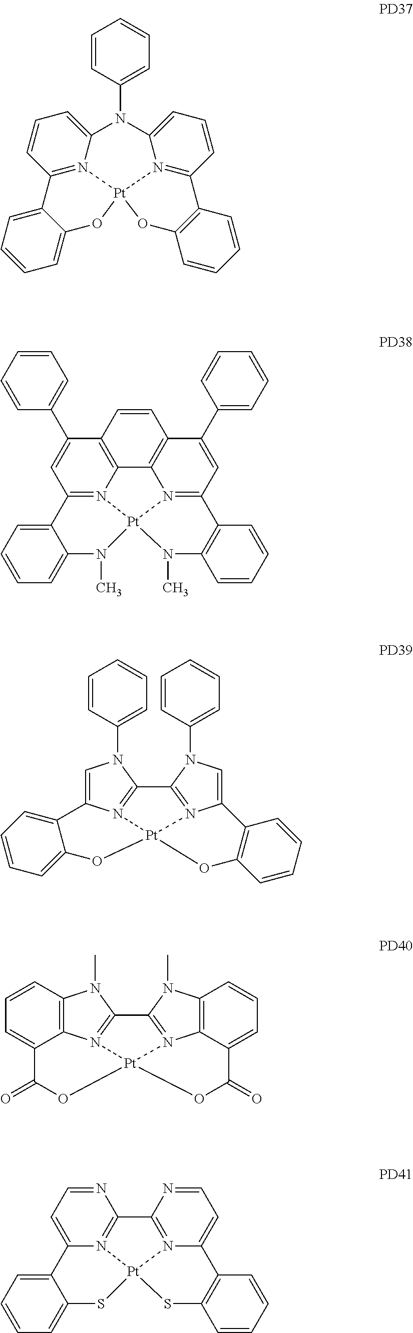

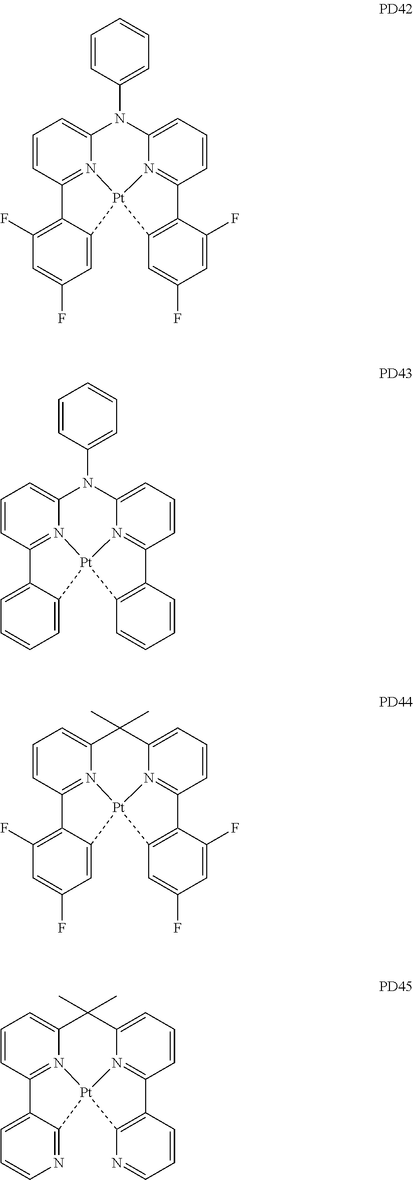

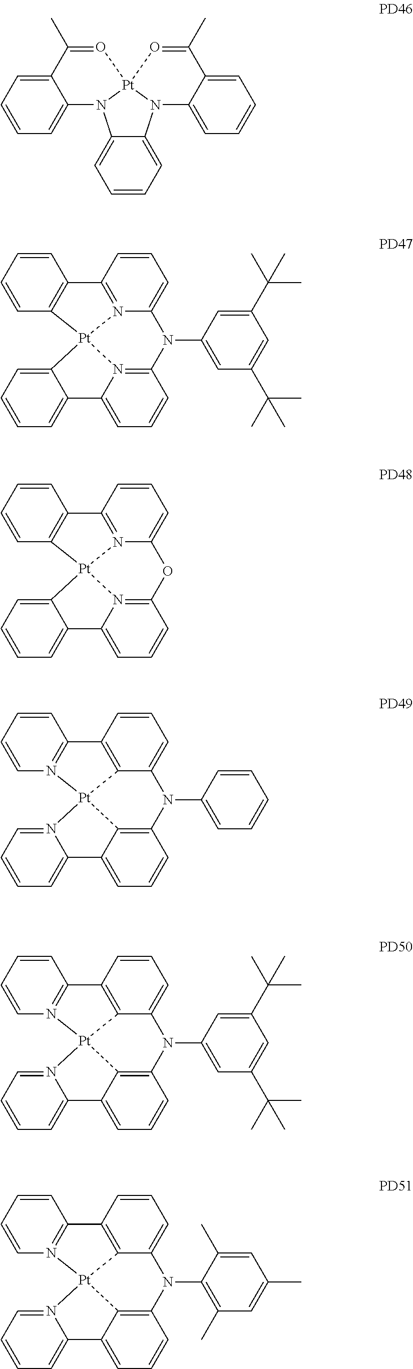

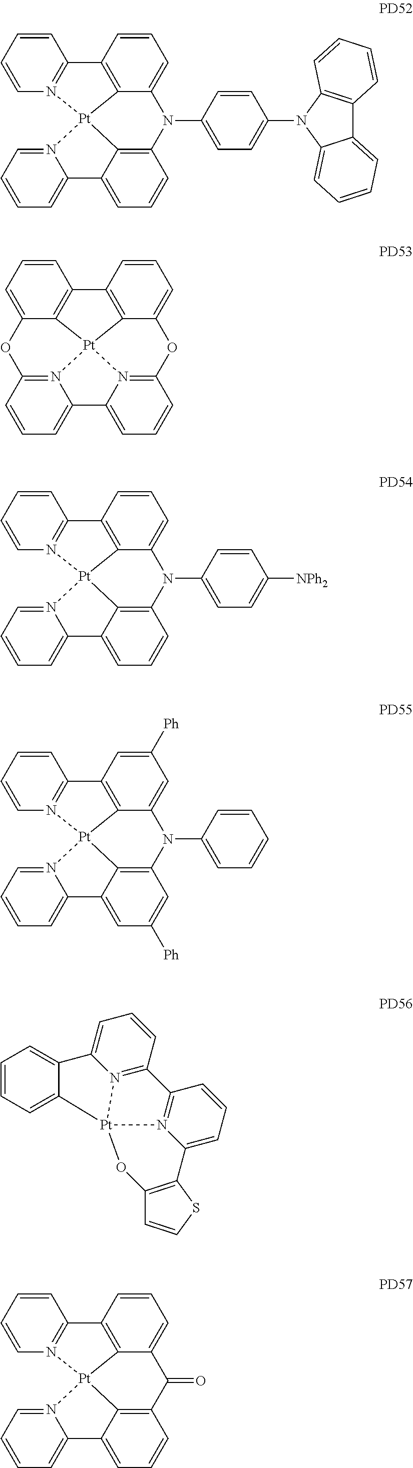

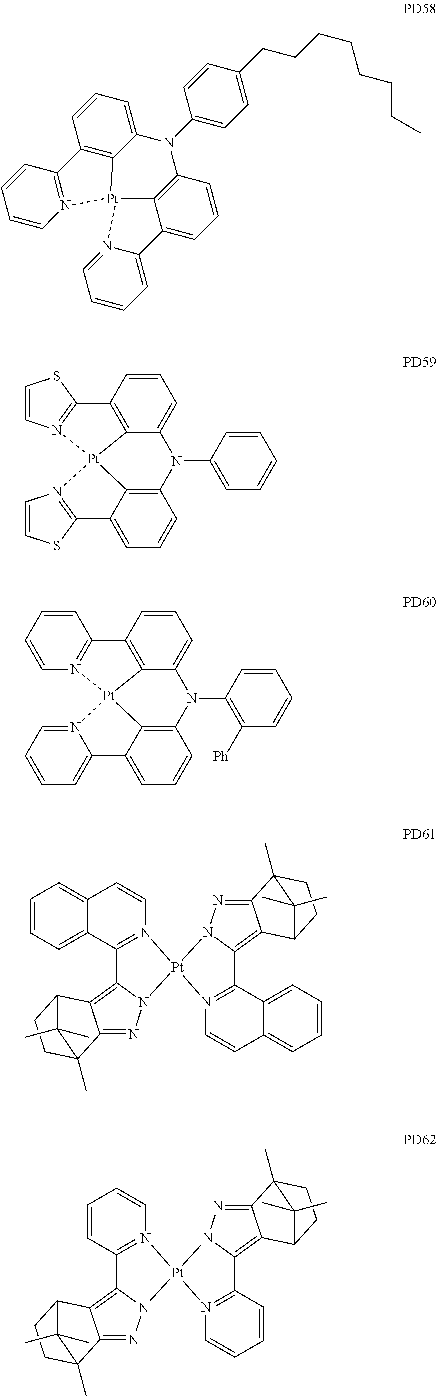

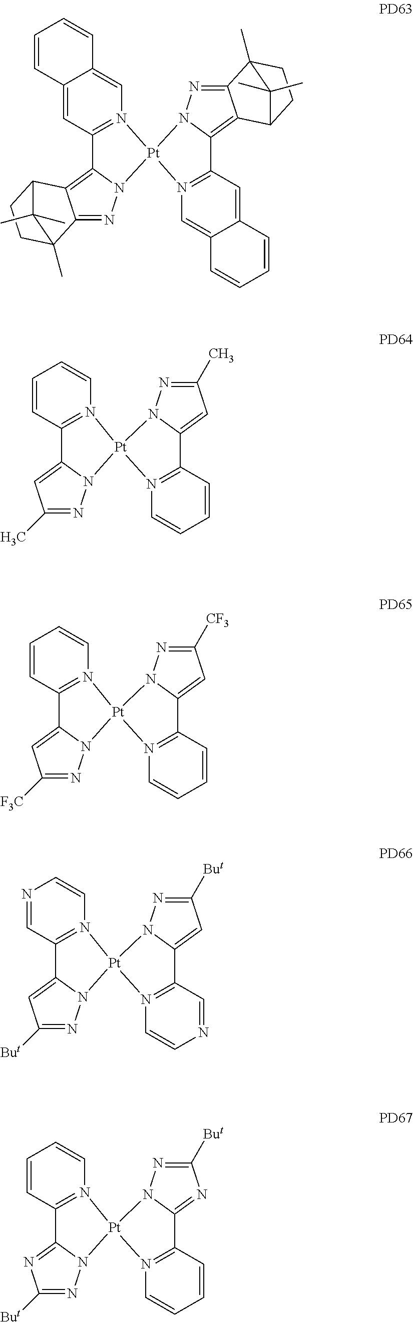

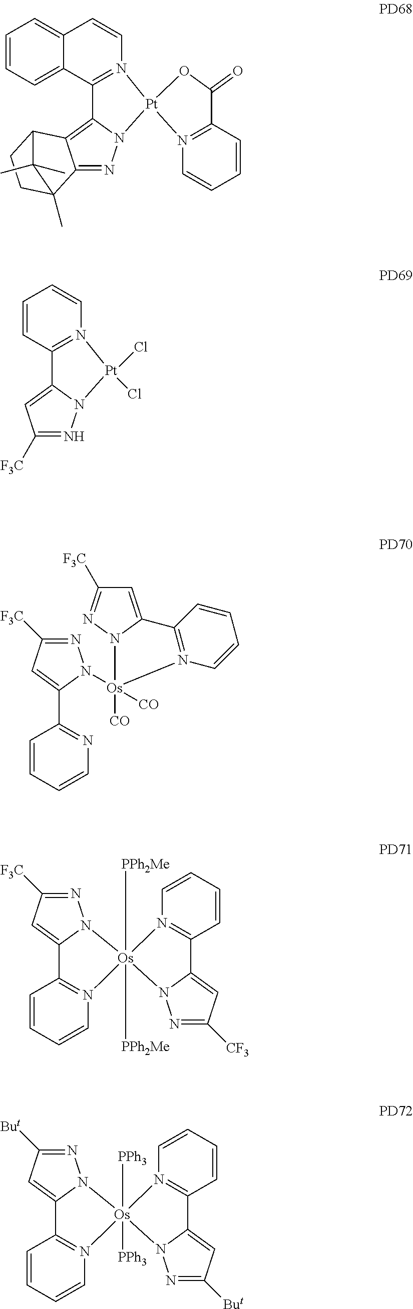

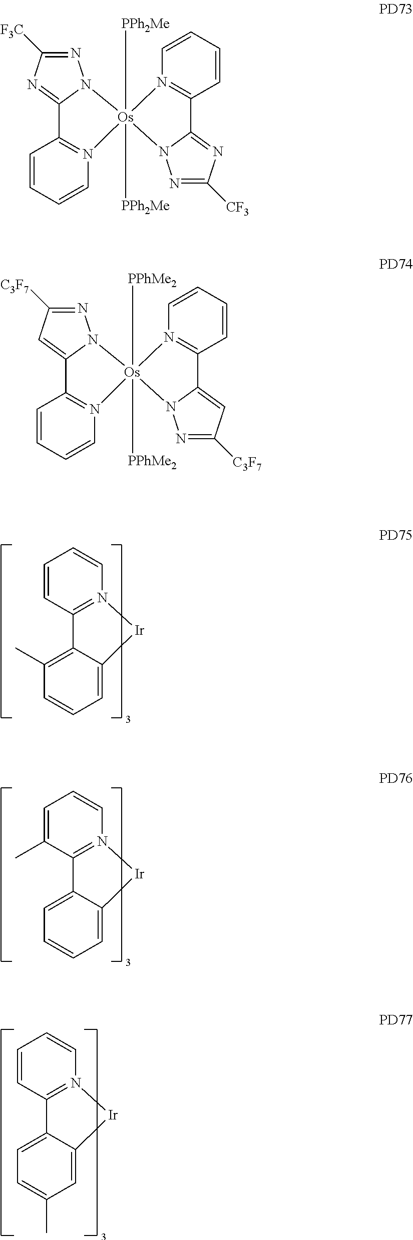

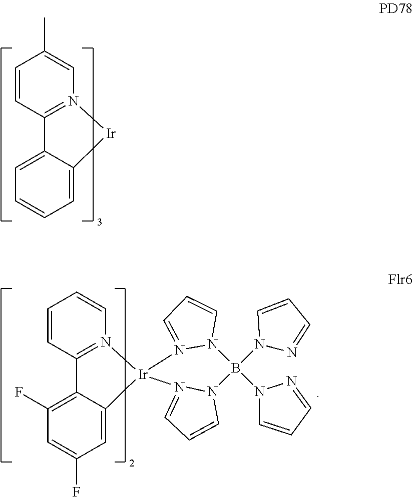

As used herein, the expression “(for example, the organic layer) including at least one condensed cyclic compound” means that “(the organic layer) including a condensed cyclic compound represented by Formula 1, or at least two different condensed cyclic compounds represented by Formula 1”.

For example, the organic layer may include Compound 1 only as the condensed cyclic compound. In this embodiment, Compound 1 may be included in the emission layer of the organic light-emitting device. In some embodiments, Compounds 1 and 2 may be included in the organic layer as the condensed cyclic compounds. In this embodiment, Compounds 1 and 2 may be present in the same layer (for example, Compounds 1 and 2 may be both present in an emission layer), or in different layers (for example, Compound 1 may be present in an emission layer, and Compound 2 may be present in a hole blocking layer).

The first electrode may be an anode, which is a hole injection electrode, and the second electrode may be a cathode, which is an electron injection electrode. In some embodiments, the first electrode may be a cathode, which is an electron injection electrode, and the second electrode may be an anode, which is a hole injection electrode.

The term “organic layer” as used herein refers to a single and/or a plurality of layers between the first electrode and the second electrode in an organic light-emitting device. The “organic layer” may include not only organic compounds but also organometallic complexes including metals.

The FIGURE illustrates a schematic cross-sectional view of an organic light-emitting device 10 according to an embodiment. Hereinafter, a structure of an organic light-emitting device according to one or more embodiments and a method of manufacturing the organic light-emitting device will be described with reference to the FIGURE. The organic light-emitting device 10 may include a first electrode 11, an organic layer 15, and a second electrode 19, which may be sequentially layered in this stated order.

A substrate may be additionally disposed under the first electrode 11 or on the second electrode 19. The substrate may be a conventional substrate used in organic light-emitting devices, e.g., a glass substrate or a transparent plastic substrate, each having excellent mechanical strength, thermal stability, transparency, surface smoothness, ease of handling, and water repellency.

The first electrode 11 may be formed by vacuum-depositing or sputtering, onto the substrate, a material for forming the first electrode 11. The first electrode 11 may be an anode. The material for forming the first electrode 11 may be selected from materials with a high work function for easy hole injection. The first electrode 11 may be a reflective electrode, a semi-transmissive electrode, or a transmissive electrode.

The material for forming the first electrode 11 may be selected from indium tin oxide (ITO), indium zinc oxide (IZO), tin oxide (SnO₂), and zinc oxide (ZnO). In some embodiments, the material for forming the first electrode 11 may be a metal, such as magnesium (Mg), aluminum (Al), aluminum-lithium (Al—Li), calcium (Ca), magnesium-indium (Mg—In), or magnesium-silver (Mg—Ag).

The first electrode 11 may have a single-layered structure or a multi-layered structure including a plurality of layers. In some embodiments, the first electrode 11 may have a triple-layered structure of ITO/Ag/ITO, but embodiments are not limited thereto.

The organic layer 15 may be on the first electrode 11.

The organic layer 15 may include a hole transport region, an emission layer, and an electron transport region.

The hole transport region may be disposed between the first electrode 11 and the emission layer.

The hole transport region may include a hole injection layer, a hole transport layer, an electron blocking layer, a buffer layer, or any combination thereof.

The hole transport region may include a hole injection layer only or a hole transport layer only. In some embodiments, the hole transport region may include a hole injection layer and a hole transport layer which are sequentially stacked on the first electrode 11. In some embodiments, the hole transport region may include a hole injection layer, a hole transport layer, and an electron blocking layer, which are sequentially stacked on the first electrode 11.

When the hole transport region includes a hole injection layer, the hole injection layer may be formed on the first electrode 11 by using one or more suitable methods, such as vacuum deposition, spin coating, casting, and Langmuir-Blodgett (LB) deposition.

When a hole injection layer is formed by vacuum deposition, for example, the vacuum deposition may be performed at a deposition temperature in a range of about 100° C. to about 500° C., at a vacuum degree in a range of about 10⁻⁸ torr to about 10⁻³ torr, and at a deposition rate in a range of about 0 Angstroms per second (Å/sec) to about 100 Å/sec, though the conditions may vary depending on a compound that is used as a hole injection material and a structure and thermal properties of a desired hole injection layer, but conditions for the vacuum deposition are not limited thereto.

When a hole injection layer is formed by spin coating, the spin coating may be performed at a coating rate in a range of about 2,000 revolutions per minute (rpm) to about 5,000 rpm, and at a temperature in a range of about 80° C. to 200° C., to facilitate removal of a solvent after the spin coating, though the conditions may vary depending on a compound that is used as a hole injection material and a structure and thermal properties of a desired hole injection layer, but conditions for the spin coating are not limited thereto.

The conditions for forming a hole transport layer and an electron blocking layer may be inferred from the conditions for forming the hole injection layer.

The hole transport region may include at least one selected from m-MTDATA, TDATA, 2-TNATA, NPB, 13-NPB, TPD, spiro-TPD, spiro-NPB, methylated-NPB, TAPC, HMTPD, 4,4′,4″-tris(N-carbazolyl)triphenylamine (TCTA), polyaniline/dodecylbenzene sulfonic acid (PANI/DBSA), poly(3,4-ethylenedioxythiophene)/poly(4-styrene sulfonate) (PEDOT/PSS), polyaniline/camphor sulfonic acid (PANI/CSA), (polyaniline)/poly(4-styrene sulfonate) (PANI/PSS), a compound represented by Formula 201, and a compound represented by Formula 202:

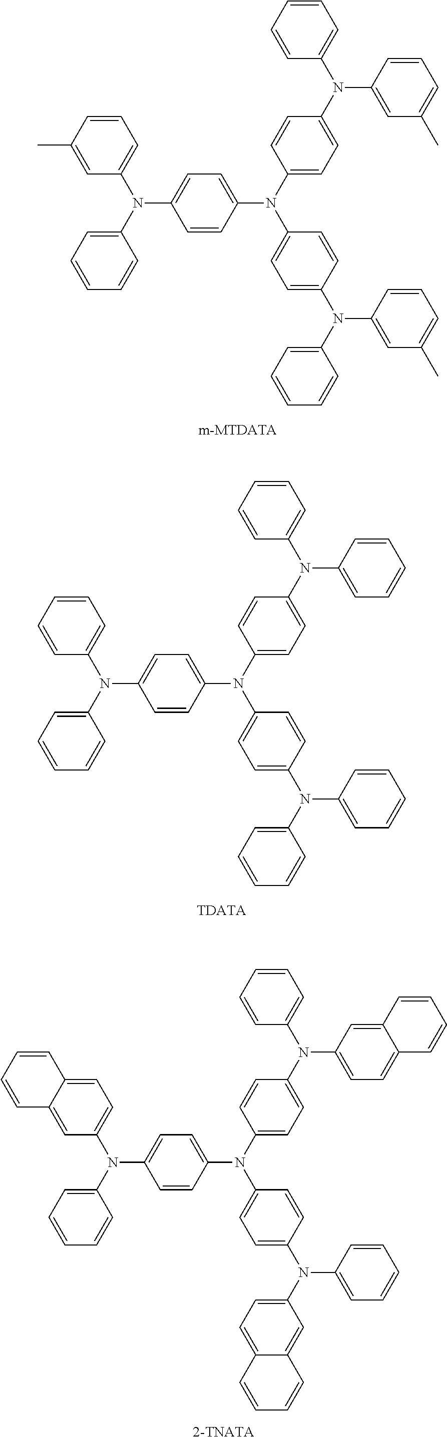

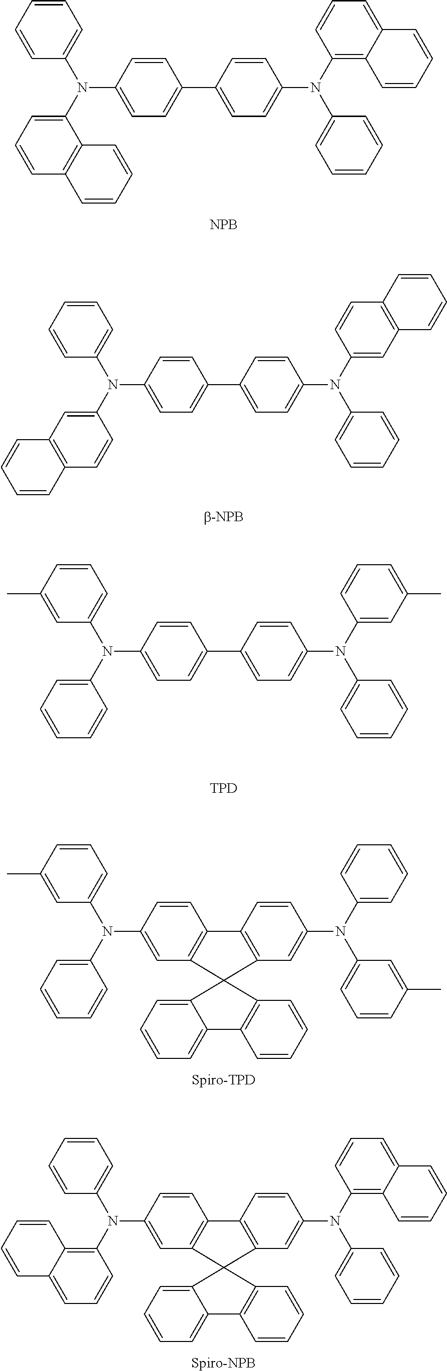

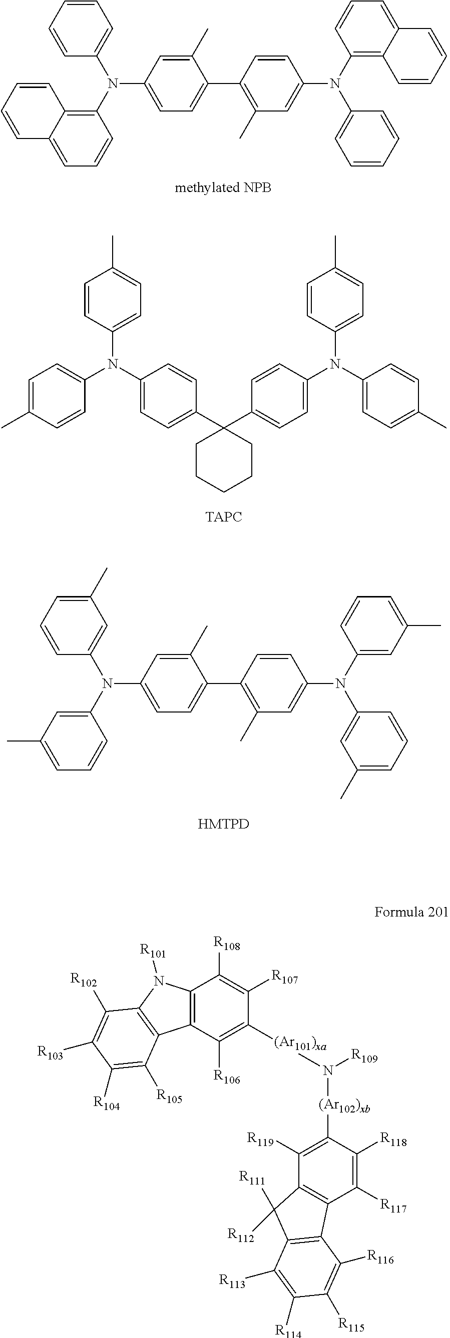

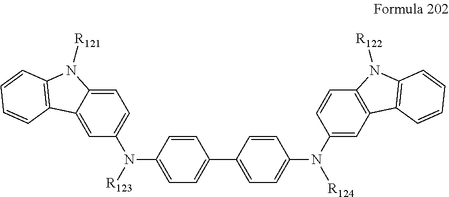

wherein, in Formula 201, Ar₁₀₁ and Ar₁₀₂ may each independently be selected from

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group; and

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

In Formula 201, xa and xb may each independently be an integer from 0 to 5. In some embodiments, xa and xb may each independently be an integer selected from 0, 1, and 2. In some embodiments, xa may be 1, and xb may be 0, but embodiments are not limited thereto.

In Formulae 201 and 202, R₁₀₁ to R₁₀₈, R₁₁₁ to R₁₁₉, and R₁₂₁ to R₁₂₄ may each independently be selected from

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (e.g., a methyl group, an ethyl group, a propyl group, a butyl group, pentyl group, or a hexyl group), and a C₁-C₁₀ alkoxy group (e.g., a methoxy group, an ethoxy group, a propoxy group, a butoxy group, or a pentoxy group);

a C₁-C₁₀ alkyl group and a C₁-C₁₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a phenyl group, a naphthyl group, an anthracenyl group, a fluorenyl group, and a pyrenyl group; and

a phenyl group, a naphthyl group, an anthracenyl group, a fluorenyl group, and a pyrenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group, but embodiments are not limited thereto.

In Formula 201, R₁₀₉ may be selected from

a phenyl group, a naphthyl group, an anthracenyl group, and a pyridinyl group; and

a phenyl group, a naphthyl group, an anthracenyl group, and a pyridinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, and a pyridinyl group.

In some embodiments, the compound represented by Formula 201 may be represented by Formula 201A, but embodiments are not limited thereto:

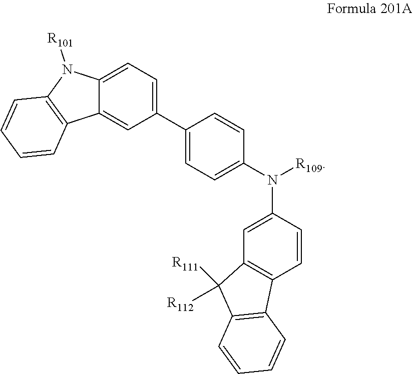

R₁₀₁, R₁₁₁, R₁₁₂, and R₁₀₉ in Formula 201A may respectively be understood by referring to the descriptions for those provided herein.

In some embodiments, the compounds represented by Formulae 201 and 202 may include Compounds HT1 to HT20, but embodiments are not limited thereto:

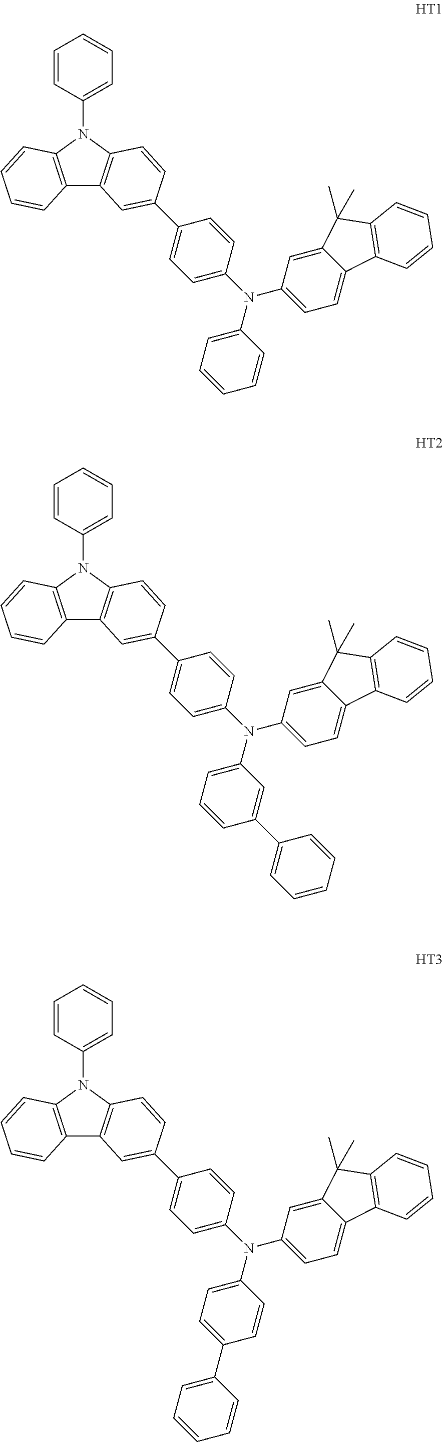 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

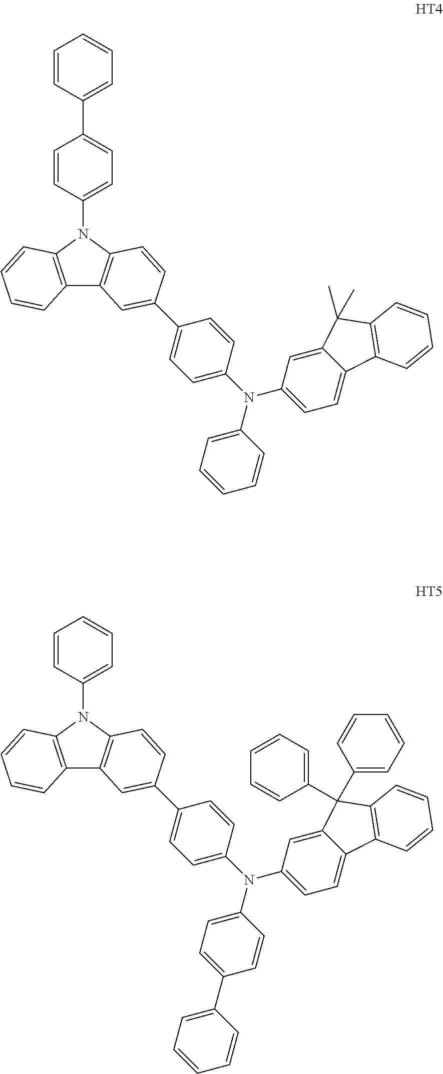 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

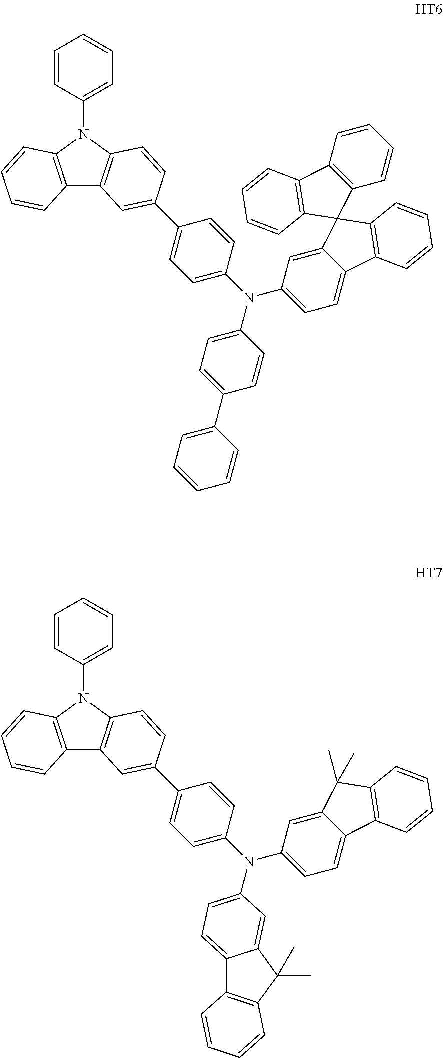 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

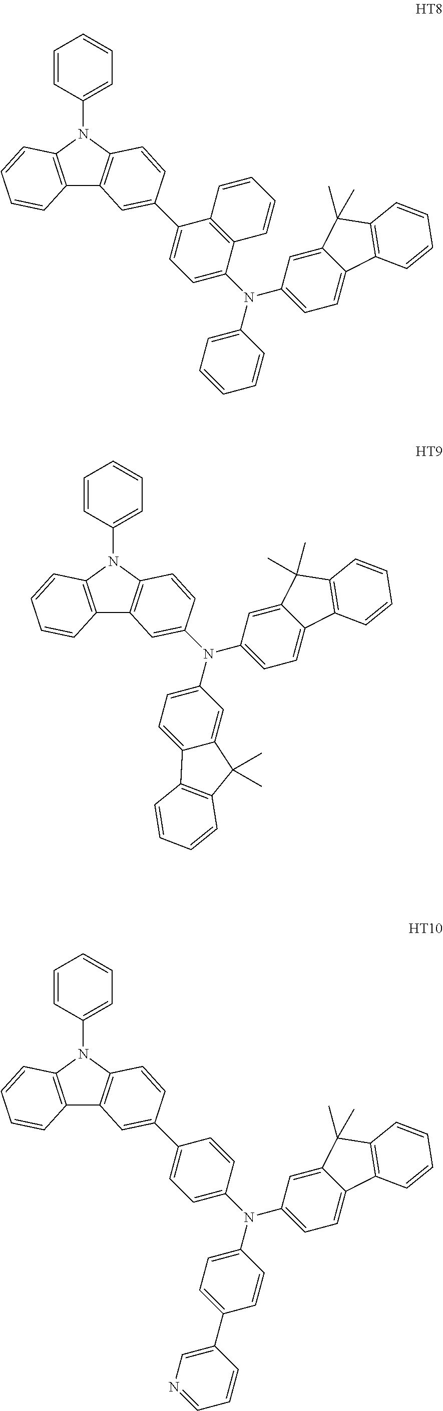 [SMILES: CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21]

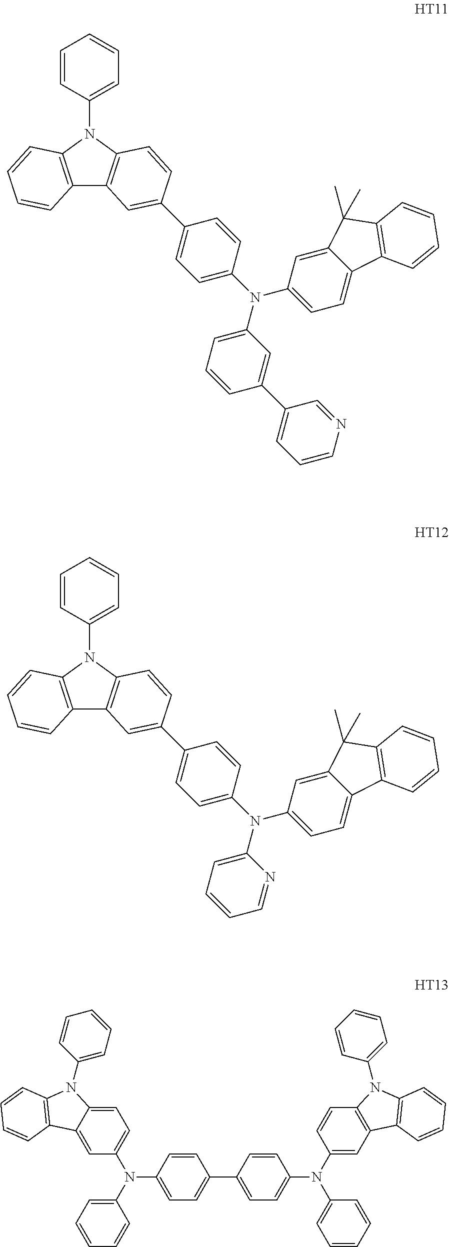 [SMILES: CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

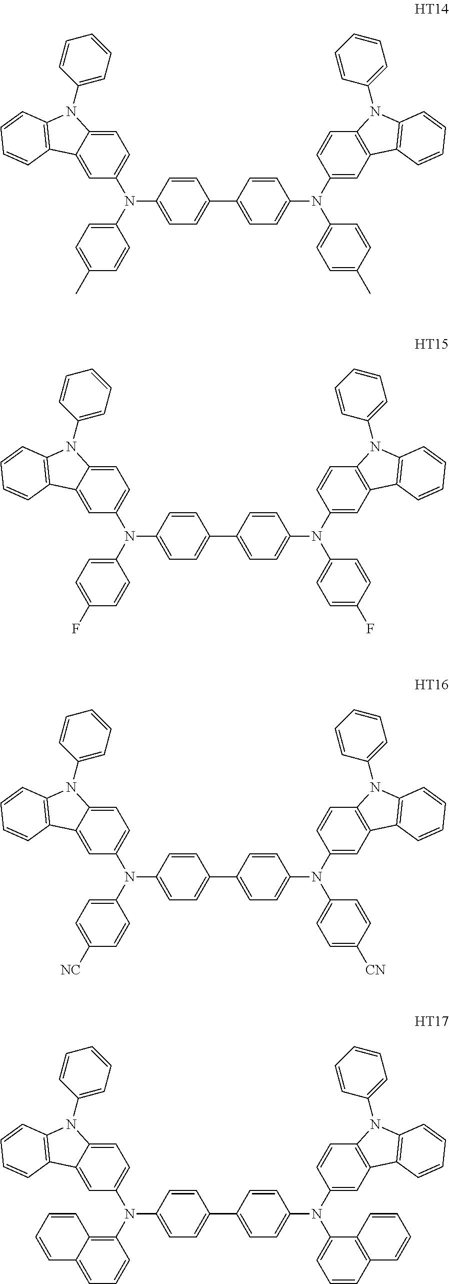 [SMILES: Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1]

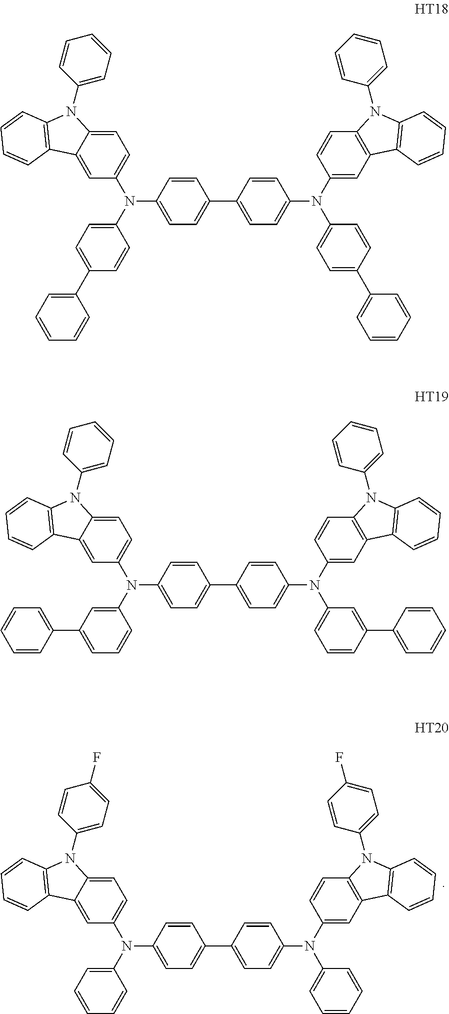 [SMILES: c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

The thickness of the hole transport region may be in a range of about 100 (Angstroms) Å to about 10,000 Å, and in some embodiments, about 100 Å to about 1,000 Å. When the hole transport region includes at least one of a hole injection layer and a hole transport layer, the thickness of the hole injection layer may be in a range of about 100 Å to about 10,000 Å, and in some embodiments, about 100 Å to about 1,000 Å, and the thickness of the hole transport layer may be in a range of about 50 Å to about 2,000 Å, and in some embodiments, about 100 Å to about 1,500 Å. While not wishing to be bound by theory, it is understood that when the thicknesses of the hole transport region, the hole injection layer, and the hole transport layer are within any of these ranges, excellent hole transport characteristics may be obtained without a substantial increase in driving voltage.

The hole transport region may include a charge generating material as well as the aforementioned materials, to improve conductive properties of the hole transport region. The charge generating material may be substantially homogeneously or non-homogeneously dispersed in the hole transport region.

The charge generating material may include, for example, a p-dopant. The p-dopant may include one of a quinone derivative, a metal oxide, and a compound containing a cyano group, but embodiments are not limited thereto. For example, non-limiting examples of the p-dopant include a quinone derivative, such as tetracyanoquinodimethane (TCNQ) or 2,3,5,6-tetrafluoro-tetracyano-1,4-benzoquinonedimethane (F4-TCNQ); a metal oxide, such as a tungsten oxide or a molybdenum oxide; and a compound containing a cyano group, such as Compound HT-D1 or Compound HT-D2, but embodiments are not limited thereto:

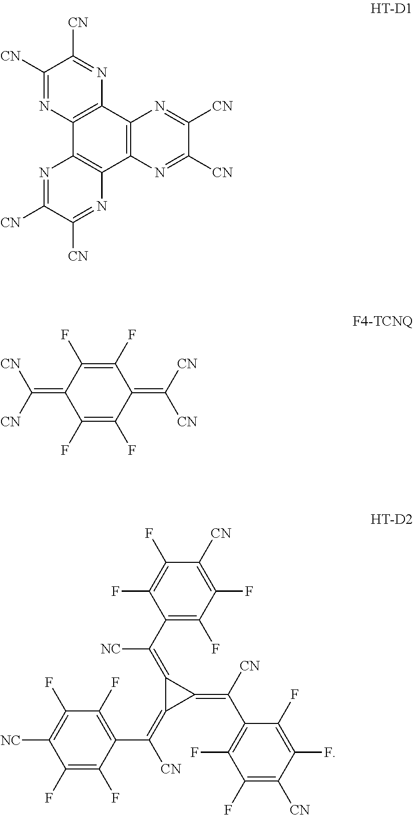

The hole transport region may further include a buffer layer.

The buffer layer may compensate for an optical resonance distance depending on a wavelength of light emitted from the emission layer to improve the efficiency of an organic light-emitting device.

An emission layer may be formed on the hole transport region by using one or more suitable methods, such as vacuum deposition, spin coating, casting, or LB deposition. When the emission layer is formed by vacuum deposition or spin coating, vacuum deposition and coating conditions for forming the emission layer may be generally similar to the those conditions for forming a hole injection layer, though the conditions may vary depending on a compound that is used.

The hole transport region may further include an electron blocking layer. The electron blocking layer may include any suitable known material, e.g., mCP, but embodiments are not limited thereto:

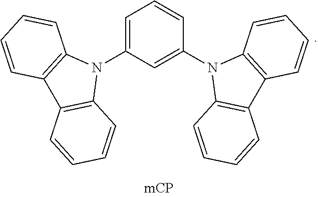 [SMILES: c1cc(-n2c3ccccc3c3ccccc32)cc(-n2c3ccccc3c3ccccc32)c1]

The thickness of the electron blocking layer may be in a range of about 50 Å to about 1,000 Å, and in some embodiments, about 70 Å to about 500 Å. While not wishing to be bound by theory, it is understood that when the thickness of the electron blocking layer is within any of these ranges, excellent electron blocking characteristics may be obtained without a substantial increase in driving voltage.

When the organic light-emitting device 10 is a full-color organic light-emitting device, the emission layer may be patterned into a red emission layer, a green emission layer, and a blue emission layer. In some embodiments, the emission layer may have a structure in which the red emission layer, the green emission layer, and/or the blue emission layer are layered to emit white light. In some embodiments, the structure of the emission layer may vary.

The emission layer may include the condensed cyclic compound represented by Formula 1.

In some embodiments, the emission layer may include the condensed cyclic compound represented by Formula 1 only.

In some embodiments, the emission layer may include the condensed cyclic compound represented by Formula 1,

i) the second compound (e.g., a compound represented by Formula H-1);

ii) the organometallic compound represented by Formula 81; or

iii) any combination of i) and ii).

The condensed cyclic compound represented by Formula 1, the second compound, and the organometallic compound represented by Formula 81 may respectively be understood by referring to the descriptions for those provided herein.

When the emission layer includes the host and the dopant, an amount of the dopant may be selected from a range of about 0.01 parts to about 20 parts by weight based on about 100 parts by weight of the emission layer, but embodiments are not limited thereto. When the amount of the dopant is within this range, light emission without quenching may be realized.

When the emission layer includes the condensed cyclic compound represented by Formula 1 and the second compound, a weight ratio of the condensed cyclic compound represented by Formula 1 to the second compound may be in a range of about 1:99 to about 99:1, for example, about 70:30 to about 30:70. In some embodiments, a weight ratio of the condensed cyclic compound represented by Formula 1 to the second compound may be in a range of about 60:40 to about 40:60. When the weight ratio of the condensed cyclic compound represented by Formula 1 to the second compound in the emission layer is within any of these ranges, the charge transport balance may be efficiently achieved in the emission layer.

The thickness of the emission layer may be in a range of about 100 Å to about 1,000 Å, and in some embodiments, about 200 Å to about 600 Å. While not wishing to be bound by theory, it is understood that when the thickness of the emission layer is within any of these ranges, improved luminescence characteristics may be obtained without a substantial increase in driving voltage.

Next, an electron transport region may be formed on the emission layer.

The electron transport region may include a hole blocking layer, an electron transport layer, an electron injection layer, or any combination thereof, but embodiments not limited thereto.

In some embodiments, the electron transport region may have a hole blocking layer/an electron transport layer/an electron injection layer structure or an electron transport layer/an electron injection layer structure, but embodiments are not limited thereto. The electron transport layer may have a single-layered structure or a multi-layered structure including two or more different materials.

The conditions for forming a hole blocking layer, an electron transport layer, and an electron injection layer may be inferred based on the conditions for forming the hole injection layer.

When the electron transport region includes a hole blocking layer, the hole blocking layer, for example, may include at least one of BCP and Bphen, but embodiments are not limited thereto:

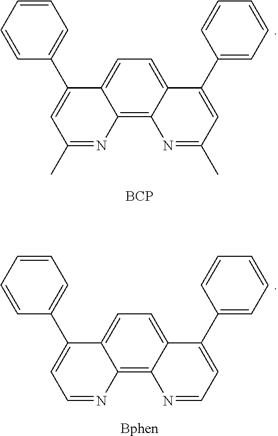

The hole blocking layer may include the condensed cyclic compound represented by Formula 1.

The thickness of the hole blocking layer may be in a range of about 20 Å to about 1,000 Å, and in some embodiments, about 30 Å to about 300 Å. When the thickness of the hole blocking layer is within any of these ranges, excellent hole blocking characteristics may be obtained without a substantial increase in driving voltage.

The electron transport layer may further include at least one selected from BCP, BPhen, Alq3, BAlq, TAZ, and NTAZ:

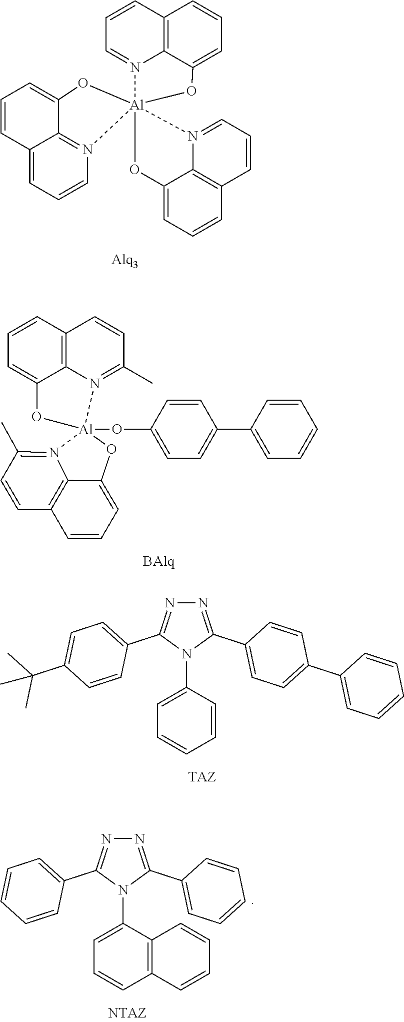

In some embodiments, the electron transport layer may include at least one selected from Compounds ET1, ET2, and ET3, but embodiments are not limited thereto:

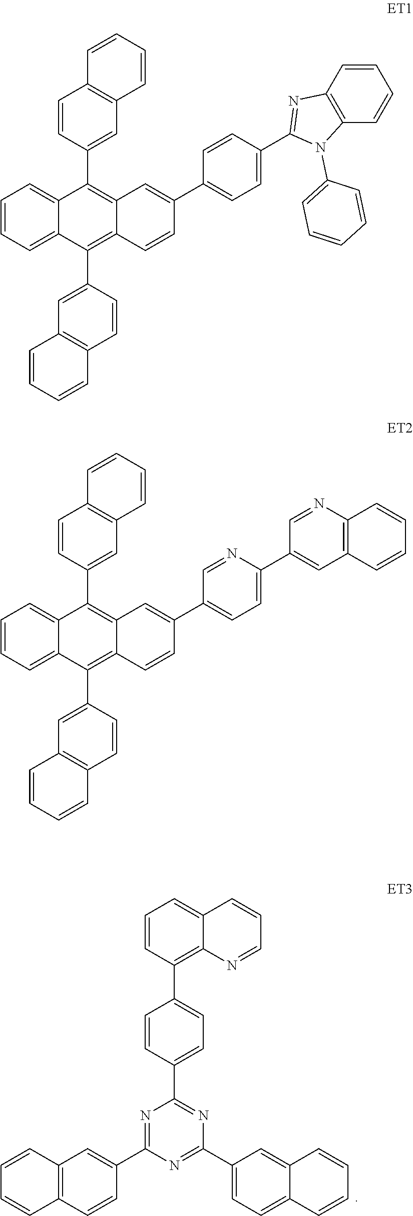 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

The thickness of the electron transport layer may be in a range of about 100 Å to about 1,000 Å, and in some embodiments, about 150 Å to about 500 Å. While not wishing to be bound by theory, it is understood that when the thickness of the electron transport layer is within any of these ranges, excellent electron transport characteristics may be obtained without a substantial increase in driving voltage.

The electron transport layer may further include a material containing metal, in addition to the materials described above.

The material containing metal may include a lithium (Li) complex. The Li complex may include, e.g., Compound ET-D1 (lithium 8-hydroxyquinolate, LiQ) or Compound ET-D2:

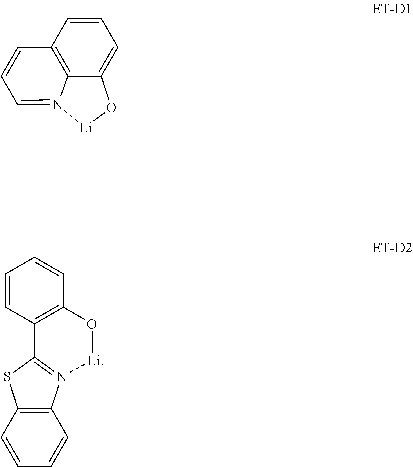

The electron transport region may include an electron injection layer that facilitates electron injection from the second electrode 19.

The electron injection layer may include at least one selected from LiQ, LiF, NaCl, CsF, Li₂O, and BaO.

The thickness of the electron injection layer may be in a range of about 1 Å to about 100 Å, for example, about 3 Å to about 90 Å. While not wishing to be bound by theory, it is understood that when the thickness of the electron injection layer is within any of these ranges, excellent electron injection characteristics may be obtained without a substantial increase in driving voltage.

The second electrode 19 may be on the organic layer 15. The second electrode 19 may be a cathode. A material for forming the second electrode 19 may be a material with a relatively low work function, such as a metal, an alloy, an electrically conductive compound, and a mixture thereof. Examples of the material for forming the second electrode 19 may include lithium (Li), magnesium (Mg), aluminum (Al), aluminum-lithium (Al—Li), calcium (Ca), magnesium-indium (Mg—In), and magnesium-silver (Mg—Ag). In some embodiments, ITO or IZO may be used to form a transmissive second electrode 19 to manufacture a top emission light-emitting device. In some embodiments, the material for forming the second electrode 19 may vary.

Hereinbefore the organic light-emitting device 10 has been described with reference to the FIGURE, but embodiments are not limited thereto.

The term “C₁-C₁₀ alkyl group” as used herein refers to a linear or branched aliphatic saturated hydrocarbon monovalent group having 1 to 60 carbon atoms. Examples thereof include a methyl group, an ethyl group, a propyl group, an iso-butyl group, a sec-butyl group, a tert-butyl group, a pentyl group, an iso-amyl group, and a hexyl group. The term “C₁-C₆₀ alkylene group” as used herein refers to a divalent group having substantially the same structure as the C₁-C₆₀ alkyl group.

The term “C₁-C₆₀ alkoxy group” as used herein refers to a monovalent group represented by —OA₁₀₁ (wherein A₁₀₁ is a C₁-C₆₀ alkyl group). Examples thereof include a methoxy group, an ethoxy group, and an iso-propyloxy group.

The term “C₂-C₆₀ alkenyl group” as used herein refers to a group formed by including at least one carbon-carbon double bond in the middle or at the terminus of the C₂-C₆₀ alkyl group. Examples thereof include an ethenyl group, a propenyl group, and a butenyl group. The term “C₂-C₆₀ alkenylene group” as used herein refers to a divalent group having substantially the same structure as the C₂-C₆₀ alkenyl group.

The term “C₂-C₆₀ alkynyl group” as used herein refers to a group formed by including at least one carbon-carbon triple bond in the middle or at the terminus of the C₂-C₆₀ alkyl group. Examples thereof include an ethenyl group and a propenyl group.

The term “C₂-C₆₀ alkynylene group” as used herein refers to a divalent group having substantially the same structure as the C₂-C₆₀ alkynyl group.

The term “C₃-C₁₀ cycloalkyl group” as used herein refers to a monovalent monocyclic saturated hydrocarbon group including 3 to 10 carbon atoms. Examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having substantially the same structure as the C₃-C₁₀ cycloalkyl group.

The term “C₁-C₁₀ heterocycloalkyl group” as used herein refers to a monovalent monocyclic group including at least one heteroatom selected from N, O, P, Si, and S as a ring-forming atom and 1 to 10 carbon atoms. Examples thereof include a tetrahydrofuranyl group and a tetrahydrothiophenyl group. The term “C₁-C₁₀ heterocycloalkylene group” as used herein refers to a divalent group having substantially the same structure as the C₁-C₁₀ heterocycloalkyl group.

The term “C₃-C₁₀ cycloalkenyl group” as used herein refers to a monovalent monocyclic group including 3 to 10 carbon atoms and at least one carbon-carbon double bond in its ring, wherein the molecular structure as a whole is non-aromatic. Examples thereof include a cyclopentenyl group, a cyclohexenyl group, and a cycloheptenyl group. The term “C₃-C₁₀ cycloalkenylene group” as used herein refers to a divalent group having substantially the same structure as the C₃-C₁₀ cycloalkenyl group.

The term “C₁-C₁₀ heterocycloalkenyl group” as used herein refers to a monovalent monocyclic group including at least one heteroatom selected from N, O, P, Si, and S as a ring-forming atom, 1 to 10 carbon atoms, and at least one double bond in its ring. Examples of the C₂-C₆₀ heterocycloalkenyl group include a 2,3-dihydrofuranyl group and a 2,3-dihydrothiophenyl group. The term “C₁-C₁₀ heterocycloalkylene group” as used herein refers to a divalent group having substantially the same structure as the C₁-C₁₀ heterocycloalkenyl group.

The term “C₆-C₆₀ aryl group” as used herein refers to a monovalent group having a carbocyclic aromatic system having 6 to 60 carbon atoms. The term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and a C₆-C₆₀ arylene group each include at least two rings, the at least two rings may be fused.

The term “C₁-C₆₀ heteroaryl group” as used herein refers to a monovalent group having an aromatic system having at least one heteroatom selected from N, O, P, Si, and S as a ring-forming atom and 1 to 60 carbon atoms. The term “C₁-C₁₀ heteroarylene group” as used herein refers to a divalent group having an aromatic system having at least one heteroatom selected from N, O, P, Si, and S as a ring-forming atom and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₁₀ heteroaryl group and the C₁-C₆₀ heteroarylene group each include at least two rings, the at least two rings may be fused.

The term “C₆-C₆₀ aryloxy group” as used herein refers to a group represented by —OA₁₀₂(where A₁₀₂ is a C₆-C₆₀ aryl group). The term “C₆-C₆₀ arylthio group” as used herein refers to a group represented by —SA₁₀₃ (where A₁₀₃ is a C₆-C₆₀ aryl group).

The term “monovalent non-aromatic condensed polycyclic group” as used herein refers to a monovalent group having two or more rings condensed and only carbon atoms (for example, the number of carbon atoms may be in a range of 8 to 60) as ring-forming atoms, wherein the molecular structure as a whole is non-aromatic. Examples of the non-aromatic condensed polycyclic group include a fluorenyl group. The term “divalent non-aromatic condensed polycyclic group” as used herein refers to a divalent group having substantially the same structure as the monovalent non-aromatic condensed polycyclic group.

The term “monovalent non-aromatic condensed heteropolycyclic group” as used herein refers to a monovalent group having at least two rings condensed and a heteroatom selected from N, O, P, Si, and S as well as carbon atoms (for example, the number of carbon atoms may be in a range of 1 to 60) as ring-forming atoms, wherein the molecular structure as a whole is non-aromatic. Examples of the monovalent non-aromatic condensed heteropolycyclic group include a carbazolyl group. The term “divalent non-aromatic condensed heteropolycyclic group” as used herein refers to a divalent group having substantially the same structure as the monovalent non-aromatic condensed heteropolycyclic group.

In Formula 1 of the present specification, at least one substituent of the substituted C₁-C₆₀ alkyl group, the substituted C₂-C₆₀ alkenyl group, the substituted C₂-Coo alkynyl group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —CD₃, —CD₂H, —CDH₂, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —CD₃, —CD₂H, —CDH₂, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), and —B(Q₁₆)(Q₁₇);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group and a monovalent non-aromatic condensed heteropolycyclic group, each substituted with at least one selected from deuterium, —CD₃, —CD₂H, —CDH₂, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), and —B(Q₂₆)(Q₂₇); and

—Si(Q₃₁)(Q₃₂)(Q₃₃), —N(Q₃₄)(Q₃₅), and —B(Q₃₆)(Q₃₇),

wherein, Q₁₁ to Q₁₇, Q₂₁ to Q₂₇, and Q₃₁ to Q₃₇ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

The symbols * and *′ as used herein, unless defined otherwise, refer to a binding site to an adjacent atom in the formula.

Hereinafter, a compound and an organic light-emitting device according to an embodiment will be described in detail with reference to Synthesis Examples and Examples, however, the present disclosure is not limited thereto. The wording “B was used instead of A” used in describing Synthesis Examples means that an amount of B used was identical to an amount of A used in terms of molar equivalents.

### EXAMPLES

### Synthesis Example 1 (Compound 31)

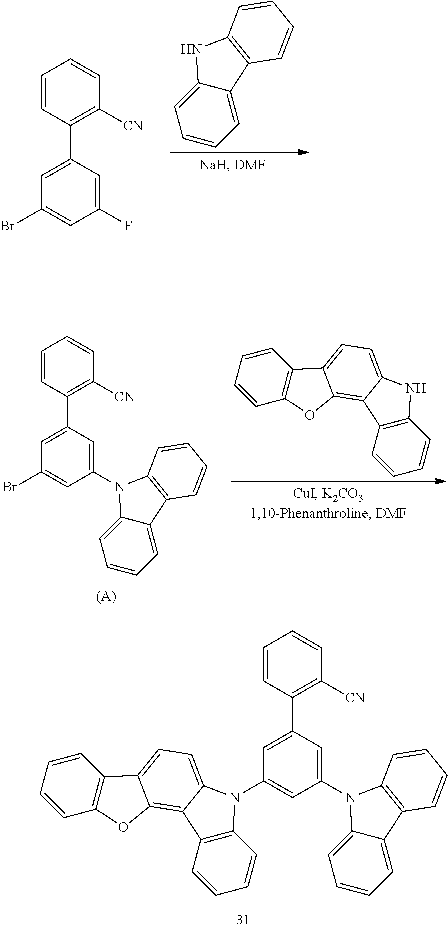

Synthesis of Intermediate (A)

7.11 grams (g) (42.52 millimoles (mmol)) of carbazole was mixed with 100 milliliters (mL) of dimethyl formamide, followed by cooling to a temperature of 0° C. 1.87 g (46.78 mmol) of sodium hydride (60% dispersion in mineral oil) was slowly added thereto, followed by stirring at a temperature of 0° C. for 30 minutes. A solution, in which 12.92 g (46.78 mmol) of 3′-bromo-5′-fluoro-[1,1′-biphenyl]-2-carbonitrile was mixed with 40 mL of dimethyl formamide, was slowly added to the resulting product for 10 minutes. Subsequently, the reaction temperature was raised to 150° C., followed by stirring for 18 hours. Once the reaction was complete, the resulting mixture was cooled to room temperature, followed by addition of an ammonium chloride aqueous solution. The organic layer was extracted by using dichloromethane, and the organic extracts were dried with magnesium sulfate. The dry organic extracts were filtered and concentrated under reduced pressure. The resulting residue was separated and purified by silica gel column chromatography to obtain a desired compound, 12.00 g of Intermediate (A) (at a yield of 67%).

LC-Mass (calculated value: 422.04 grams per mole (g/mol), measured value: M+1=423 g/mol)

### Synthesis of Compound 31

12.00 g (28.35 mmol) of Intermediate (A), 7.29 g (28.35 mmol) of 5H-benzofuro[3,2-c]carbazole, 1.62 g (8.50 mmol) of copper(I) iodide, 19.59 g (141.74 mmol) of potassium carbonate, and 2.55 g (14.17 mmol) of 1,10-phenanthroline were mixed with 81 mL of dimethyl formamide, followed by stirring under reflux for 24 hours. Once the reaction was complete, the resulting mixture was cooled to room temperature, and was filtered through silica gel under reduced pressure. The obtained filtrate was concentrated under reduced pressure, and the resulting product was separated and purified by silica gel column chromatography to obtain a desired compound, 11.90 g of Compound 31 (at a yield of 70%).

LC-Mass (calculated value: 599.20 g/mol, measured value: M+1=560 g/mol)

### Synthesis Example 2 (Compound 14)

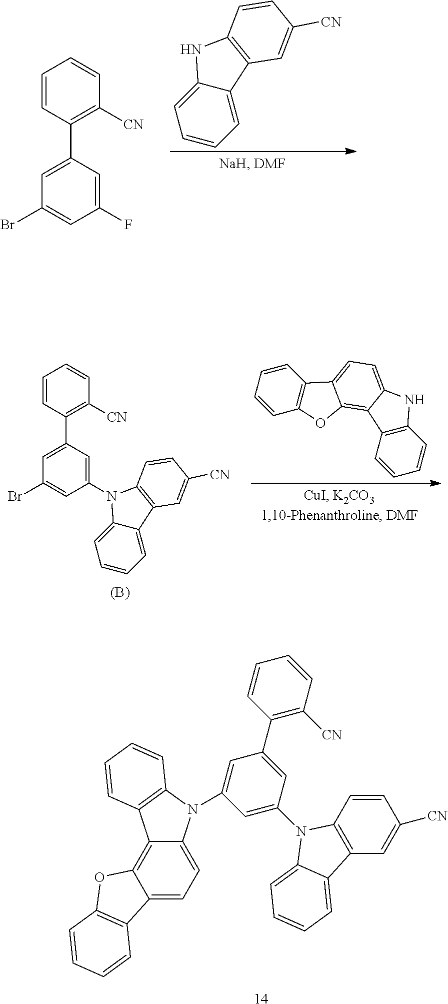

### Synthesis of Intermediate (B)

7.72 g (40.15 mmol) of 9H-carbazole-3-carbonitrile was mixed with 100 mL of dimethyl formamide, followed by cooling to a temperature of 0° C. 1.77 g (44.17 mmol) of sodium hydride (60% dispersion in mineral oil) was slowly added thereto, followed by stirring at a temperature of 0° C. for 30 minutes. A solution, in which 12.19 g (44.17 mmol) of 3′-bromo-5′-fluoro-[1,1′-biphenyl]-2-carbonitrile was mixed with 40 mL of dimethyl formamide, was slowly added to the resulting product for 10 minutes. Subsequently, the reaction temperature was raised to a temperature of 150° C., followed by stirring for 18 hours. Once the reaction was complete, the resulting mixture was cooled to room temperature, followed by addition of an ammonium chloride aqueous solution. The organic layer was extracted by using dichloromethane, and the organic extracts were dried with magnesium sulfate. The dry organic extracts were filtered and concentrated under reduced pressure. The resulting residue was separated and purified by silica gel column chromatography to obtain a desired compound, 8.51 g of Intermediate (B) (at a yield of 47%).

LC-Mass (calculated value: 447.04 g/mol, measured value: M+1=448 g/mol)

### Synthesis of Compound 14

8.25 g (18.41 mmol) of Intermediate (B), 4.74 g (18.41 mmol) of 5H-benzofuro[3,2-c]carbazole, 1.05 g (5.52 mmol) of copper(I) iodide, 12.72 g (92.05 mmol) of potassium carbonate, and 1.66 g (9.20 mmol) of 1,10-phenanthroline were mixed with 50 mL of dimethyl formamide, followed by stirring under reflux for 24 hours. Once the reaction was complete, the resulting mixture was cooled to room temperature and filtered through silica gel under reduced pressure. The obtained filtrate was concentrated under reduced pressure, and the resulting product was separated and purified by silica gel column chromatography to obtain a desired compound, 7.52 g of Compound 14 (at a yield of 65%).

LC-Mass (calculated value: 624.20 g/mol, measured value: M+1=625 g/mol) Synthesis Example 3 (Compound 32)

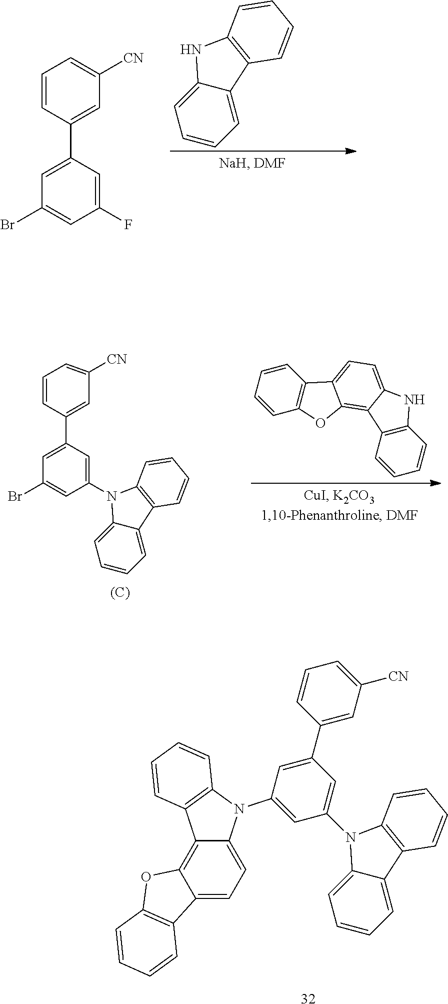

### Synthesis of Intermediate (C)

11.56 g (44.94 mmol) of carbazole was mixed with 100 mL of dimethyl formamide, followed by cooling to a temperature of 0° C. 1.87 g (46.78 mmol) of sodium hydride (60% dispersion in mineral oil) was slowly added thereto, followed by stirring at a temperature of 0° C. for 30 minutes. A solution, in which 12.92 g (46.78 mmol) of 3′-bromo-5′-fluoro-[1,1′-biphenyl]-3-carbonitrile was mixed with 40 mL of dimethyl formamide, was slowly added to the resulting product for 10 minutes. Subsequently, the reaction temperature was raised to a temperature of 150° C., followed by stirring for 18 hours. Once the reaction was complete, the resulting mixture was cooled to room temperature, followed by addition of an ammonium chloride aqueous solution. The organic layer was extracted by using dichloromethane, and the organic extracts were dried with magnesium sulfate. The dry organic extracts were filtered and concentrated under reduced pressure. The resulting residue was separated and purified by silica gel column chromatography to obtain a desired compound, 13.20 g of Intermediate (C) (at a yield of 73%).

LC-Mass (calculated value: 422.04 g/mol, measured value: M+1=423 g/mol)

### Synthesis of Compound 32

12.00 g (28.35 mmol) of Intermediate (C), 7.29 g (28.35 mmol) of 5H-benzofuro[3,2-c]carbazole, 1.62 g (8.50 mmol) of copper(I) iodide, 19.59 g (141.74 mmol) of potassium carbonate, and 2.55 g (14.17 mmol) of 1,10-phenanthroline were mixed with 81 mL of dimethyl formamide, followed by stirring under reflux for 18 hours. Once the reaction was complete, the resulting mixture was cooled to room temperature, and was filtered through silica gel under reduced pressure. The obtained filtrate was concentrated under reduced pressure, and the resulting product was separated and purified by silica gel column chromatography to obtain a desired compound, 12.35 g of Compound 32 (at a yield of 73%).

LC-Mass (calculated value: 599.20 g/mol, measured value: M+1=560 g/mol) Synthesis Example 4 (Compound 34)

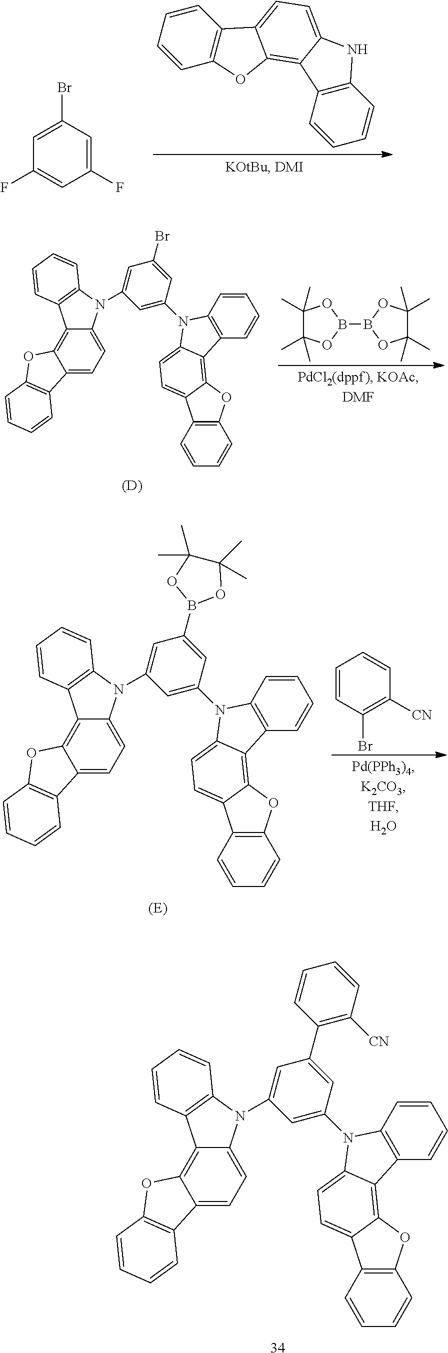

### Synthesis of Intermediate (D)

11.56 g (44.94 mmol) of carbazole and 5.30 g (47.19 mmol) of potassium tert-butoxide were mixed with 23 mL of 1,3-dimethyl-2-imidazolidinone at a temperature of 120° C. Subsequently, the temperature was raised to 140° C. Next, 4.34 g (22.47 mmol) of 1-bromo-3,5-difluorobenzene was added thereto, followed by stirring for 2 hours. Once the reaction was complete, the resulting mixture was cooled to room temperature, followed by addition of a sodium chloride aqueous solution. The organic layer was extracted by using dichloromethane, and the organic extracts were dried with magnesium sulfate. Subsequently, the dry organic extracts were filtered and concentrated under reduced pressure. The resulting residue was separated and purified by silica gel column chromatography to obtain a desired compound, 13.50 g of Intermediate (D) (at a yield of 90%).

LC-Mass (calculated value: 666.09 g/mol, measured value: M+1=667 g/mol)

### Synthesis of Intermediate (E)

13.08 g (19.59 mmol) of Intermediate (D), 7.46 g (29.39 mmol) of 4,4,4′,4′,5,5,5′,5′-octamethyl-2,2′-bi(1,3,2-dioxaborolane), 0.86 g (1.18 mmol) of PdCl₂(dppf).CH₂Cl₂, and 5.77 g (58.77 mmol) of potassium acetate were mixed with 100 mL of dimethyl formamide, followed by stirring at a temperature of 100° C. for 24 hours. Once the reaction was complete, the resulting mixture was cooled to room temperature and filtered through silica gel under reduced pressure. The obtained filtrate was concentrated under reduced pressure, and the resulting product was separated and purified by silica gel column chromatography and recrystallized using dichloromethane/n-hexane to obtain a desired compound, 11.08 g of Intermediate (E) (at a yield of 79%).

LC-Mass (calculated value: 714.27 g/mol, measured value: M+1=715 g/mol)

### Synthesis of Compound 34

10.78 g (15.08 mmol) of Intermediate (E), 2.11 g (11.60 mmol) of 2-bromobenzonitrile, 1.34 g (1.16 mmol) of Pd(PPh₃)₄, and 3.21 g (23.20 mmol) of potassium carbonate were mixed with a mixture solution of 30 mL of tetrahydrofuran and 12 mL of water, followed by stirring under reflux. Once the reaction was complete, the resulting mixture was cooled to room temperature and extracted. The combined organic extracts were filtered through silica gel under reduced pressure. The obtained filtrate was concentrated under reduced pressure, and the resulting product was separated and purified by silica gel column chromatography to obtain a desired compound, 3.60 g of Compound 34 (at a yield of 45%).

LC-Mass (calculated value: 689.21 g/mol, measured value: M+1=690 g/mol)

### Synthesis Example 5 (Compound 180)

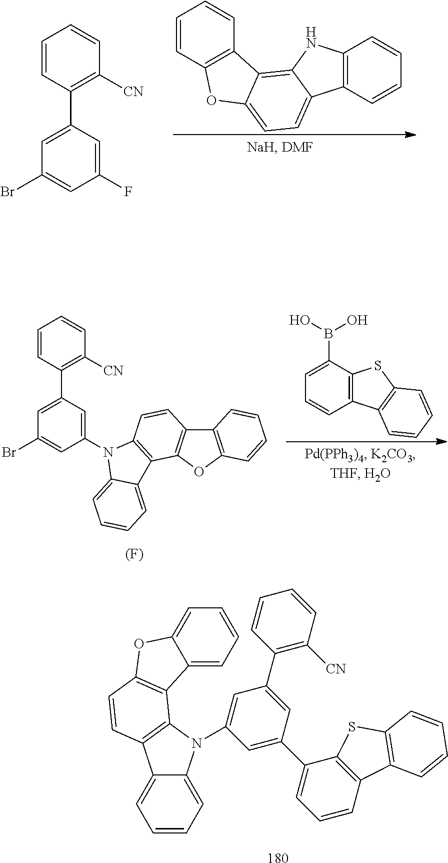

### Synthesis of Intermediate (F)

9.02 g (35.06 mmol) of 12H-benzofuro[3,2-a]carbazole was mixed with 100 mL of dimethyl formamide, followed by cooling to a temperature of 0° C. 1.54 g (38.57 mmol) of sodium hydride (60% dispersion in mineral oil) was slowly added thereto, followed by stirring at a temperature of 0° C. for 30 minutes. A solution, in which 10.65 g (38.57 mmol) of 3′-bromo-5′-fluoro-[1,1′-biphenyl]-2-carbonitrile was mixed with 40 mL of dimethyl formamide, was slowly added to the resulting product for 10 minutes. Subsequently, the reaction temperature was raised to a temperature of 150° C., followed by stirring for 18 hours. Once the reaction was complete, the resulting mixture was cooled to room temperature, followed by addition of an ammonium chloride aqueous solution. The organic layer was extracted by using dichloromethane, and the organic extract was dried with magnesium sulfate. Subsequently, the dry organic extract was filtered and concentrated under reduced pressure. The resulting residue was separated and purified by silica gel column chromatography to obtain a desired compound, 11.80 g of Intermediate (F) (at a yield of 66%).

LC-Mass (calculated value: 512.05 g/mol, measured value: M+1=513 g/mol)

### Synthesis of Compound 180

10.82 g (21.08 mmol) of Intermediate (F), 6.73 g (29.51 mmol) of dibenzo[b,d]thiophen-4-ylboronic acid, 2.44 g (2.11 mmol) of Pd(PPh₃)₄, and 5.83 g (42.16 mmol) of potassium carbonate were mixed with a solution containing 55 mL of tetrahydrofuran and 25 mL of water, followed by stirring under reflux. Once the reaction was complete, the resulting mixture was cooled to room temperature and extracted, and the resultant was filtered through silica gel under reduced pressure. The obtained filtrate was concentrated under reduced pressure, and the resulting product was separated and purified by silica gel column chromatography to obtain a desired compound, 5.35 g of Compound 180 (at a yield of 41%).

LC-Mass (calculated value: 616.16 g/mol, measured value: M+1=617 g/mol)

### Comparative Synthesis Example (Compound A)

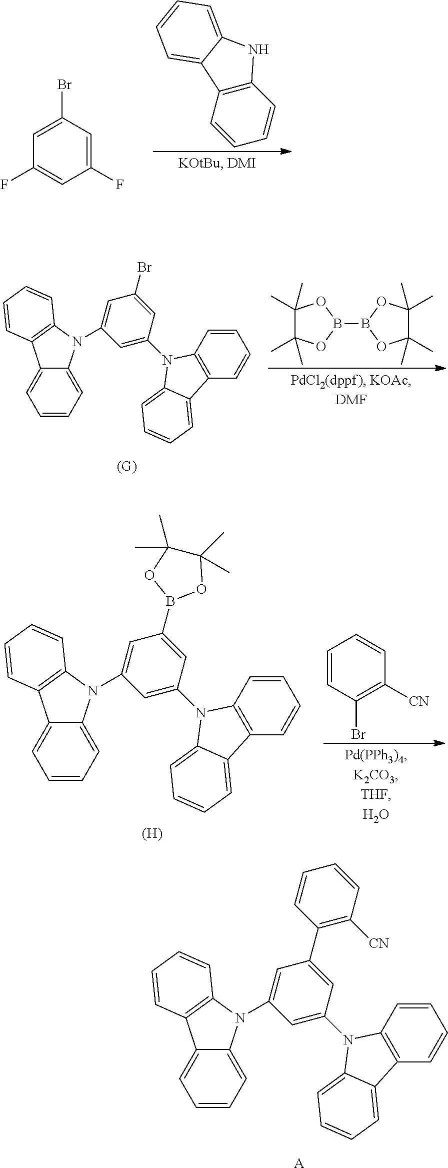

### Synthesis of Intermediate (G)

10.29 g (61.55 mmol) of carbazole and 7.25 g (64.63 mmol) of potassium tert-butoxide were mixed with 30 mL of 1,3-dimethyl-2-imidazolidinone at a temperature of 120° C. Subsequently, the temperature was raised to 140° C. Next, 5.94 g (30.78 mmol) of 1-bromo-3,5-difluorobenzene was added thereto, followed by stirring for 2 hours. Once the reaction was complete, the resulting mixture was cooled to room temperature, followed by addition of a sodium chloride aqueous solution. The organic layer was extracted by using dichloromethane, and the organic extract was dried with magnesium sulfate. Subsequently, the dry organic extract was filtered and concentrated under reduced pressure. The resulting residue was separated and purified by silica gel column chromatography to obtain a desired compound, 14.10 g of Intermediate (G) (at a yield of 93%).

LC-Mass (calculated value: 486.07 g/mol, measured value: M+1=487 g/mol)

### Synthesis of Intermediate (H)

13.68 g (28.07 mmol) of Intermediate (G), 10.69 g (42.10 mmol) of 4,4,4′,4′,5,5,5′,5′-octamethyl-2,2′-bi(1,3,2-dioxaborolane), 1.23 g (1.68 mmol) of PdCl₂(dppf).CH₂Cl₂, and 8.26 g (84.20 mmol) of potassium acetate were mixed with 140 mL of dimethyl formamide, followed by stirring at a temperature of 100° C. for 24 hours. Once the reaction was complete, the resulting mixture was cooled to room temperature and filtered through silica gel under reduced pressure. The obtained filtrate was concentrated under reduced pressure, and the resulting product was separated and purified by silica gel column chromatography and recrystallized using dichloromethane/n-hexane to obtain a desired compound, 10.62 g of Intermediate (H) (at a yield of 71%).

LC-Mass (calculated value: 534.25 g/mol, measured value: M+1=535 g/mol)

### Synthesis of Compound A

10.28 g (19.23 mmol) of Intermediate (H), 2.50 g (13.74 mmol) of 2-bromobenzonitrile, 1.59 g (1.37 mmol) of Pd(PPh₃)₄, and 3.80 g (22.47 mmol) of potassium carbonate were mixed with a mixture solution of 35 mL of tetrahydrofuran and 13 mL of water, followed by stirring under reflux. Once the reaction was complete, the resulting mixture was cooled to room temperature, and extracted, and the resultant was filtered through silica gel under reduced pressure. The obtained filtrate was concentrated under reduced pressure, and the resulting product was separated and purified by silica gel column chromatography to obtain a desired compound, 4.80 g of Compound A (at a yield of 69%).

LC-Mass (calculated value: 509.19 g/mol, measured value: M+1=510 g/mol)

### Evaluation Example 1: Thermal Characteristics Evaluation

Thermal analysis (N₂ atmosphere, temperature range: from room temperature to 800° C. (10° C./min)-TGA, from room temperature to 400° C.-differential scanning calorimetry (DSC), Pan Type: Pt Pan in disposable Al Pan (TGA) and disposable Al pan (DSC)) was performed on Compounds 31 and A by using thermogravimetric analysis (TGA) and DSC. The evaluation results are shown in Table 2.

TABLE 2

| Compound No. | T_{g} (° C.) | T_{d} (0.1%, ° C.) |
|---|---|---|
| 31 | 139 | 359 |
| A | 105 | 335 |

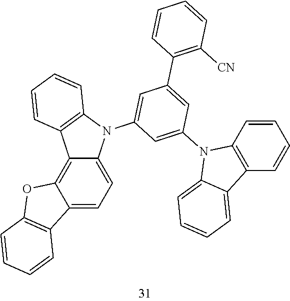 [SMILES: N#Cc1ccccc1-c1cc(-n2c3ccccc3c3ccccc32)cc(-n2c3ccccc3c3c4oc5ccccc5c4ccc32)c1]

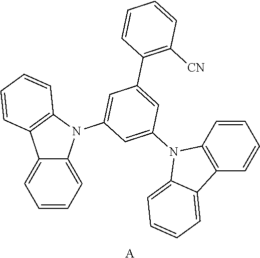

According to Table 2, it was found that Compounds 31 has excellent thermal stability, as compared with Compound A.

### Example 1

As a first electrode (an anode), a glass substrate having an ITO electrode deposited thereon at a thickness of 1,500 Å was washed with distilled water in the presence of ultrasound waves. Once the washing with distilled water was complete, ultrasound wave washing was performed on the substrate by using a solvent, such as iso-propyl alcohol, acetone, or methanol. Subsequently, the substrate was dried, transferred to a plasma washer, washed for 5 minutes using oxygen plasma, and mounted in a vacuum depositor.

Compound HT3 and Compound HT-D2 were co-deposited on the ITO electrode of the glass substrate to form a hole injection layer having a thickness of 100 Å. Subsequently, Compound HT3 was deposited on the hole injection layer to form a hole transport layer having a thickness of 1,300 Å. mCP was next deposited on the hole transport layer to form an electron blocking layer having a thickness of 100 Å, thereby forming a hole transport region.

Subsequently, Compound 31 (host) and FIr6 (dopant, 10 percent by weight (weight %)) were co-deposited on the hole transport region to form an emission layer having a thickness of about 400 Å.

BCP was vacuum-deposited on the emission layer to form a hole blocking layer having a thickness of 100 Å. Compound ET3 and LiQ were then co-deposited on the hole blocking layer to form an electron transport layer having a thickness of 300 Å. Next, LiQ was deposited on the electron transport layer to form an electron injection layer having a thickness of 10 Å, and then, aluminum (Al) second electrode (a cathode) having a thickness of 1,200 Å was formed on the electron injection layer, thereby completing the manufacture of an organic light-emitting device.

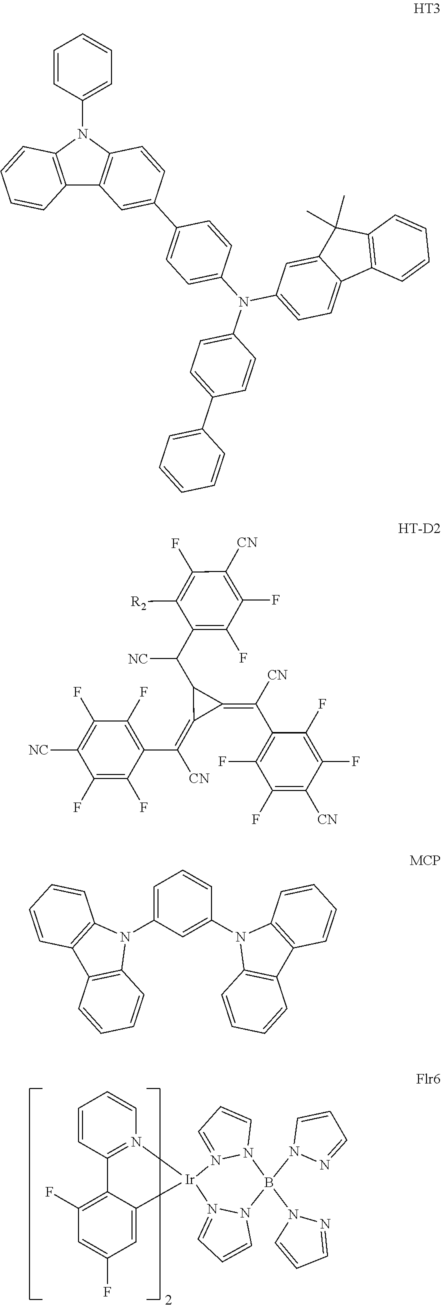

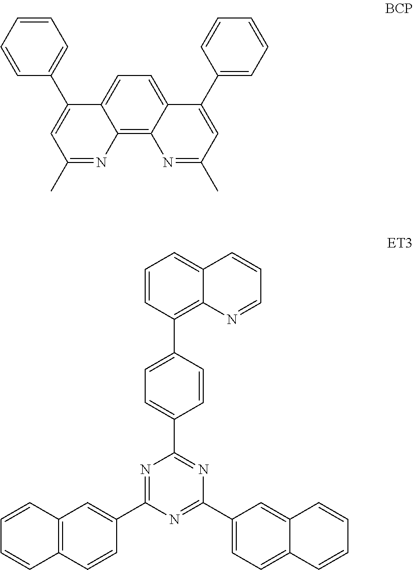 [SMILES: Cc1cc(-c2ccccc2)c2ccc3c(-c4ccccc4)cc(C)nc3c2n1 ; c1ccc2cc(-c3nc(-c4ccc(-c5cccc6cccnc56)cc4)nc(-c4ccc5ccccc5c4)n3)ccc2c1]

### Examples 2 to 5 and Comparative Example 1

Organic light-emitting devices were manufactured in substantially the same manner as in Example 1, except that the compounds shown in Table 3 were used as a host instead of Compound 31 in the formation of an emission layer.

### Evaluation Example 2: Evaluation of Characteristics of Organic Light-Emitting Device

Current density changes, luminance changes, and emission efficiencies of the organic light-emitting device prepared in Examples 1 to 5 and Comparative Example 1 were measured by applying various voltages thereto. The measurement method was as described in the following, and the results thereof are shown in Table 3. In Table 3, the driving voltage, current efficiency, and durability of Examples 1 to 5 are indicated relative to 100% of those of Comparative Example 1, respectively.

(1) Measurement of Current Density Changes Depending on Changes of Applied Voltages

Current values of the prepared organic light-emitting devices were measured by measuring values of current in a unit device thereof using a current voltmeter (Keithley 2400) while increasing the applied voltage from 0 volts (V) to 10 V. The result was obtained by dividing a current value by an area.

(2) Measurement of Luminance Changes Depending on Changes of Applied Voltages

Luminance values of the prepared organic light-emitting devices were measured by using a luminance meter (Minolta Cs-1000A) while increasing the applied voltage from 0 V to 10 V.

(3) Measurement of Emission Efficiency

The luminance values measured from (2) and current density values measured from (1), and applied voltages were used in calculating the current efficiency (cd/A) under a condition of an identical current density (10 mA/cm²).

(4) Measurement of Durability

The time (hour) for the luminance of the organic light-emitting device to decline to 95% of its initial luminance was evaluated.

TABLE 3

| | | Driving | Current | | |
|---|---|---|---|---|---|
| | | voltage | efficiency | Durability | |
| | | (relative | (relative | (relative | |
| | Host | value, %) | value, %) | value, %) | Color |
| Example 1 | Compound | 97 | 104 | 121 | Blue |
| | 31 | | | | |
| Example 2 | Compound | 105 | 105 | 117 | Blue |
| | 14 | | | | |
| Example 3 | Compound | 100 | 101 | 105 | Blue |
| | 32 | | | | |
| Example 4 | Compound | 95 | 104 | 125 | Blue |
| | 34 | | | | |
| Example 5 | Compound | 99 | 103 | 110 | Blue |
| | 180 | | | | |
| Comparative | Compound | 100 | 100 | 100 | Blue |
| Example 1 | A | | | | |

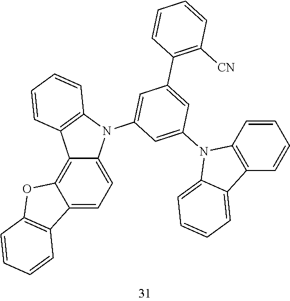 [SMILES: N#Cc1ccccc1-c1cc(-n2c3ccccc3c3ccccc32)cc(-n2c3ccccc3c3c4oc5ccccc5c4ccc32)c1]

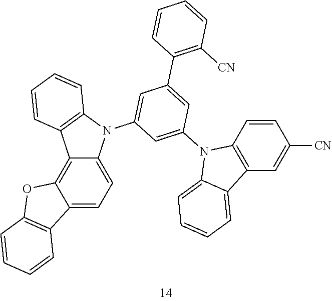 [SMILES: N#Cc1ccc2c(c1)c1ccccc1n2-c1cc(-c2ccccc2C#N)cc(-n2c3ccccc3c3c4oc5ccccc5c4ccc32)c1]

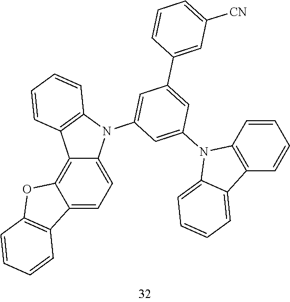 [SMILES: N#Cc1cccc(-c2cc(-n3c4ccccc4c4ccccc43)cc(-n3c4ccccc4c4c5oc6ccccc6c5ccc43)c2)c1]

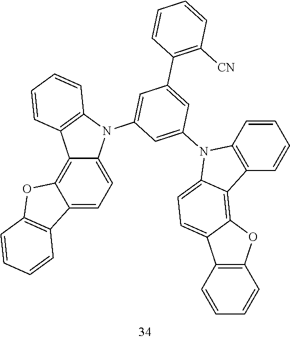 [SMILES: N#Cc1ccccc1-c1cc(-n2c3ccccc3c3c4oc5ccccc5c4ccc32)cc(-n2c3ccccc3c3c4oc5ccccc5c4ccc32)c1]

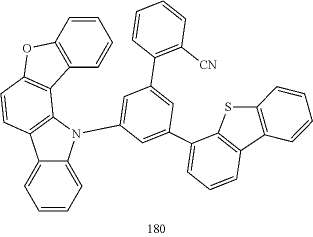 [SMILES: N#Cc1ccccc1-c1cc(-c2cccc3c2sc2ccccc23)cc(-n2c3ccccc3c3ccc4oc5ccccc5c4c32)c1]

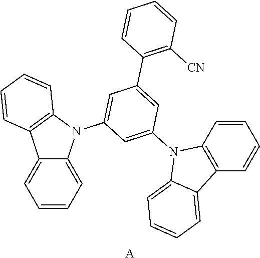

According to Table 3, the organic light-emitting device of Examples 1 to 5 were found to have similar or unexpectedly better driving voltage and current efficiency as well as improved durability (lifespan), as compared with the organic light-emitting device of Comparative Example 1.

As apparent from the foregoing description, the condensed cyclic compound according to embodiments has excellent electric characteristics and thermal stability. Accordingly, an organic light-emitting device including the condensed cyclic compound may have a low driving voltage, high efficiency, high power, high quantum yield, and long lifespan.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

What is claimed is:

1. A condensed cyclic compound represented by Formula 1: 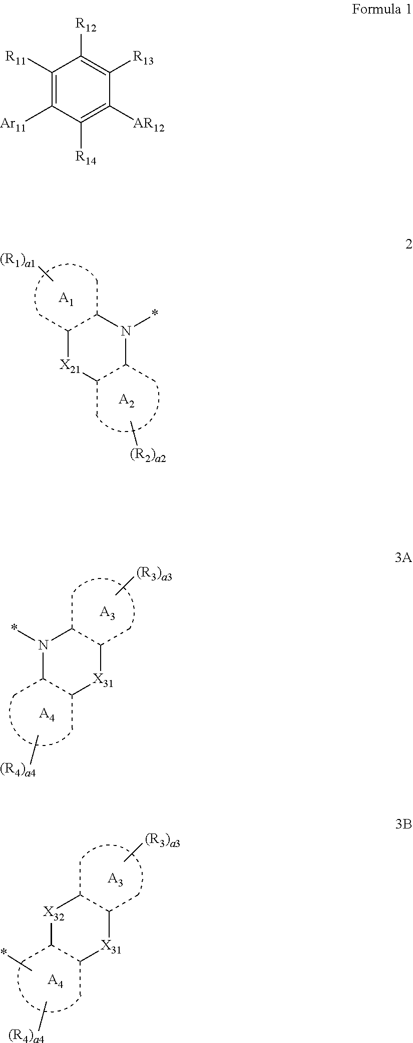
wherein in Formulae 1, 2, 3A, and 3B,
Ar₁₁ is a group represented by Formula 2,
Ar₁₂ is a group represented by Formula 3A or 3B,
A₁ is a dibenzofuran group, a dibenzothiophene group, a carbazole group, a fluorene group, a dibenzosilole group, an azadibenzofuran group, an azadibenzothiophene group, an azacarbazole group, an azafluorene group, or an azadibenzosilole group,
A₂ to A₄ are each independently a C₆-C₆₀ carbocyclic group or a C₁-C₆₀ heterocyclic group,
X₂₁ is selected from a single bond, O, S, N(R₂₁), C(R₂₁)(R₂₂), and Si(R₂₁)(R₂₂),
X₃₁ is selected from a single bond, O, S, N(R₃₁), C(R₃₁)(R₃₂), and Si(R₃₁)(R₃₂), and X₃₂ is selected from a single bond, O, S, N(R₃₃), C(R₃₃)(R₃₄), and Si(R₃₃)(R₃₄), provided that at least one selected from X₃₁ and X₃₂ in Formula 3B is not a single bond,
R₁₁ to R₁₃ are each independently selected from a phenyl group, a biphenyl group, a terphenyl group, and a tetraphenyl group, each unsubstituted or substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₁-C₆₀ alkoxy group, a phenyl group, a biphenyl group, a terphenyl group, and a tetraphenyl group, wherein at least one selected from R₁₁ to R₁₃ is independently selected from a cyano group-containing phenyl group, a cyano group-containing biphenyl group, a cyano group-containing terphenyl group, and a cyano group-containing tetraphenyl group,
R₁ to R₄, R₁₄, R₂₁, R₂₂, and R₃₁ to R₃₄ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇),
a1 to a4 are each independently an integer from 0 to 10,
* indicates a binding site to an adjacent atom, and
at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is selected from:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), and —B(Q₁₆)(Q₁₇);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycycic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂), —N(Q₂₄)(Q₂₅), and —B(Q₂₆)(Q₂₇); and
—Si(Q₃₁)(Q₃₂)(Q₃₃), —N(Q₃₄)(Q₃₅), and —B(Q₃₈)(Q₃₇),
wherein Q₁ to Q₁₀, Q₁₁ to Q₁₇, Q₂₁ to Q₂₇, and Q₃₁ to Q₃₇ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

2. The condensed cyclic compound of claim 1, wherein
A₁ is a dibenzofuran group, a dibenzothiophene group, a carbazole group, a fluorene group, or a dibenzosilole group, and
A₂ to A₄ are each independently a benzene group, a naphthalene group, a dibenzofuran group, a dibenzothiophene group, a carbazole group, a fluorene group, or a dibenzosilole group.

3. The condensed cyclic compound of claim 1, wherein Ar₁₁ is selected from groups represented by Formulae 2-1 to 2-6: 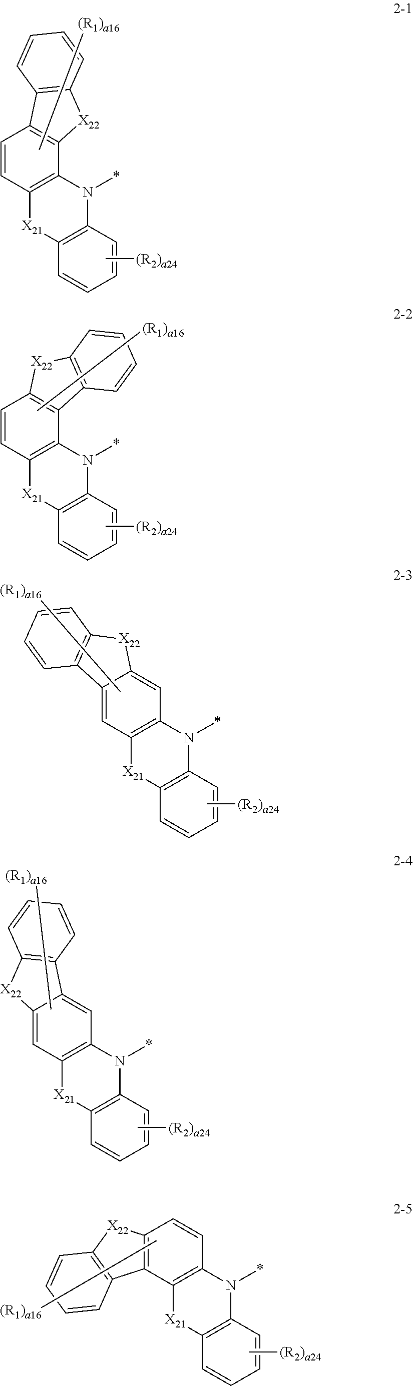 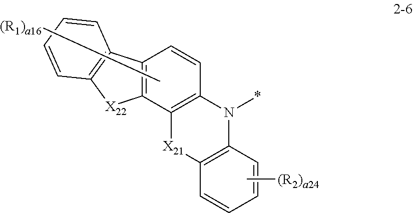
wherein, in Formulae 2-1 to 2-6,
X₂₂ is selected from O, S, N(R₂₃), C(R₂₃)(R₂₄), and Si(R₂₃)(R₂₄),
X₂₁, R₁, and R₂ are each as defined in claim 1,
R₂₃ and R₂₄ are each defined the same as R₂₁ in claim 1,
a16 is an integer from 0 to 6,
a24 is an integer from 0 to 4, and
* indicates a binding site to an adjacent atom.

4. The condensed cyclic compound of claim 3, wherein X₂₁ in Formulae 2-1 to 2-6 is a single bond, and X₂ is O or S.

5. The condensed cyclic compound of claim 1, wherein Ar₁₂ is selected from groups represented by Formulae 3A-1 to 3A-7 and 3B-1 to 3B-7: 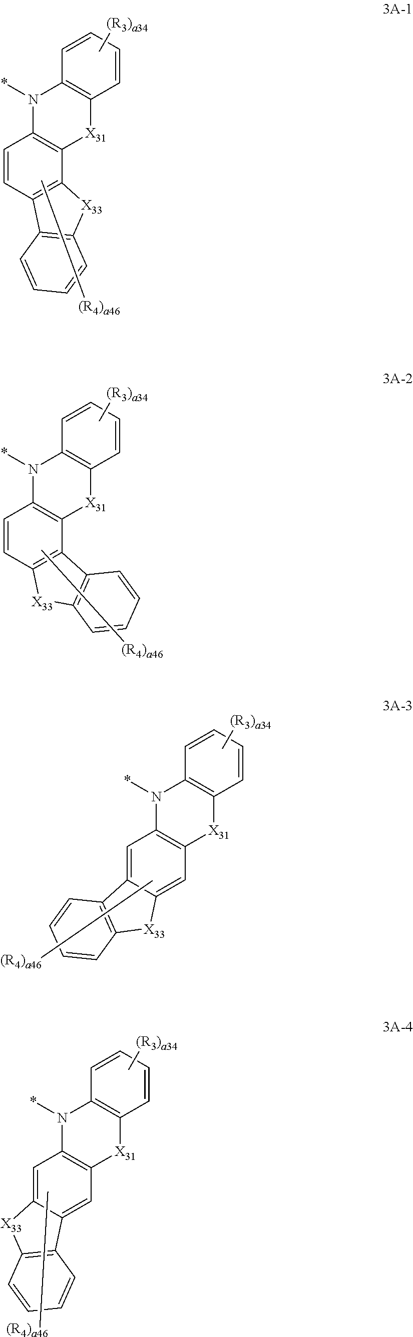 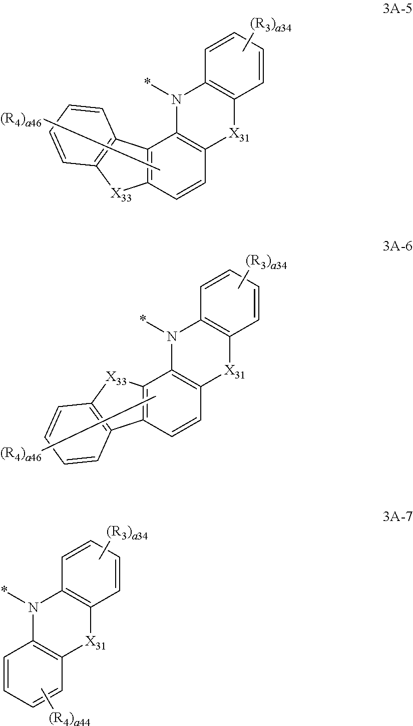 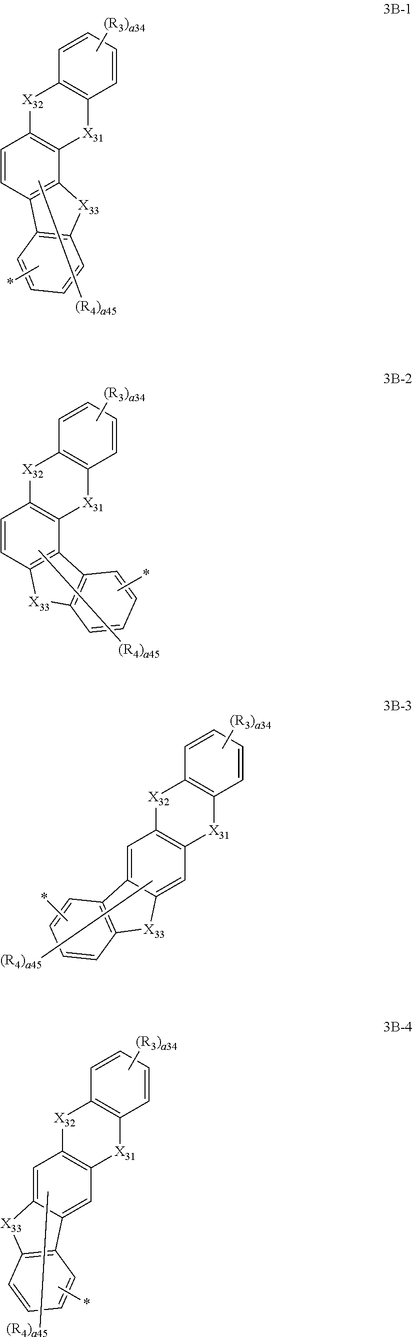 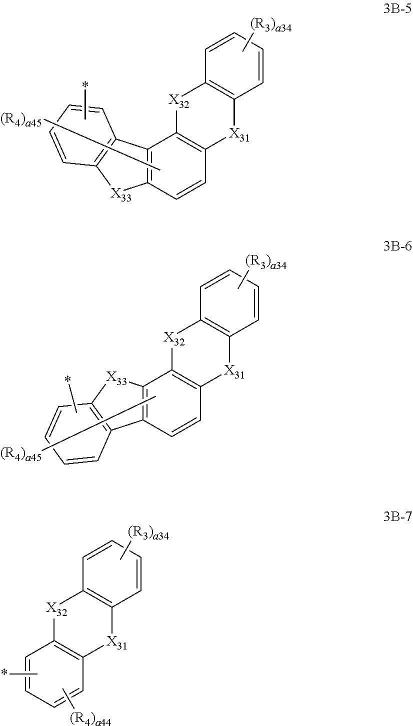
wherein Formulae 3A-1 to 3A-7 and 3B-1 to 3B-7,
X₃₃ is selected from O, S, N(R₃₅), C(R₃₅)(R₃₆), and Si(R₃₅)(R₃₆),
X₃₁, X₃₂, R₃, and R₄ are each as defined in claim 1,
R₃₅ and R₃₆ are each defined the same as R₃₁ in claim 1,
a34 is an integer from 0 to 4,
a45 is an integer from 0 to 5,
a43 is an integer from 0 to 3, and
* indicates a binding site to an adjacent atom.

6. The condensed cyclic compound of claim 1, wherein X₃₁ in Formulae 3A-1 to 3A-7 is a single bond.

7. The condensed cyclic compound of claim 1, wherein Ar₁₂ is selected from groups represented by Formulae 3A-1(1) to 3A-6(1), 3A-7(1) to 3A-7(3), and 3B-7(1) to 3B-7(14): 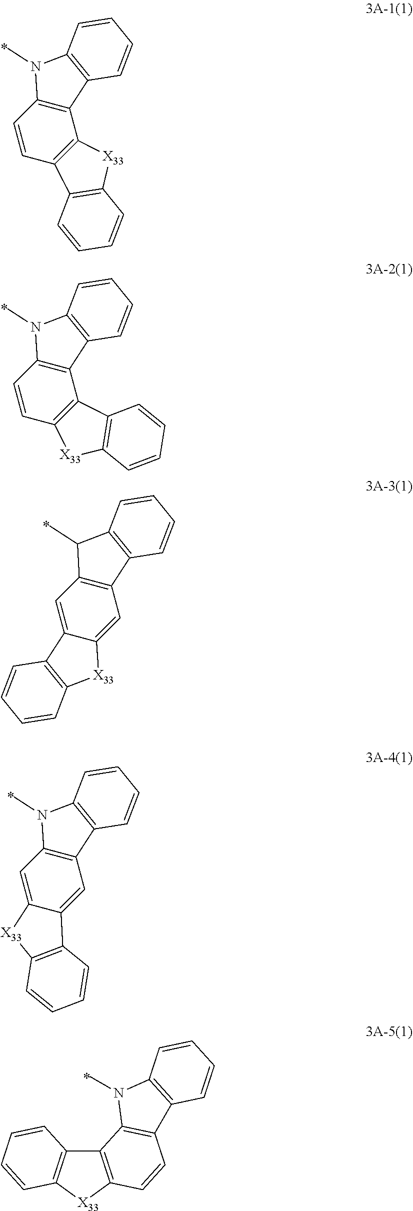 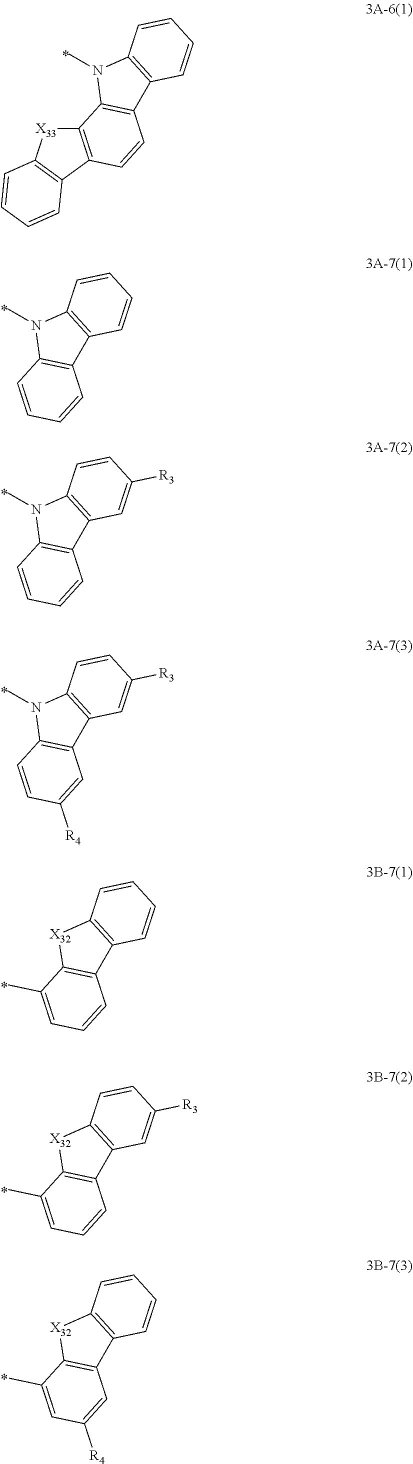 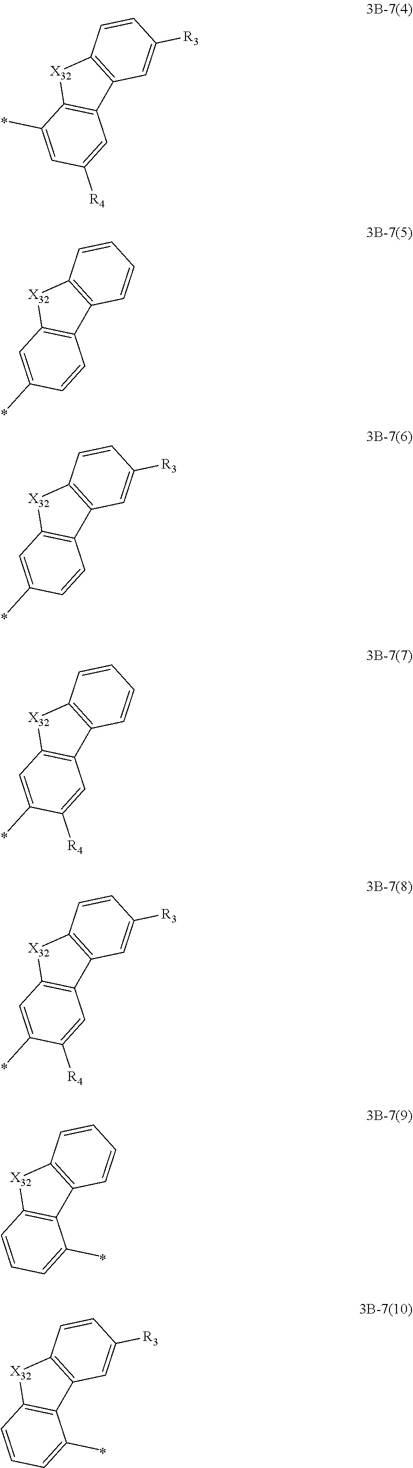 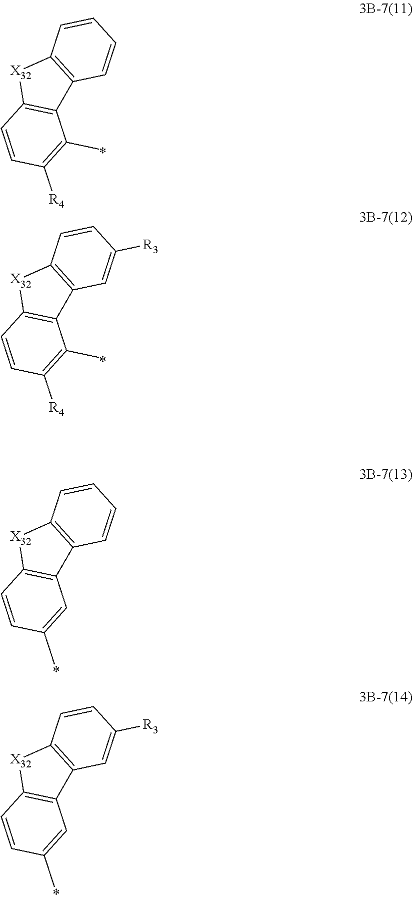
wherein, in Formulae 3A-1(1) to 3A-6(1), 3A-7(1) to 3A-7(3), and 3B-7(1) to 3B-7(14),
X₃₂ is selected from O, S, N(R₃₃), C(R₃₃)(R₃₄), and Si(R₃₃)(R₃₄),
X₃₃ is selected from O, S, N(R₃₅), C(R₃₅)(R₃₆), and Si(R₃₅)(R₃₆),
R₃ and R₄ are each as defined in claim 1, provided that each of R₃ and R₄ is not hydrogen,
R₃₃ and R₃₄ are each as defined in claim 1,
R₃₅ and R₃₆ are each defined the same as R₃₁ in claim 1, and
* indicates a binding site to an adjacent atom.

8. The condensed cyclic compound of claim 7, wherein at least one selected from R₃ and R₄ in Formulae 3A-7(2), 3A-7(3) and 3B-7(2) to 3B-7(4), 3B-7(6) to 3B-7(8), 3B-7(10) to 3B-7(12), and 3B-7(14) is a cyano group.

9. The condensed cyclic compound of claim 1, wherein R₁₁ to R₁₃ are each independently selected from a phenyl group, a biphenyl group, a terphenyl group, and a tetraphenyl group, each unsubstituted or substituted with at least one selected from deuterium, a cyano group, a C₁-C₂₀ alkyl group, a phenyl group, a biphenyl group, a terphenyl group, and a tetraphenyl group, wherein at least one selected from R₁₁ to R₁₃ is independently selected from groups represented by Formulae 4-1 to 4-4: 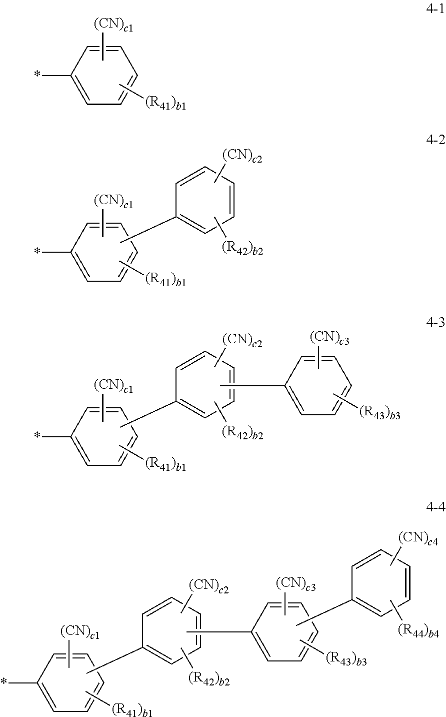
wherein, in Formulae 4-1 to 4-4,
R₄₁ to R₄₄ are each independently selected from hydrogen, deuterium, a C₁-C₂₀ alkyl group, a phenyl group, a biphenyl group, and a terphenyl group,
b1 to b4 and c1 to c4 are each independently an integer from 0 to 4,
* indicates a binding site to an adjacent atom, and
c1 in Formula 4-1, c1+c2 in Formula 4-2, c1+c2+c3 in Formula 4-3, and c1+c2+c3+c4 in Formula 4 are each 1 or greater.

10. The condensed cyclic compound of claim 1, wherein at least one selected from R₁₁ to R₁₃ is independently selected from groups represented by Formulae 4-2A to 4-2C, 4-3A to 4-3I, and 4-4A to 4-4AA: 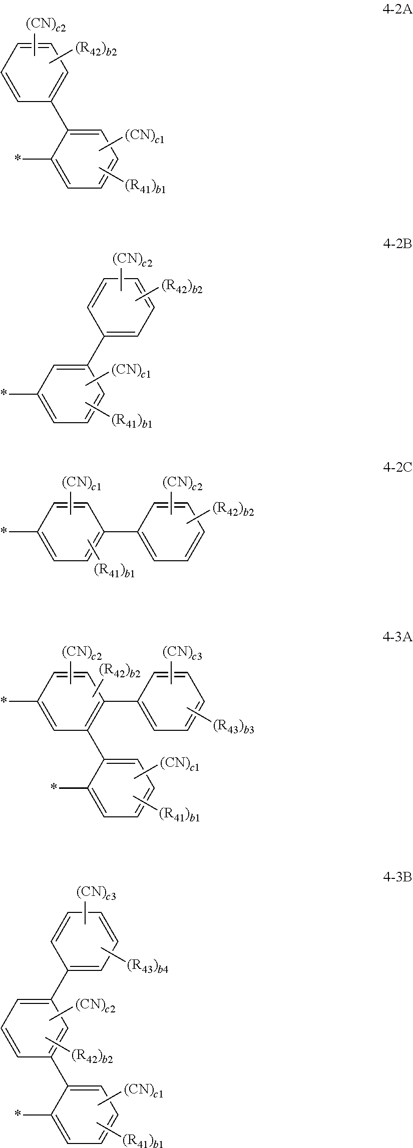 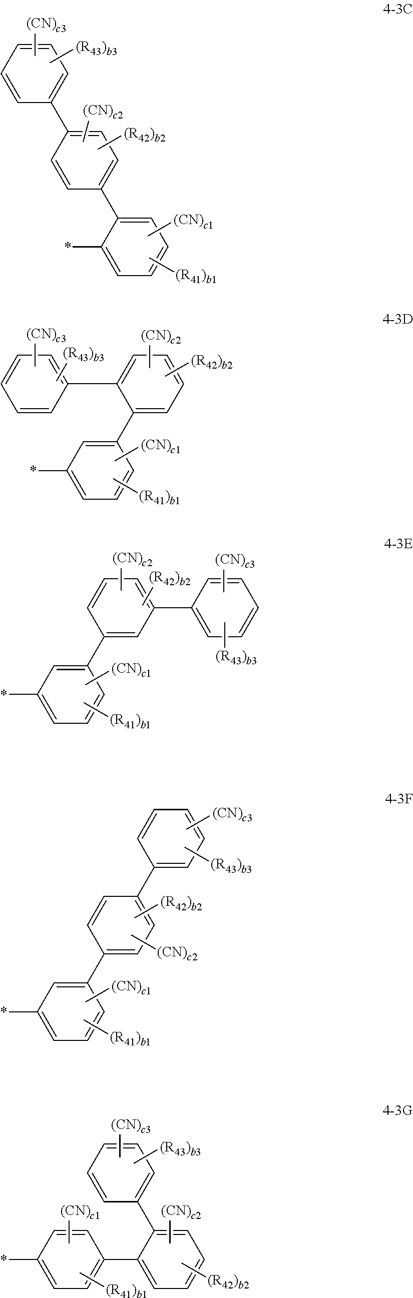 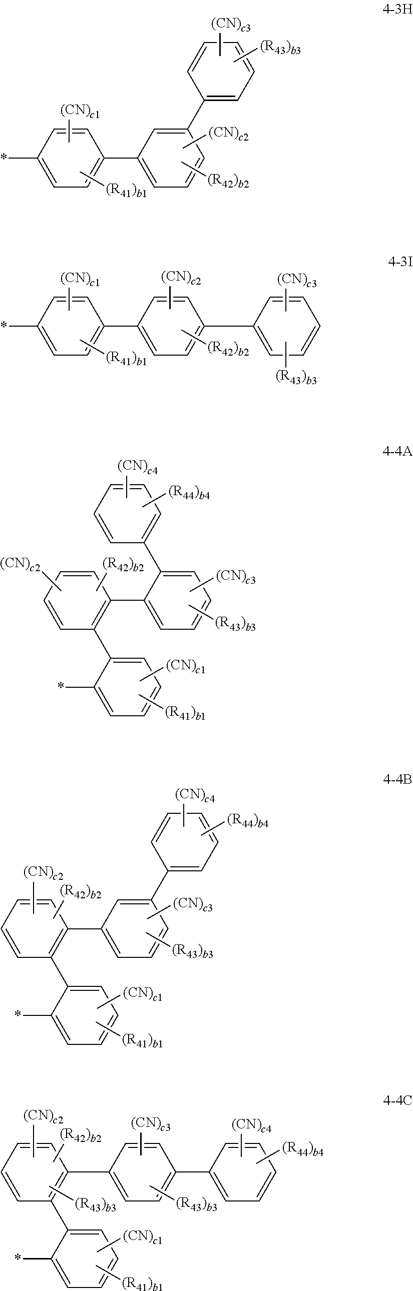 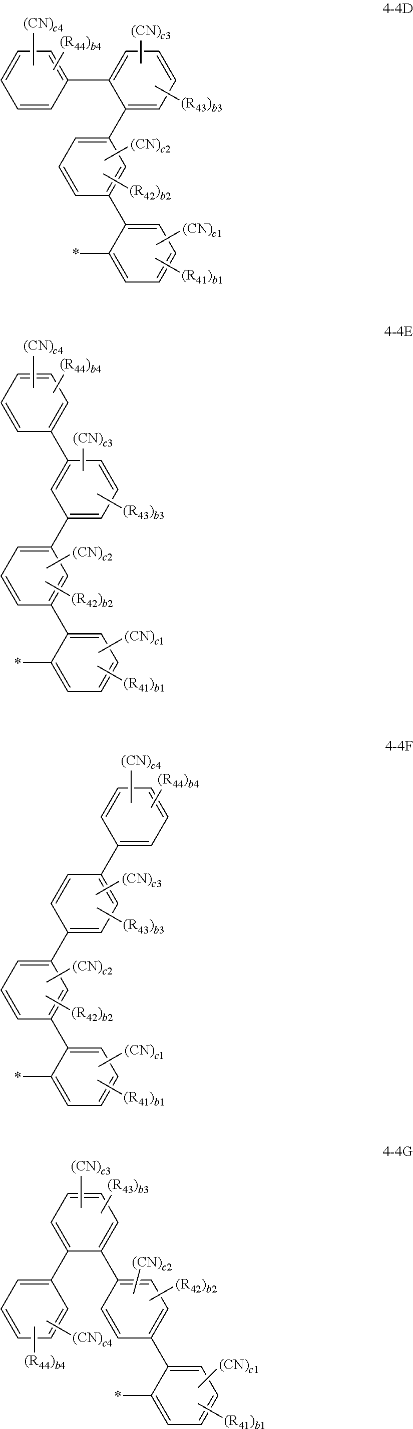 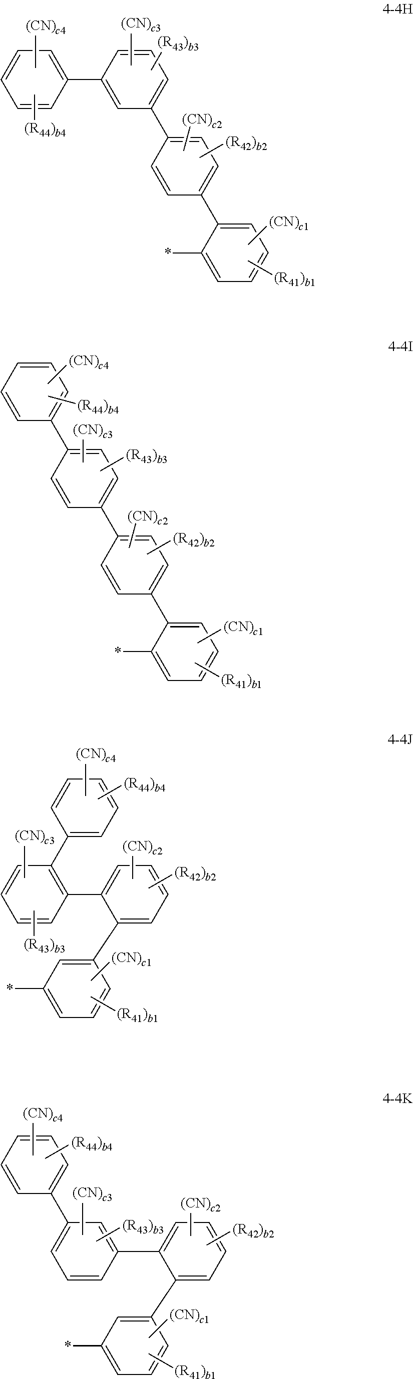 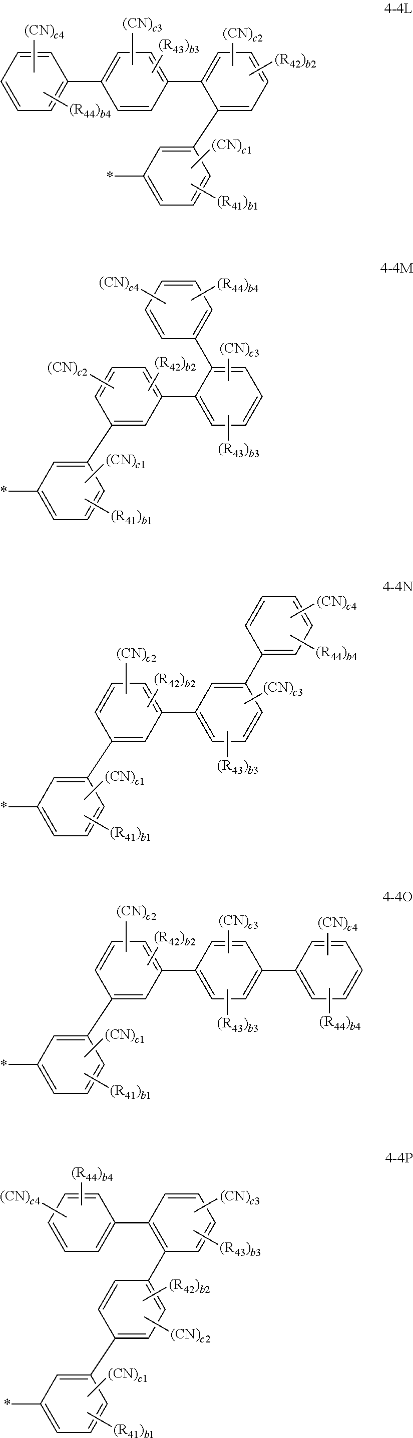 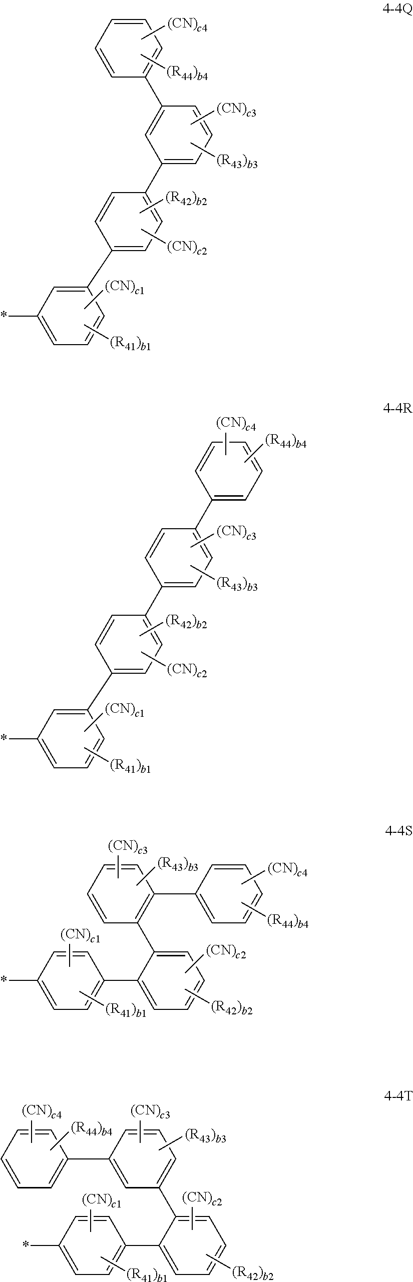 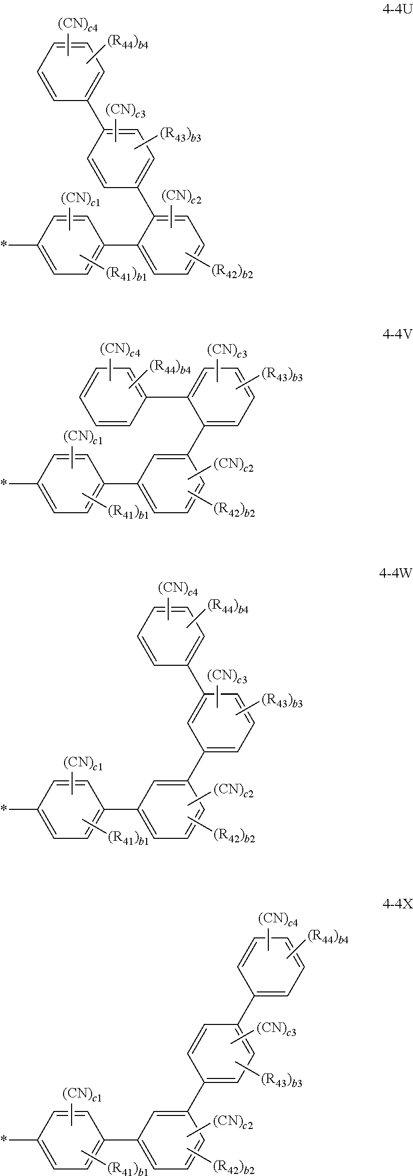 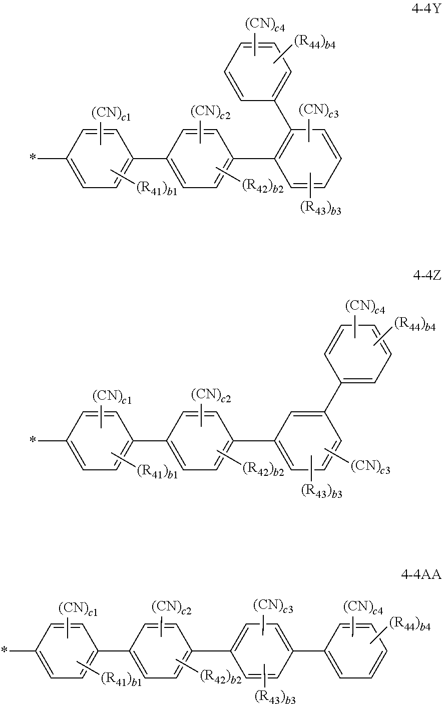
wherein, in Formulae 4-2A to 4-2C, 4-3A to 4-3I, and 4-4A to 4-4AA,
R₄₁ to R₄ are each independently selected from hydrogen, deuterium, a C₁-C₂₀ alkyl group, a phenyl group, a biphenyl group, and a terphenyl group,
b1 to b4 and c1 to c4 are each independently an integer from 0 to 4,
* indicates a binding site to an adjacent atom, and
c1+c2 in Formulae 4-2A to 4-2C, c1+c2+c3 in Formulae 4-3A to 4-3I, and c1+c2+c3+c4 in Formulae 4-4A to 4-4AA are each 1 or greater.

11. The condensed cyclic compound of claim 1, wherein at least one selected from R₁₁ to R₁₃ is independently selected from groups represented by Formulae 4-1(1) to 4-1(8) and 4-2(1) to 4-2(78): 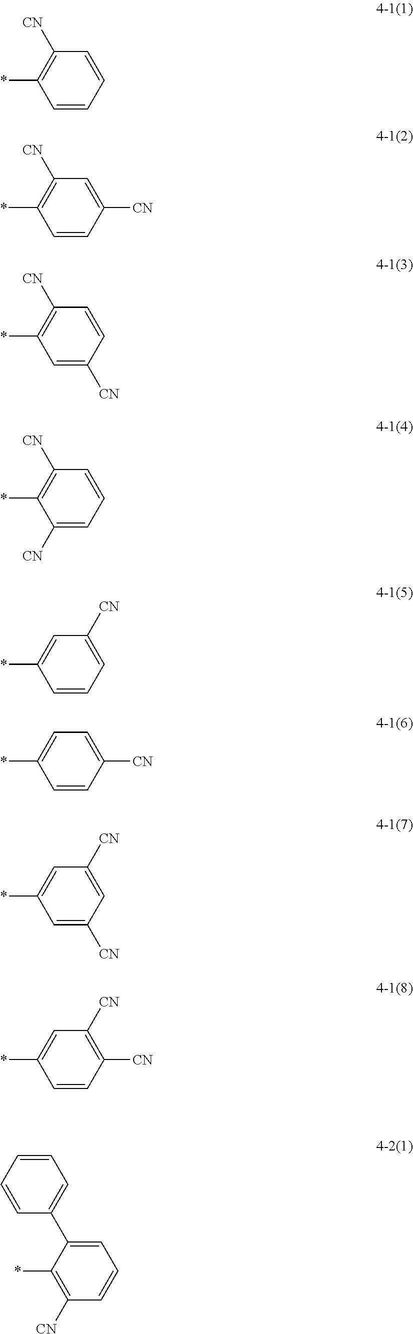 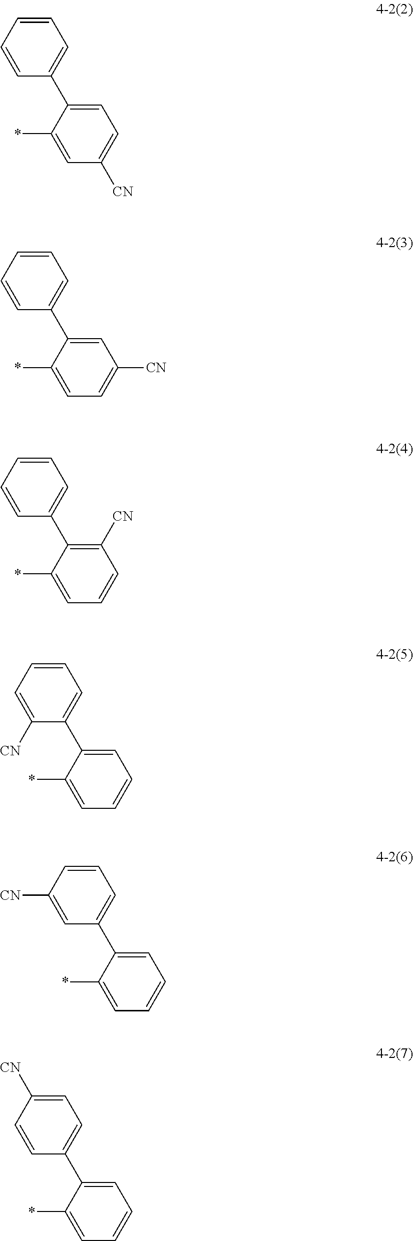 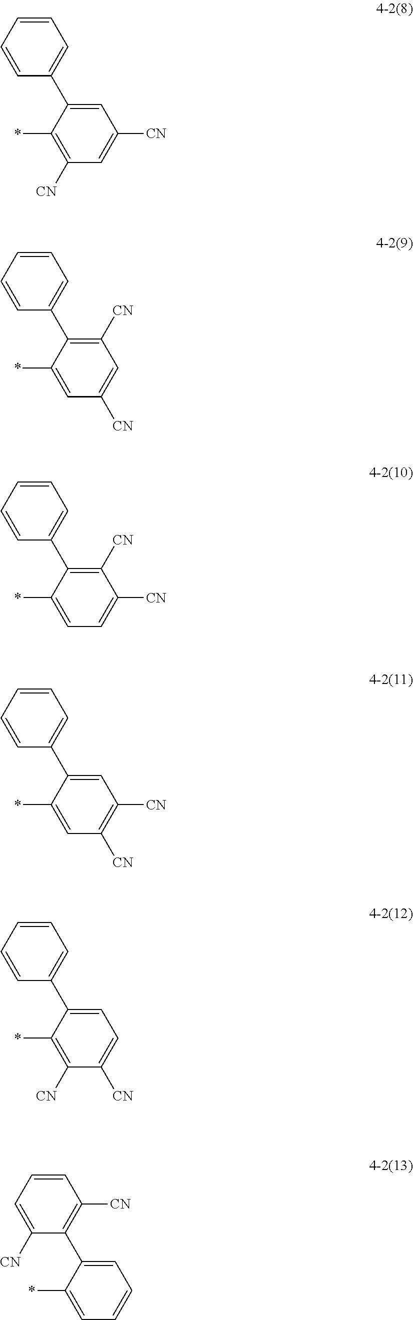 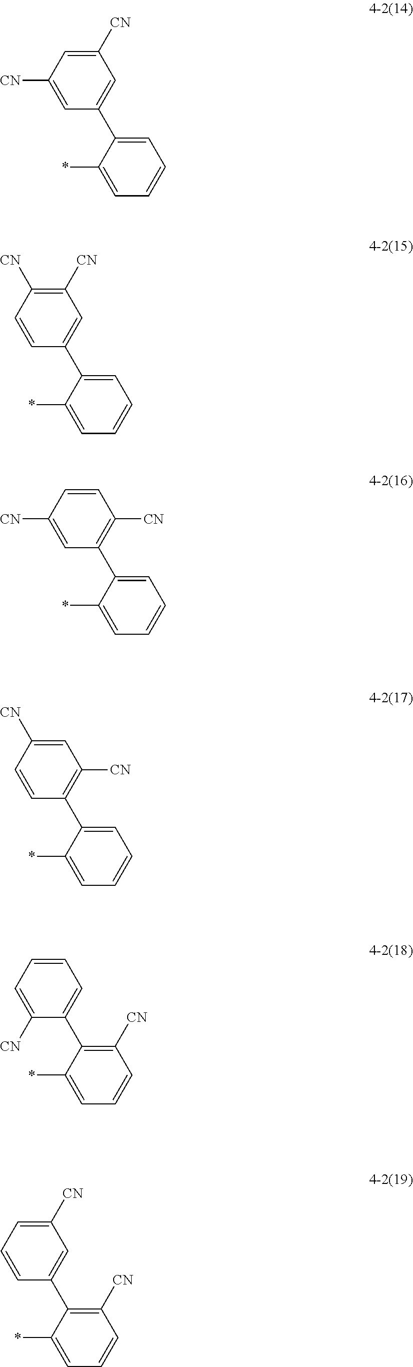 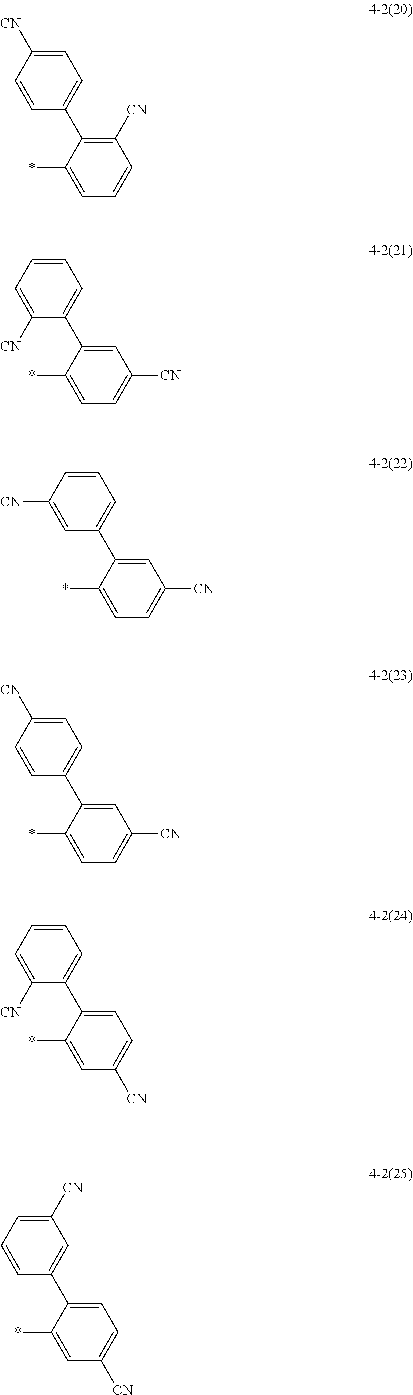 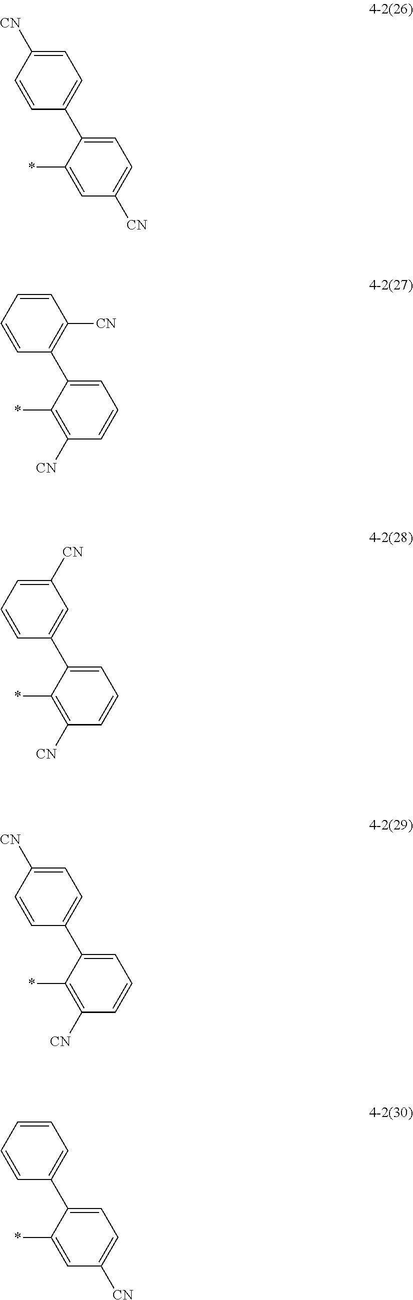 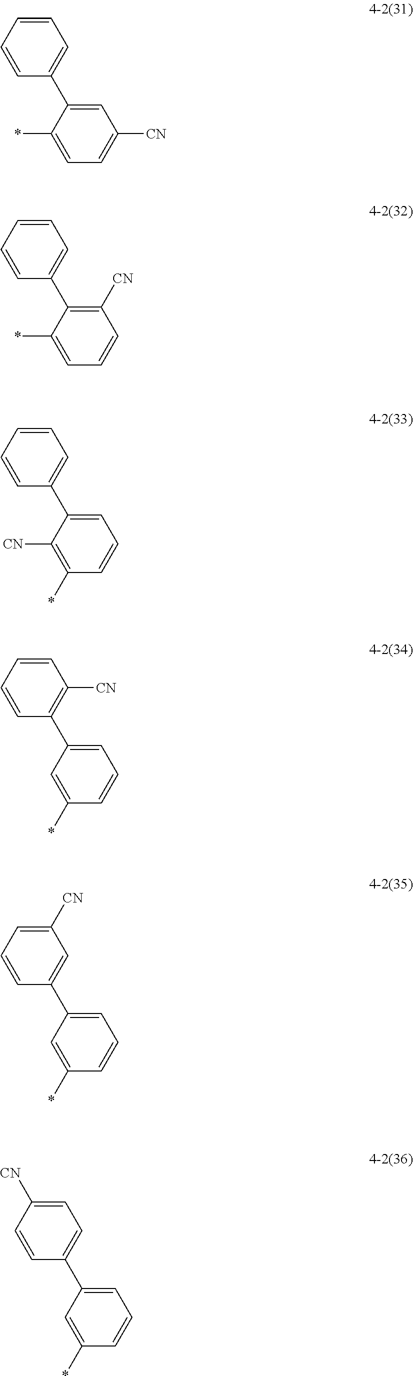 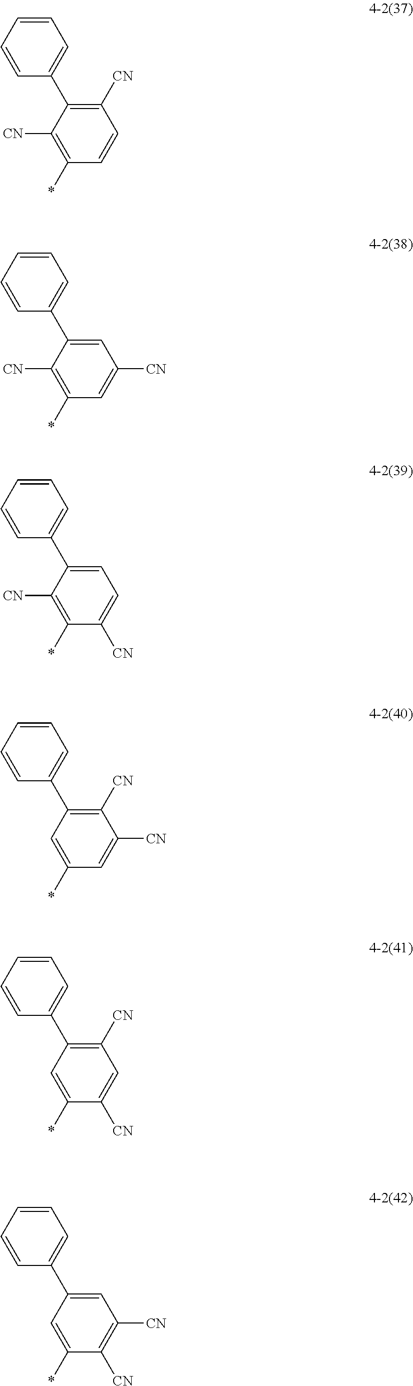 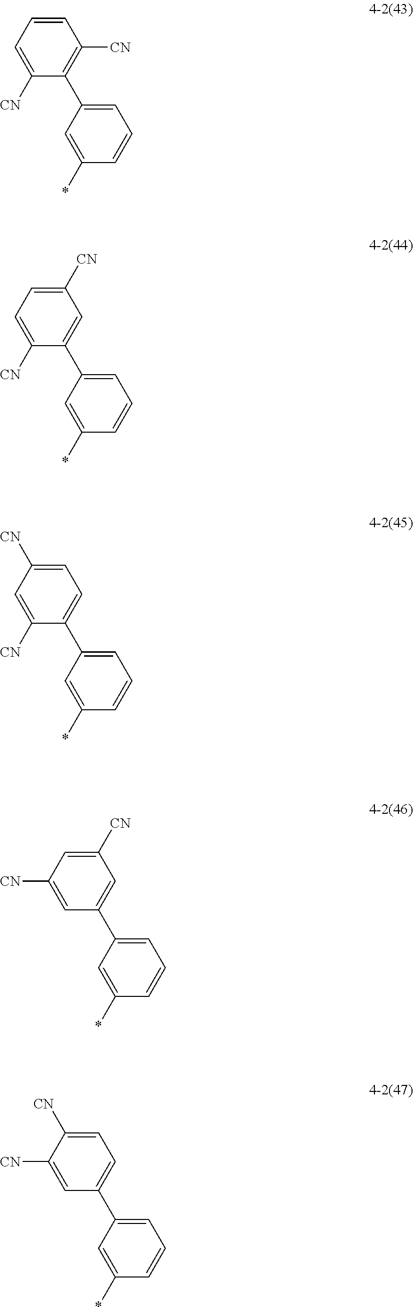 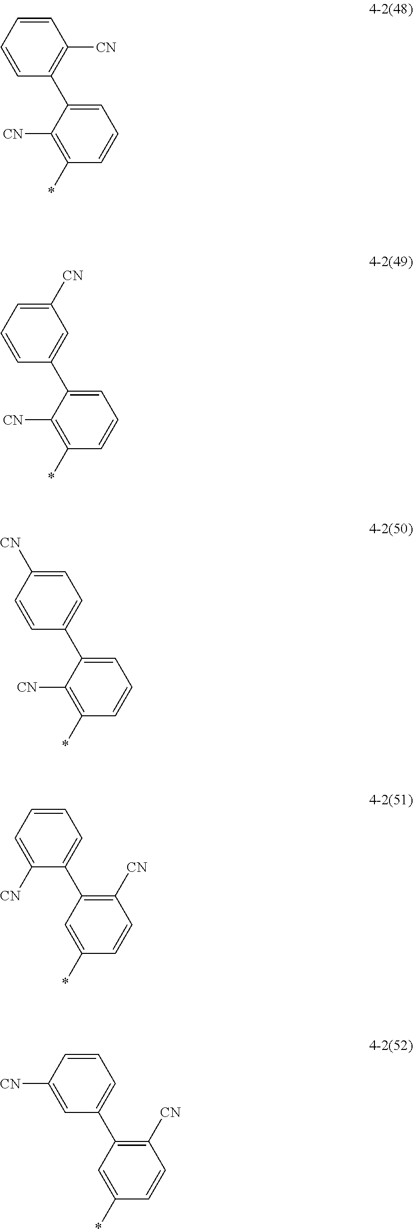 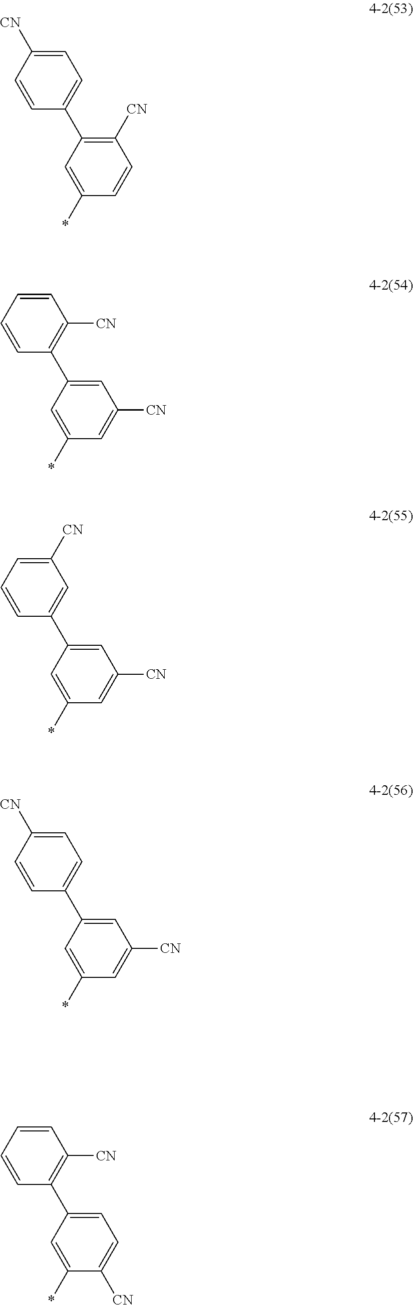 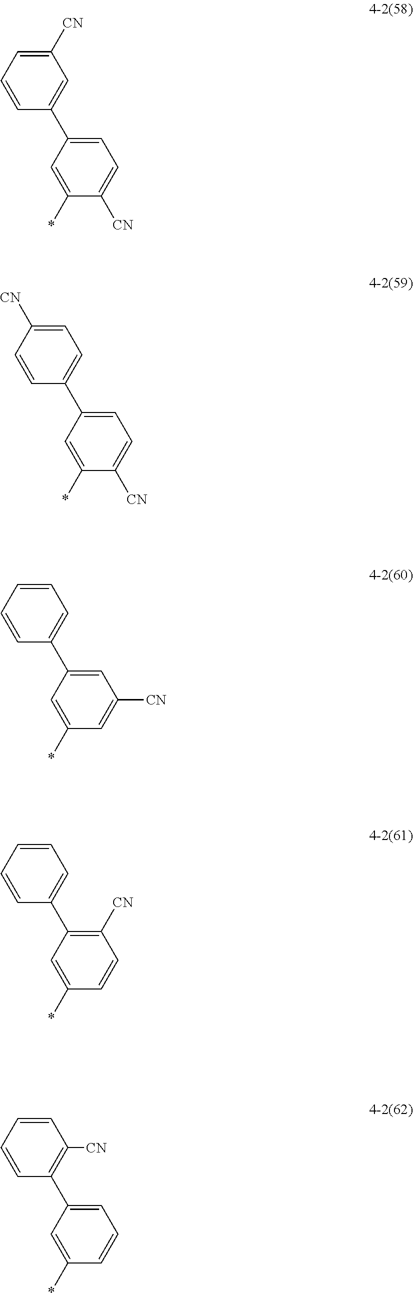 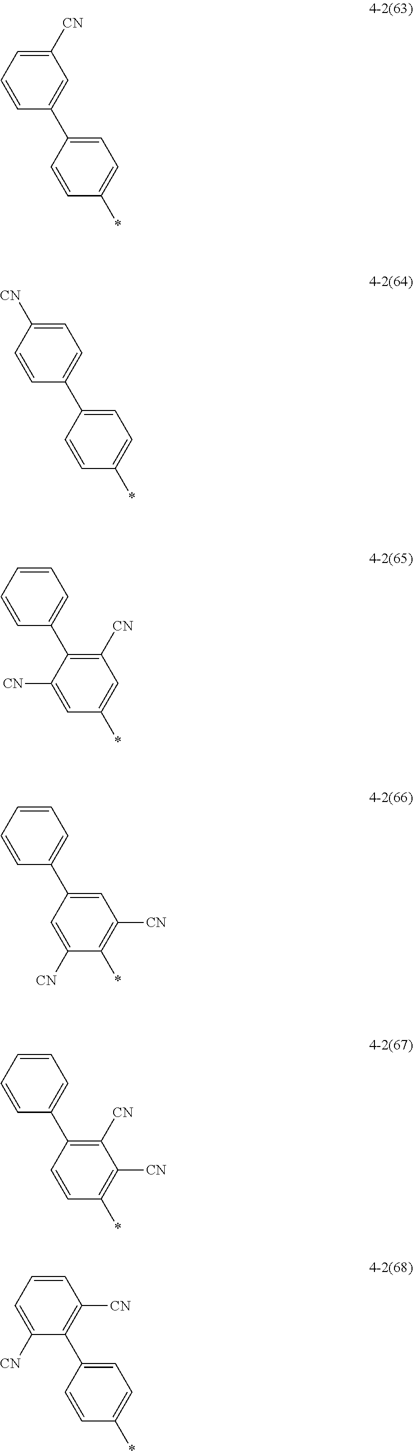 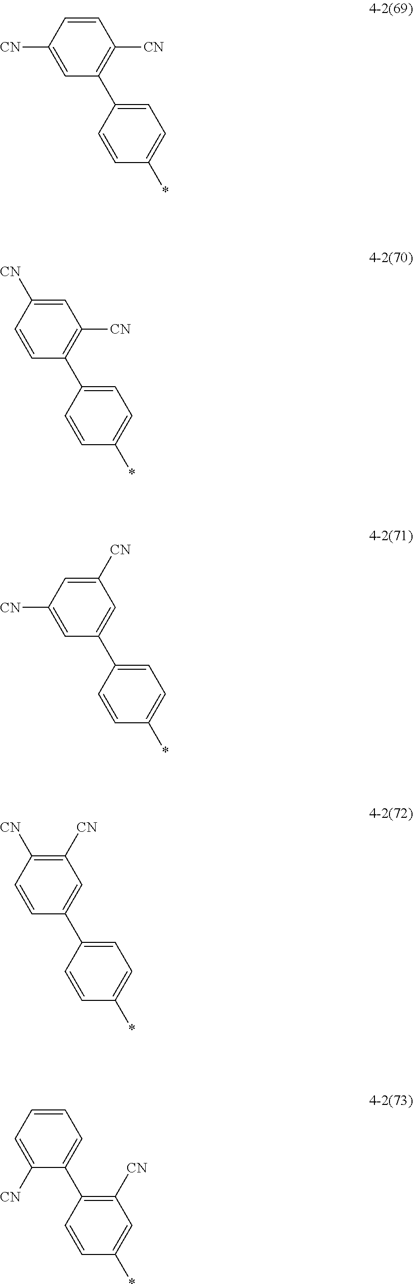 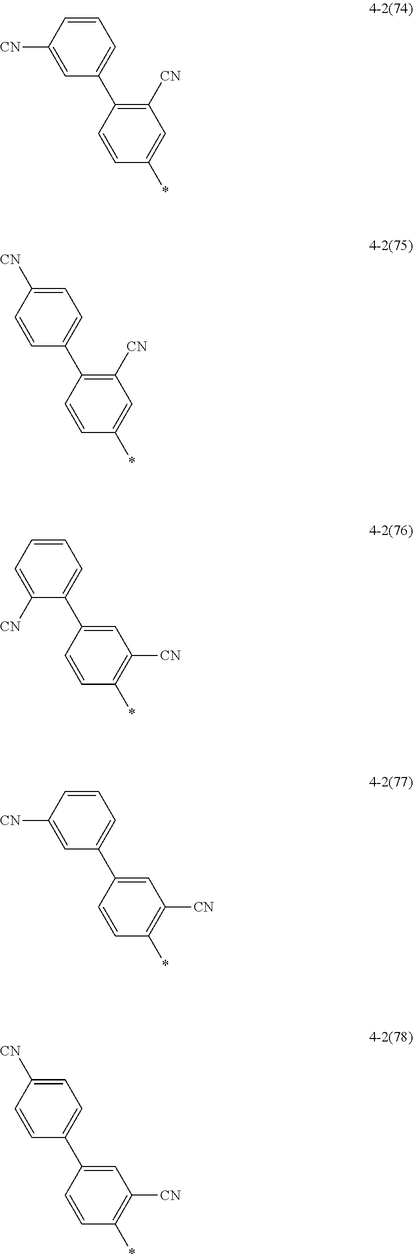
wherein, in Formulae 4-1(1) to 4-1(8) and 4-2(1) to 4-2(78), * indicates a binding site to an adjacent atom.

12. The condensed cyclic compound of claim 1, wherein R₁₁, R₁₃, and R₁₄ are each hydrogen, and
R₁₂ is a cyano group-containing phenyl group, a cyano group-containing biphenyl group, a cyano group-containing terphenyl group, or a cyano group-containing tetraphenyl group.

13. The condensed cyclic compound of claim 1, wherein the number of cyano groups in Formula 1 is 1, 2, 3, 4, or 5.

14. The condensed cyclic compound of claim 1, wherein the condensed cyclic compound is selected from Compounds 1 to 428:

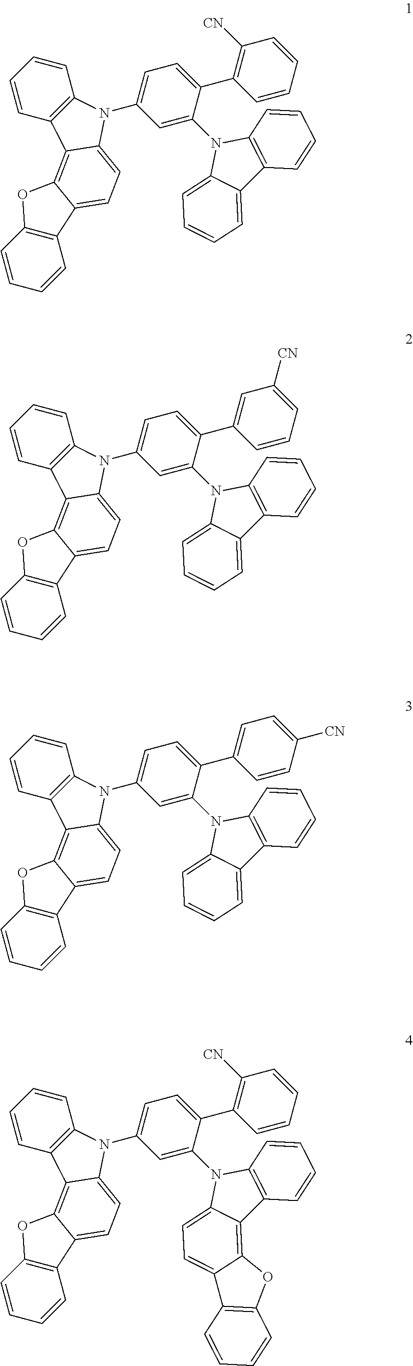

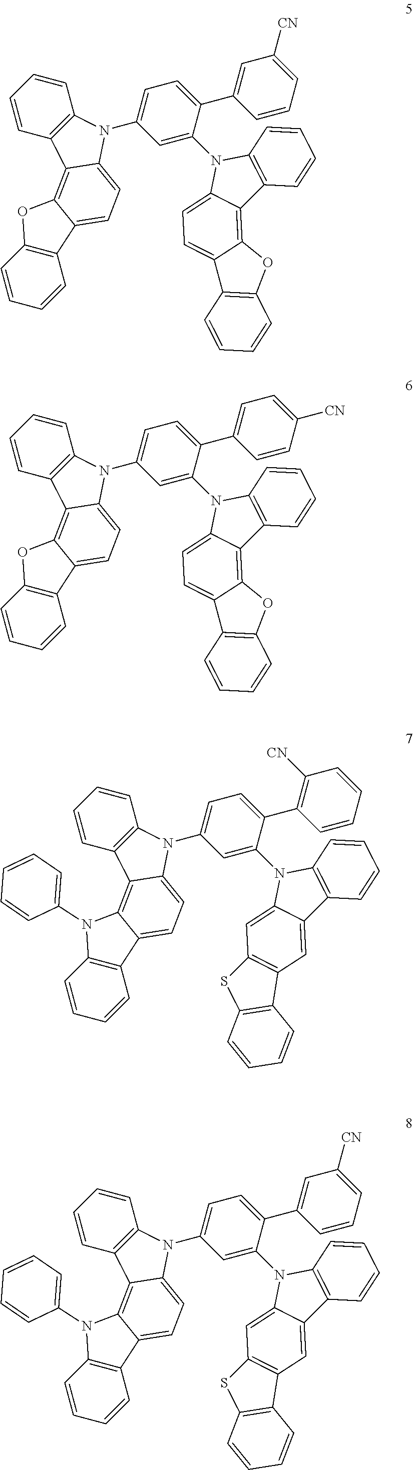

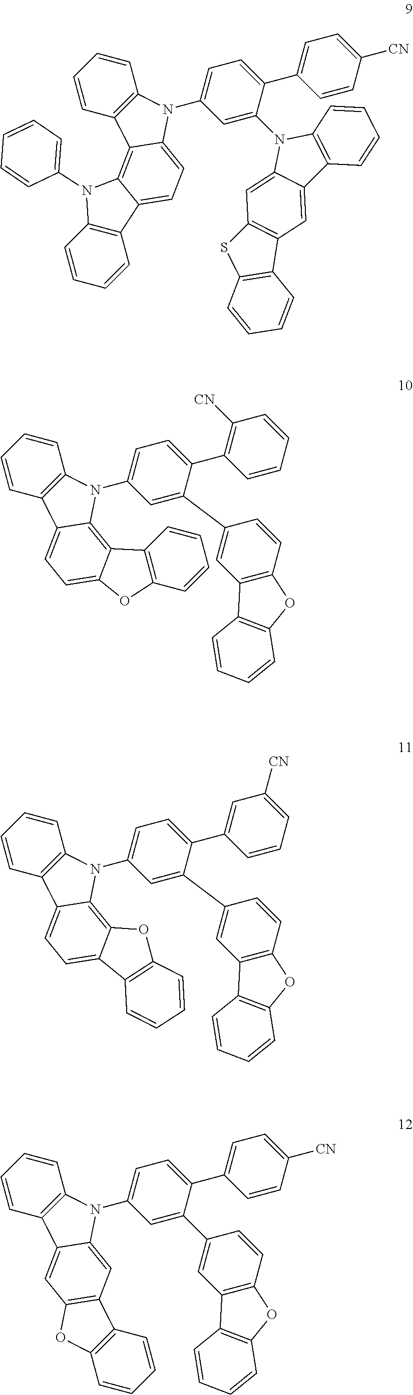

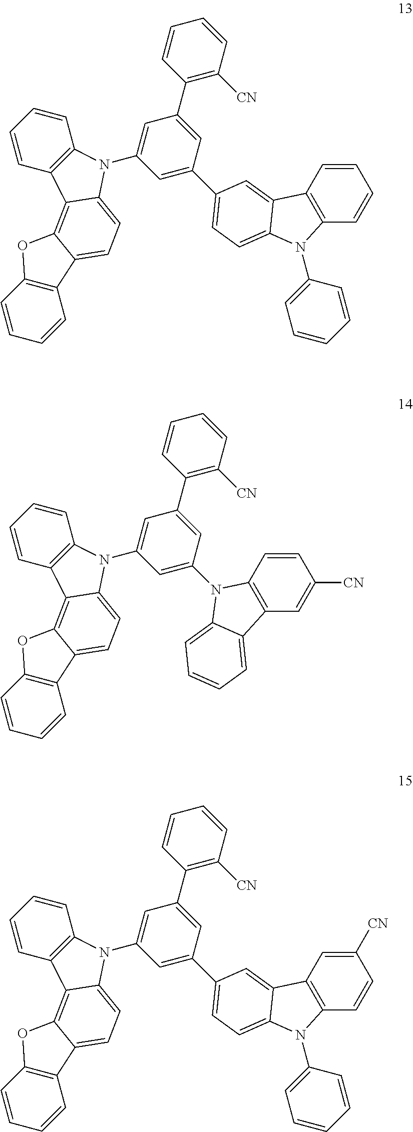 [SMILES: N#Cc1ccccc1-c1cc(-c2ccc3c(c2)c2ccccc2n3-c2ccccc2)cc(-n2c3ccccc3c3c4oc5ccccc5c4ccc32)c1 ; N#Cc1ccc2c(c1)c1ccccc1n2-c1cc(-c2ccccc2C#N)cc(-n2c3ccccc3c3c4oc5ccccc5c4ccc32)c1 ; N#Cc1ccc2c(c1)c1cc(-c3cc(-c4ccccc4C#N)cc(-n4c5ccccc5c5c6oc7ccccc7c6ccc54)c3)ccc1n2-c1ccccc1]

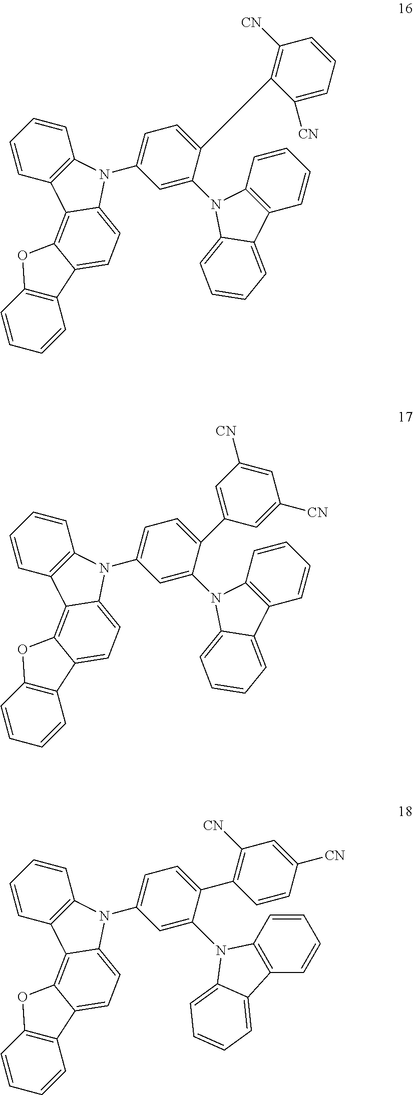

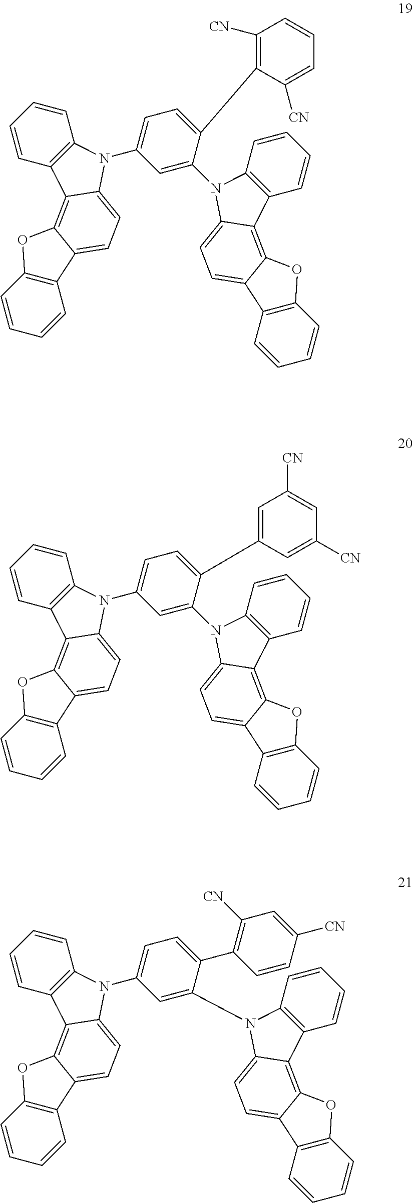

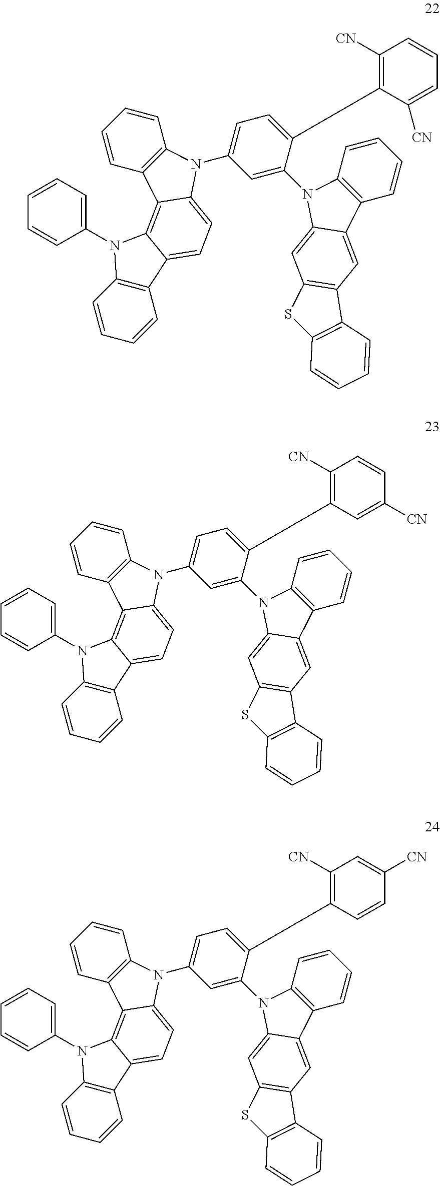

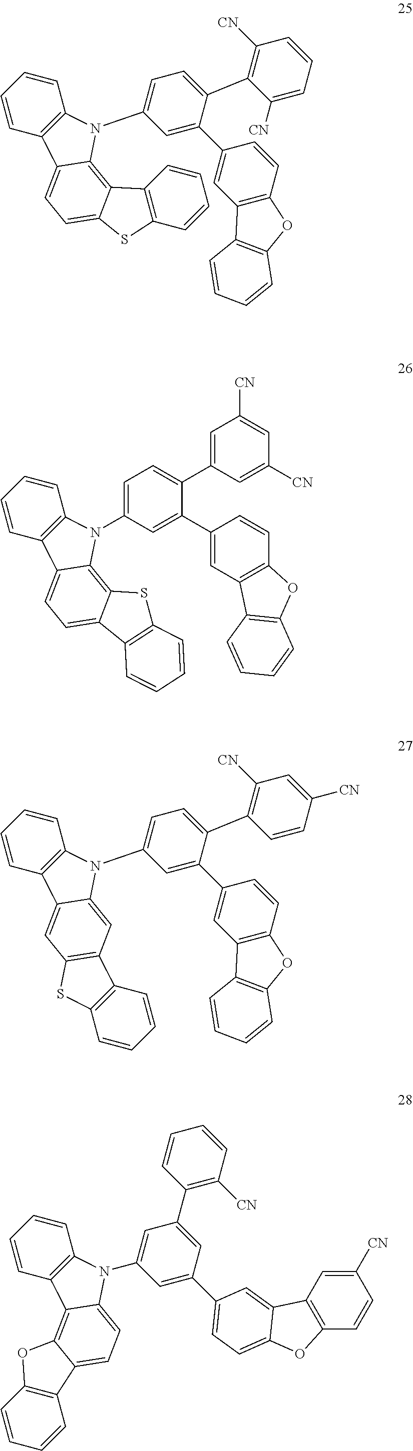

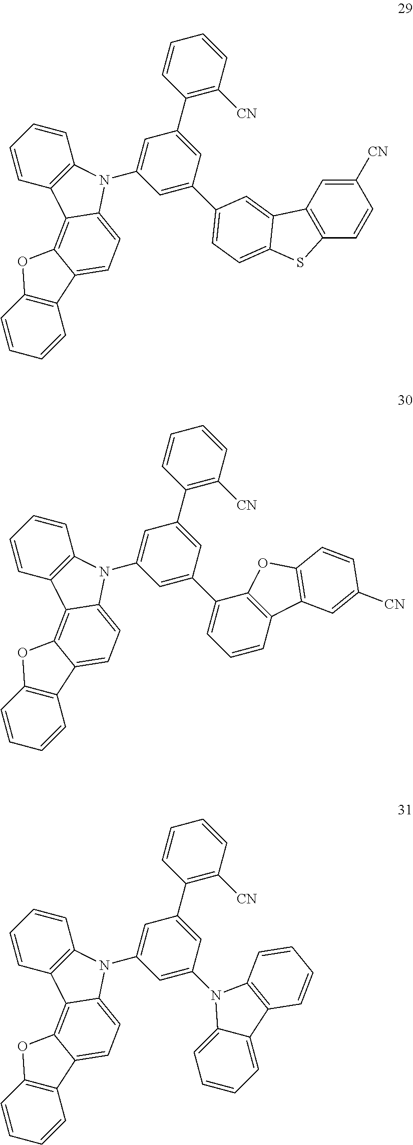 [SMILES: N#Cc1ccc2sc3ccc(-c4cc(-c5ccccc5C#N)cc(-n5c6ccccc6c6c7oc8ccccc8c7ccc65)c4)cc3c2c1 ; N#Cc1ccc2oc3c(-c4cc(-c5ccccc5C#N)cc(-n5c6ccccc6c6c7oc8ccccc8c7ccc65)c4)cccc3c2c1 ; N#Cc1ccccc1-c1cc(-n2c3ccccc3c3ccccc32)cc(-n2c3ccccc3c3c4oc5ccccc5c4ccc32)c1]

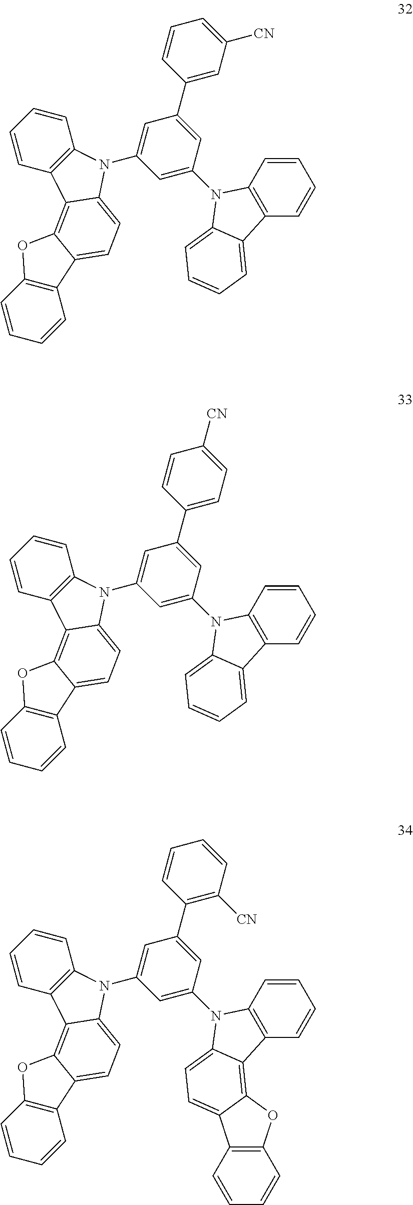 [SMILES: N#Cc1cccc(-c2cc(-n3c4ccccc4c4ccccc43)cc(-n3c4ccccc4c4c5oc6ccccc6c5ccc43)c2)c1 ; N#Cc1ccc(-c2cc(-n3c4ccccc4c4ccccc43)cc(-n3c4ccccc4c4c5oc6ccccc6c5ccc43)c2)cc1 ; N#Cc1ccccc1-c1cc(-n2c3ccccc3c3c4oc5ccccc5c4ccc32)cc(-n2c3ccccc3c3c4oc5ccccc5c4ccc32)c1]

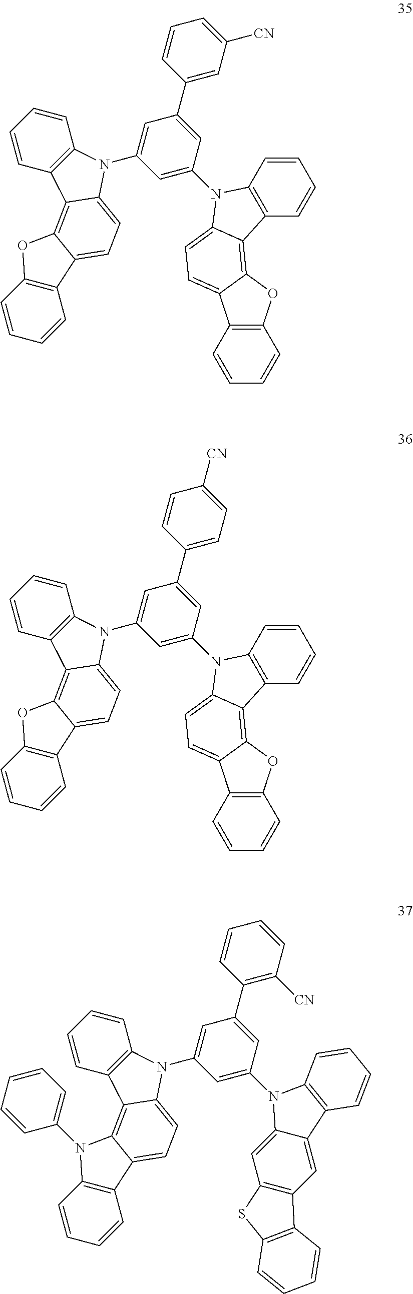 [SMILES: N#Cc1cccc(-c2cc(-n3c4ccccc4c4c5oc6ccccc6c5ccc43)cc(-n3c4ccccc4c4c5oc6ccccc6c5ccc43)c2)c1 ; N#Cc1ccc(-c2cc(-n3c4ccccc4c4c5oc6ccccc6c5ccc43)cc(-n3c4ccccc4c4c5oc6ccccc6c5ccc43)c2)cc1 ; N#Cc1ccccc1-c1cc(-n2c3ccccc3c3cc4c(cc32)sc2ccccc24)cc(-n2c3ccccc3c3c2ccc2c4ccccc4n(-c4ccccc4)c23)c1]

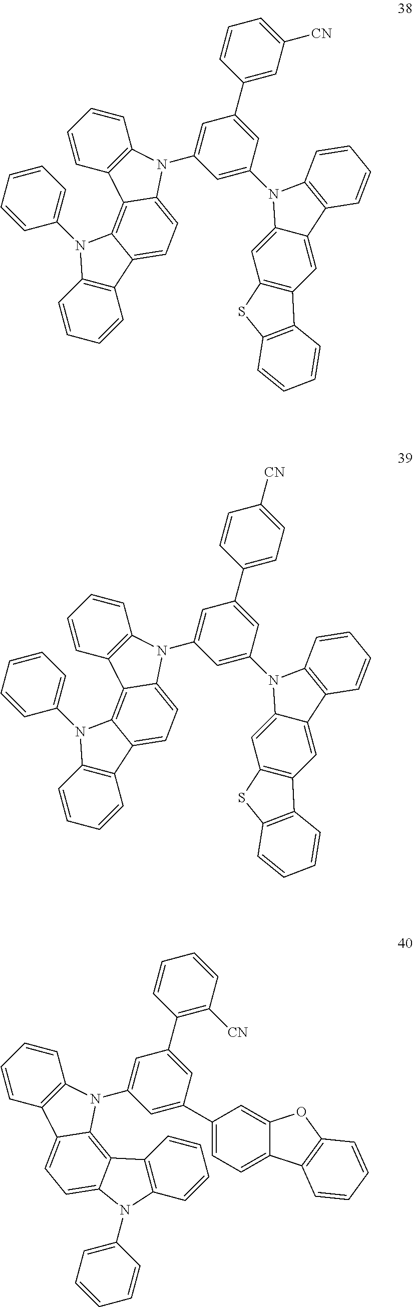 [SMILES: N#Cc1cccc(-c2cc(-n3c4ccccc4c4cc5c(cc43)sc3ccccc35)cc(-n3c4ccccc4c4c3ccc3c5ccccc5n(-c5ccccc5)c34)c2)c1 ; N#Cc1ccc(-c2cc(-n3c4ccccc4c4cc5c(cc43)sc3ccccc35)cc(-n3c4ccccc4c4c3ccc3c5ccccc5n(-c5ccccc5)c34)c2)cc1 ; N#Cc1ccccc1-c1cc(-c2ccc3c(c2)oc2ccccc23)cc(-n2c3ccccc3c3ccc4c(c5ccccc5n4-c4ccccc4)c32)c1]

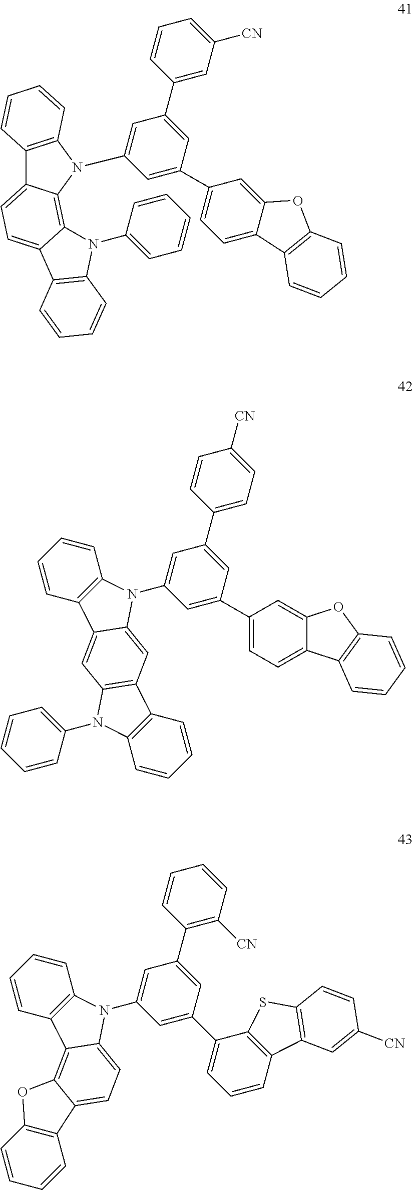 [SMILES: N#Cc1cccc(-c2cc(-c3ccc4c(c3)oc3ccccc34)cc(-n3c4ccccc4c4ccc5c6ccccc6n(-c6ccccc6)c5c43)c2)c1 ; N#Cc1ccc(-c2cc(-c3ccc4c(c3)oc3ccccc34)cc(-n3c4ccccc4c4cc5c(cc43)c3ccccc3n5-c3ccccc3)c2)cc1 ; N#Cc1ccc2sc3c(-c4cc(-c5ccccc5C#N)cc(-n5c6ccccc6c6c7oc8ccccc8c7ccc65)c4)cccc3c2c1]

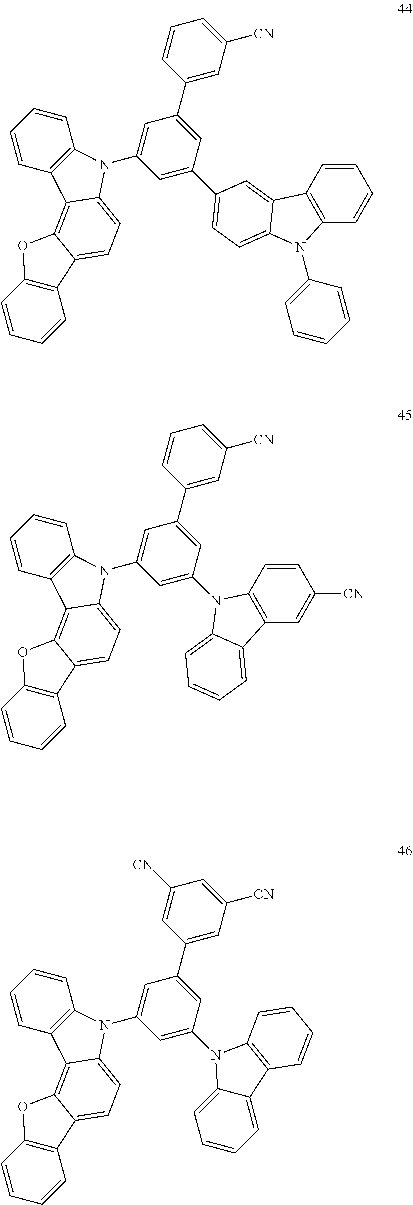

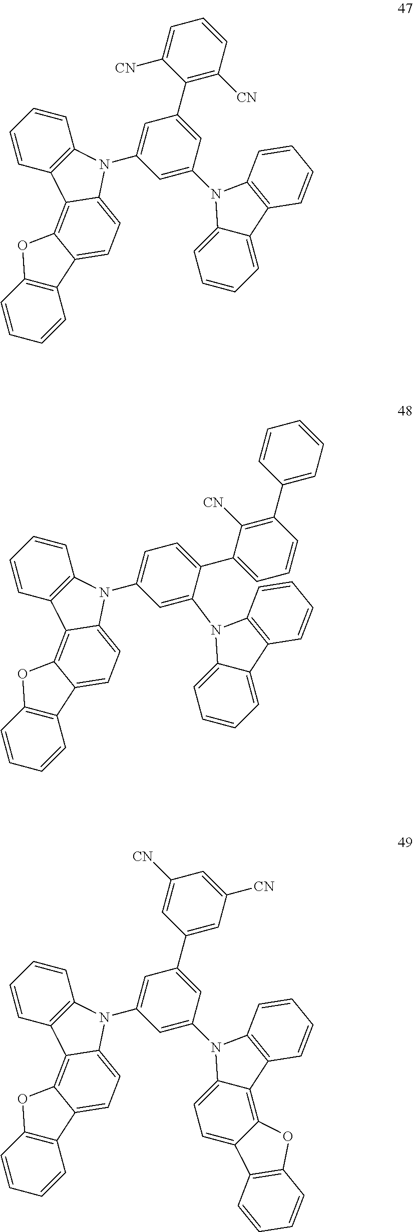

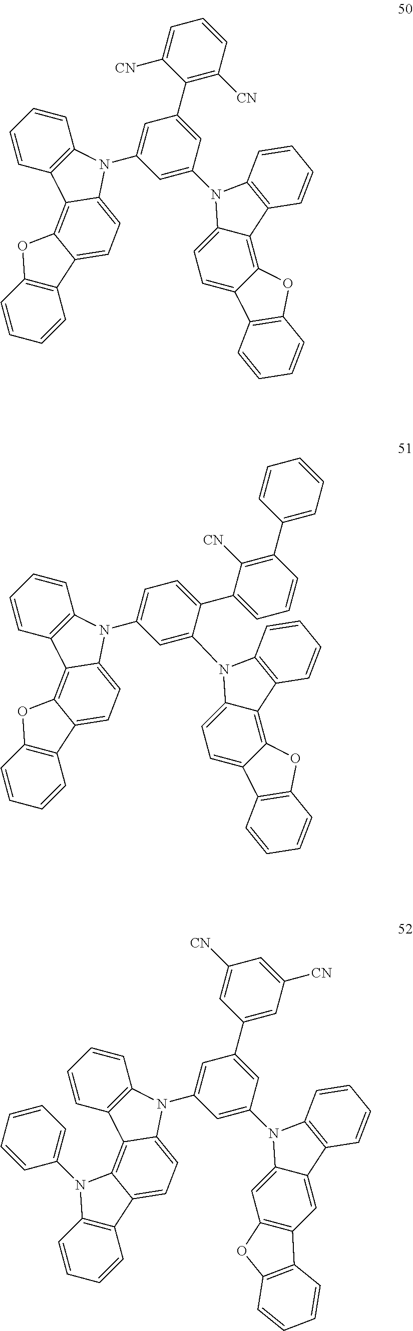

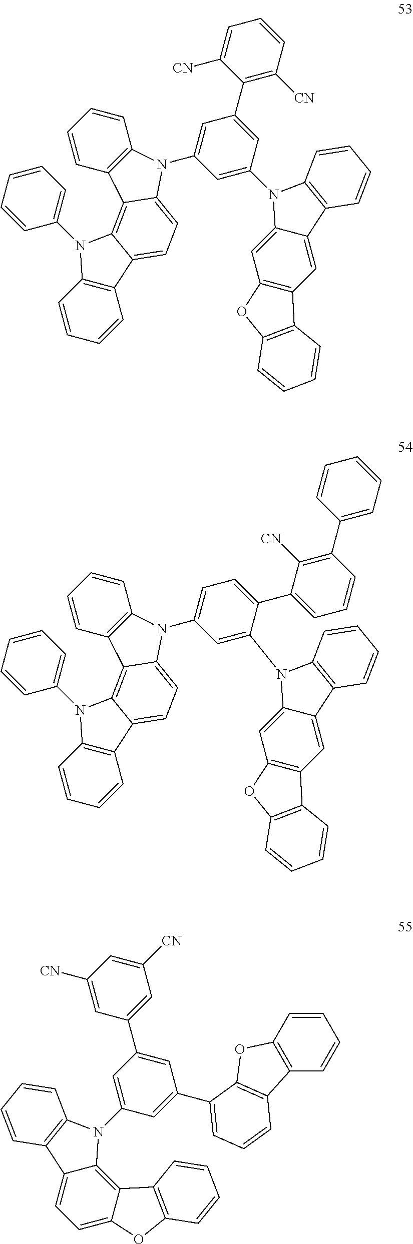

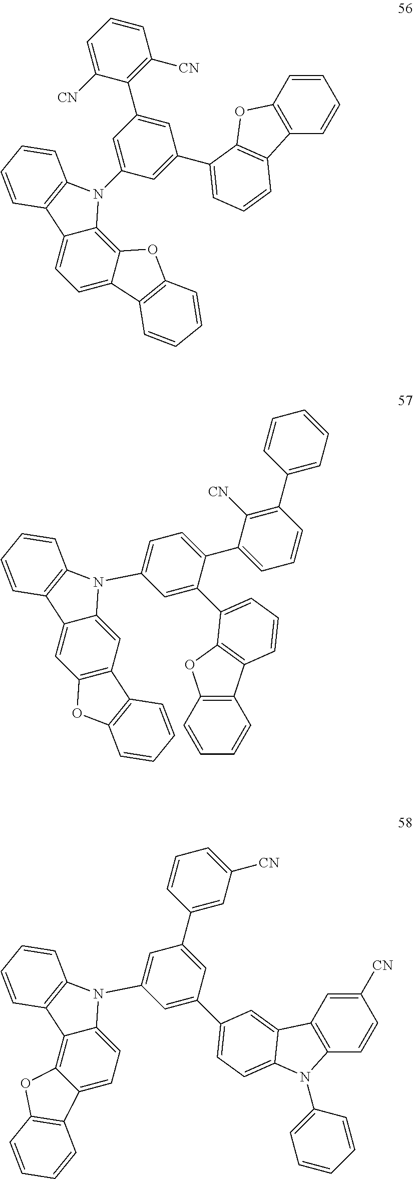

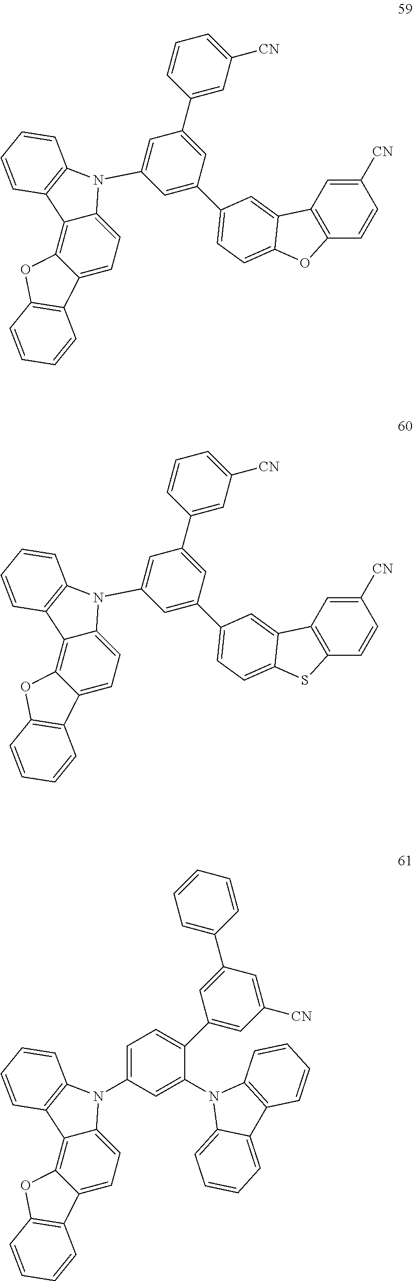 [SMILES: N#Cc1cccc(-c2cc(-c3ccc4oc5ccc(C#N)cc5c4c3)cc(-n3c4ccccc4c4c5oc6ccccc6c5ccc43)c2)c1 ; N#Cc1cccc(-c2cc(-c3ccc4sc5ccc(C#N)cc5c4c3)cc(-n3c4ccccc4c4c5oc6ccccc6c5ccc43)c2)c1 ; N#Cc1cc(-c2ccccc2)cc(-c2ccc(-n3c4ccccc4c4c5oc6ccccc6c5ccc43)cc2-n2c3ccccc3c3ccccc32)c1]

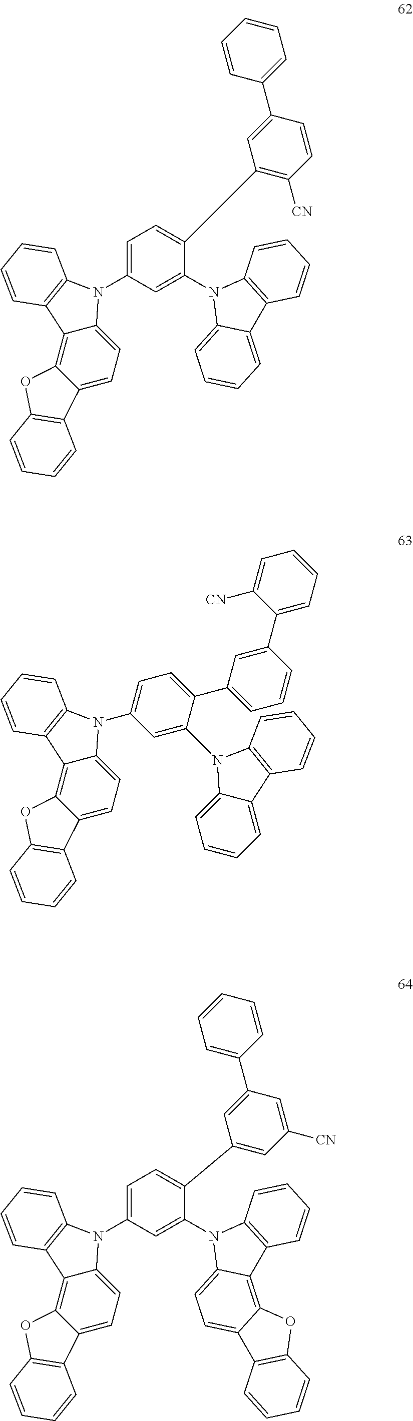

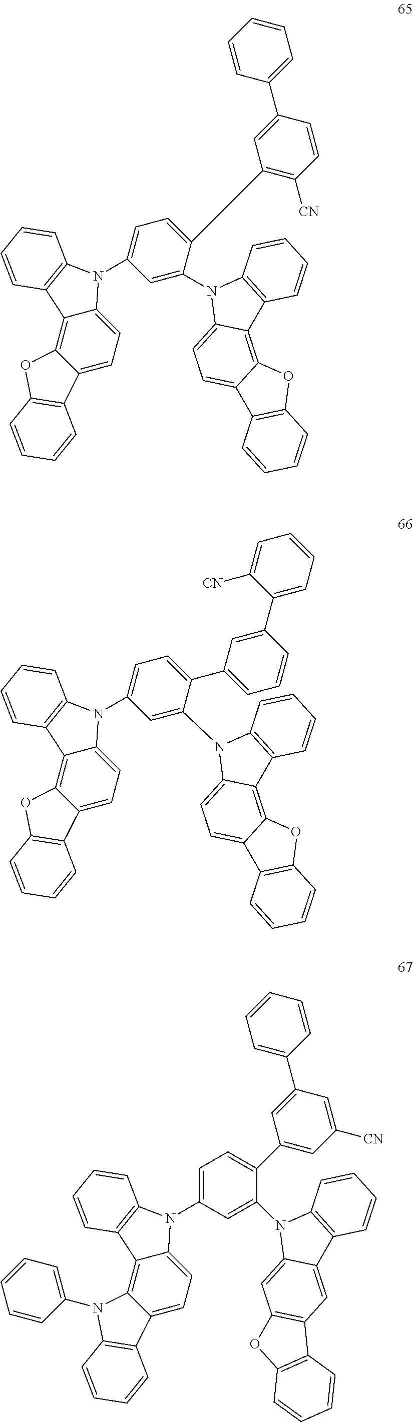

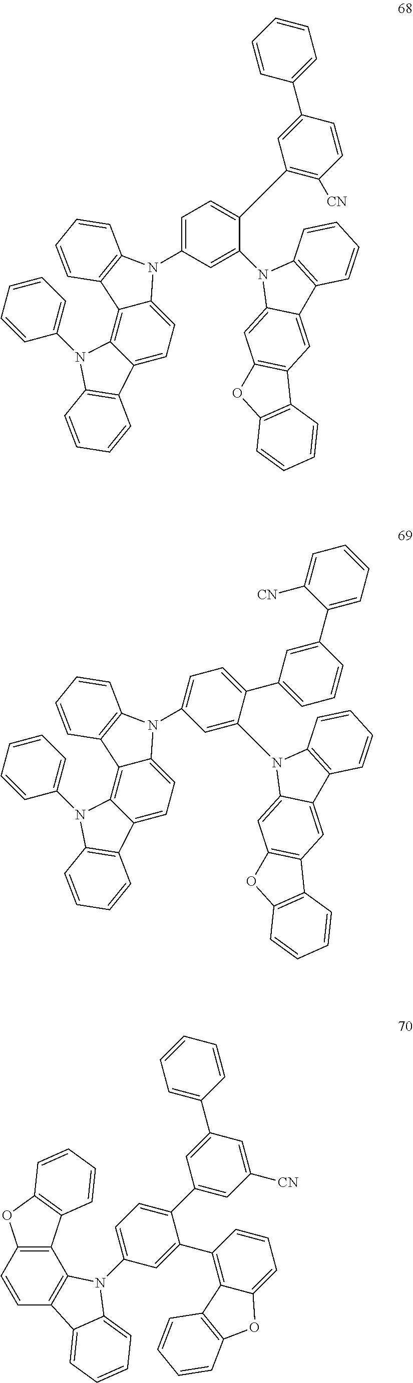

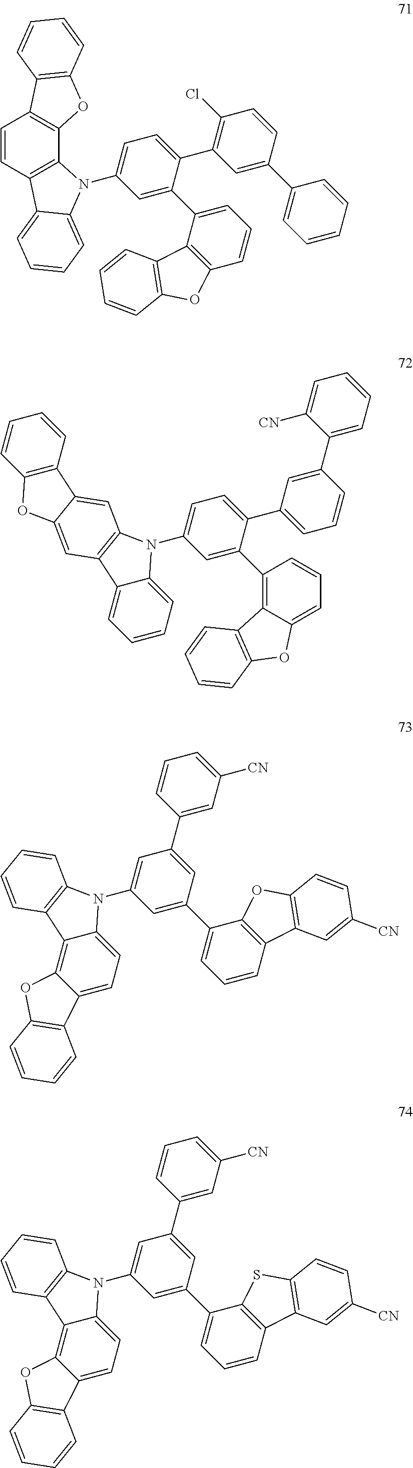

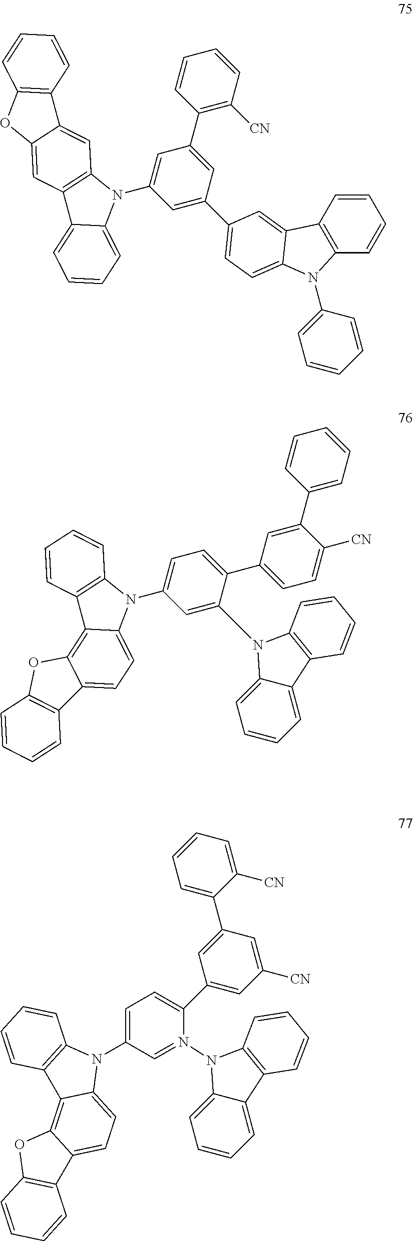

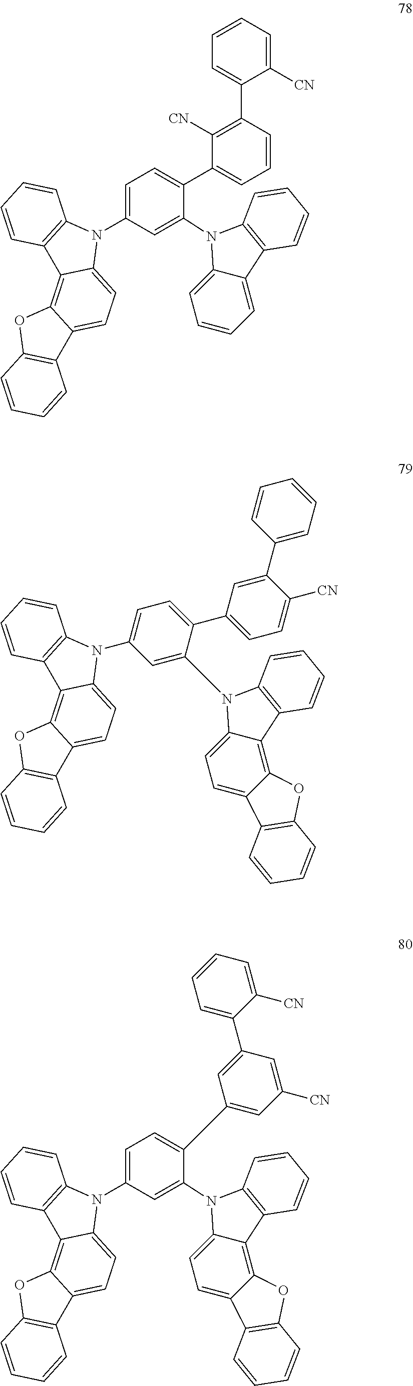

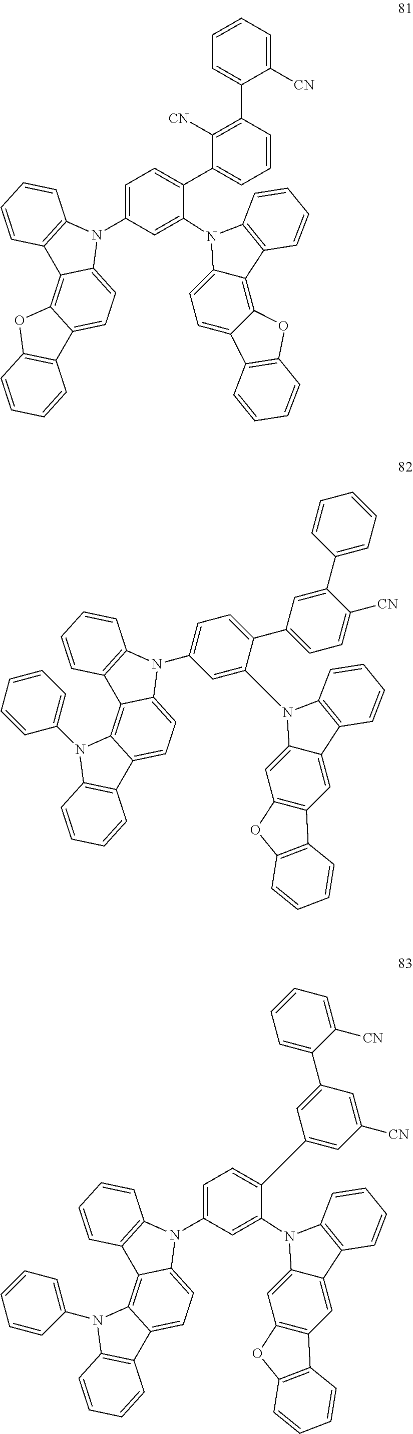

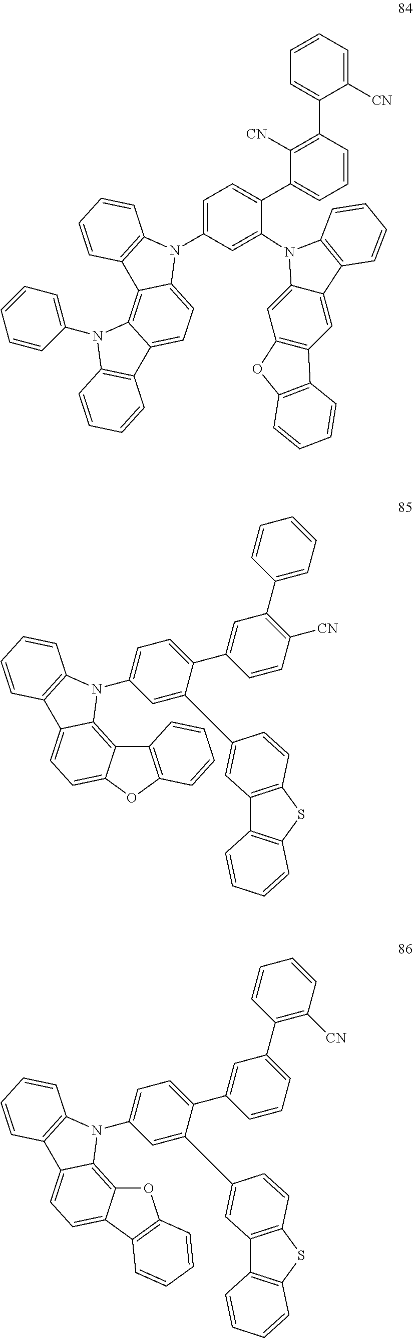

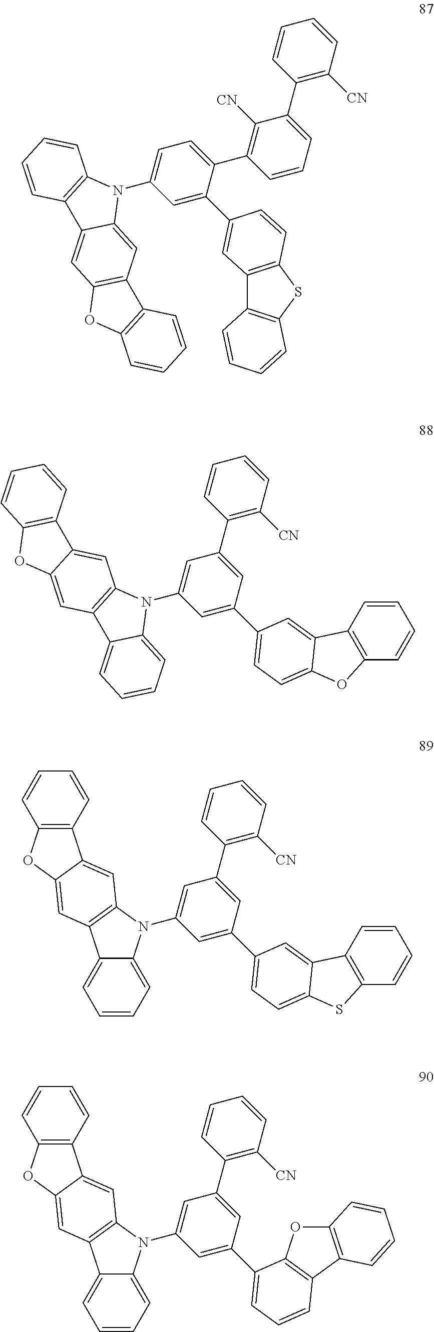

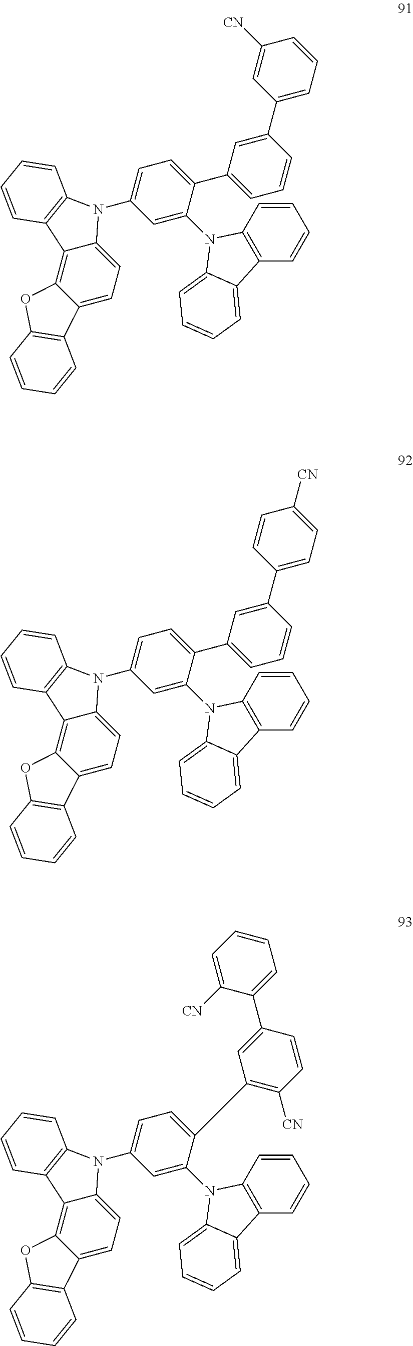

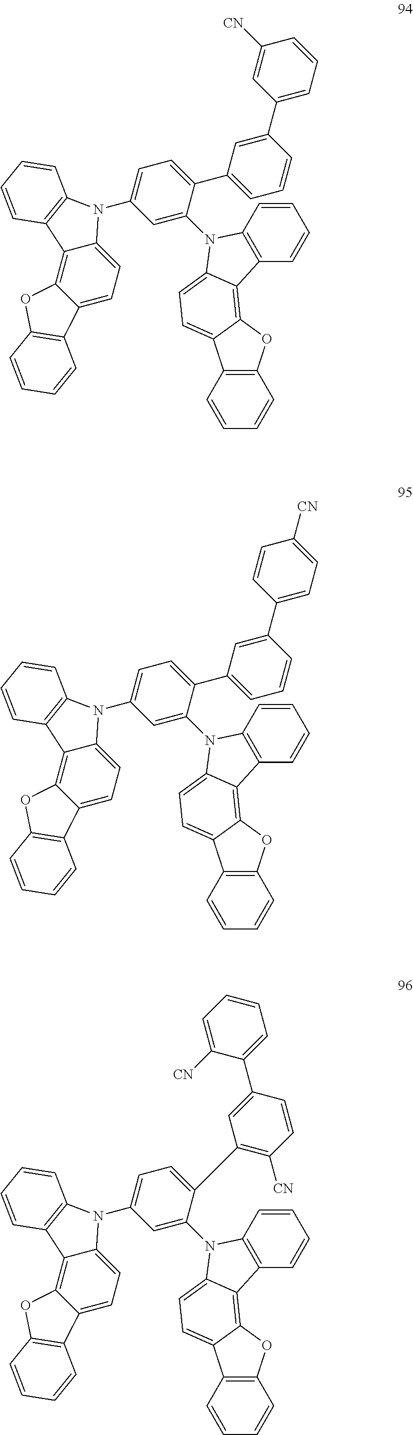

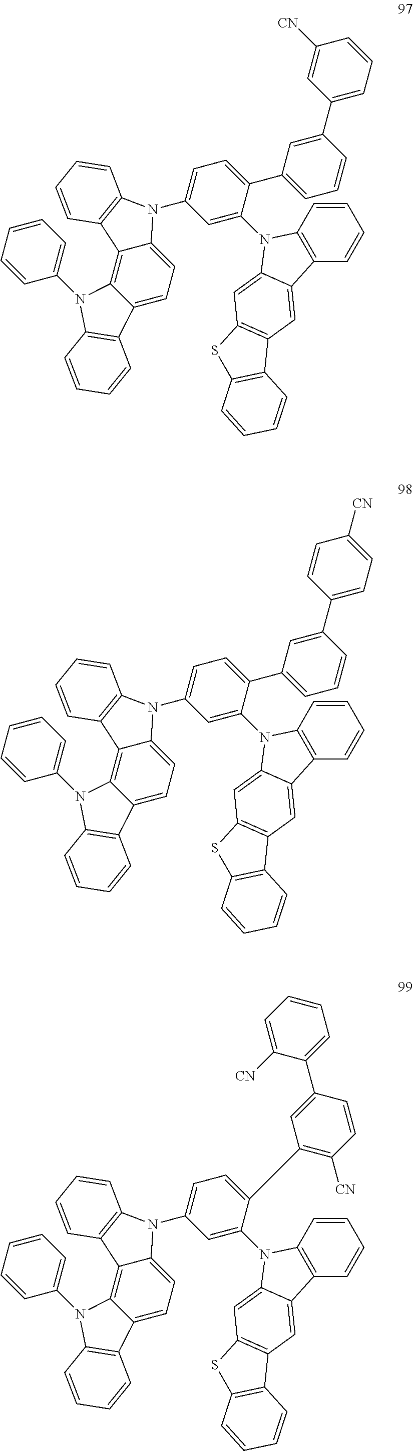

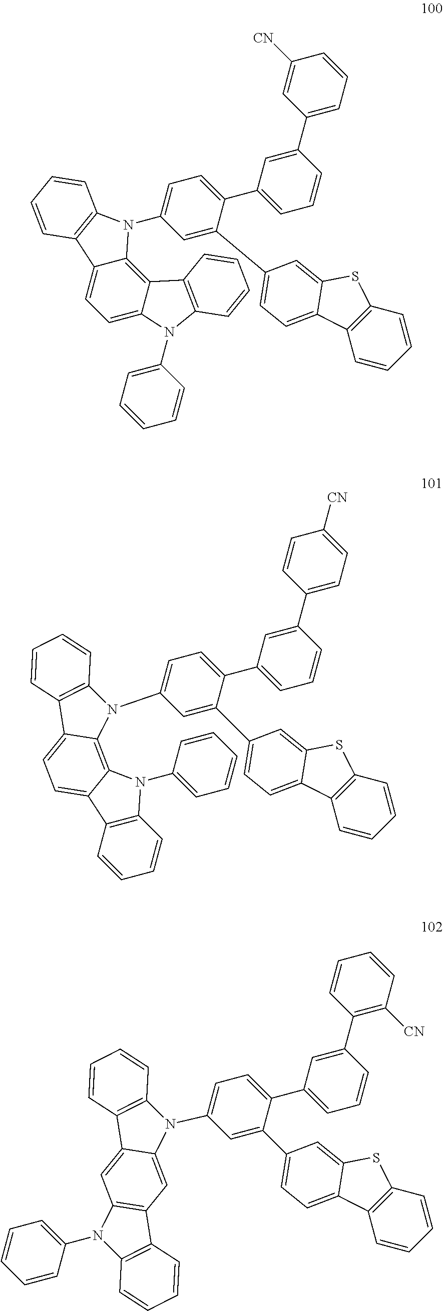

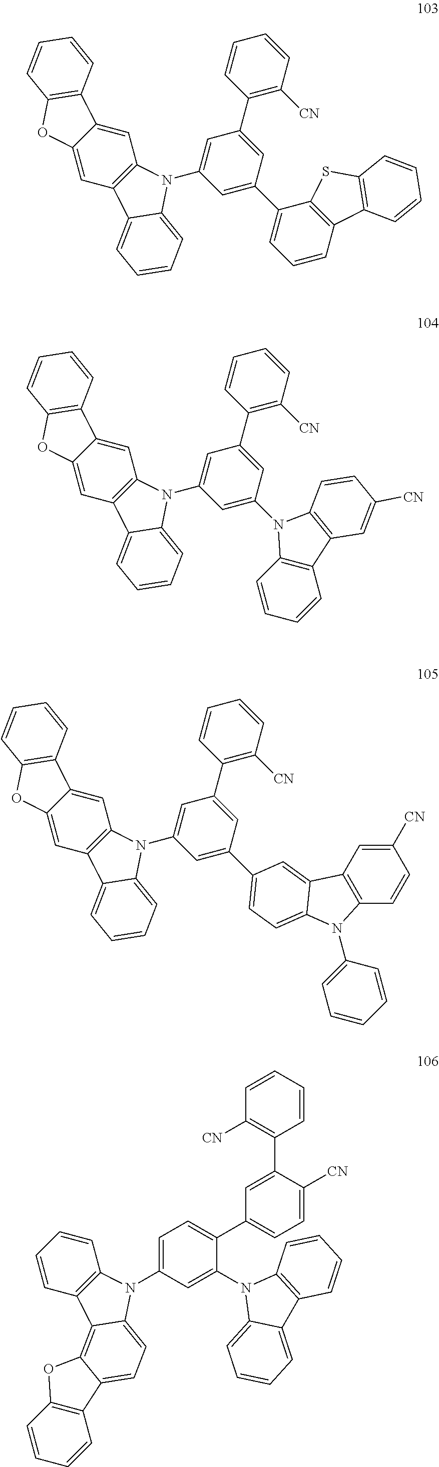

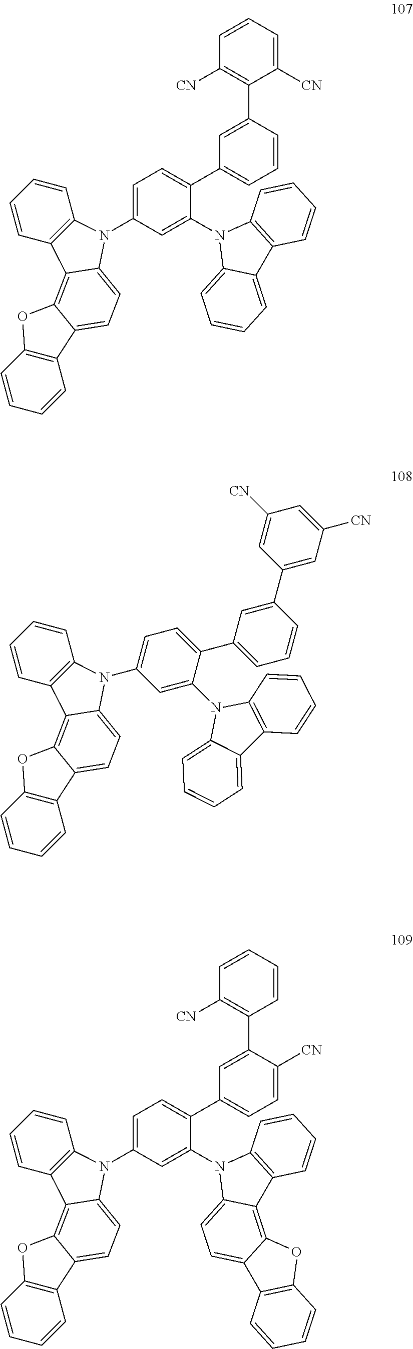

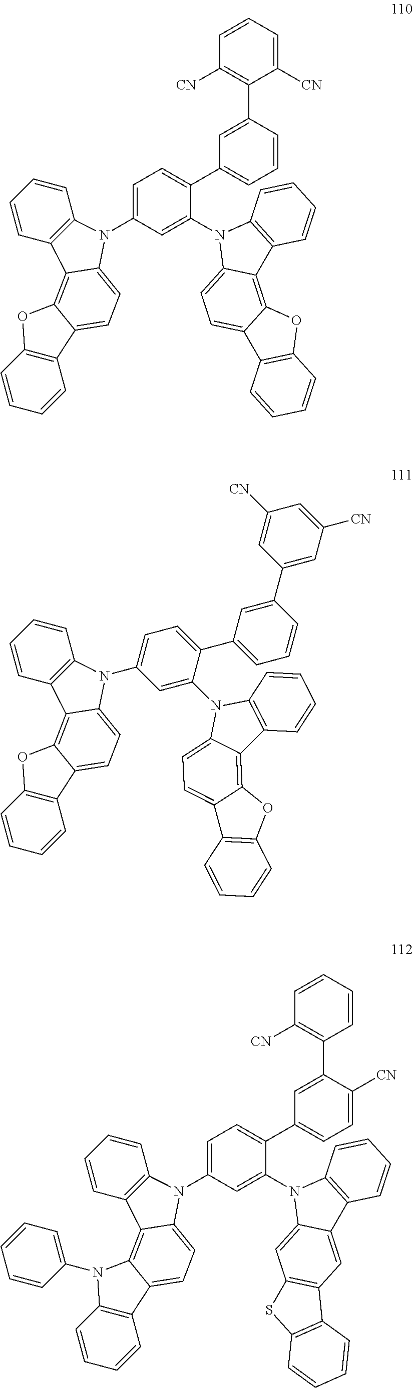

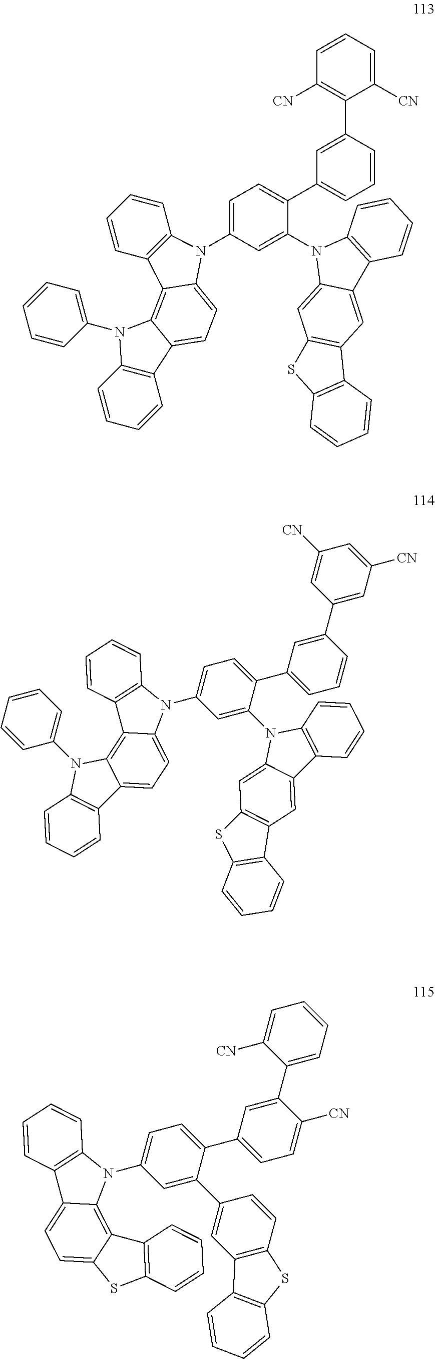

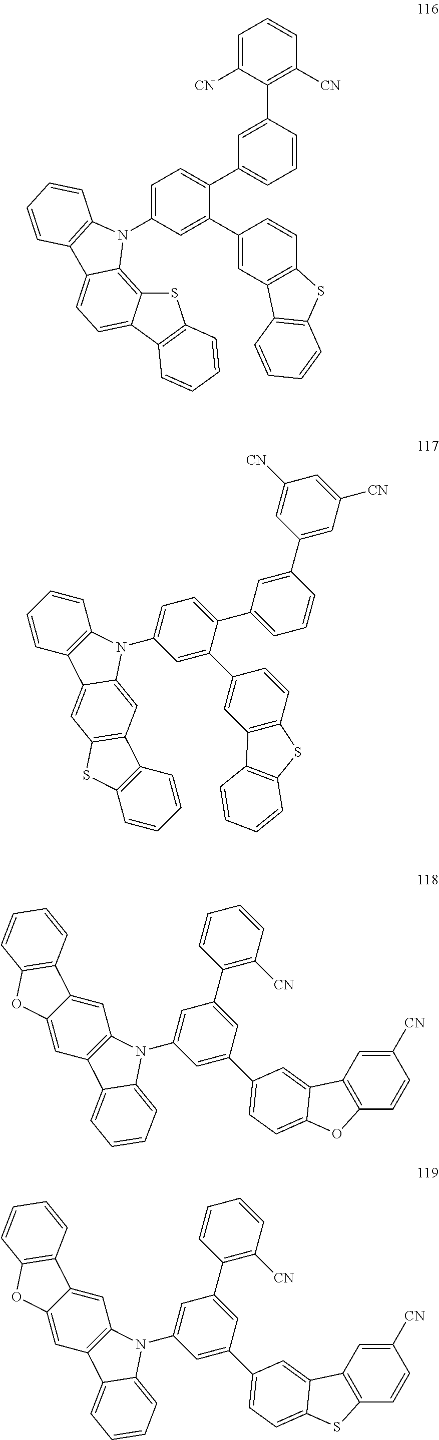

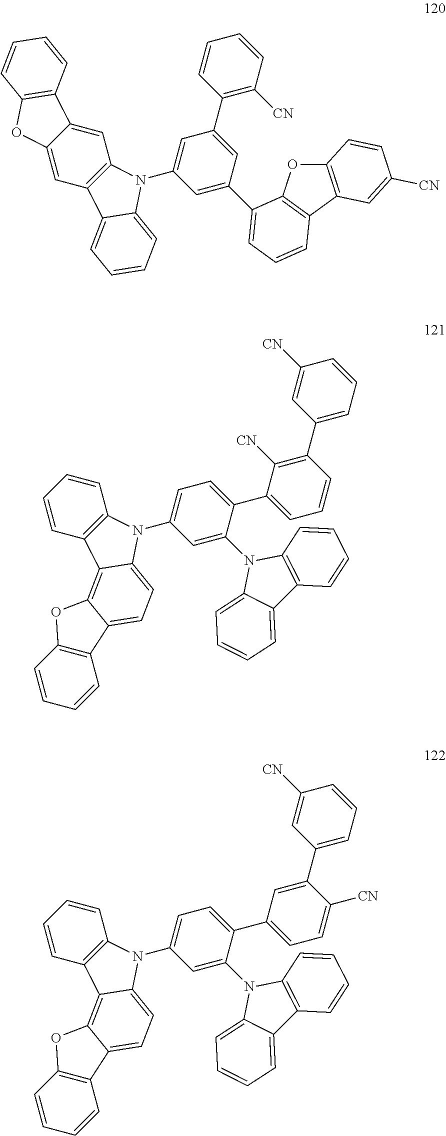

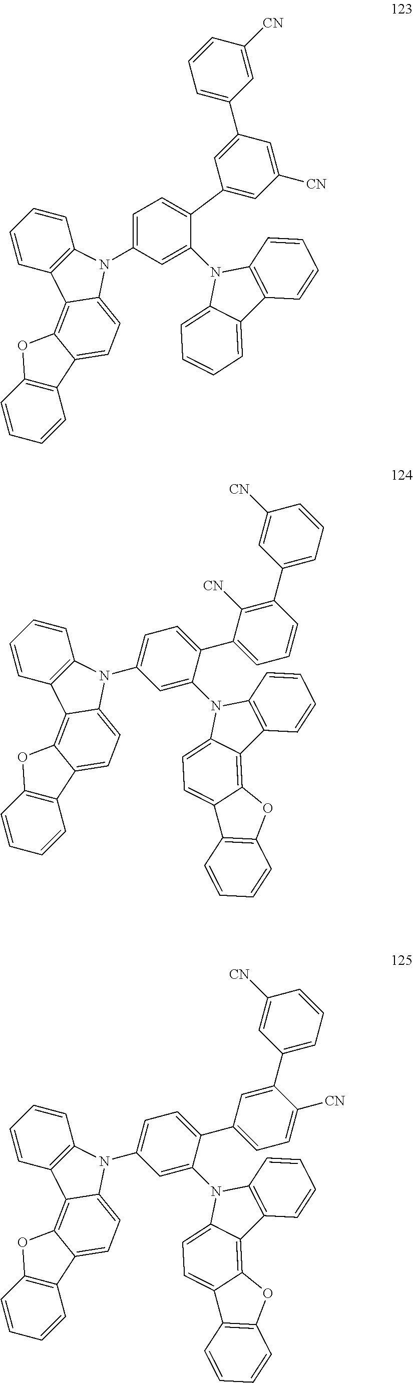

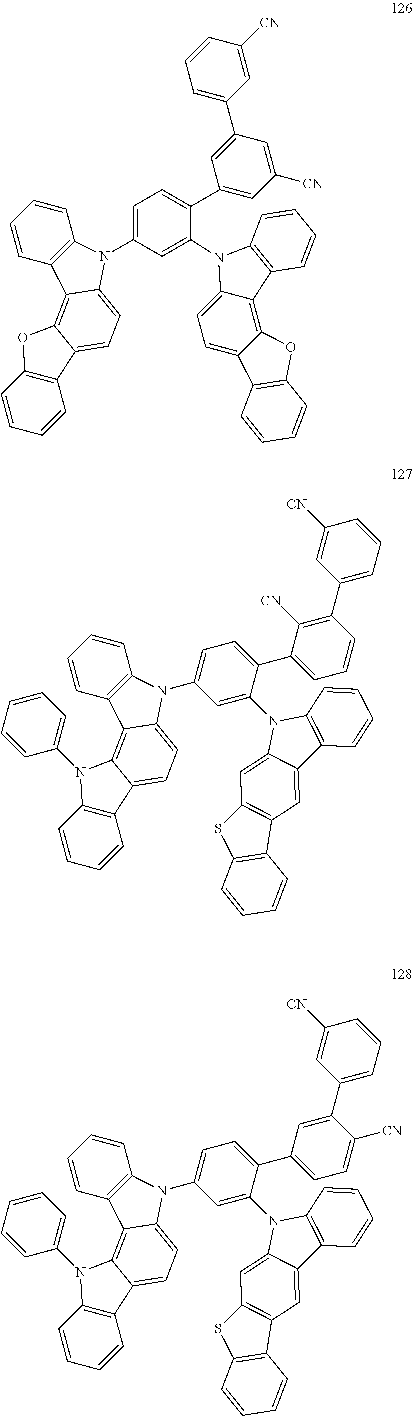

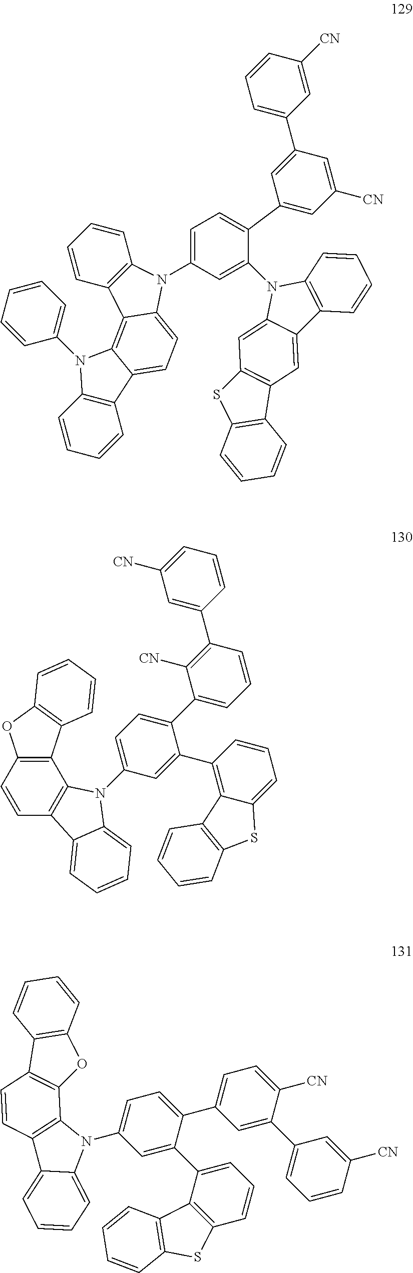

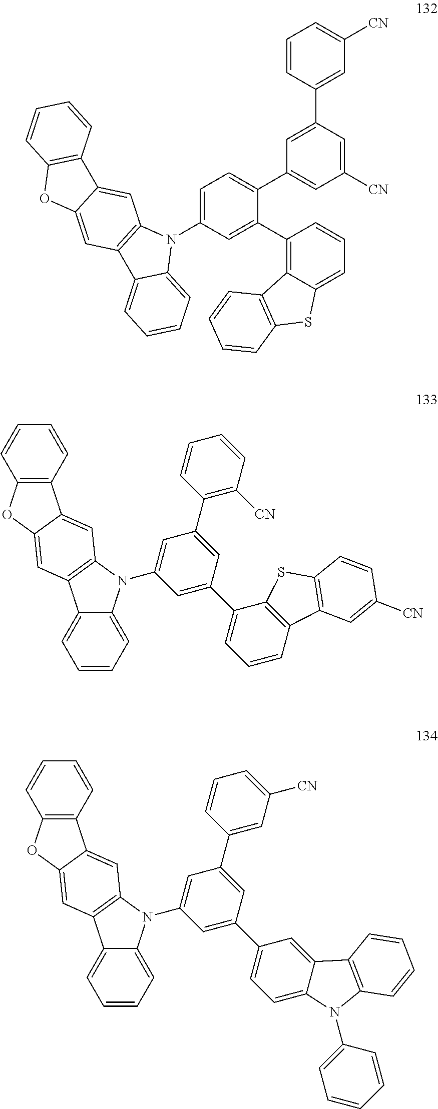 [SMILES: N#Cc1cccc(-c2cc(C#N)cc(-c3ccc(-n4c5ccccc5c5cc6oc7ccccc7c6cc54)cc3-c3cccc4sc5ccccc5c34)c2)c1 ; N#Cc1ccc2sc3c(-c4cc(-c5ccccc5C#N)cc(-n5c6ccccc6c6cc7oc8ccccc8c7cc65)c4)cccc3c2c1 ; N#Cc1cccc(-c2cc(-c3ccc4c(c3)c3ccccc3n4-c3ccccc3)cc(-n3c4ccccc4c4cc5oc6ccccc6c5cc43)c2)c1]

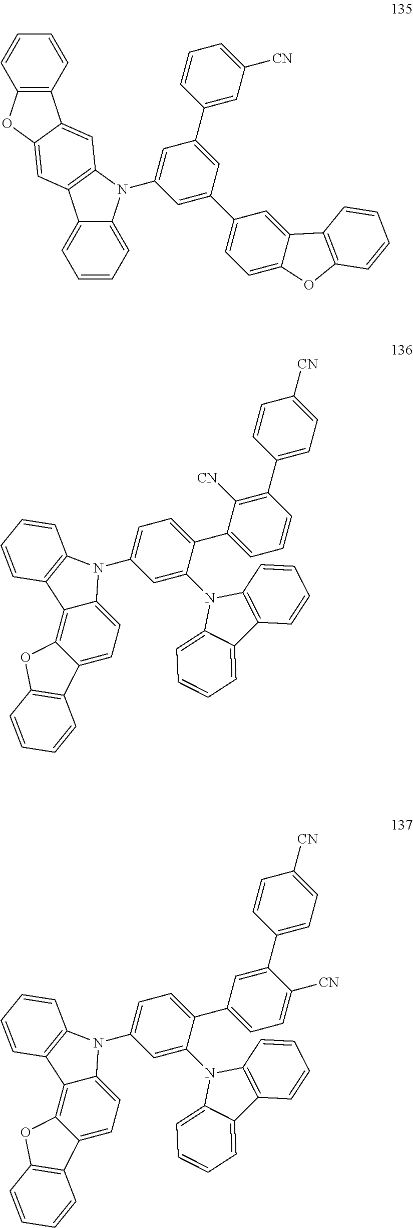

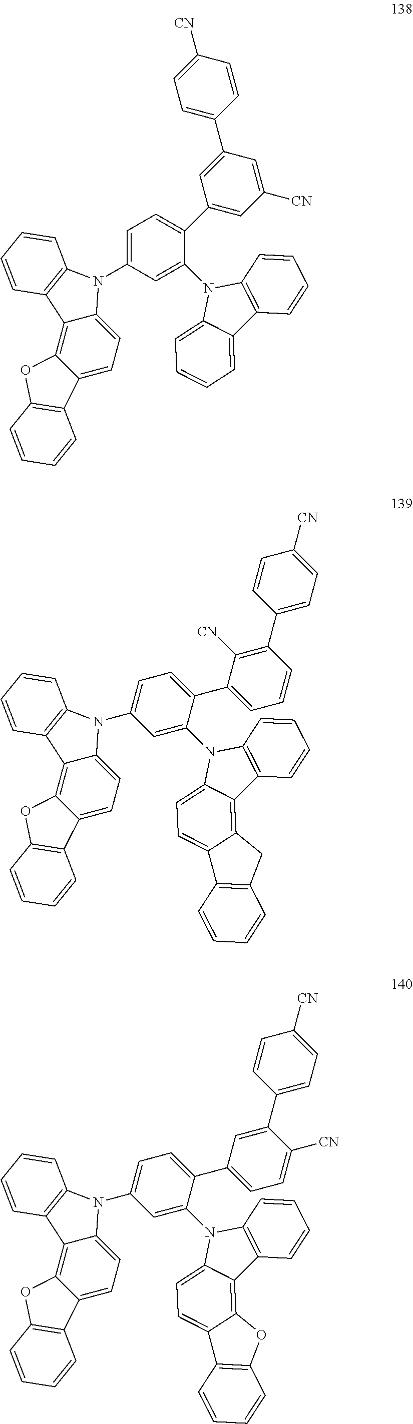

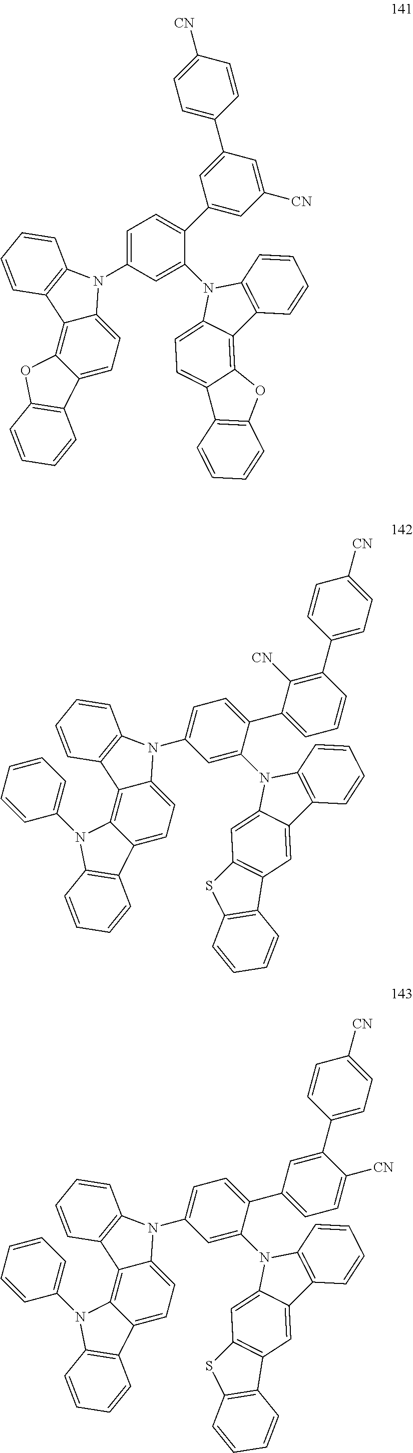

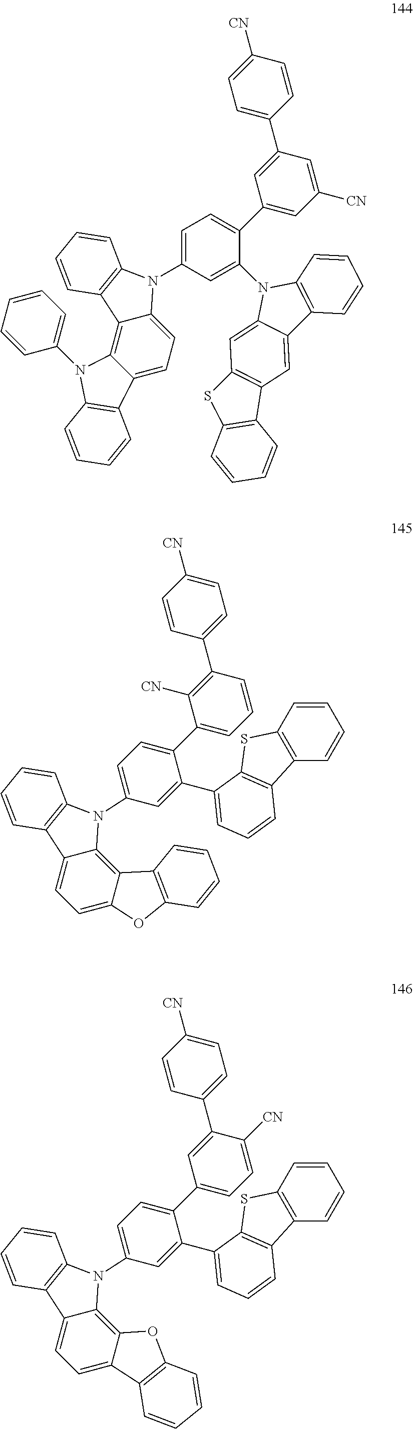

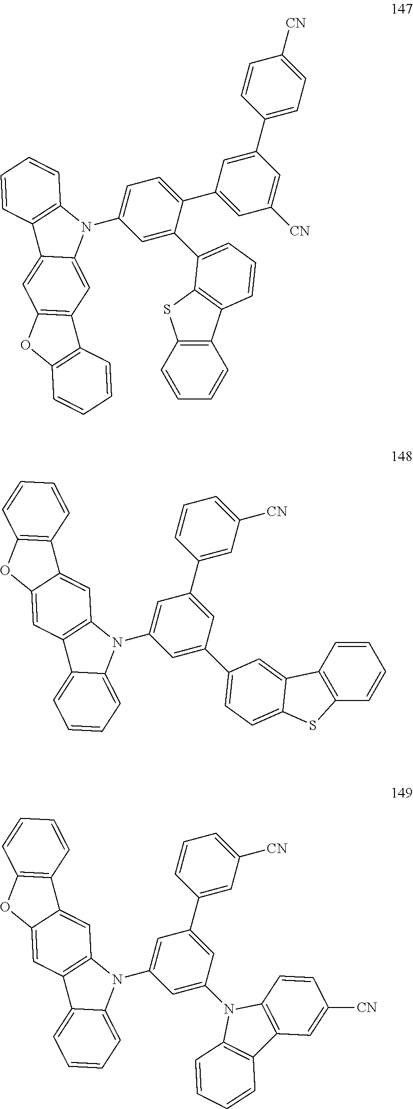 [SMILES: N#Cc1ccc(-c2cc(C#N)cc(-c3ccc(-n4c5ccccc5c5cc6oc7ccccc7c6cc54)cc3-c3cccc4c3sc3ccccc34)c2)cc1 ; N#Cc1cccc(-c2cc(-c3ccc4sc5ccccc5c4c3)cc(-n3c4ccccc4c4cc5oc6ccccc6c5cc43)c2)c1 ; N#Cc1cccc(-c2cc(-n3c4ccccc4c4cc(C#N)ccc43)cc(-n3c4ccccc4c4cc5oc6ccccc6c5cc43)c2)c1]

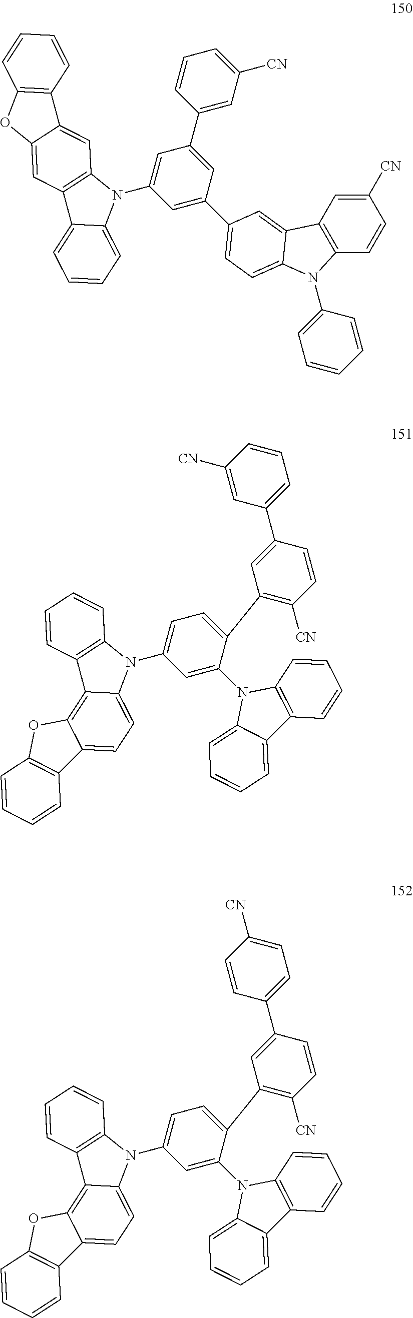

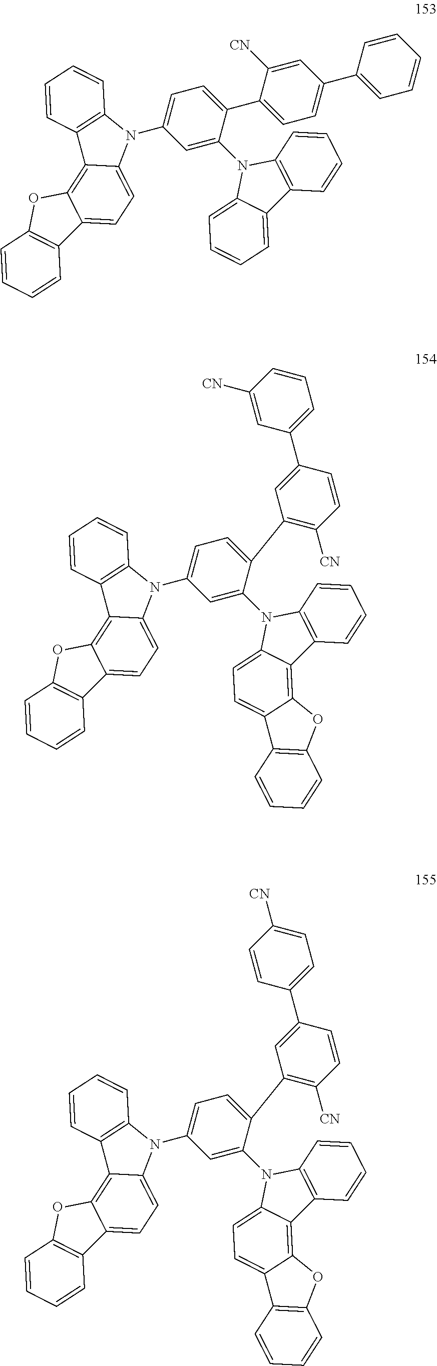

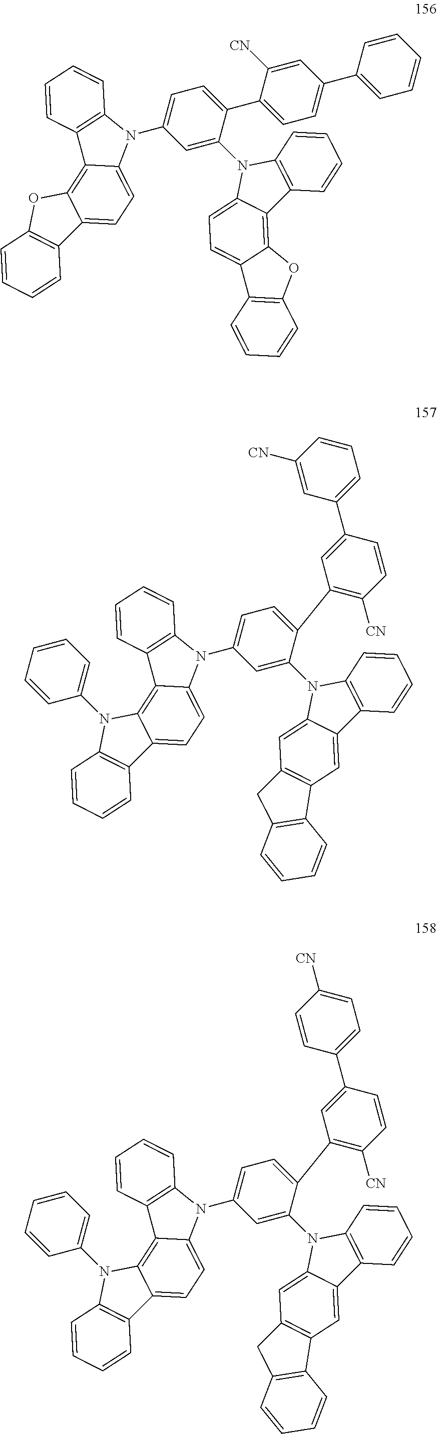

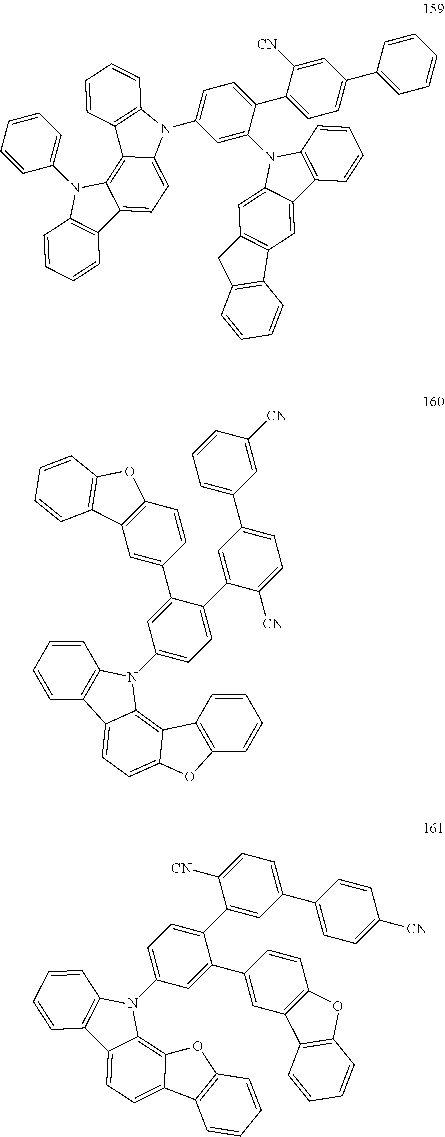

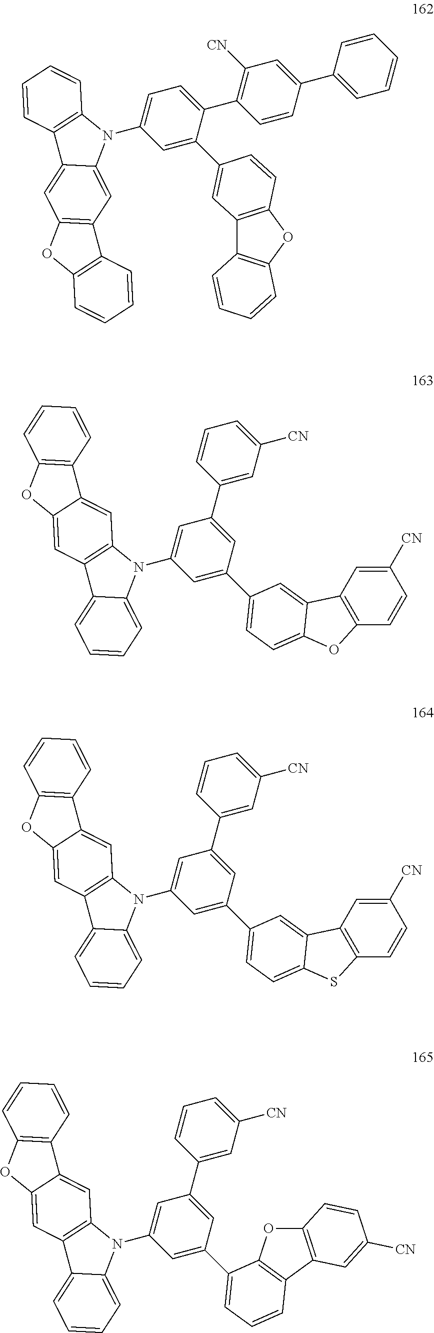

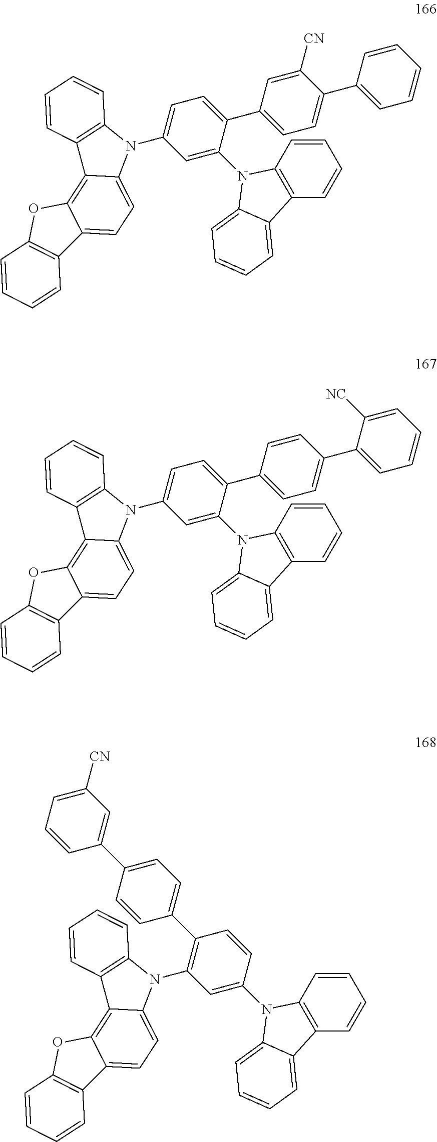 [SMILES: N#Cc1cc(-c2ccc(-n3c4ccccc4c4c5oc6ccccc6c5ccc43)cc2-n2c3ccccc3c3ccccc32)ccc1-c1ccccc1 ; N#Cc1ccccc1-c1ccc(-c2ccc(-n3c4ccccc4c4c5oc6ccccc6c5ccc43)cc2-n2c3ccccc3c3ccccc32)cc1 ; N#Cc1cccc(-c2ccc(-c3ccc(-n4c5ccccc5c5ccccc54)cc3-n3c4ccccc4c4c5oc6ccccc6c5ccc43)cc2)c1]

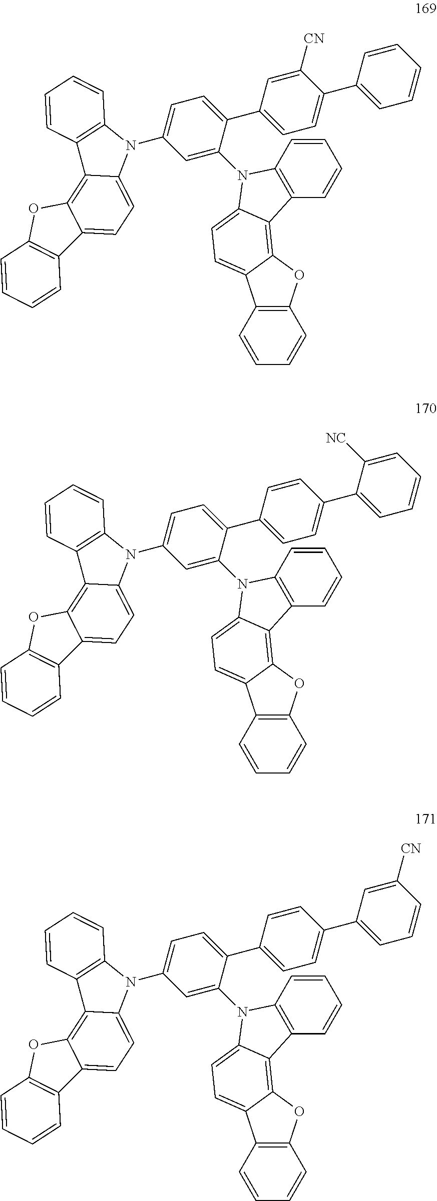 [SMILES: N#Cc1cc(-c2ccc(-n3c4ccccc4c4c5oc6ccccc6c5ccc43)cc2-n2c3ccccc3c3c4oc5ccccc5c4ccc32)ccc1-c1ccccc1 ; N#Cc1ccccc1-c1ccc(-c2ccc(-n3c4ccccc4c4c5oc6ccccc6c5ccc43)cc2-n2c3ccccc3c3c4oc5ccccc5c4ccc32)cc1 ; N#Cc1cccc(-c2ccc(-c3ccc(-n4c5ccccc5c5c6oc7ccccc7c6ccc54)cc3-n3c4ccccc4c4c5oc6ccccc6c5ccc43)cc2)c1]

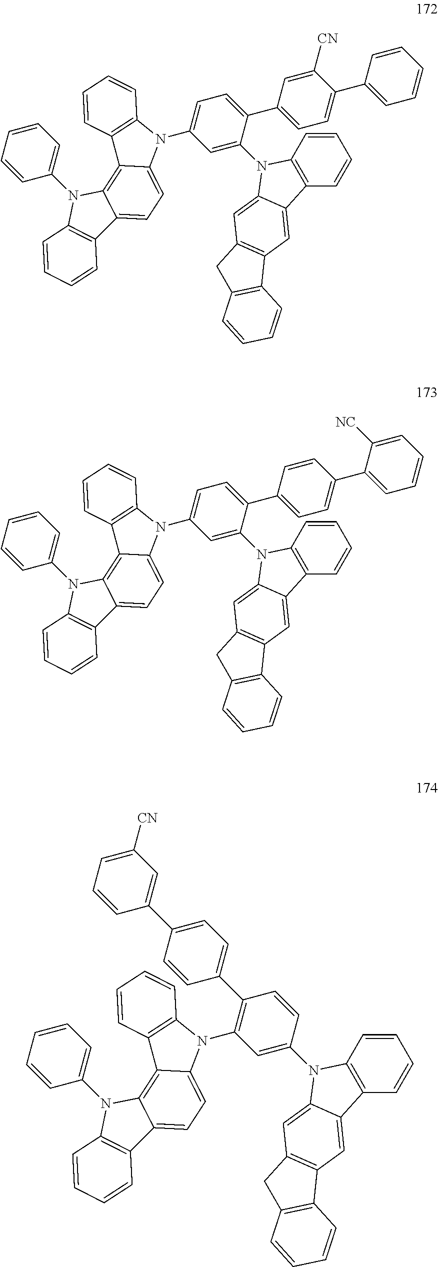 [SMILES: N#Cc1cc(-c2ccc(-n3c4ccccc4c4c3ccc3c5ccccc5n(-c5ccccc5)c34)cc2-n2c3ccccc3c3cc4c(cc32)Cc2ccccc2-4)ccc1-c1ccccc1 ; N#Cc1ccccc1-c1ccc(-c2ccc(-n3c4ccccc4c4c3ccc3c5ccccc5n(-c5ccccc5)c34)cc2-n2c3ccccc3c3cc4c(cc32)Cc2ccccc2-4)cc1 ; N#Cc1cccc(-c2ccc(-c3ccc(-n4c5ccccc5c5cc6c(cc54)Cc4ccccc4-6)cc3-n3c4ccccc4c4c3ccc3c5ccccc5n(-c5ccccc5)c34)cc2)c1]

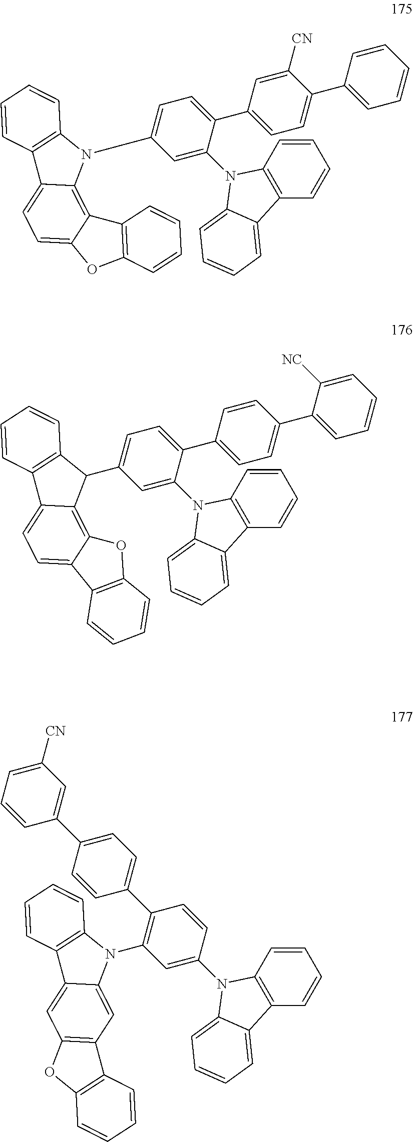 [SMILES: N#Cc1cc(-c2ccc(Cn3c4ccccc4c4ccc5oc6ccccc6c5c43)cc2-n2c3ccccc3c3ccccc32)ccc1-c1ccccc1 ; N#Cc1ccccc1-c1ccc(-c2ccc(C3c4ccccc4-c4ccc5c(oc6ccccc65)c43)cc2-n2c3ccccc3c3ccccc32)cc1 ; N#Cc1cccc(-c2ccc(-c3ccc(-n4c5ccccc5c5ccccc54)cc3-n3c4ccccc4c4cc5oc6ccccc6c5cc43)cc2)c1]

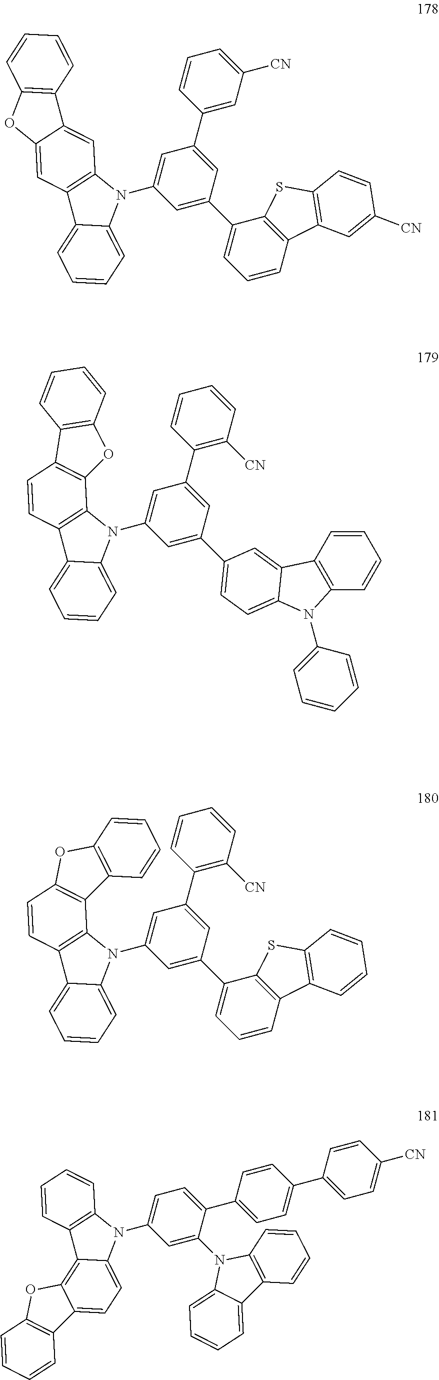 [SMILES: N#Cc1cccc(-c2cc(-c3cccc4c3sc3ccc(C#N)cc34)cc(-n3c4ccccc4c4cc5oc6ccccc6c5cc43)c2)c1 ; N#Cc1ccccc1-c1cc(-c2ccc3c(c2)c2ccccc2n3-c2ccccc2)cc(-n2c3ccccc3c3ccc4c5ccccc5oc4c32)c1 ; N#Cc1ccccc1-c1cc(-c2cccc3c2sc2ccccc23)cc(-n2c3ccccc3c3ccc4oc5ccccc5c4c32)c1 ; N#Cc1ccc(-c2ccc(-c3ccc(-n4c5ccccc5c5c6oc7ccccc7c6ccc54)cc3-n3c4ccccc4c4ccccc43)cc2)cc1]

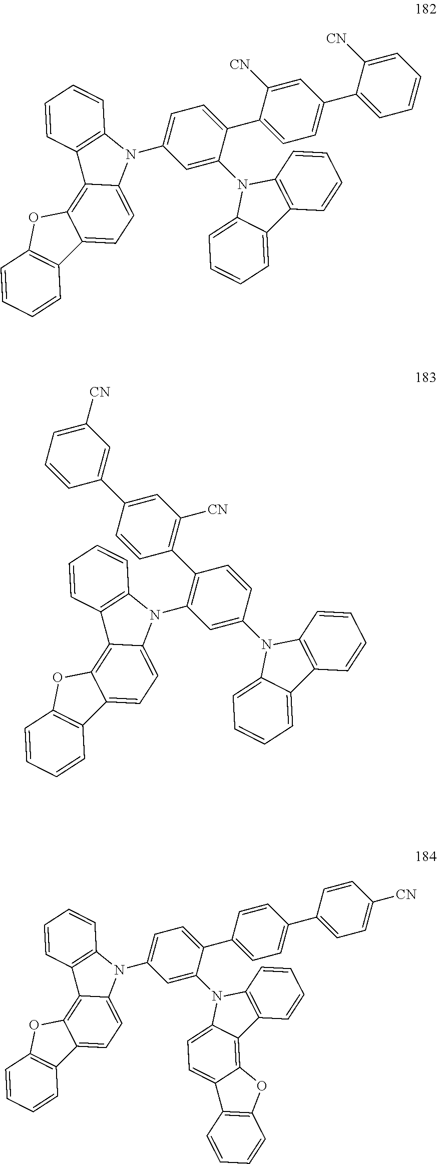

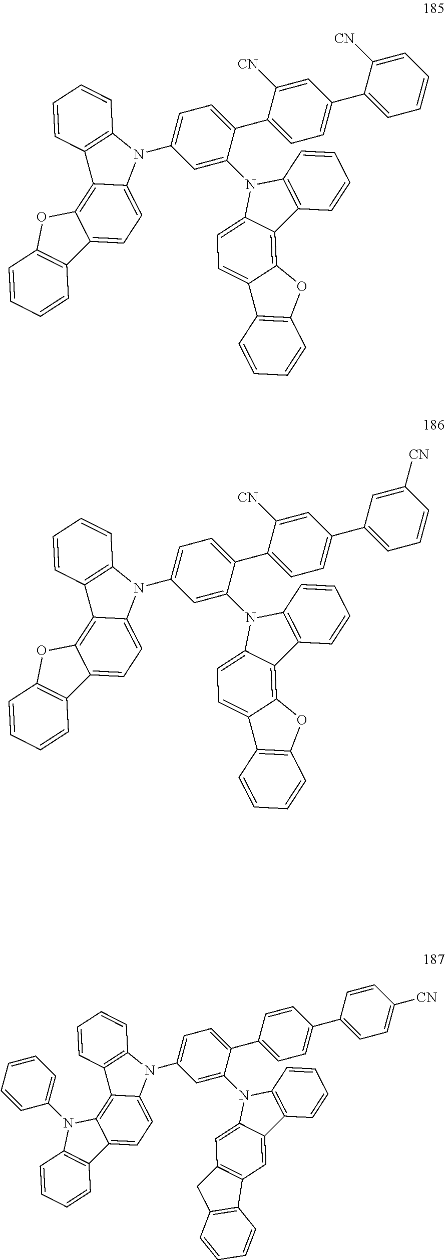

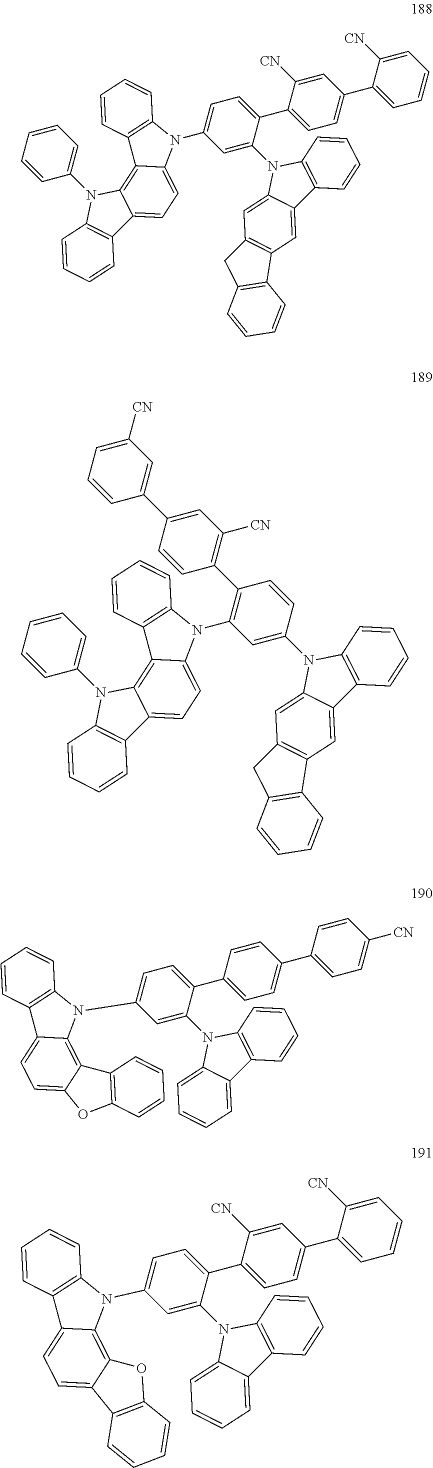

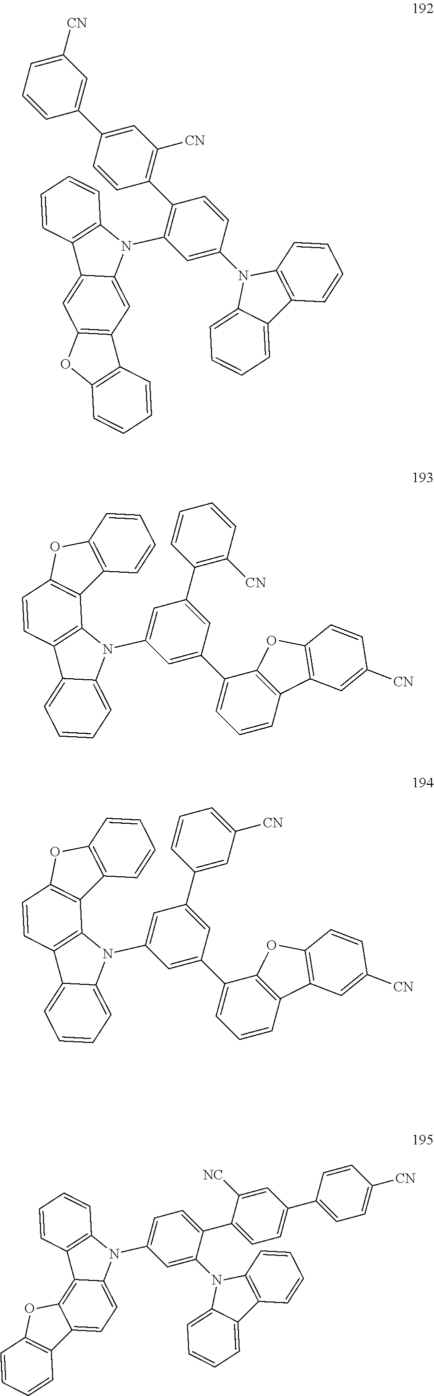 [SMILES: N#Cc1cccc(-c2ccc(-c3ccc(-n4c5ccccc5c5ccccc54)cc3-n3c4ccccc4c4cc5oc6ccccc6c5cc43)c(C#N)c2)c1 ; N#Cc1ccc2oc3c(-c4cc(-c5ccccc5C#N)cc(-n5c6ccccc6c6ccc7oc8ccccc8c7c65)c4)cccc3c2c1 ; N#Cc1cccc(-c2cc(-c3cccc4c3oc3ccc(C#N)cc34)cc(-n3c4ccccc4c4ccc5oc6ccccc6c5c43)c2)c1 ; N#Cc1ccc(-c2ccc(-c3ccc(-n4c5ccccc5c5c6oc7ccccc7c6ccc54)cc3-n3c4ccccc4c4ccccc43)c(C#N)c2)cc1]

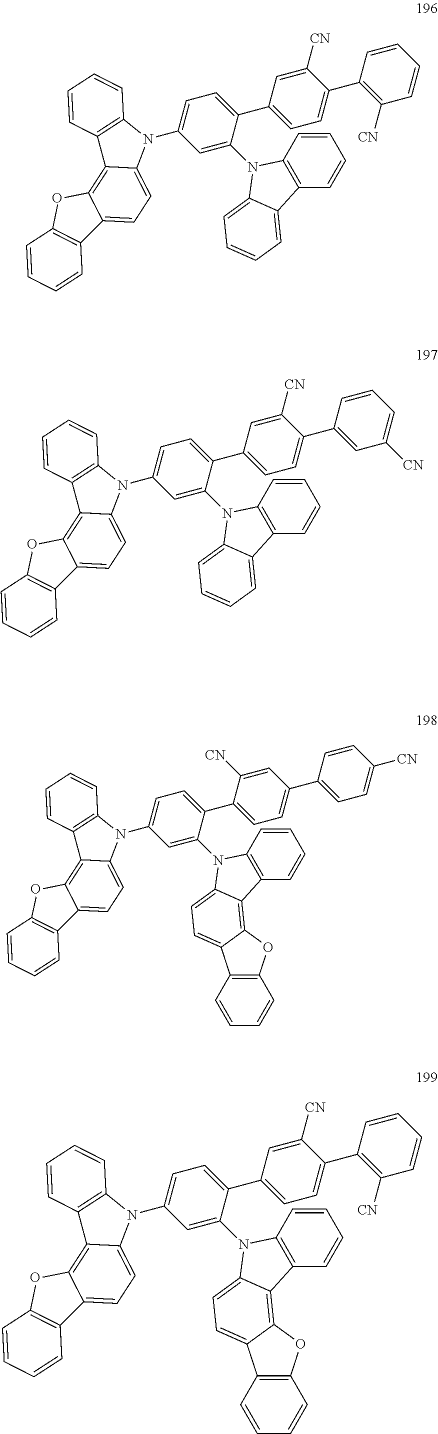

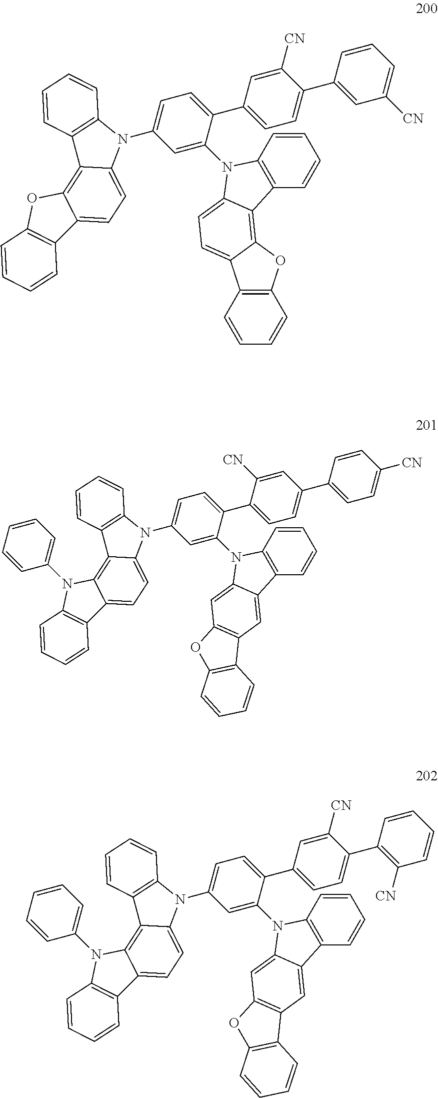

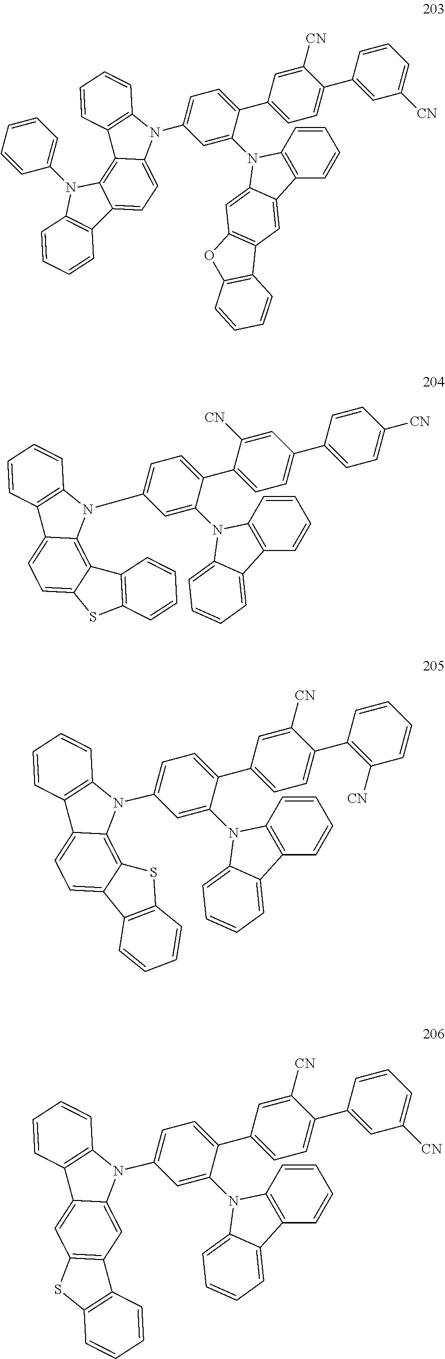

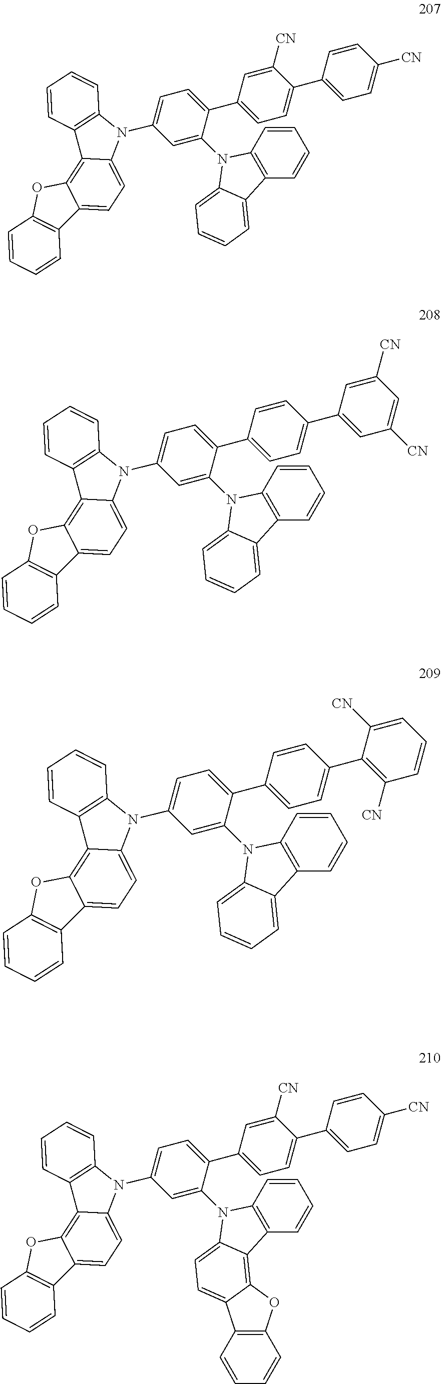

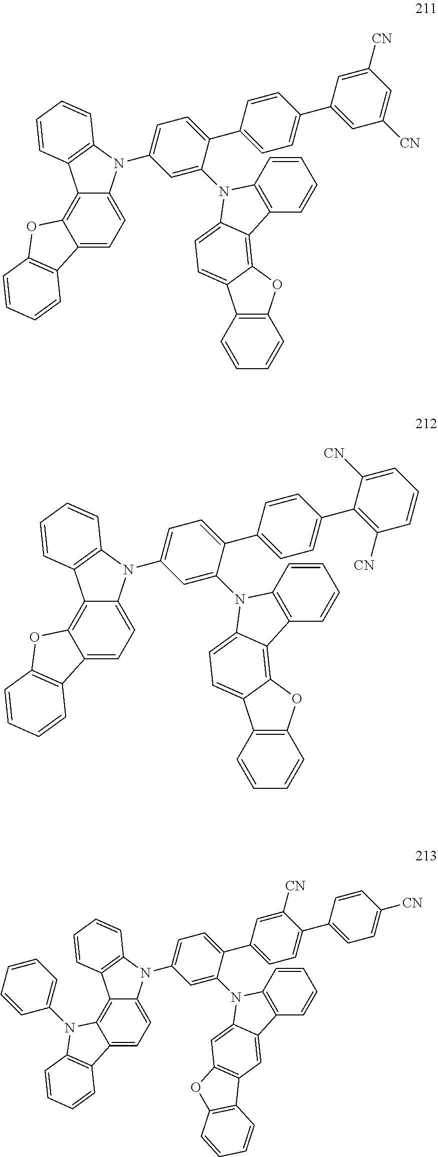

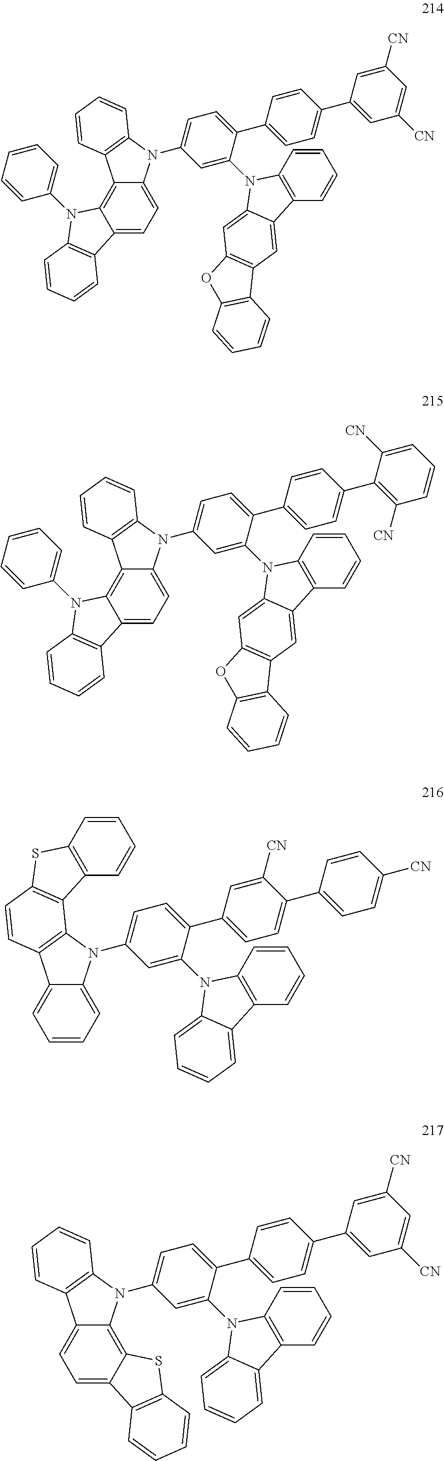

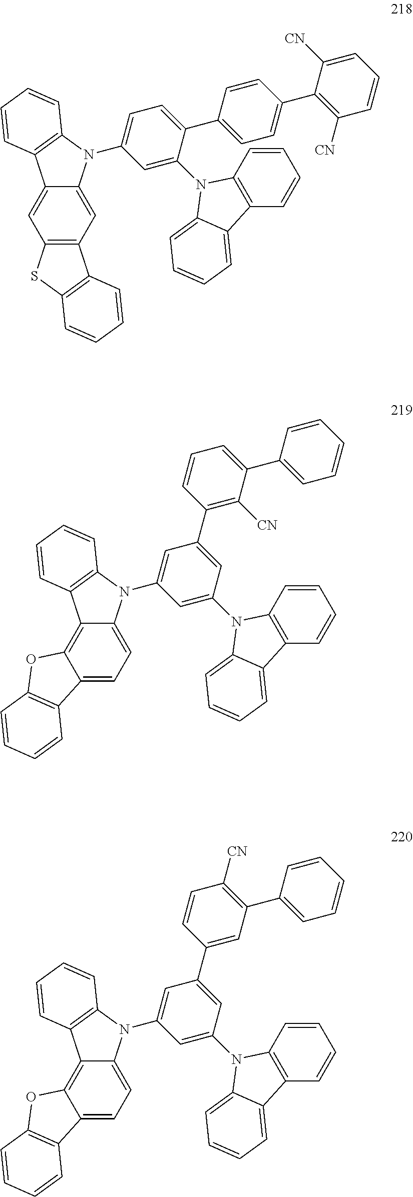

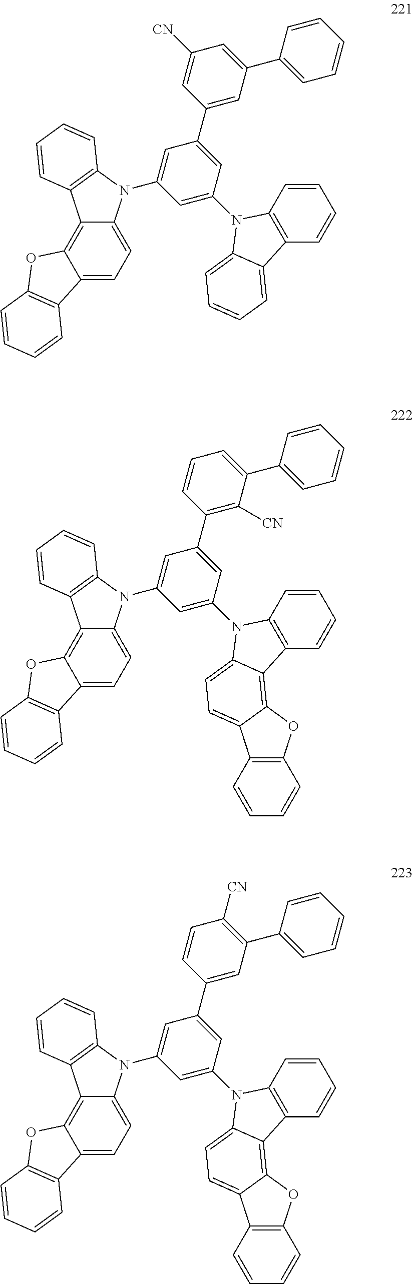

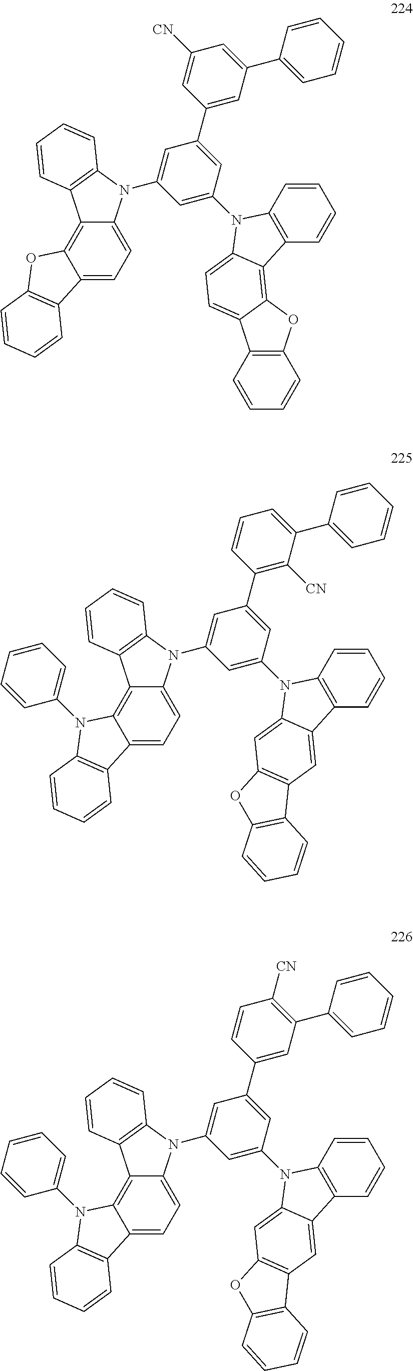

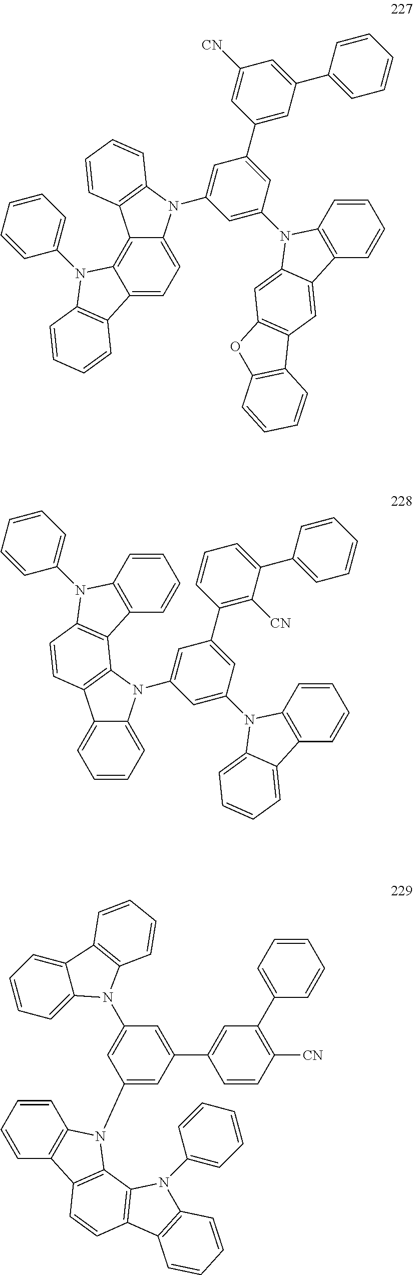

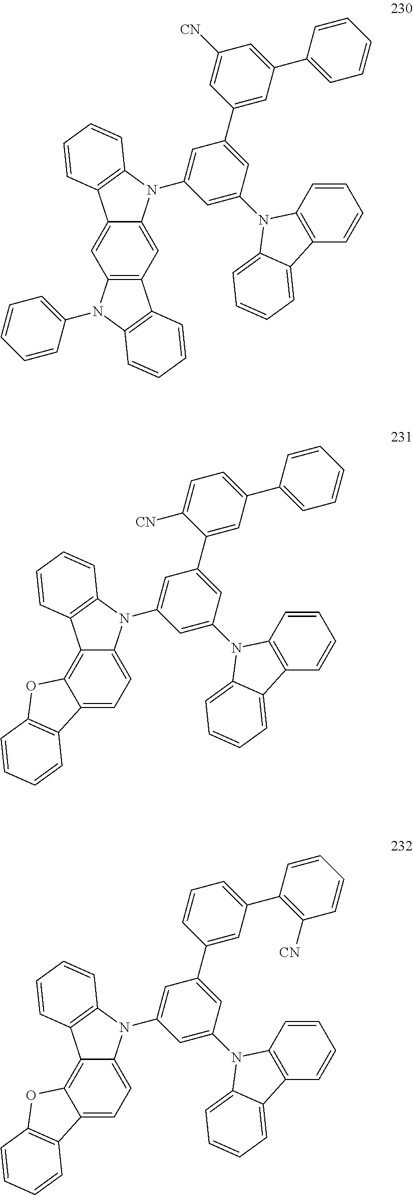

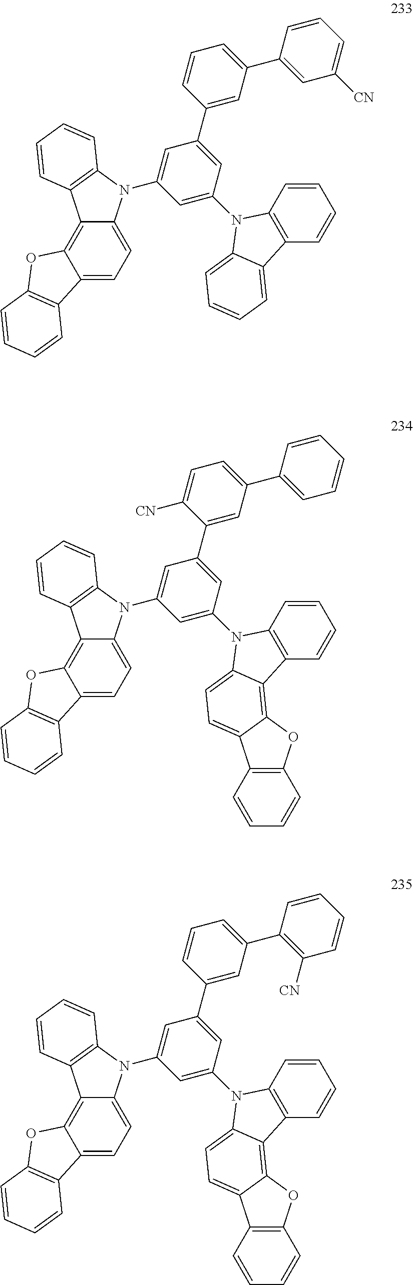

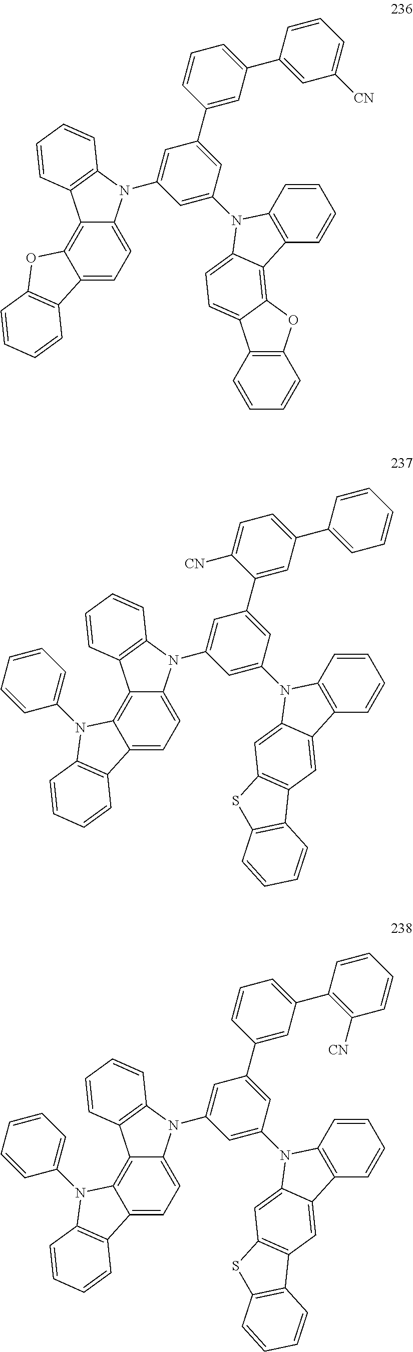

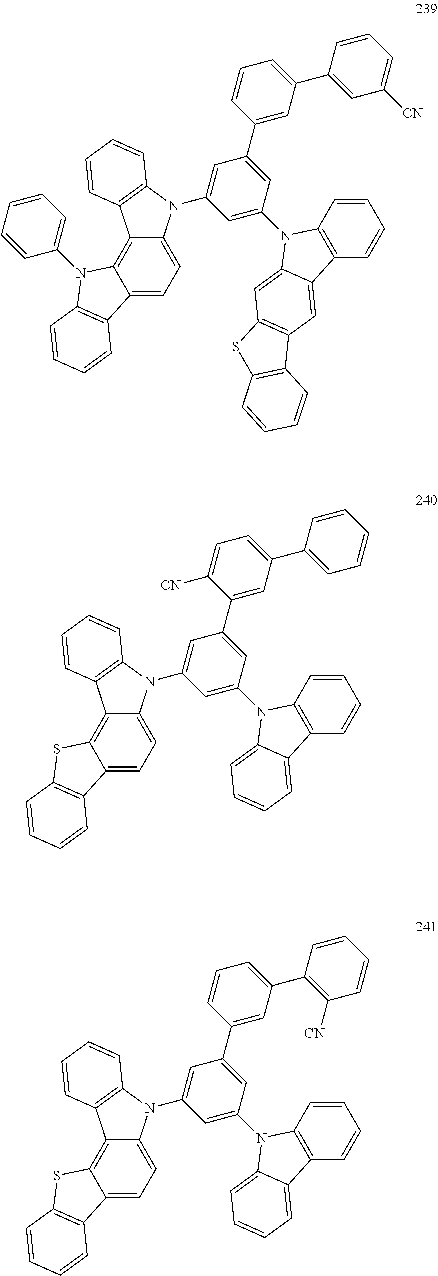

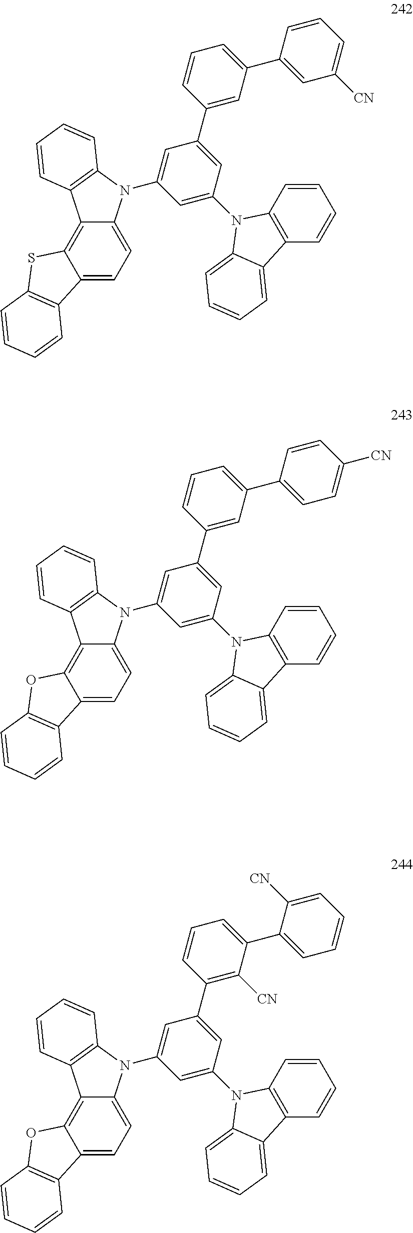

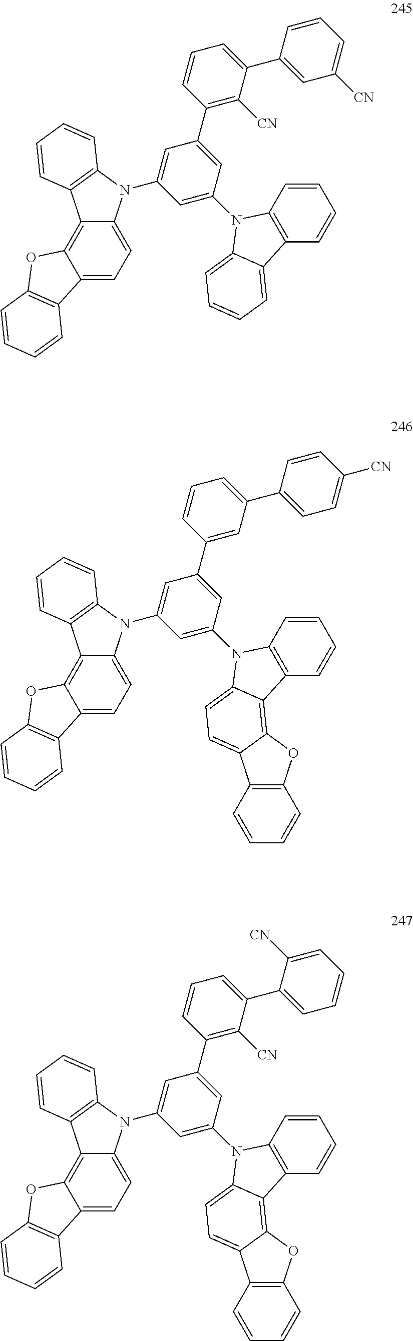

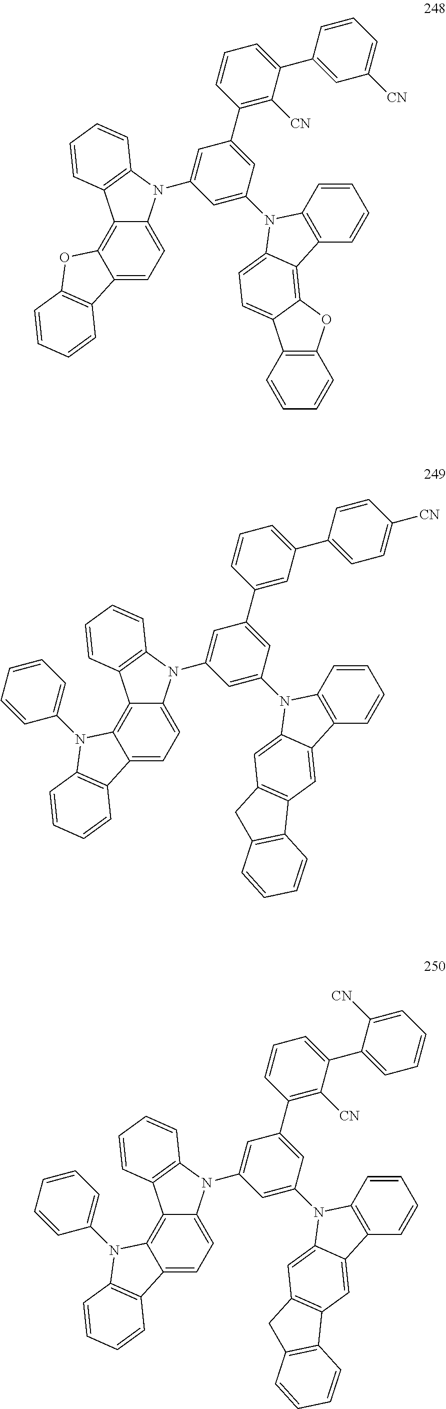

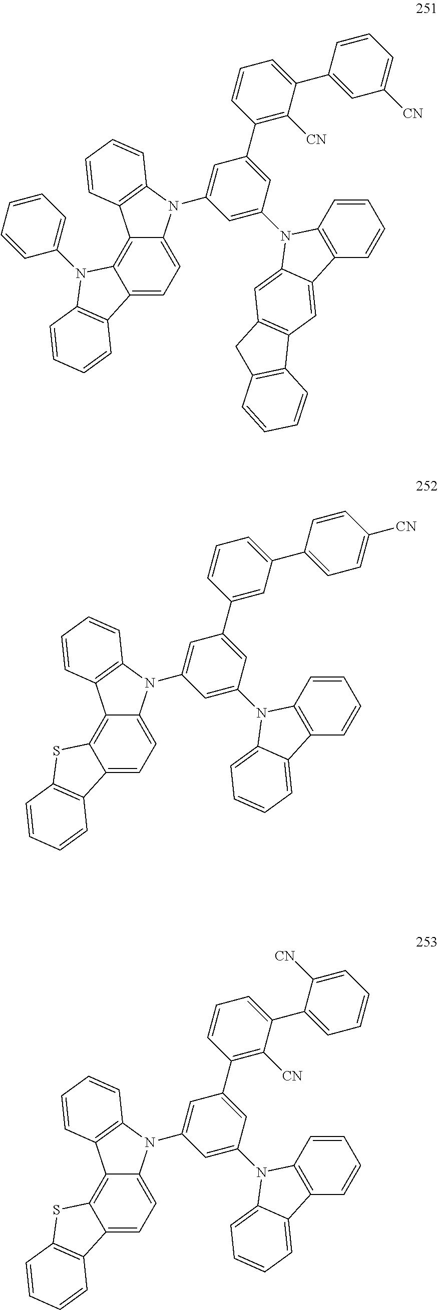

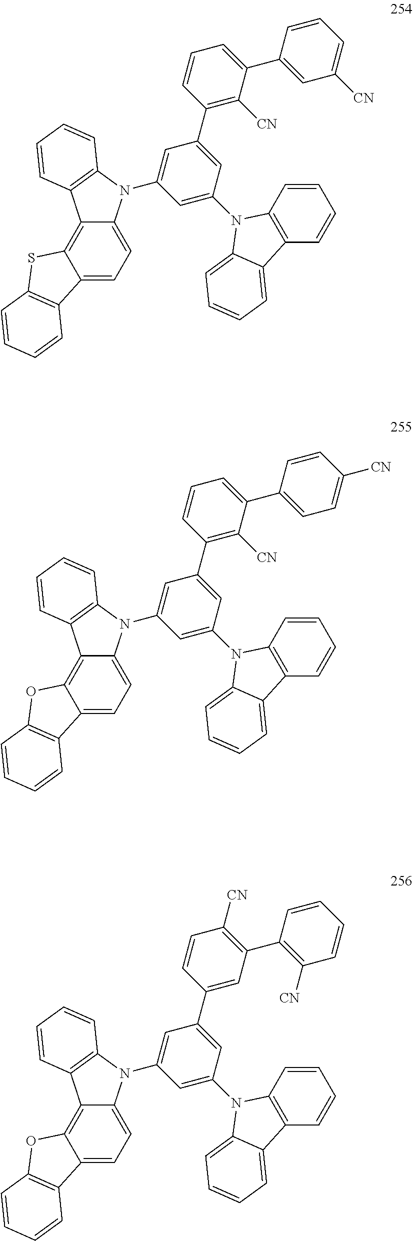

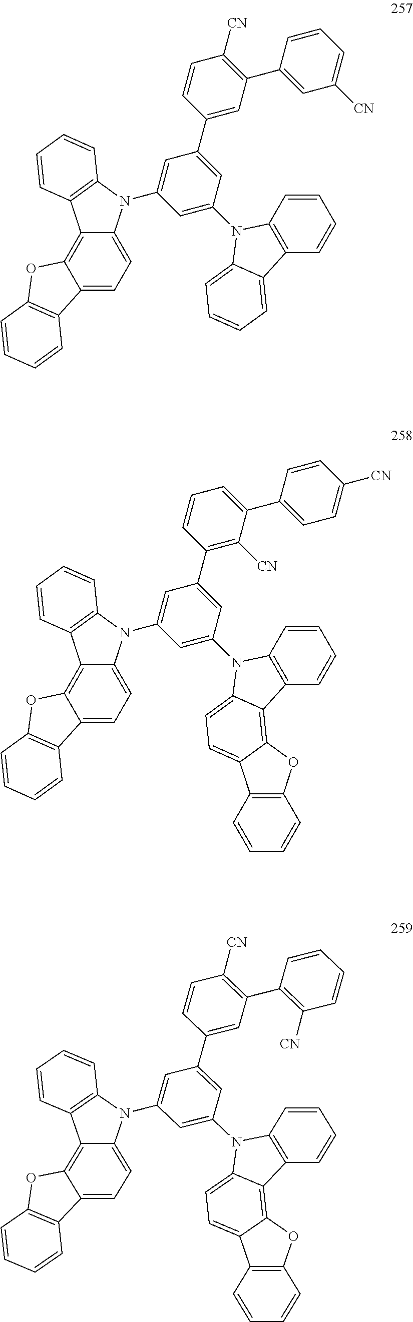

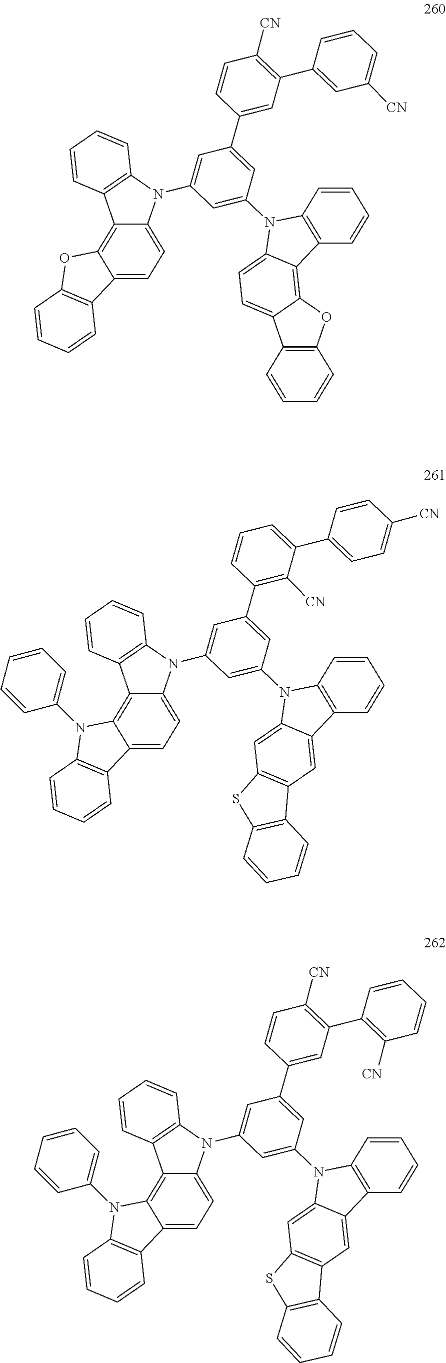

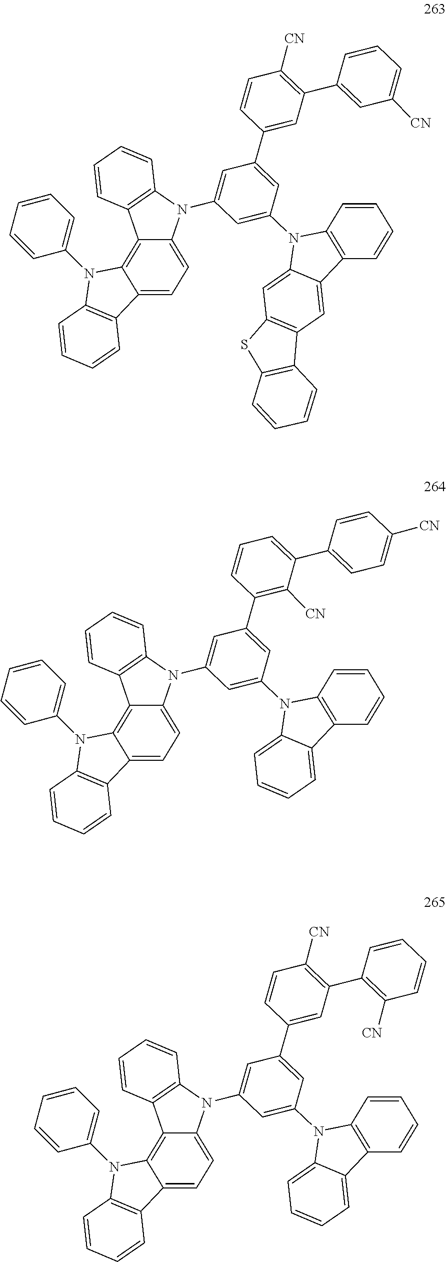

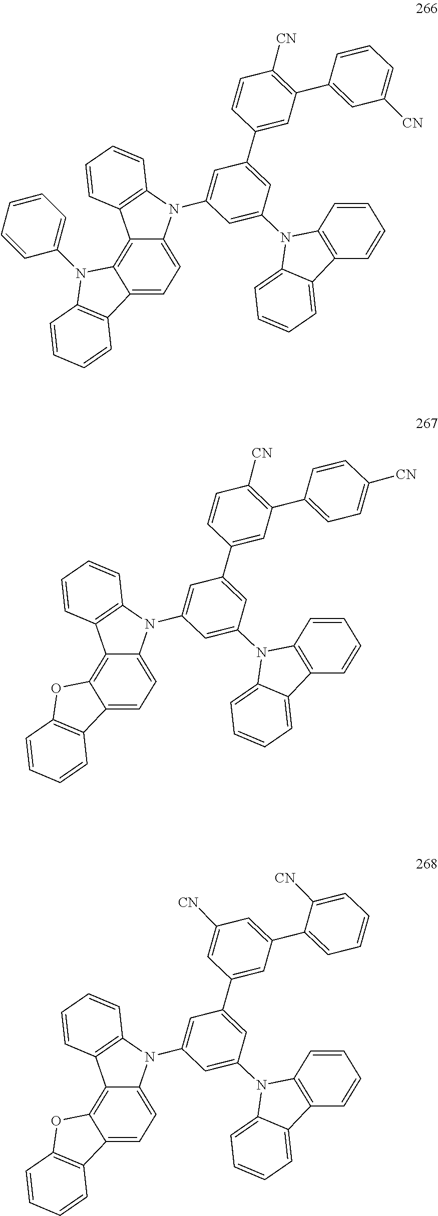

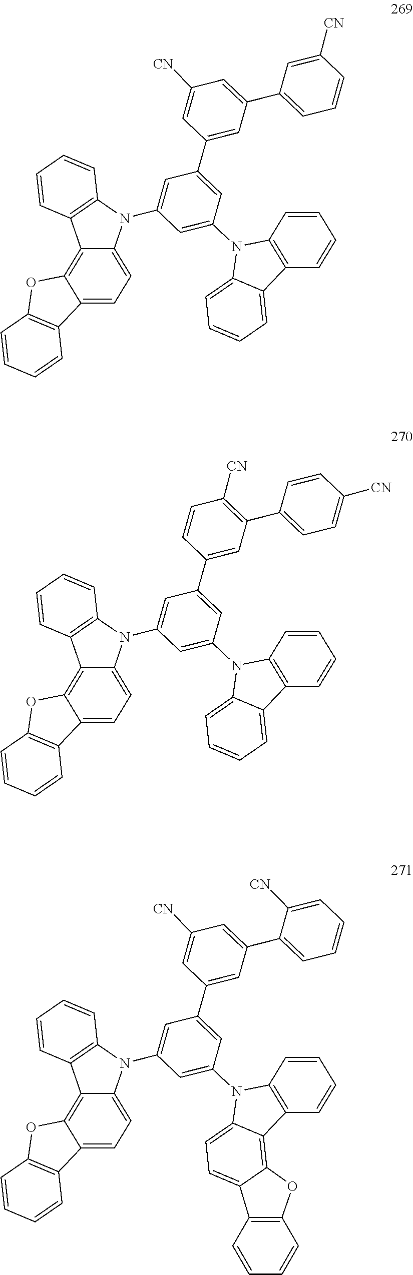

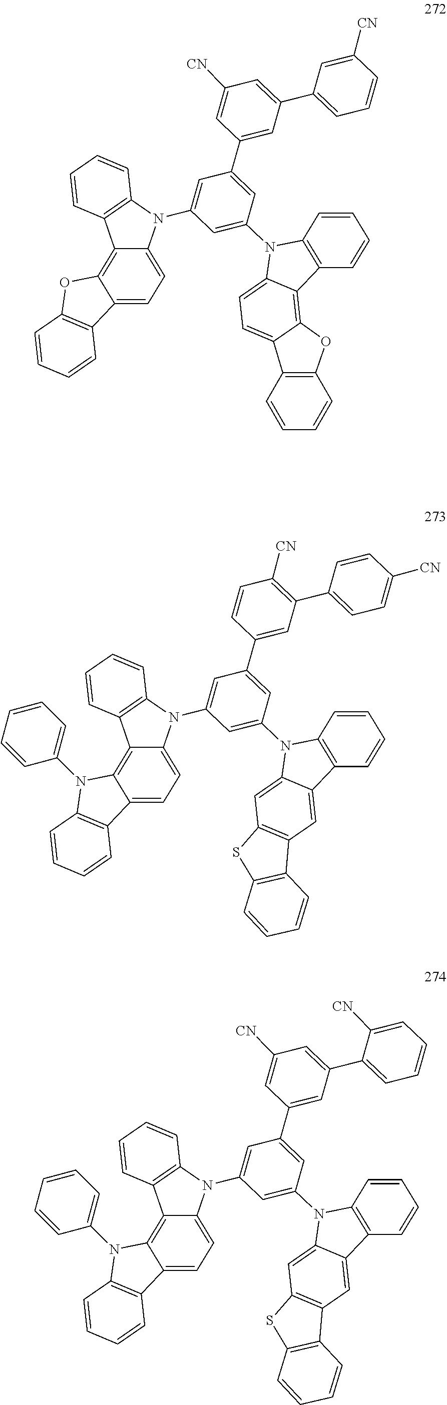

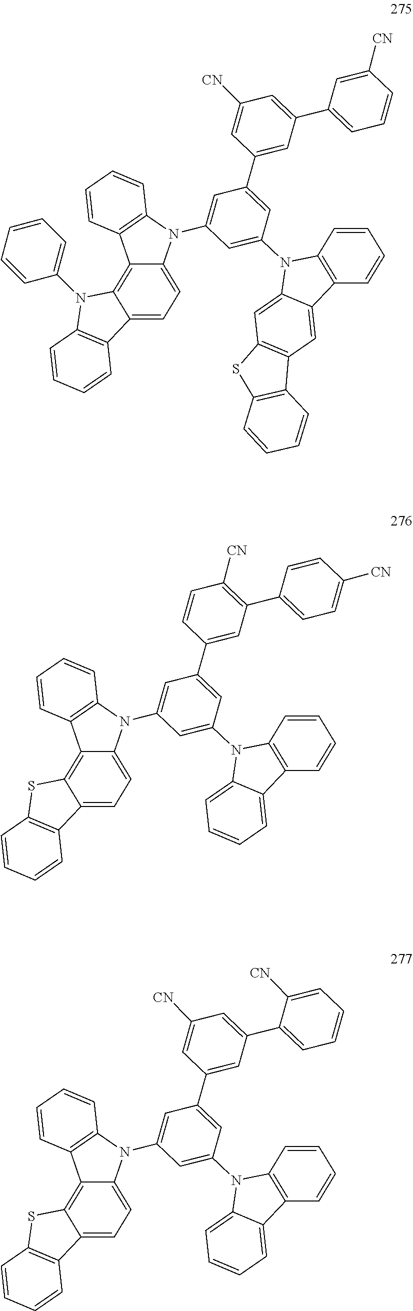

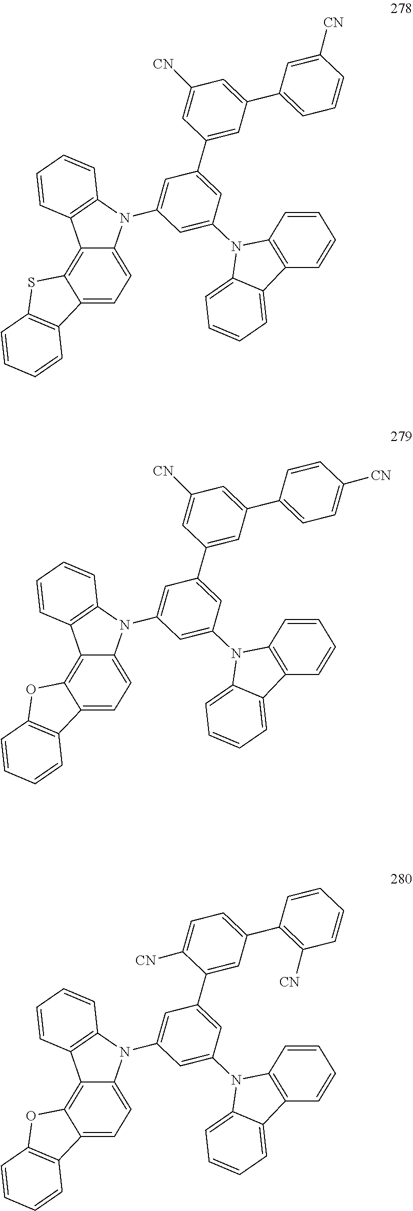

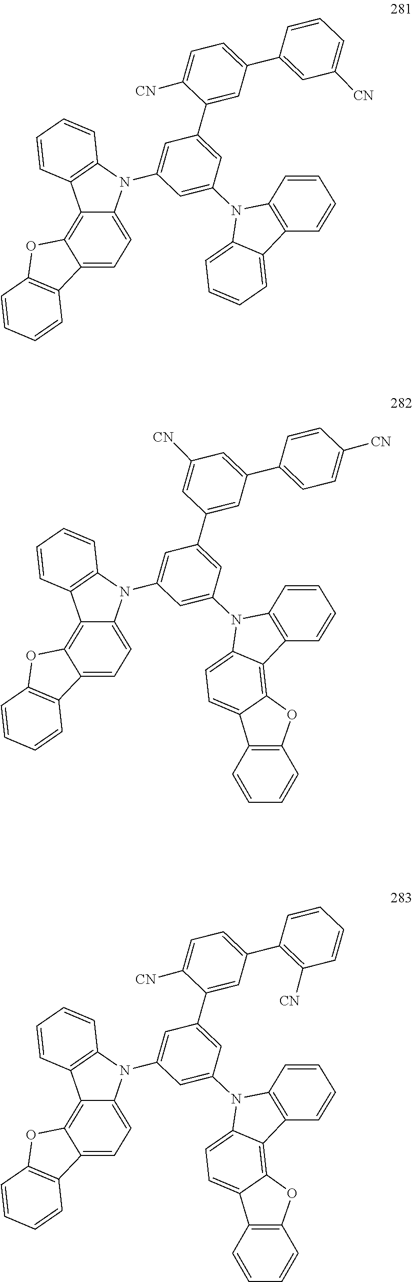

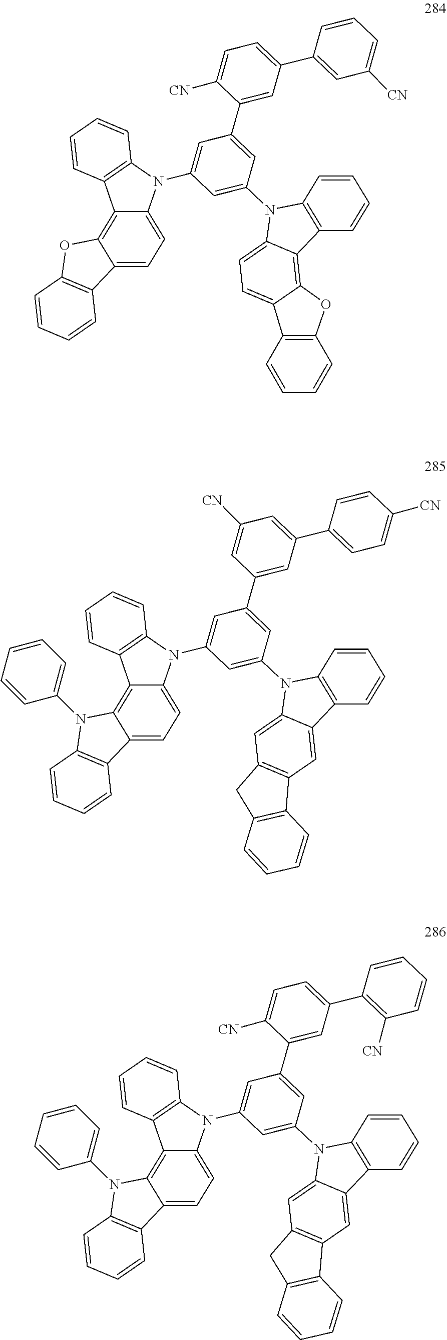

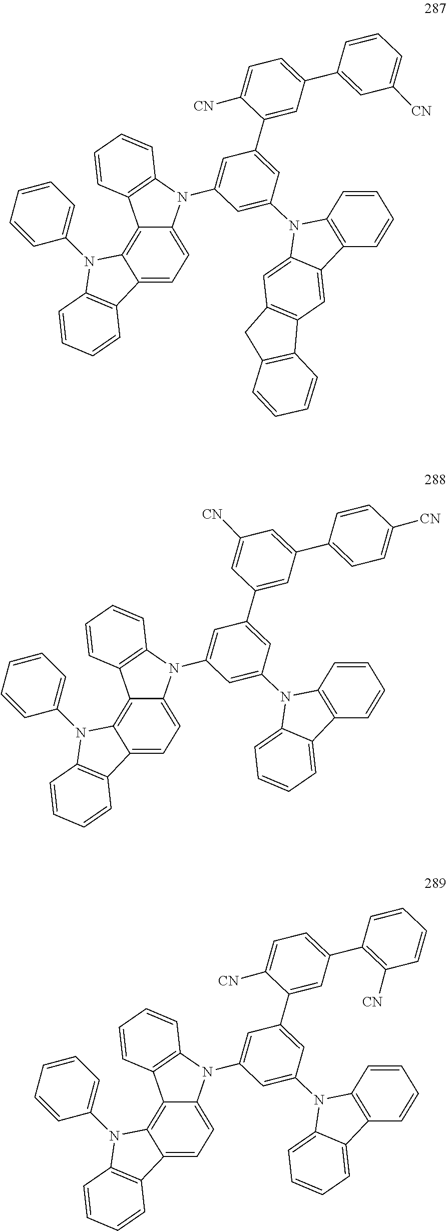

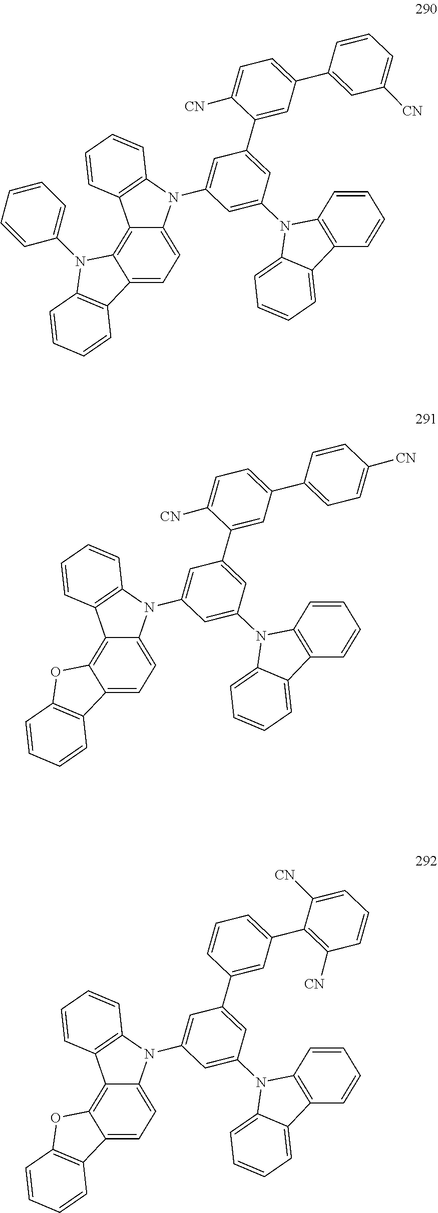

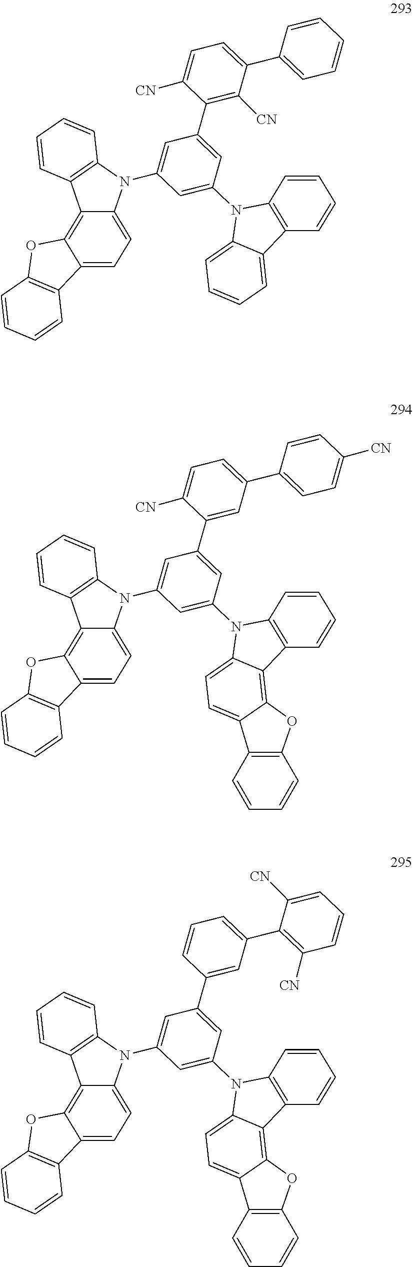

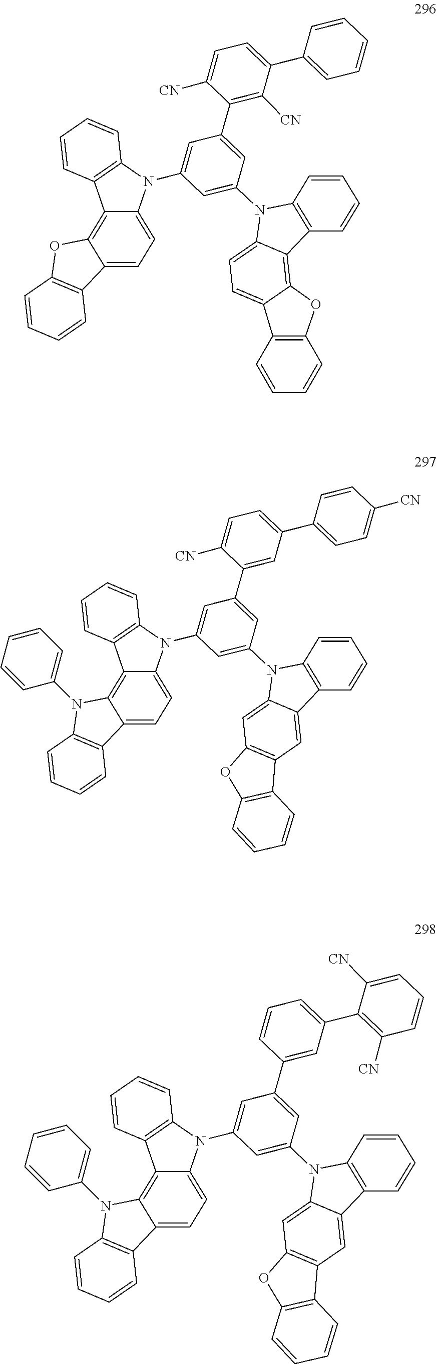

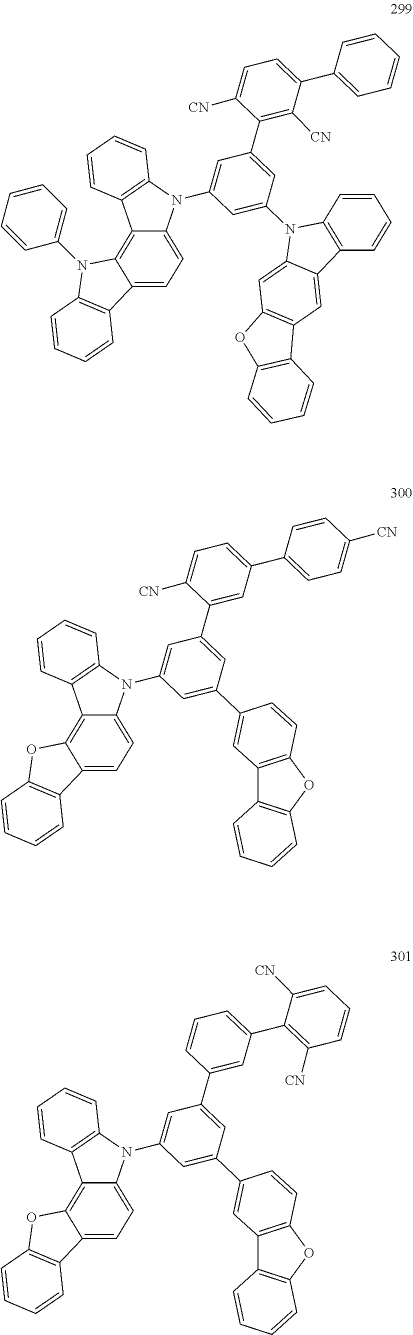

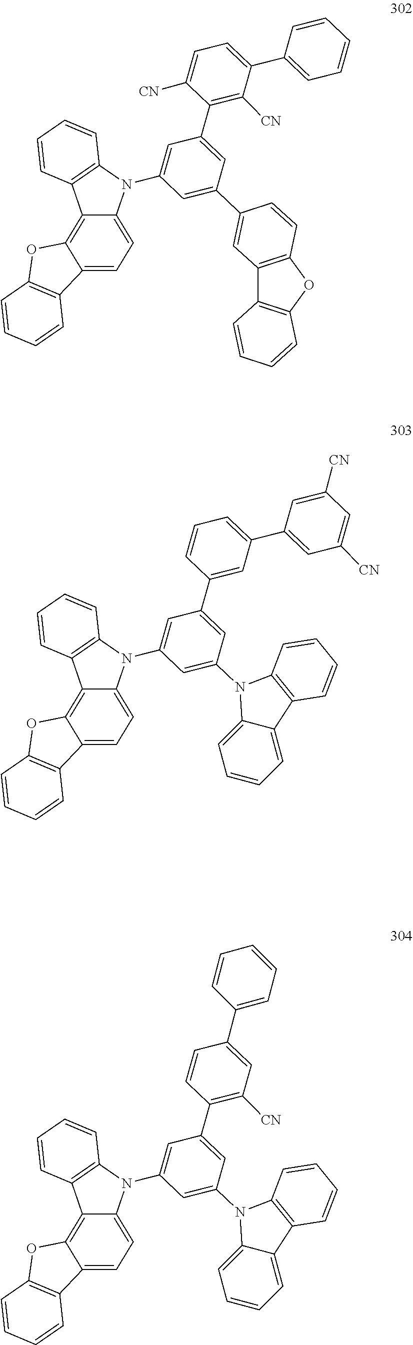

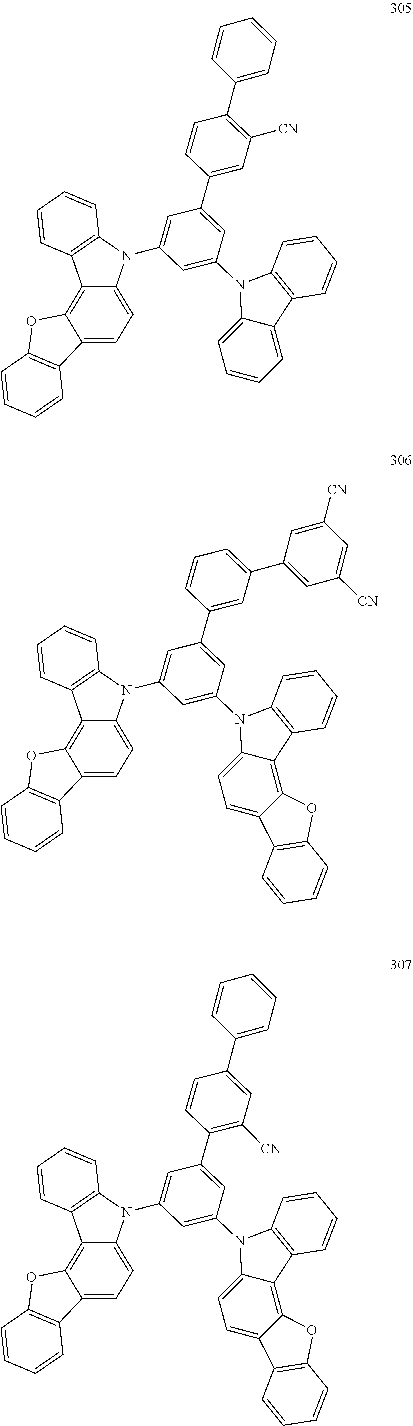 [SMILES: N#Cc1cc(-c2cc(-n3c4ccccc4c4ccccc43)cc(-n3c4ccccc4c4c5oc6ccccc6c5ccc43)c2)ccc1-c1ccccc1 ; N#Cc1cc(C#N)cc(-c2cccc(-c3cc(-n4c5ccccc5c5c6oc7ccccc7c6ccc54)cc(-n4c5ccccc5c5c6oc7ccccc7c6ccc54)c3)c2)c1 ; N#Cc1cc(-c2ccccc2)ccc1-c1cc(-n2c3ccccc3c3c4oc5ccccc5c4ccc32)cc(-n2c3ccccc3c3c4oc5ccccc5c4ccc32)c1]

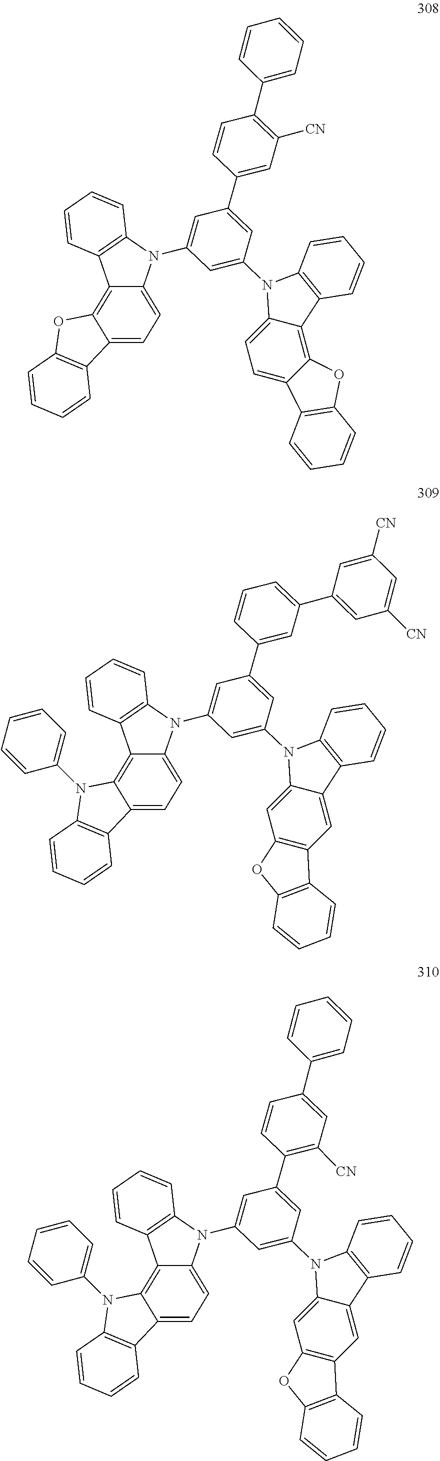 [SMILES: N#Cc1cc(-c2cc(-n3c4ccccc4c4c5oc6ccccc6c5ccc43)cc(-n3c4ccccc4c4c5oc6ccccc6c5ccc43)c2)ccc1-c1ccccc1 ; N#Cc1cc(C#N)cc(-c2cccc(-c3cc(-n4c5ccccc5c5cc6c(cc54)oc4ccccc46)cc(-n4c5ccccc5c5c4ccc4c6ccccc6n(-c6ccccc6)c45)c3)c2)c1 ; N#Cc1cc(-c2ccccc2)ccc1-c1cc(-n2c3ccccc3c3cc4c(cc32)oc2ccccc24)cc(-n2c3ccccc3c3c2ccc2c4ccccc4n(-c4ccccc4)c23)c1]

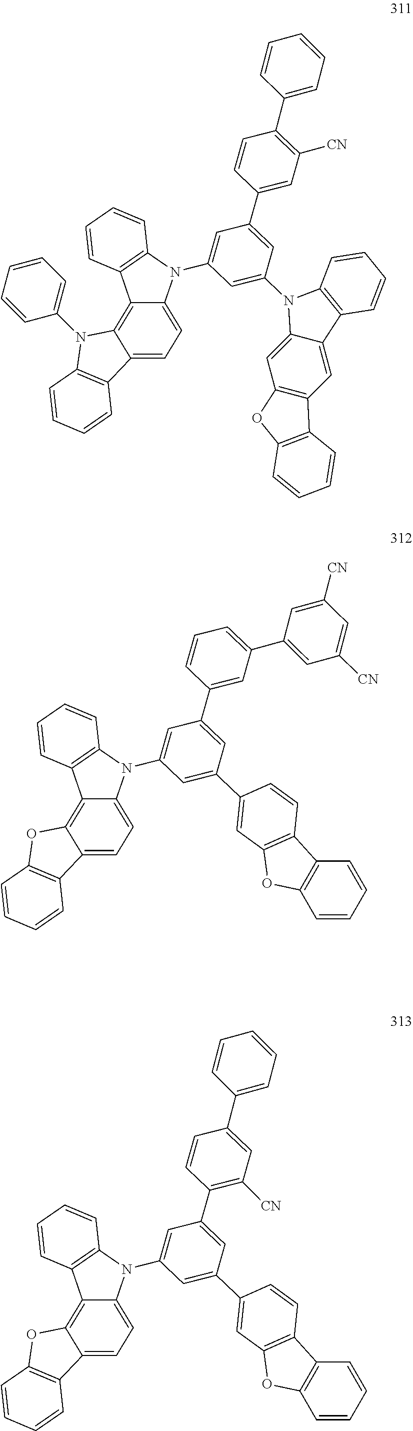 [SMILES: N#Cc1cc(-c2cc(-n3c4ccccc4c4cc5c(cc43)oc3ccccc35)cc(-n3c4ccccc4c4c3ccc3c5ccccc5n(-c5ccccc5)c34)c2)ccc1-c1ccccc1 ; N#Cc1cc(C#N)cc(-c2cccc(-c3cc(-c4ccc5c(c4)oc4ccccc45)cc(-n4c5ccccc5c5c6oc7ccccc7c6ccc54)c3)c2)c1 ; N#Cc1cc(-c2ccccc2)ccc1-c1cc(-c2ccc3c(c2)oc2ccccc23)cc(-n2c3ccccc3c3c4oc5ccccc5c4ccc32)c1]

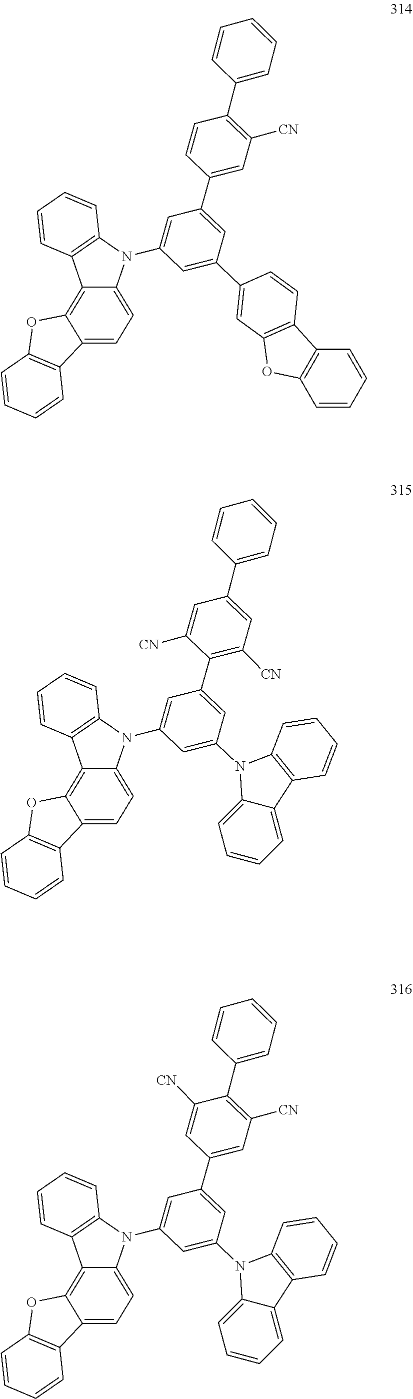

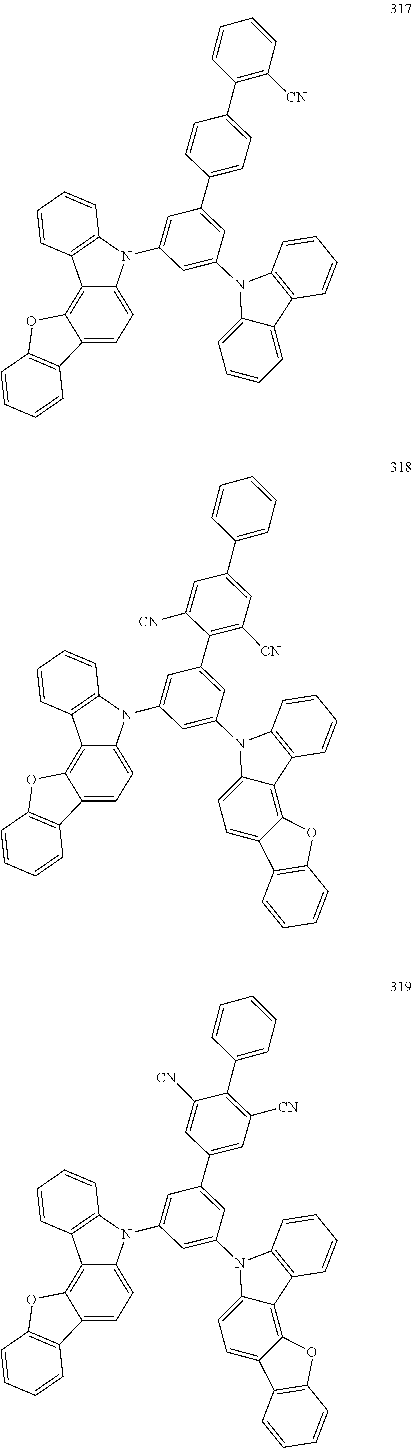

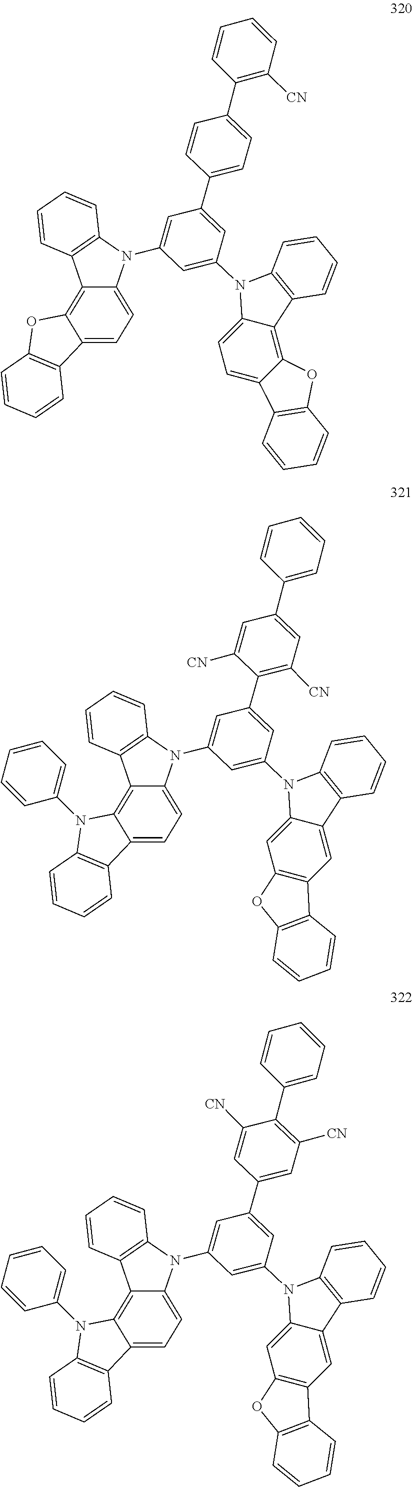

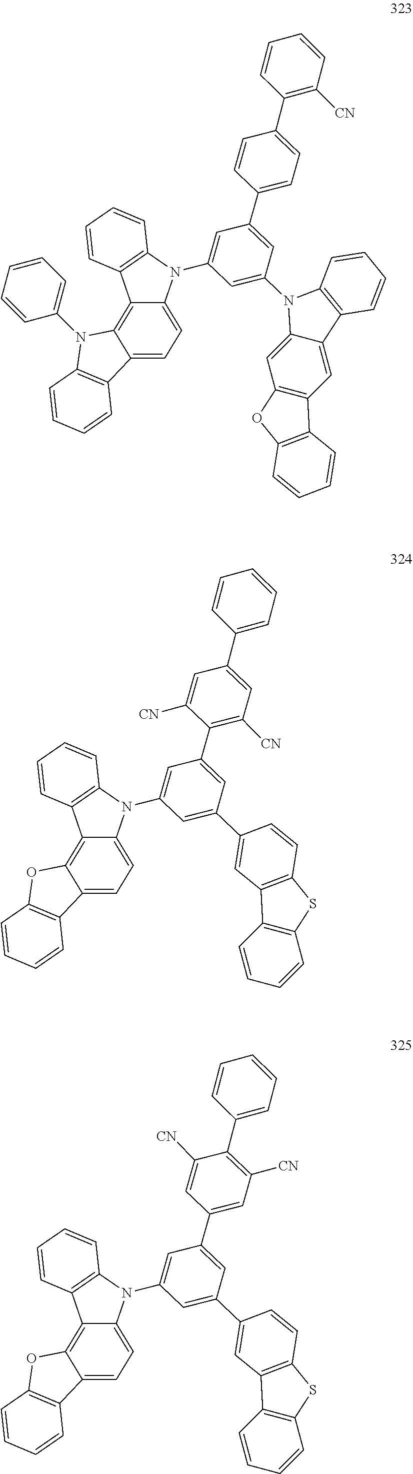

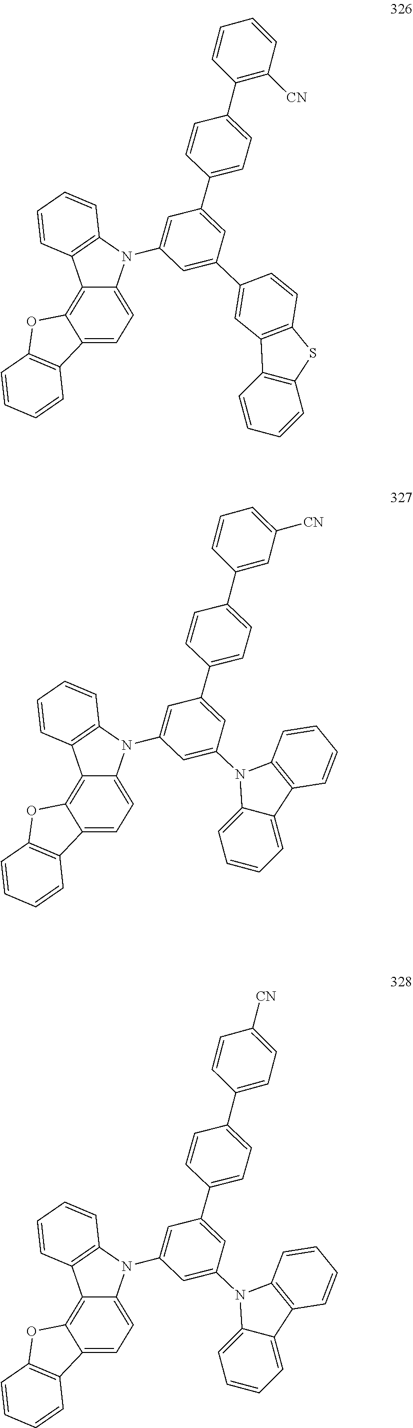 [SMILES: N#Cc1ccccc1-c1ccc(-c2cc(-c3ccc4sc5ccccc5c4c3)cc(-n3c4ccccc4c4c5oc6ccccc6c5ccc43)c2)cc1 ; N#Cc1cccc(-c2ccc(-c3cc(-n4c5ccccc5c5ccccc54)cc(-n4c5ccccc5c5c6oc7ccccc7c6ccc54)c3)cc2)c1 ; N#Cc1ccc(-c2ccc(-c3cc(-n4c5ccccc5c5ccccc54)cc(-n4c5ccccc5c5c6oc7ccccc7c6ccc54)c3)cc2)cc1]

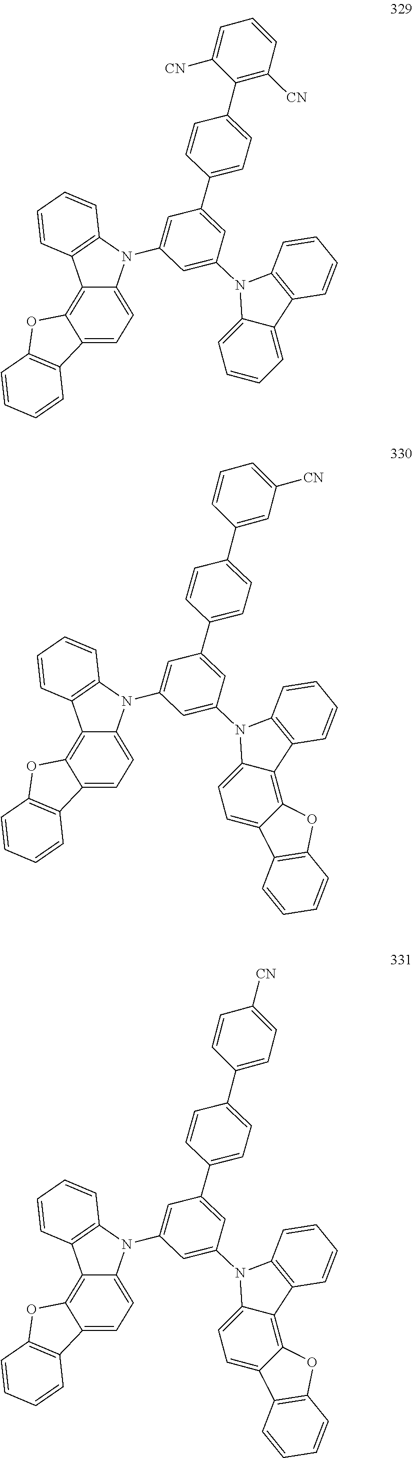

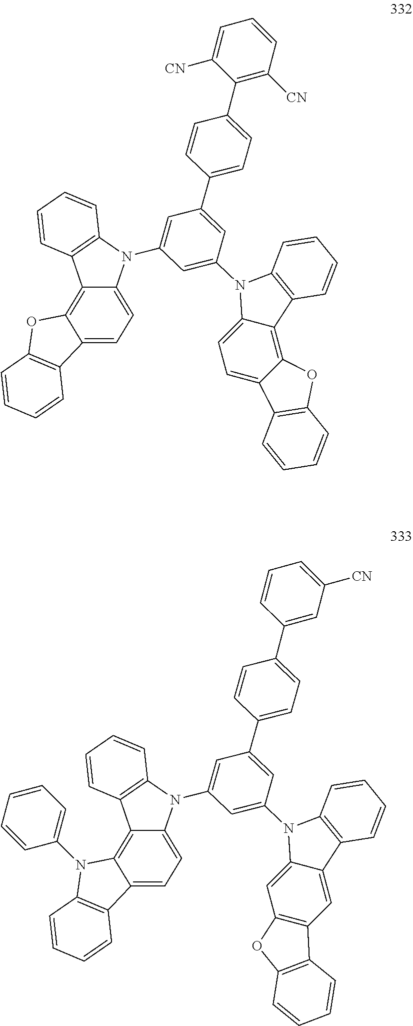

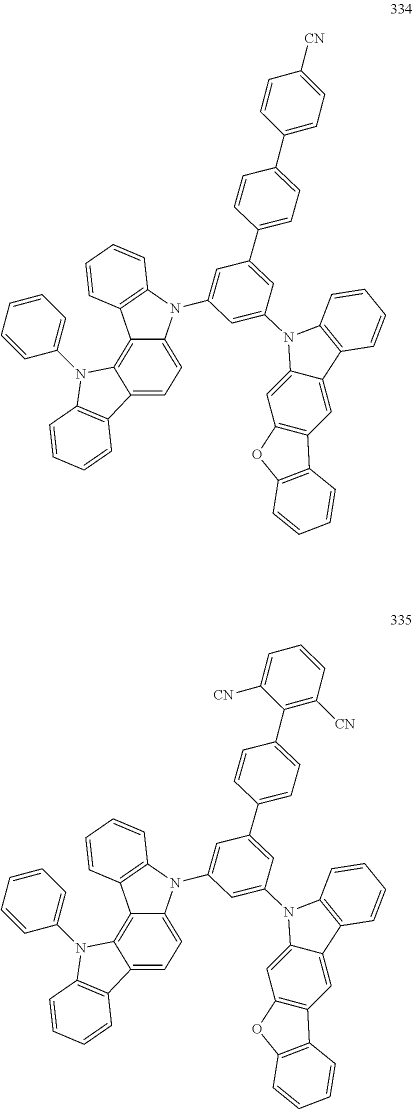

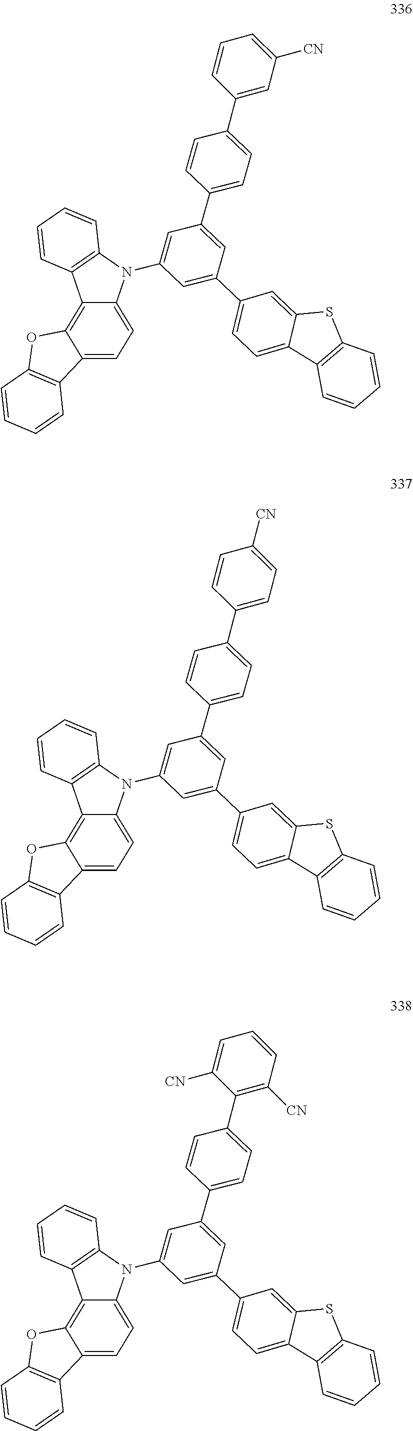

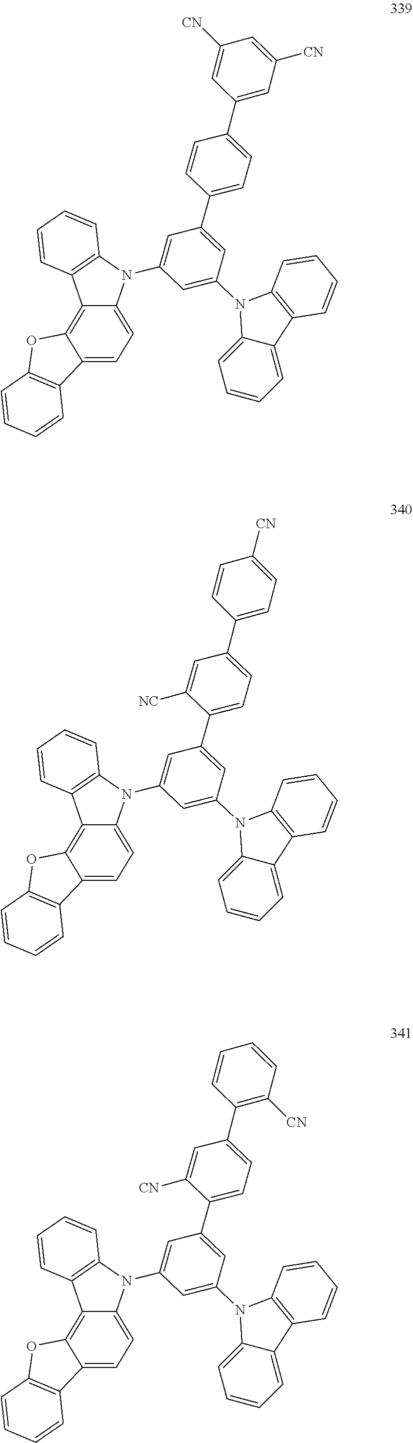

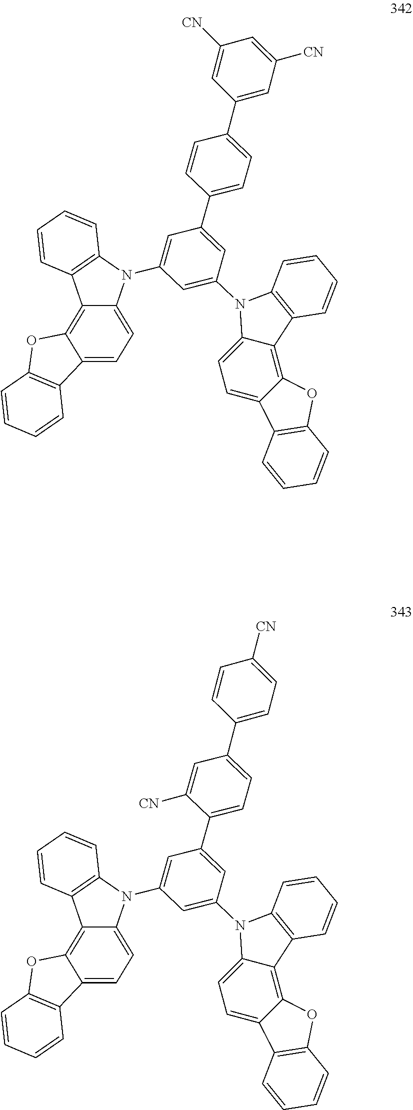

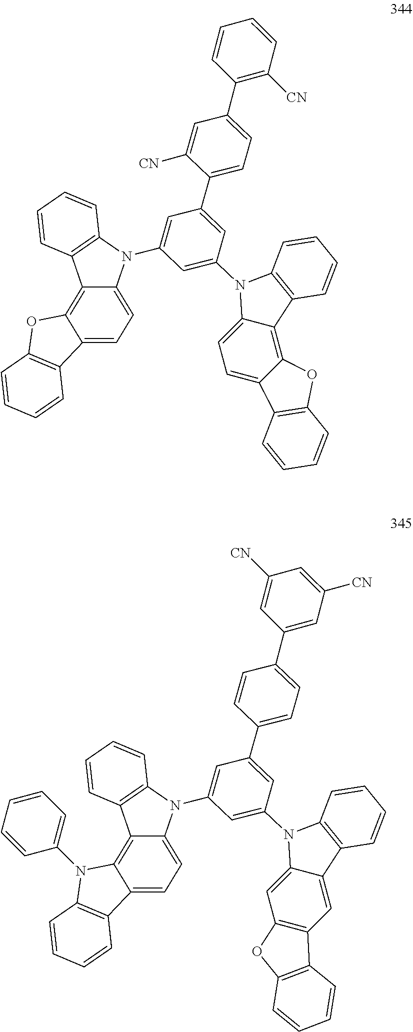

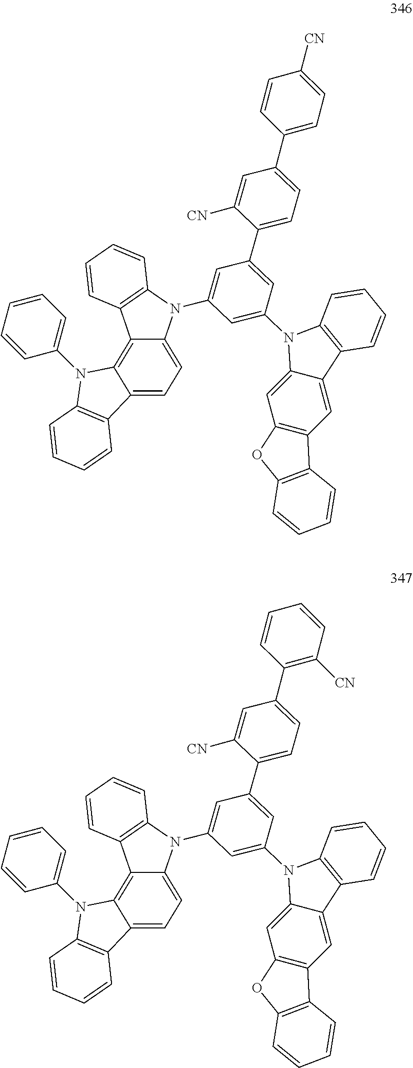

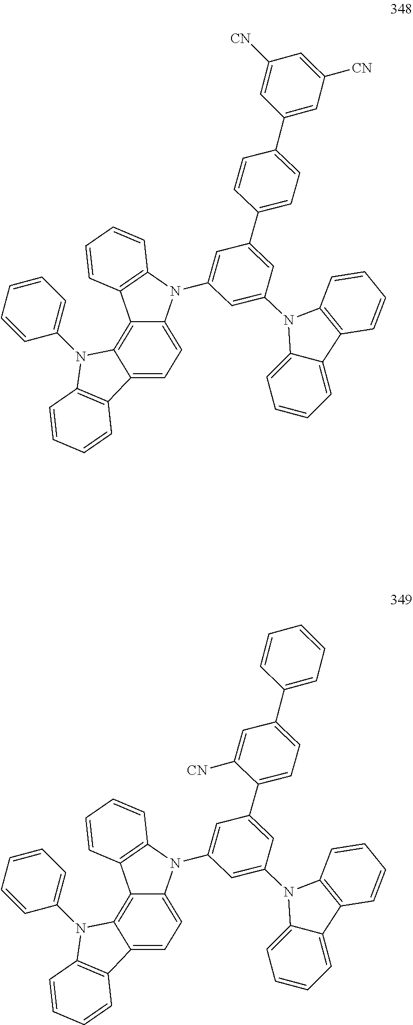

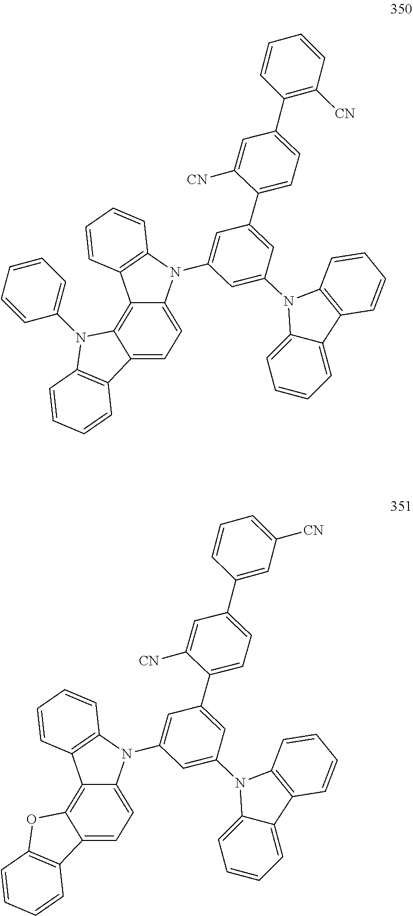

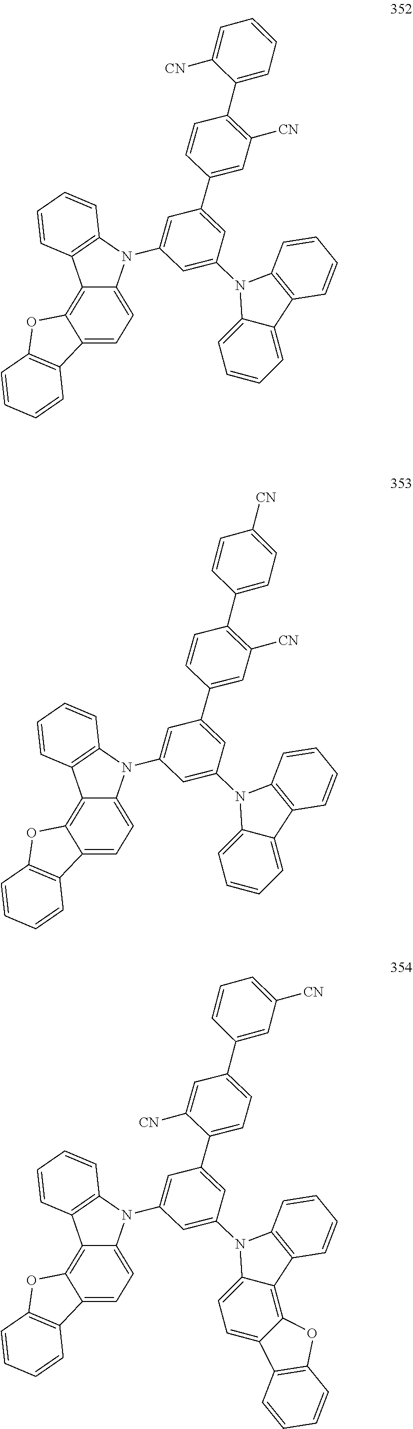

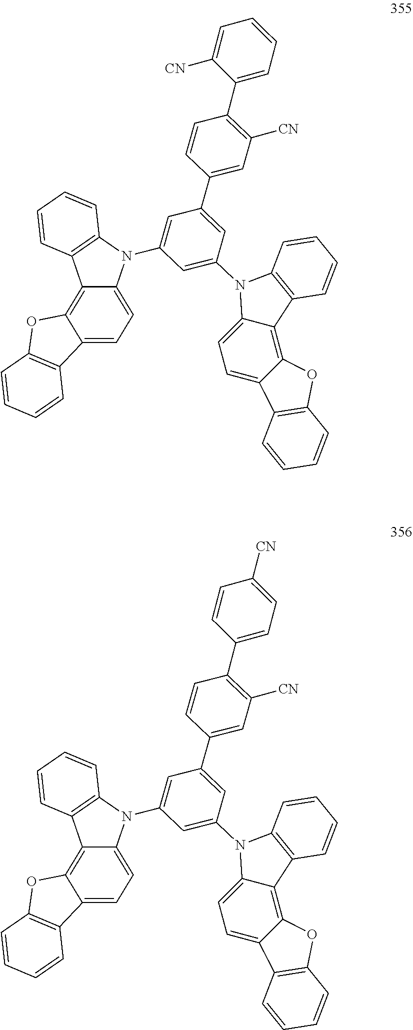

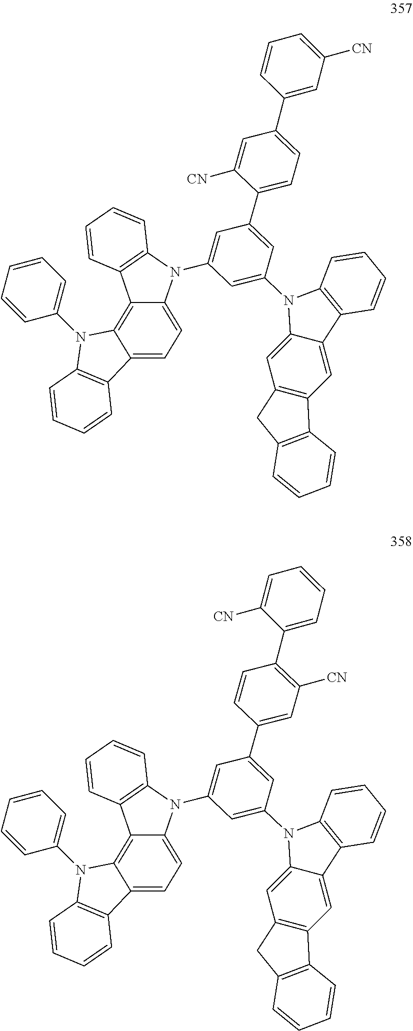

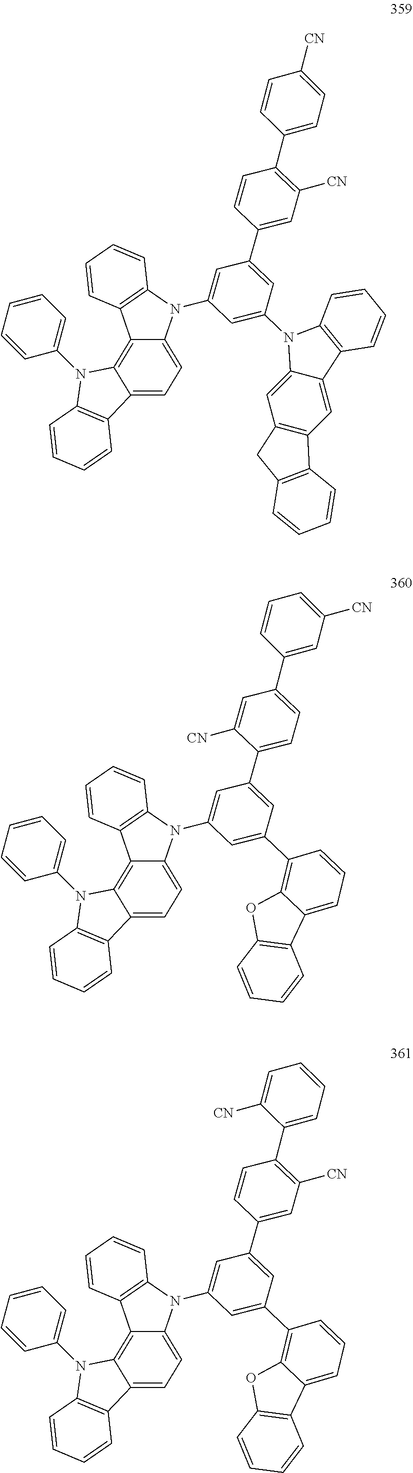

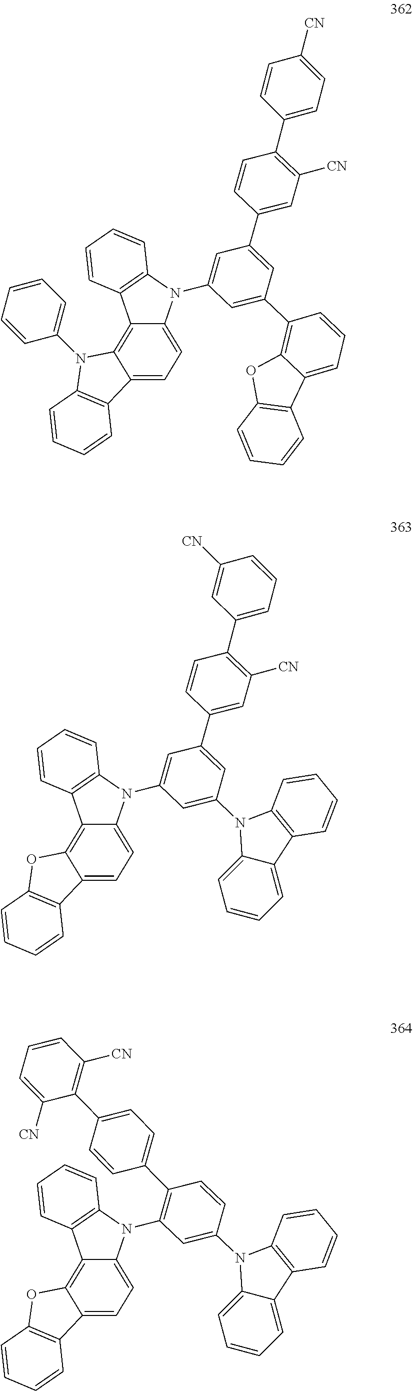

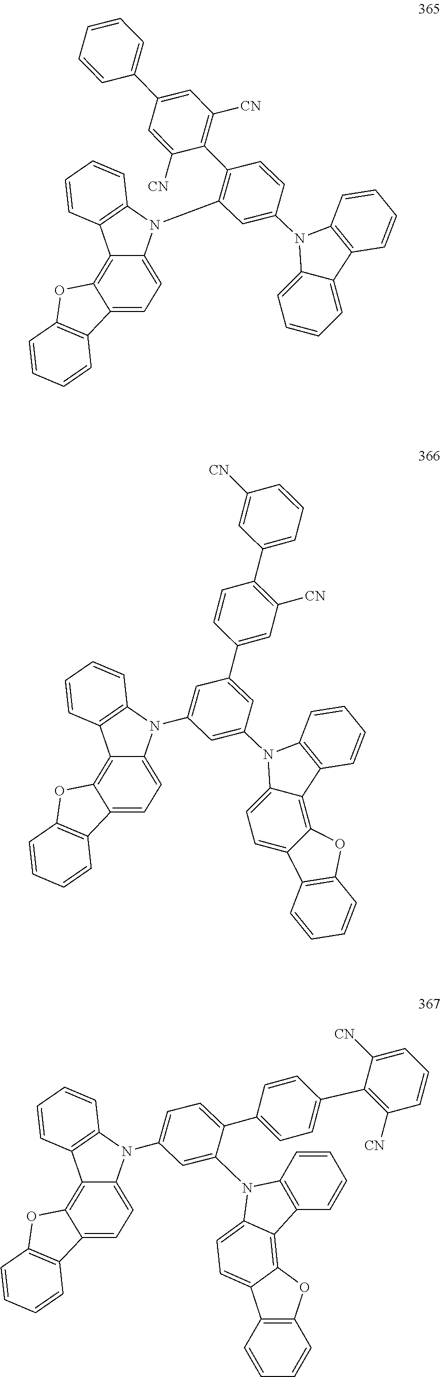

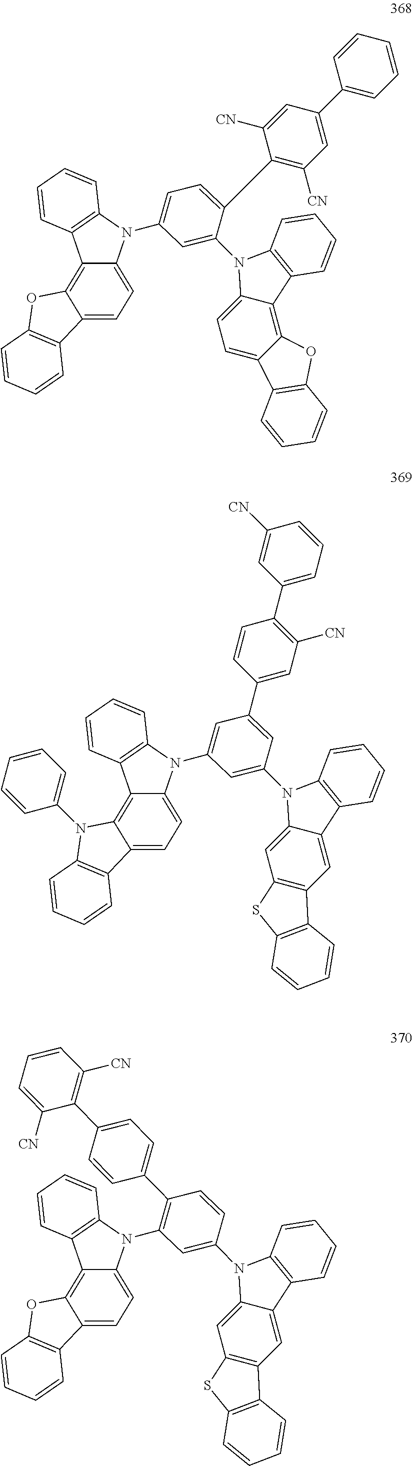

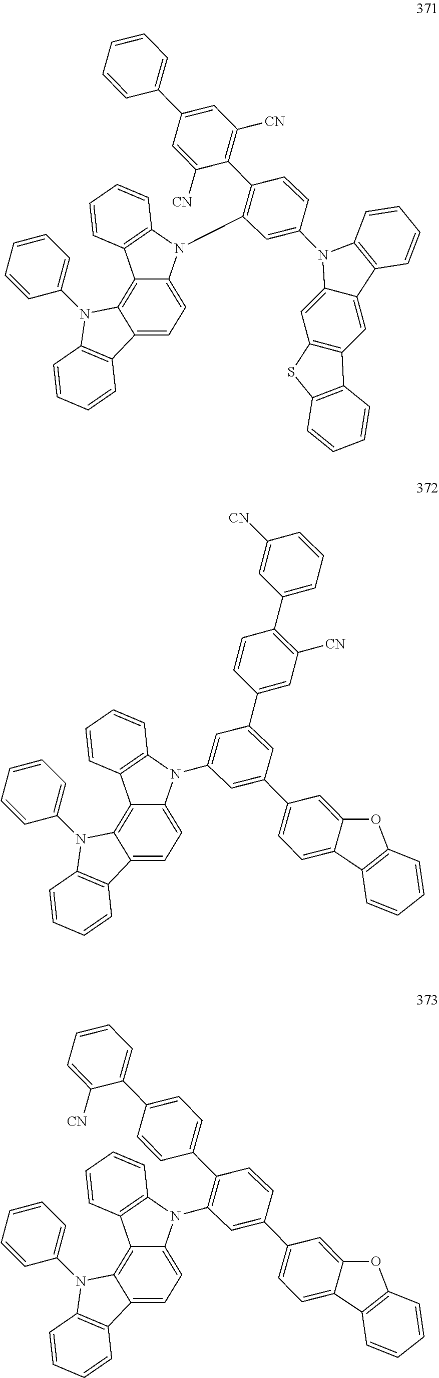

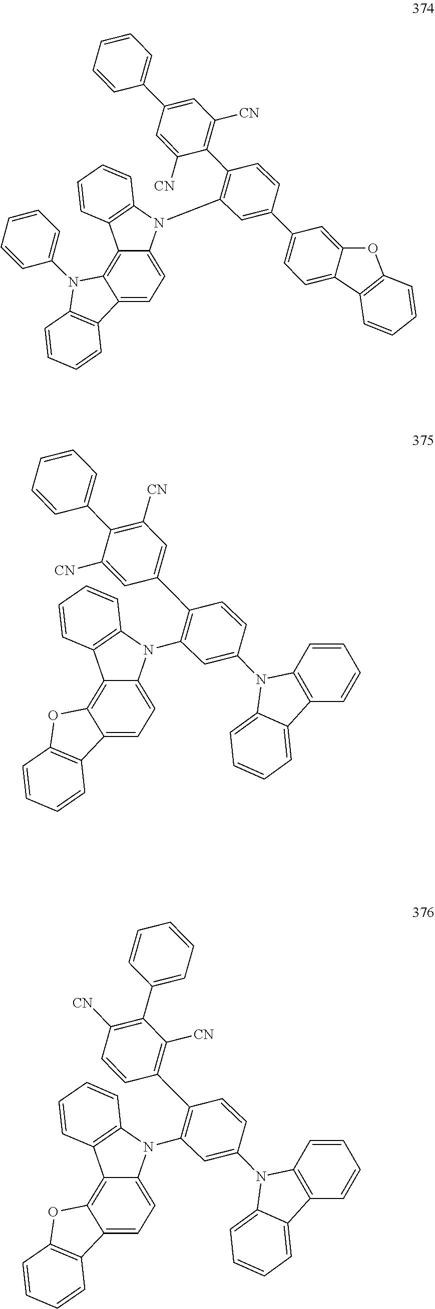

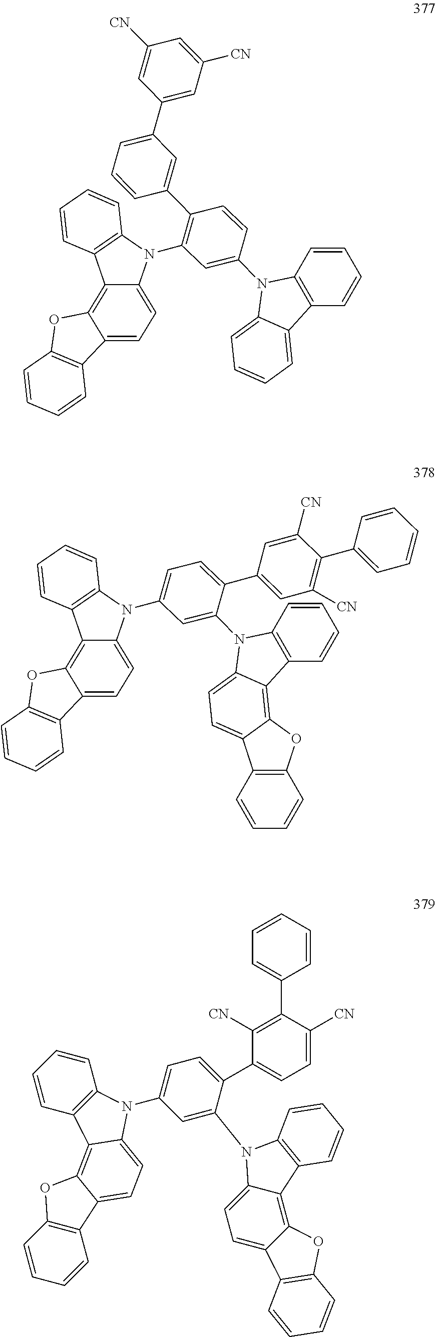

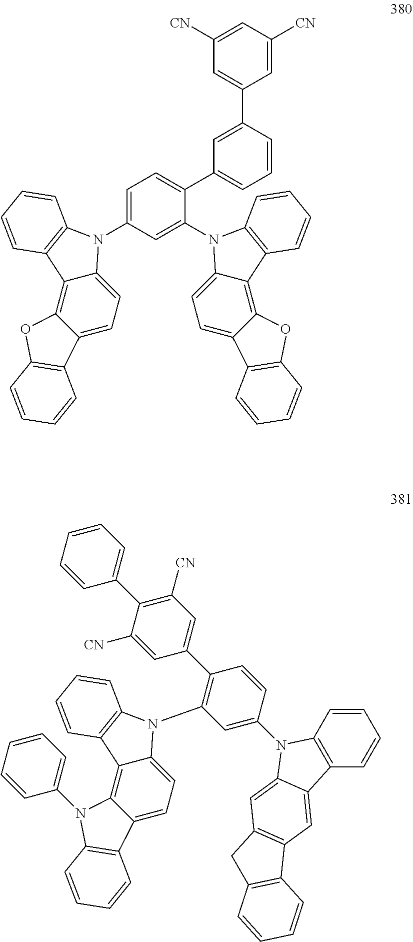

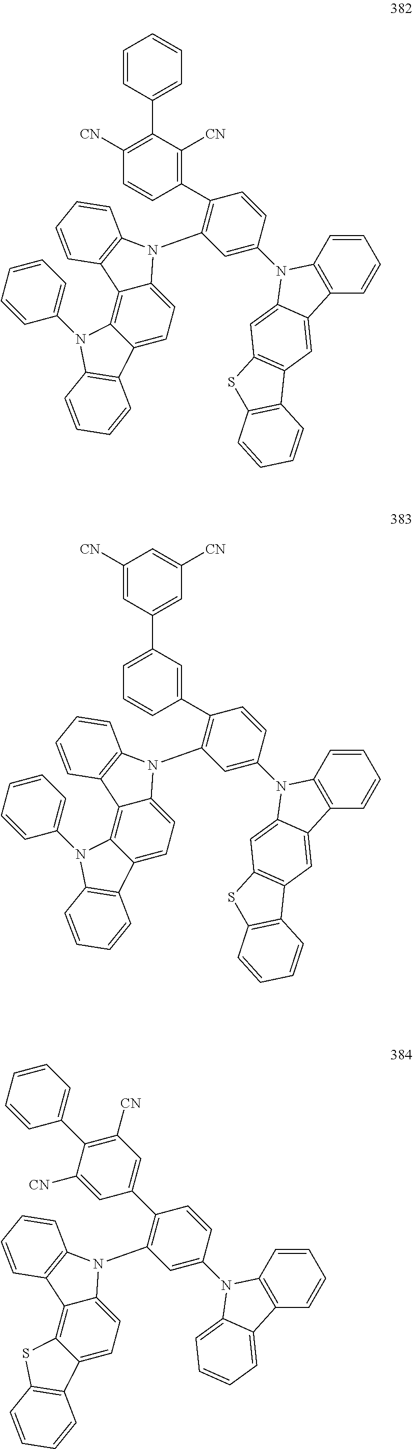

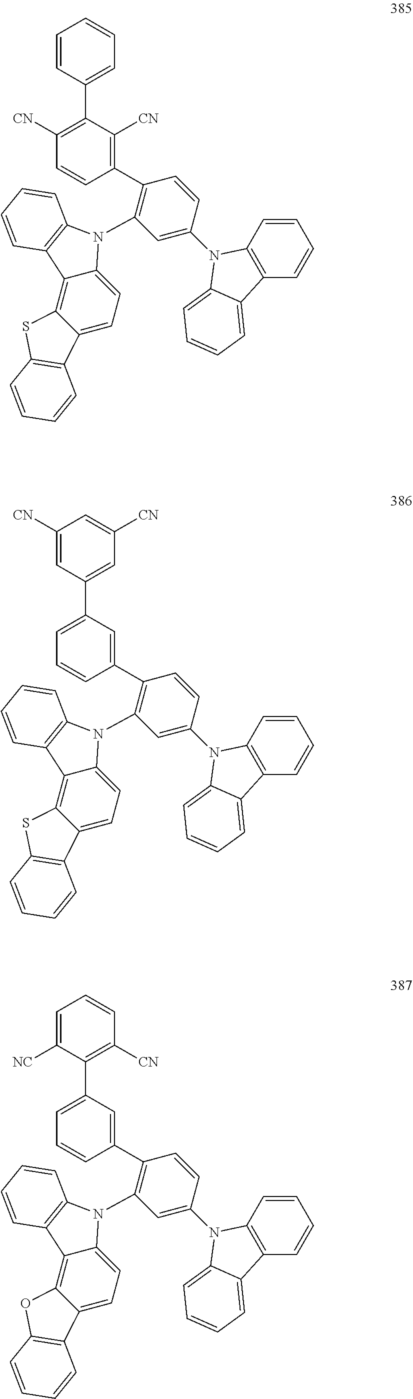

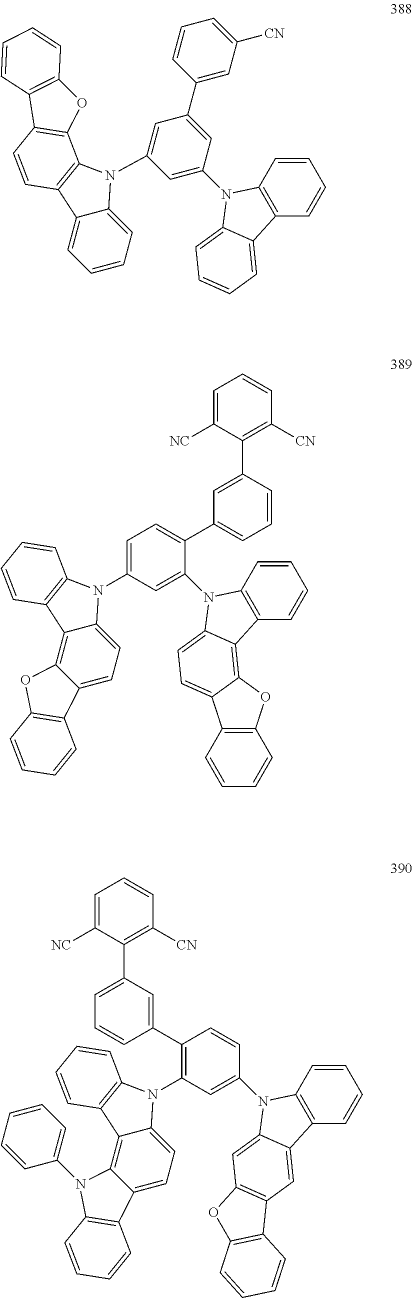 [SMILES: N#Cc1cccc(-c2cc(-n3c4ccccc4c4ccccc43)cc(-n3c4ccccc4c4ccc5c6ccccc6oc5c43)c2)c1 ; N#Cc1cccc(C#N)c1-c1cccc(-c2ccc(-n3c4ccccc4c4c5oc6ccccc6c5ccc43)cc2-n2c3ccccc3c3c4oc5ccccc5c4ccc32)c1 ; N#Cc1cccc(C#N)c1-c1cccc(-c2ccc(-n3c4ccccc4c4cc5c(cc43)oc3ccccc35)cc2-n2c3ccccc3c3c2ccc2c4ccccc4n(-c4ccccc4)c23)c1]

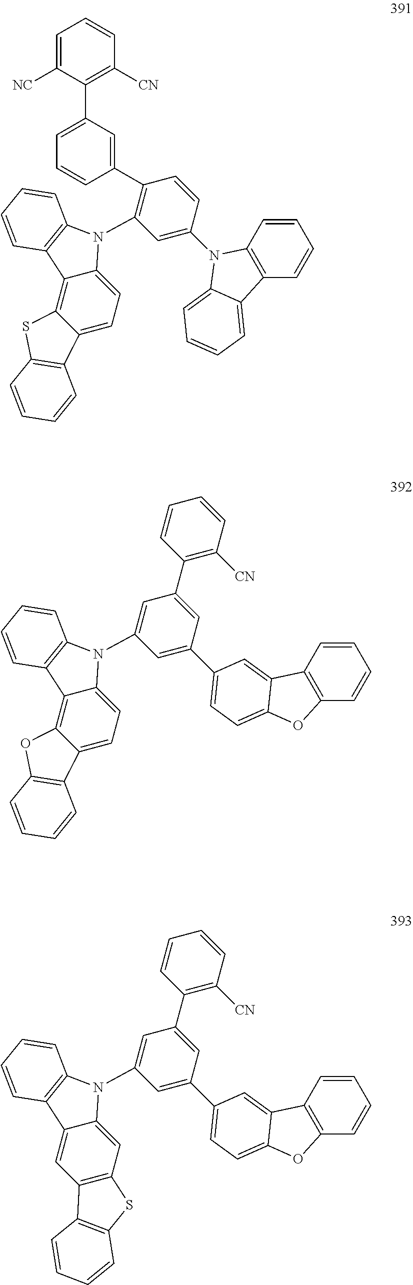 [SMILES: N#Cc1cccc(C#N)c1-c1cccc(-c2ccc(-n3c4ccccc4c4ccccc43)cc2-n2c3ccccc3c3c4sc5ccccc5c4ccc32)c1 ; N#Cc1ccccc1-c1cc(-c2ccc3oc4ccccc4c3c2)cc(-n2c3ccccc3c3c4oc5ccccc5c4ccc32)c1 ; N#Cc1ccccc1-c1cc(-c2ccc3oc4ccccc4c3c2)cc(-n2c3ccccc3c3cc4c(cc32)sc2ccccc24)c1]

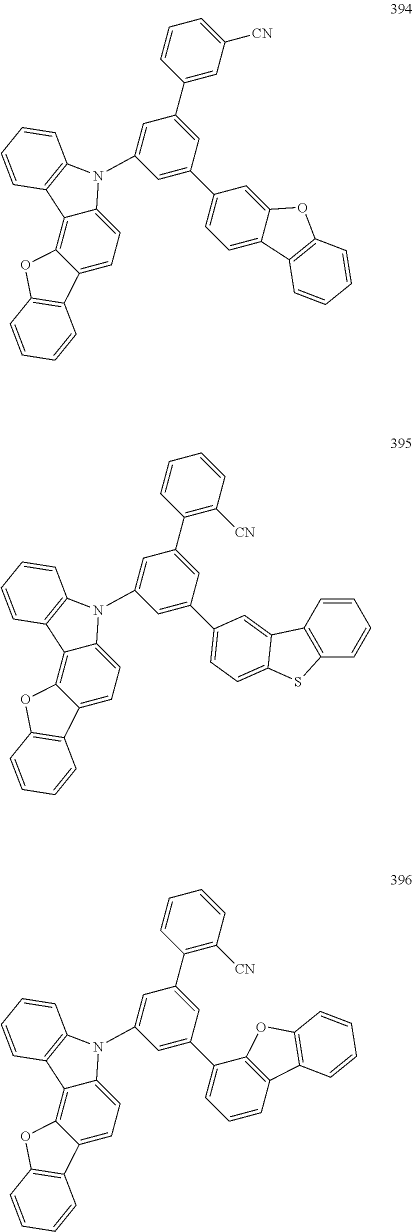 [SMILES: N#Cc1cccc(-c2cc(-c3ccc4c(c3)oc3ccccc34)cc(-n3c4ccccc4c4c5oc6ccccc6c5ccc43)c2)c1 ; N#Cc1ccccc1-c1cc(-c2ccc3sc4ccccc4c3c2)cc(-n2c3ccccc3c3c4oc5ccccc5c4ccc32)c1 ; N#Cc1ccccc1-c1cc(-c2cccc3c2oc2ccccc23)cc(-n2c3ccccc3c3c4oc5ccccc5c4ccc32)c1]

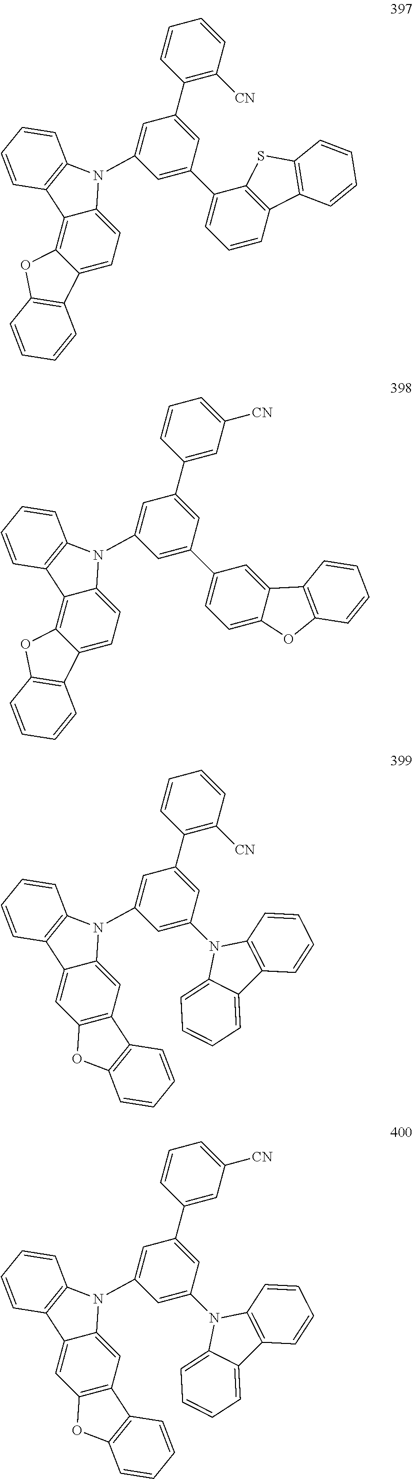

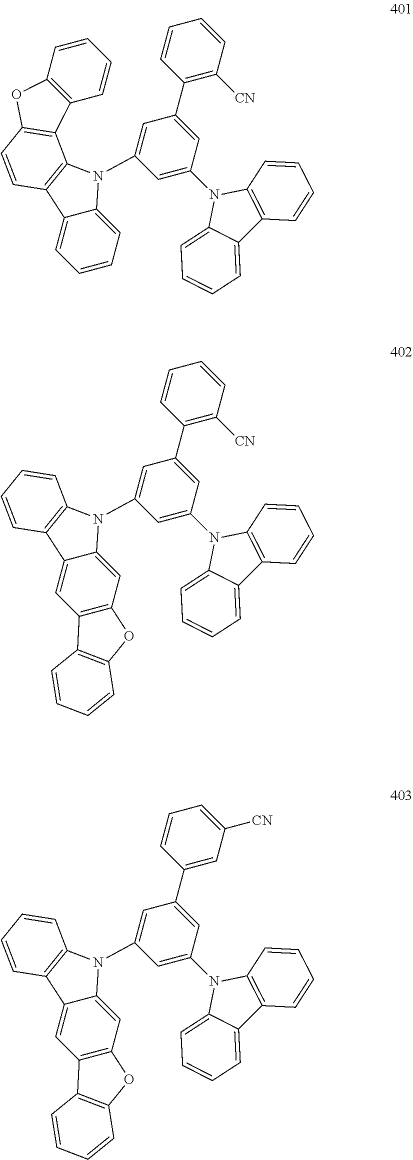 [SMILES: N#Cc1ccccc1-c1cc(-n2c3ccccc3c3ccccc32)cc(-n2c3ccccc3c3ccc4oc5ccccc5c4c32)c1 ; N#Cc1ccccc1-c1cc(-n2c3ccccc3c3ccccc32)cc(-n2c3ccccc3c3cc4c(cc32)oc2ccccc24)c1 ; N#Cc1cccc(-c2cc(-n3c4ccccc4c4ccccc43)cc(-n3c4ccccc4c4cc5c(cc43)oc3ccccc35)c2)c1]

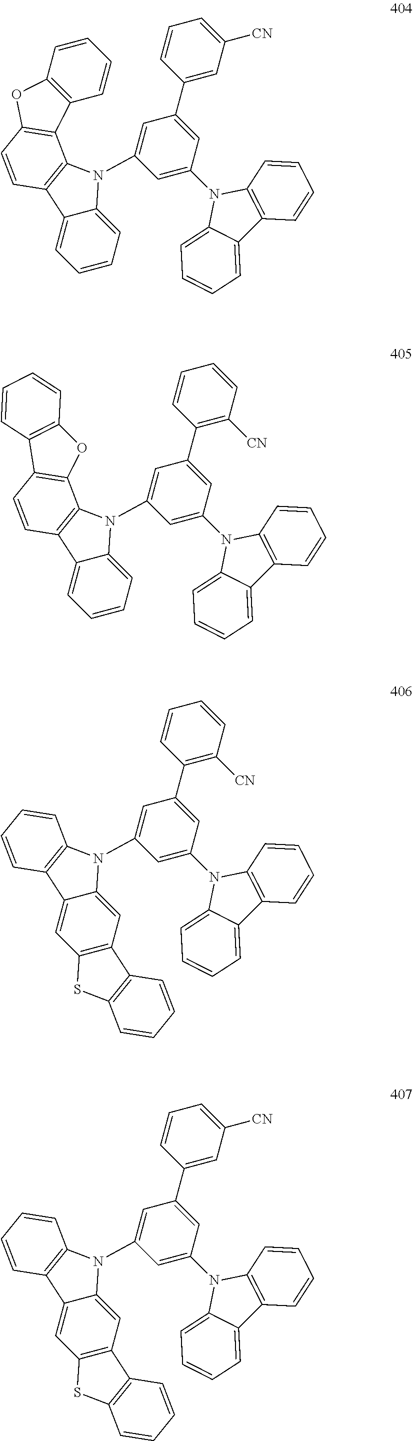 [SMILES: N#Cc1cccc(-c2cc(-n3c4ccccc4c4ccccc43)cc(-n3c4ccccc4c4ccc5oc6ccccc6c5c43)c2)c1 ; N#Cc1ccccc1-c1cc(-n2c3ccccc3c3ccccc32)cc(-n2c3ccccc3c3ccc4c5ccccc5oc4c32)c1 ; N#Cc1ccccc1-c1cc(-n2c3ccccc3c3ccccc32)cc(-n2c3ccccc3c3cc4sc5ccccc5c4cc32)c1 ; N#Cc1cccc(-c2cc(-n3c4ccccc4c4ccccc43)cc(-n3c4ccccc4c4cc5sc6ccccc6c5cc43)c2)c1]

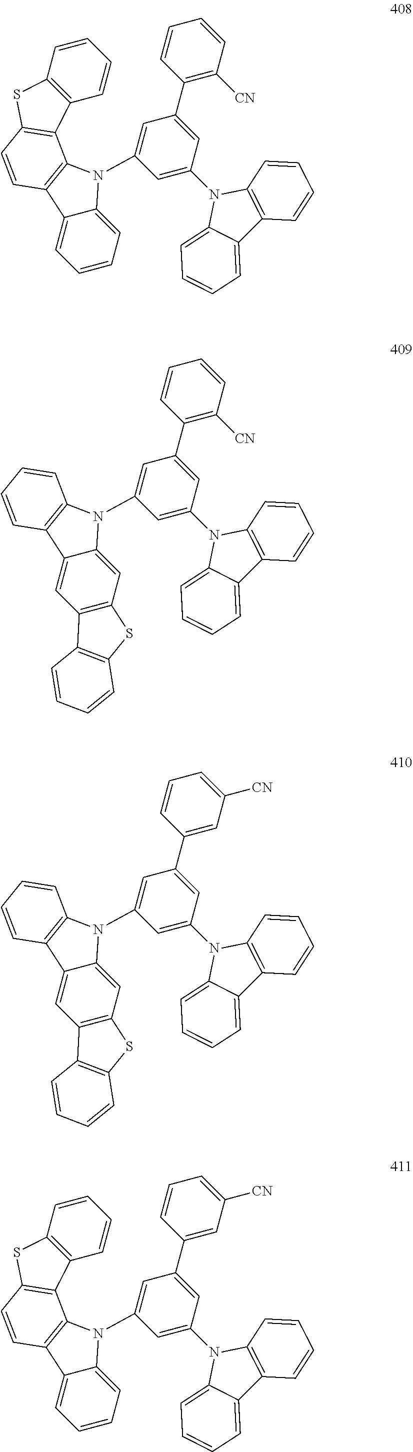 [SMILES: N#Cc1ccccc1-c1cc(-n2c3ccccc3c3ccccc32)cc(-n2c3ccccc3c3ccc4sc5ccccc5c4c32)c1 ; N#Cc1ccccc1-c1cc(-n2c3ccccc3c3ccccc32)cc(-n2c3ccccc3c3cc4c(cc32)sc2ccccc24)c1 ; N#Cc1cccc(-c2cc(-n3c4ccccc4c4ccccc43)cc(-n3c4ccccc4c4cc5c(cc43)sc3ccccc35)c2)c1 ; N#Cc1cccc(-c2cc(-n3c4ccccc4c4ccccc43)cc(-n3c4ccccc4c4ccc5sc6ccccc6c5c43)c2)c1]

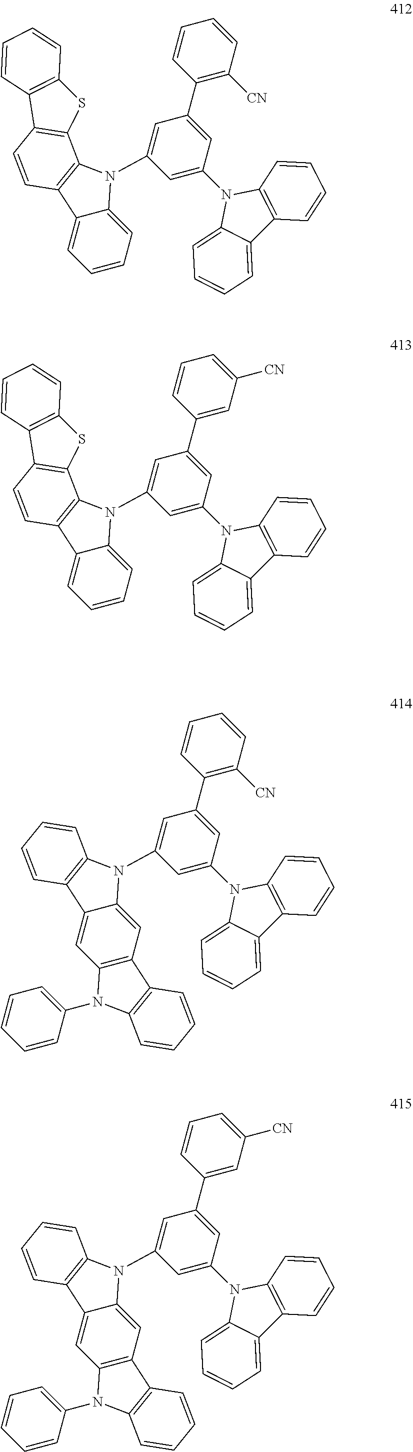 [SMILES: N#Cc1ccccc1-c1cc(-n2c3ccccc3c3ccccc32)cc(-n2c3ccccc3c3ccc4c5ccccc5sc4c32)c1 ; N#Cc1cccc(-c2cc(-n3c4ccccc4c4ccccc43)cc(-n3c4ccccc4c4ccc5c6ccccc6sc5c43)c2)c1 ; N#Cc1ccccc1-c1cc(-n2c3ccccc3c3ccccc32)cc(-n2c3ccccc3c3cc4c(cc32)c2ccccc2n4-c2ccccc2)c1 ; N#Cc1cccc(-c2cc(-n3c4ccccc4c4ccccc43)cc(-n3c4ccccc4c4cc5c(cc43)c3ccccc3n5-c3ccccc3)c2)c1]

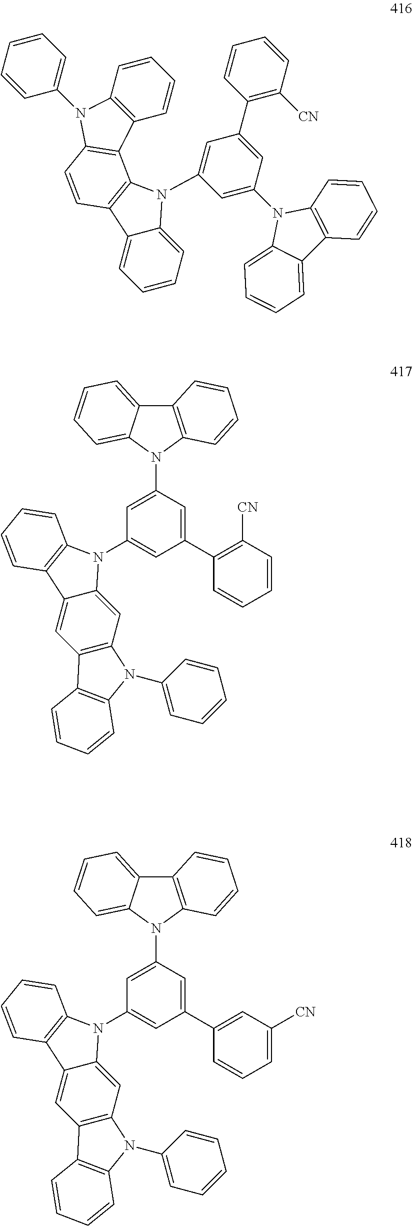 [SMILES: N#Cc1ccccc1-c1cc(-n2c3ccccc3c3ccccc32)cc(-n2c3ccccc3c3ccc4c(c5ccccc5n4-c4ccccc4)c32)c1 ; N#Cc1ccccc1-c1cc(-n2c3ccccc3c3ccccc32)cc(-n2c3ccccc3c3cc4c5ccccc5n(-c5ccccc5)c4cc32)c1 ; N#Cc1cccc(-c2cc(-n3c4ccccc4c4ccccc43)cc(-n3c4ccccc4c4cc5c6ccccc6n(-c6ccccc6)c5cc43)c2)c1]

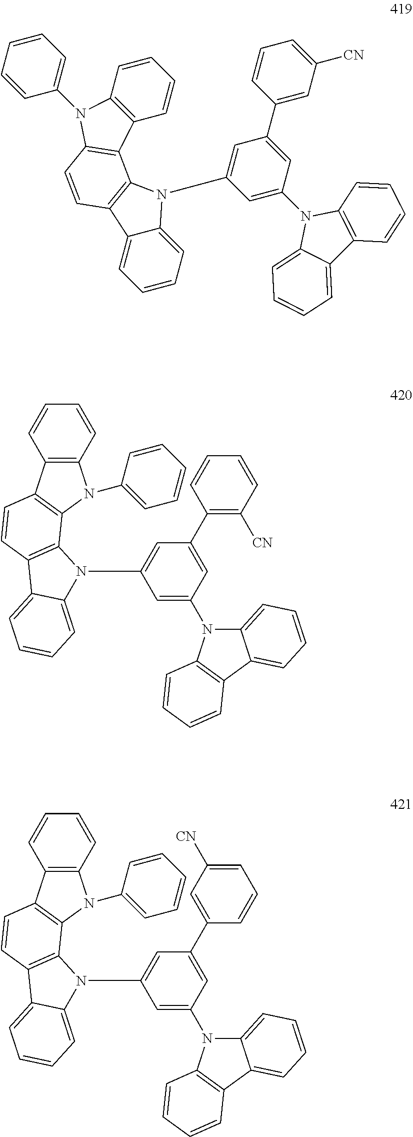

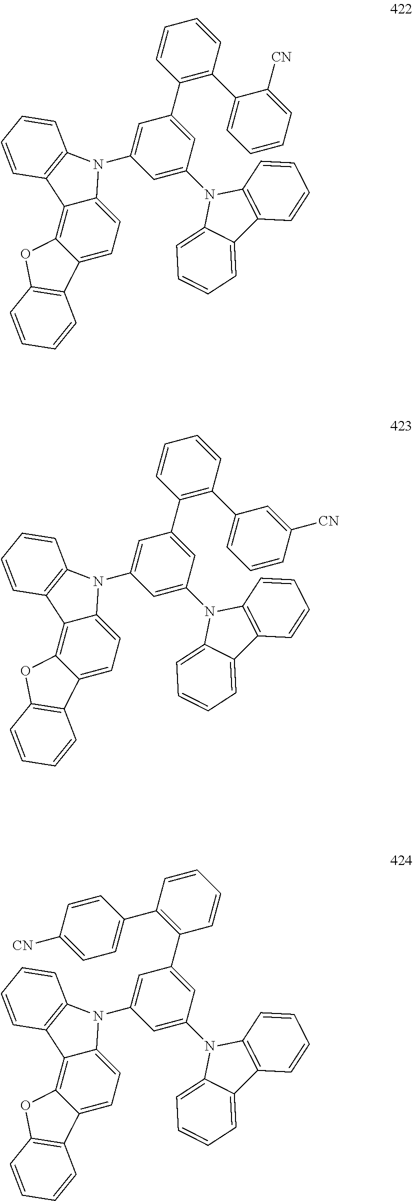

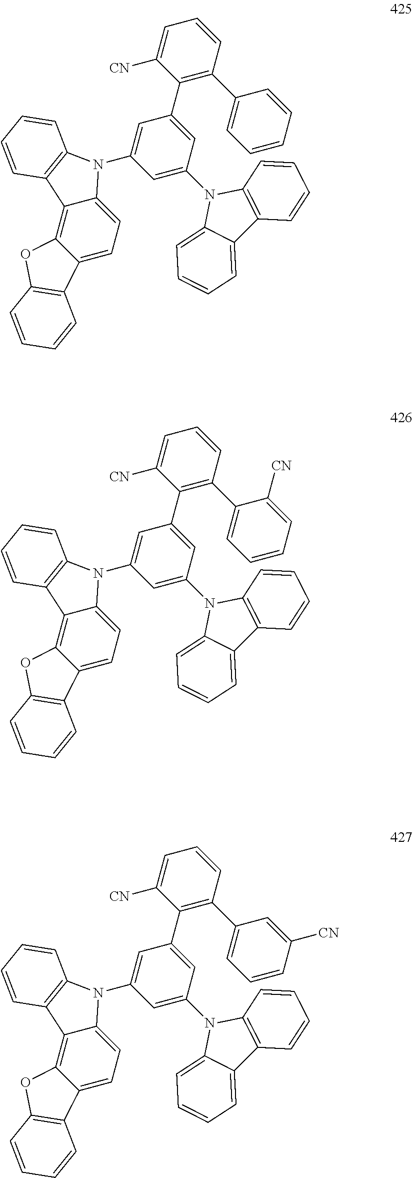

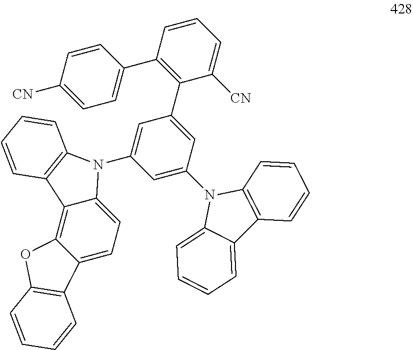

15. A composition comprising:
a first compound and a second compound,
wherein the first compound is the condensed cyclic compound of claim 1, and
wherein the second compound comprises at least one selected from a carbazole group, a dibenzofuran group, a dibenzothiophene group, an indenocarbazole group, an indolocarbazole group, a benzofurocarbazole group, a benzothienocarbazole group, an acridine group, a dihydroacridine group, and a triindolobenzene group and does not comprise an electron withdrawing group, wherein the electron withdrawing group is selected from —F, —CFH₂, —CF₂H, —CF₃, —CN, and —NO₂;
a C₁-C₆₀ alkyl group substituted with at least one selected from —F, —CFH₂, —CF₂H, —CF₃, —CN, and —NO₂;
a C₁-C₆₀ heteroaryl group and a monovalent non-aromatic condensed polycyclic heterocyclic group, each comprising *═N—*′ as a ring-forming moiety; and
a C₁-C₆₀ heteroaryl group and a monovalent non-aromatic condensed polycyclic heterocyclic group, each comprising *═N—*′ as a ring-forming moiety and each substituted with at least one selected from deuterium, —F, —CFH₂, —CF₂H, —CF₃, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

16. The composition of claim 15, wherein the second compound is represented by Formula H-1: 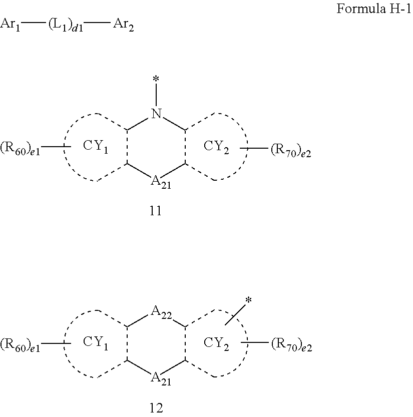
wherein, in Formulae H-1, 11, and 12,
L₁ is selected from
a single bond, a phenylene group, a naphthylene group, a fluorenylene group, a carbazolylene group, a dibenzofuranylene group, and a dibenzothiophenylene group; and
a phenylene group, a naphthylene group, a fluorenylene group, a carbazolylene group, a dibenzofuranylene group, and a dibenzothiophenylene group, each substituted with at least one selected from deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a biphenyl group, and —Si(Q₁₁)(Q₁₂)(Q₁₃),
d1 is an integer from 1 to 10; and when d1 is 2 or greater, at least two L₁ groups are identical to or different from each other,
Ar₁ is selected from groups represented by Formulae 11 and 12,
Ar₂ is selected from
groups represented by Formulae 11 and 12, a phenyl group, and a naphthyl group; and
a phenyl group and a naphthyl group, each substituted with at least one selected from deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, and a biphenyl group,
CY₁ and CY₂ are each independently selected from a benzene group, a naphthalene group, a fluorene group, a carbazole group, a benzocarbazole group, an indolocarbazole group, a dibenzofuran group, a dibenzothiophene group, and a dibenzosilole group,
A₂₁ is selected from a single bond, O, S, N(R₅₁), C(R₅₁)(R₅₂), and Si(R₅₁)(R₅₂),
A₂₂ is selected from a single bond, O, S, N(R₅₃), C(R₅₃)(R₅₄), and Si(R₅₃)(R₅₄),
at least one selected from A₂₁ and A₂₂ in Formula 12 is not a single bond,
R₅₁ to R₅₄, R₆₀, and R₇₀ are each independently selected from
hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;
a C₁-C₂₀ alkyl group and a C₁-C₂₀ alkoxy group, each substituted with at least one selected from deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, and a dibenzothiophenyl group;
a phenyl group, a naphthyl group, a fluorenyl group, a carbazolyl group, a dibenzofuranyl group, and a dibenzothiophenyl group;
a phenyl group, a naphthyl group, a fluorenyl group, a carbazolyl group, a dibenzofuranyl group, and a dibenzothiophenyl group, each substituted with at least one selected from deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, and a biphenyl group; and
Si(Q₁)(Q₂)(Q₃),
e1 and e2 are each independently an integer from 0 to 10,
wherein Q₁ to Q₃ and Q₁₁ to Q₁₃ are each independently selected from hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, a dibenzothiophenyl group, and a biphenyl group, and
* indicates a binding site to an adjacent atom.

17. The composition of claim 16, wherein
Ar₁ is selected from groups represented by Formulae 11-1 to 11-8 and 12-1 to 12-8, and
Ar₂ is selected from
groups represented by Formulae 11-1 to 11-8 and 12-1 to 12-8, a phenyl group, and a naphthyl group; and
a phenyl group and a naphthyl group, each substituted with at least one selected from deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, and a biphenyl group: 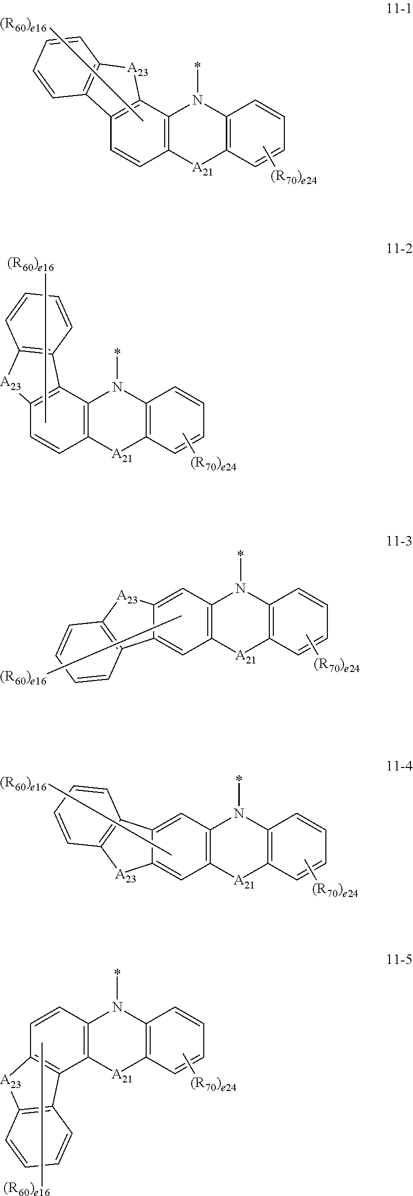 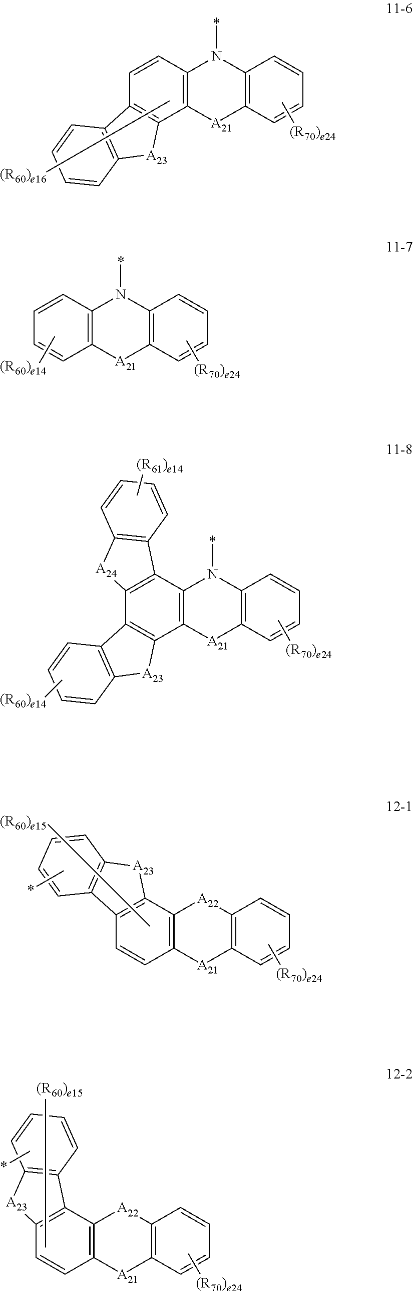 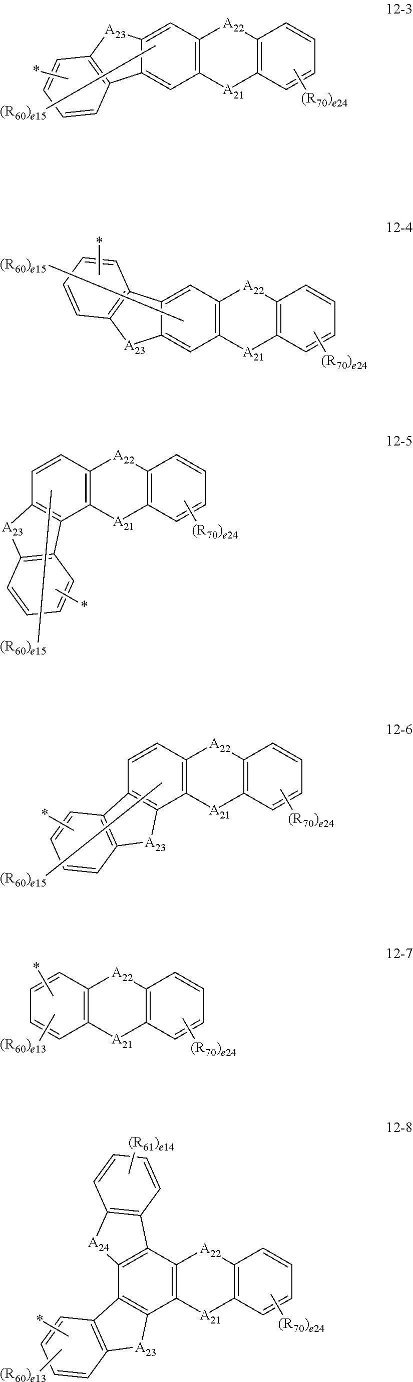
wherein, in Formulae 11-1 to 11-8 and 12-1 to 12-8,
A₂₃ is selected from O, S, N(R₅₅), C(R₅₅)(R₅₆), and Si(R₅₅)(R₅₆),
A₂₄ is selected from O, S, N(R₅₇), C(R₅₇)(R₅₈), and Si(R₅₇)(R₅₈),
A₂₁, A₂₂, R₆₀, and R₇₀ are each as defined in claim 14,
R₅₅ to R₅₈ are each defined the same as R₅₁ in claim 14,
e16 is an integer from 0 to 6,
e15 is an integer from 0 to 5,
e14 is an integer from 0 to 4,
e13 is an integer from 0 to 3,
e24 is an integer from 0 to 4, and
* indicates a binding site to an adjacent atom.

18. An organic light-emitting device comprising:
a first electrode;
a second electrode; and
an organic layer disposed between the first electrode and the second electrode,
wherein the organic layer comprises an emission layer, and
wherein the organic layer comprises the condensed cyclic compound represented by Formula 1 of claim 1.

19. The organic light-emitting device of claim 18, wherein the emission layer comprises the condensed cyclic compound represented by Formula 1.

20. The organic light-emitting device of claim 18, wherein the emission layer comprises a host and a dopant, wherein the host comprises the condensed cyclic compound represented by Formula 1, and wherein the dopant comprises a phosphorescent dopant.
